(19) United States
(12) Patent Application Publication
(10) Pub. No.: US 2025/0333742 A1
(43) Pub. Date: Oct. 30, 2025

(54) COMPOSITIONS AND METHODS FOR TREATING METABOLIC DYSFUNCTION-ASSOCIATED STEATOTIC LIVER DISEASE

(71) Applicant: Insitro, Inc., South San Francisco, CA (US)

(72) Inventors: David John LLOYD, Lafayette, CA (US); Santhosh SATAPATI, Dublin, CA (US); Arijit BHOWMICK, Astoria, NY (US); Sumit MUKHERJEE, Issaquah, WA (US); Hari SOMINENI, Quincy, MA (US); Tima Sheren Dehghani HARDMAN, Brisbane, CA (US)

(73) Assignee: Insitro, Inc., South San Francisco, CA (US)

(21) Appl. No.: 19/189,062

(22) Filed: Apr. 24, 2025

Related U.S. Application Data
(60) Provisional application No. 63/638,729, filed on Apr. 25, 2024.

(51) Int. Cl. C12N 15/113; A61K 9/00; A61K 31/713; A61K 47/51; A61K 48/00; A61P 3/10

(52) U.S. Cl. CPC C12N 15/1137; A61K 9/0019; A61K 31/713; A61K 47/51; A61K 48/0008; A61K 48/005; A61K 48/0075; A61P 3/10; C12N 2310/11; C12N 2310/33

(57) ABSTRACT

Described are compositions and methods for inhibition of glucokinase (GCK) gene expression and protein production. RNA interference (RNAi) agents for inhibiting the expression of GCK gene are described. The GCK RNAi agents disclosed herein may be targeted to cells, such as hepatocytes, for example, by using conjugated targeting ligands. Pharmaceutical compositions comprising one or more GCK RNAi agents optionally with one or more additional therapeutics are also described.

### CROSS-REFERENCE TO RELATED APPLICATIONS

This application claims priority to and the benefit of U.S. Provisional Application No. 63/638,729, filed on Apr. 25, 2024, the contents of which is incorporated herein in its entirety.

### REFERENCE TO AN ELECTRONIC SEQUENCE LISTING

The contents of the electronic sequence listing (208782003500SEQLIST.xml; Size: 3, 972, 210 bytes; and Date of Creation: Apr. 21, 2025) is herein incorporated by reference in its entirety.

### FIELD

The present disclosure relates to RNA interference (RNAi) agents to inhibit the expression of glucokinase (GCK), an enzyme that facilitates the conversion of glucose to glucose-6-phosphate. The disclosure also relates to compositions that include GCK RNAi agents and methods of use thereof, including the use of such GCK RNAi agents in the treatment of metabolic dysfunction-associated steatotic liver disease (MASLD) and/or metabolic dysfunction-associated steatohepatitis (MASH).

### BACKGROUND

Metabolic dysfunction-associated steatotic liver disease (MASLD), previously referred to as non-alcoholic fatty liver disease (NAFLD), is caused by a build-up of fat in the liver, occasionally appearing as multiple nodular areas, and includes a range of disease states, from isolated lipid accumulation or steatosis (metabolic dysfunction-associated steatotic liver, MASL) through to its active inflammatory form, metabolic dysfunction-associated steatohepatitis (MASH), previously referred to as non-alcoholic steatohepatitis (NASH). Persistence of MASH leads, in some cases, to cirrhosis (scarring of the liver), liver failure and liver cancer. MASLD and MASH are both linked to being overweight or obese, insulin resistance, high blood sugar (hyperglycemia), and high levels of fats (particularly triglycerides) in the blood. MASLD is also a risk factor for type 2 diabetes. Lifestyle modification remains the cornerstone of management of MASLD and MASH including losing weight, keeping a healthy diet, limiting over the counter drugs, and avoiding alcohol. Additional treatment options include medications to reduce blood pressure or control diabetes. If MASLD or MASH leads to cirrhosis, treatment of the complications of cirrhosis with medicines, minor medical procedures, and surgery may be necessary. Individuals with liver failure or liver cancer may need a liver transplant to restore health.

MASLD is increasingly common around the world, especially in Western nations where it is the leading cause of liver disease. In the United States, it is the most common form of chronic liver disease, affecting about one-quarter of the population. It is a cause of end-stage liver disease, with increased mortality secondary to cirrhosis and its complications. There continues to be a need or safe and effective treatments for MASLD and MASH.

### SUMMARY

Provided herein are RNA interference (RNAi) agents to inhibit the expression of glucokinase (GCK).

Described herein is an RNAi agent that inhibits expression of glucokinase (GCK) for use in the treatment of metabolic dysfunction-associated steatotic liver disease (MASLD) or metabolic dysfunction-associated steatotic liver (MASL) in a subject, wherein the RNAi agent comprises (i) an antisense strand and a sense strand that at least partially complements the antisense strand, and (ii) a targeting ligand conjugated to the antisense strand or the sense strand, wherein the targeting ligand targets a hepatocyte.

Described herein is an RNAi agent that inhibits expression of glucokinase (GCK) comprising an antisense strand and a sense strand that at least partially complements the antisense strand, and a targeting ligand conjugated to the antisense strand or the sense strand, wherein the targeting ligand targets a hepatocyte.

Described herein is an RNAi agent that inhibits expression of glucokinase (GCK) comprising an antisense strand and a sense strand that at least partially complements the antisense strand, wherein: (a) the antisense strand comprises at least 14 contiguous bases from any one of SEQ ID NOS: 1-96; and/or (b) the sense strand comprises at least 14 contiguous bases from any one of SEQ ID NOS: 97-192.

In some implementations, one or more bases of the antisense stand and/or the sense strand are modified. In some implementations, the antisense strand has a modification pattern according to nsNfsnnnNfnnnnnnnNfnNfnnnnnsnsn. In some implementations, the sense strand has a modification pattern according to nsnsnnnnNfnNfNfNfnnnnnnnnsnsn. In some implementations, the sense strand has a modification pattern according to nsnsnnnnNfnNfNfNfnnnnnnnnnn.

In some implementations, (a) the antisense strand comprises at least 14 contiguous bases from any one of SEQ ID NOS: 193-288; and/or (b) the sense strand comprises at least 14 contiguous bases from any one of SEQ ID NOS: 289-384 and 385-480. In some implementations, the antisense strand comprises at least 14 contiguous bases that complement a sequence in an mRNA molecule that encodes GCK (SEQ ID NO: 481). In some implementations, the antisense strand and the sense strand form a duplex of at least 14 bases in length. In some implementations, the antisense strand and the sense strand form a duplex of between 15 and 30 bases in length. In some implementations, the antisense strand or a subsequence thereof differs by 0, 1, 2, 3, or 4 nucleotides from an equal length portion of any one of SEQ ID NOS: 1-96 and 193-288, wherein the subsequence is at least 14, 15, 16, 17, 18, 19, 20, 21, 22, or 23 bases in length.

In some implementations, the antisense strand comprises, in order, nucleotides 1-23 of any one of SEQ ID NOS: 1-96 and 193-288. In some implementations, the antisense strand comprises at least 14 contiguous bases from any one of SEQ ID NOS: 1-96 and 193-288. In some implementations, the antisense strand comprises, in order, nucleobases 1-14 of any one of SEQ ID NOS: 1-96 and 193-288. In some implementations, the antisense strand comprises at least 14 contiguous bases from any one of SEQ ID NOS: 8, 11, 14, 16, 35, 36, 38, 39, 41, 42, 52, 77, 84, 86, 87, 88, 89, 90, 93, 200, 203, 206, 208, 227, 228, 230, 231, 233, 234, 244, 269, 276, 278, 279, 280, 281, 282, and 285. In some implementations, the antisense strand comprises at least 14 contiguous bases from any one of SEQ ID NOS: 16, 35, 52, 84, 86, 89, 90, 208, 227, 244, 276, 278, 281, and 282.

In some implementations, the sense strand or a subsequence thereof differs by 0, 1, 2, 3, or 4 nucleotides from an equal length portion of any one of SEQ ID NOS: 97-192, 289-384, and 385-480, wherein the subsequence is at least 14, 15, 16, 17, 18, 19, 20, or 21 bases in length. In some implementations, the sense strand comprises, in order, nucleotides 1-21 of any one of SEQ ID NOS: 97-192, 289-384, and 385-480. In some implementations, the sense strand comprises at least 14 contiguous bases from any one of SEQ ID NOS: 97-192, 289-384, and 385-480. In some implementations, the sense strand comprises, in order, nucleobases 1-14 of any one of SEQ ID NOS: 97-192, 289-384, and 385-480. In some implementations, the sense strand comprises at least 14 contiguous bases from any one of SEQ ID NOS:104, 107, 110, 112, 131, 132, 134, 135, 137, 138, 148, 173, 180, 182, 183, 184, 185, 186, 189, 296, 299, 302, 304, 323, 324, 326, 327, 329, 330, 340, 365, 372, 374, 375, 376, 377, 378, 381, 392, 395, 398, 400, 419, 420, 422, 423, 425 426, 436, 461, 468, 470, 471, 472, 473, 474, and 477. In some implementations, the sense strand comprises at least 14 contiguous bases from any one of SEQ ID NOS: 112, 131, 148, 180, 182, 185, 186, 304, 323, 340, 372, 374, 377, 378, 400, 419, 436, 468, 470, 473, and 474.

In some implementations, the antisense strand comprises at least 16 contiguous bases that complement a sequence in an mRNA molecule that encodes GCK (SEQ ID NO: 481). In some implementations, the antisense strand and the sense strand form a duplex of at least 16 bases in length. In some implementations, the antisense strand comprises at least 16 contiguous bases from any one of SEQ ID NOS: 1-96 and 193-288. In some implementations, the antisense strand comprises at least 16 contiguous bases from any one of SEQ ID NOS: 8, 11, 14, 16, 35, 36, 38, 39, 41, 42, 52, 77, 84, 86, 87, 88, 89, 90, 93, 200, 203, 206, 208, 227, 228, 230, 231, 233, 234, 244, 269, 276, 278, 279, 280, 281, 282, and 285. In some implementations, the antisense strand comprises, in order, nucleobases 1-16, 1-17, 1-18, 1-19, 1-20, 1-21, 1-22, 1-23, 2-17, 2-18, 2-19, 2-20, 2-21, 2-22, 2-23, 3-18, 3-19, 3-20, 3-21, 3-22, 3-23, 4-19, 4-20, 4-21, 4-22, 4-23, 5-21, 5-22, 5-23, 6-20, 6-21, 6-22, 6-23, 7-22, 7-23, or 8-23 of any one of SEQ ID NOS: 1-96 and 193-288. In some implementations, the antisense strand comprises a nucleotide sequence according to any one of SEQ ID NOS: 1-96 and 193-288.

In some implementations, one or more nucleotides are modified. In some implementations, the antisense strand comprises a nucleotide sequence according to any one of SEQ ID NOS: 8, 11, 14, 16, 35, 36, 38, 39, 41, 42, 52, 77, 84, 86, 87, 88, 89, 90, and 93. In some implementations, the antisense strand comprises a nucleotide sequence according to any one of SEQ ID NOS: 16, 35, 52, 84, 86, 89, and 90. In some implementations, one or more nucleotides are modified. In some implementations, the antisense strand has a modification pattern according to nsNfsnnnNfnnnnnnnNfnNfnnnnnsnsn. In some implementations, the antisense strand comprises a modified nucleotide sequence according to any one of SEQ ID NOS: 193-288. In some implementations, the antisense strand comprises a modified nucleotide sequence according to any one of SEQ ID NOS: 200, 203, 206, 208, 227, 228, 230, 231, 233, 234, 244, 269, 276, 278, 279, 280, 281, 282, and 285. In some implementations, the antisense strand comprises a modified nucleotide sequence according to any one of SEQ ID NOS: 208, 227, 244, 276, 278, 281, and 282.

In some implementations, the sense strand comprises at least 16 contiguous bases from any one of SEQ ID NOS: 97-192, 289-384, and 385-480. In some implementations, the sense strand comprises at least 16 contiguous bases from any one of SEQ ID NOS: 104, 107, 110, 112, 131, 132, 134, 135, 137, 138, 148, 173, 180, 182, 183, 184, 185, 186, 189, 296, 299, 302, 304, 323, 324, 326, 327, 329, 330, 340, 365, 372, 374, 375, 376, 377, 378, 381, 392, 395, 398, 400, 419, 420, 422, 423, 425 426, 436, 461, 468, 470, 471, 472, 473, 474, and 477. In some implementations, the sense strand comprises at least 16 contiguous bases from any one of SEQ ID NOS: 112, 131, 148, 180, 182, 185, 186, 304, 323, 340, 372, 374, 377, 378, 400, 419, 436, 468, 470, 473, and 474. In some implementations, the sense strand comprises, in order, nucleobases 1-16, 1-17, 1-18, 1-19, 1-20, 1-21, 1-22, 1-23, 2-17, 2-18, 2-19, 2-20, 2-21, 2-22, 2-23, 3-18, 3-19, 3-20, 3-21, 3-22, 3-23, 4-19, 4-20, 4-21, 4-22, 4-23, 5-21, 5-22, 5-23, 6-20, 6-21, 6-22, 6-23, 7-22, 7-23, or 8-23 of any one of SEQ ID NOS: 97-192, 289-384, and 385-480.

In some implementations, the sense strand comprises a nucleobase sequence according to any one of SEQ ID NOS: 97-192, 289-384, and 385-480. In some implementations, one or more nucleotides are modified. In some implementations, the sense strand comprises a nucleobase sequence according to any one of SEQ ID NOS: 104, 107, 110, 112, 131, 132, 134, 135, 137, 138, 148, 173, 180, 182, 183, 184, 185, 186, and 189. In some implementations, the sense strand comprises a nucleobase sequence according to any one of SEQ ID NOS: 112, 131, 148, 180, 182, 185, and 186. In some implementations, one or more nucleotides are modified. In some implementations, the sense strand has a modification pattern according to nsnsnnnnNfnNfNfNfnnnnnnnnsnsn. In some implementations, the sense strand has a modification pattern according to nsnsnnnnNfnNfNfNfnnnnnnnnnn.

In some implementations, the sense strand comprises a modified nucleotide sequence according to any one of SEQ ID NOS: 289-384 and 385-480. In some implementations, the sense strand comprises a modified nucleotide sequence according to any one of SEQ ID NOS: 296, 299, 302, 304, 323, 324, 326, 327, 329, 330, 340, 365, 372, 374, 375, 376, 377, 378, 381, 392, 395, 398, 400, 419, 420, 422, 423, 425 426, 436, 461, 468, 470, 471, 472, 473, 474, and 477. In some implementations, the sense strand comprises a modified nucleotide sequence according to any one of SEQ ID NOS: 304, 323, 340, 372, 374, 377, 378, 400, 419, 436, 468, 470, 473, and 474.

In some implementations, (a) the sense strand comprises a nucleobase sequence according to SEQ ID NO: 97 and the antisense strand comprises a nucleobase sequence according to SEQ ID NO: 1;
(b) the sense strand comprises a nucleobase sequence according to SEQ ID NO: 98 and the antisense strand comprises a nucleobase sequence according to SEQ ID NO: 2;
(c) the sense strand comprises a nucleobase sequence according to SEQ ID NO: 99 and the antisense strand comprises a nucleobase sequence according to SEQ ID NO: 3;
(d) the sense strand comprises a nucleobase sequence according to SEQ ID NO: 100 and the antisense strand comprises a nucleobase sequence according to SEQ ID NO: 4;
(e) the sense strand comprises a nucleobase sequence according to SEQ ID NO: 101 and the antisense strand comprises a nucleobase sequence according to SEQ ID NO: 5;
(f) the sense strand comprises a nucleobase sequence according to SEQ ID NO: 102 and the antisense strand comprises a nucleobase sequence according to SEQ ID NO: 6;
(g) the sense strand comprises a nucleobase sequence according to SEQ ID NO: 103 and the antisense strand comprises a nucleobase sequence according to SEQ ID NO: 7;
(h) the sense strand comprises a nucleobase sequence according to SEQ ID NO: 104 and the antisense strand comprises a nucleobase sequence according to SEQ ID NO: 8;
(i) the sense strand comprises a nucleobase sequence according to SEQ ID NO: 105 and the antisense strand comprises a nucleobase sequence according to SEQ ID NO: 9;
(j) the sense strand comprises a nucleobase sequence according to SEQ ID NO: 106 and the antisense strand comprises a nucleobase sequence according to SEQ ID NO: 10;
(k) the sense strand comprises a nucleobase sequence according to SEQ ID NO: 107 and the antisense strand comprises a nucleobase sequence according to SEQ ID NO: 11;
(l) the sense strand comprises a nucleobase sequence according to SEQ ID NO: 108 and the antisense strand comprises a nucleobase sequence according to SEQ ID NO: 12;
(m) the sense strand comprises a nucleobase sequence according to SEQ ID NO: 109 and the antisense strand comprises a nucleobase sequence according to SEQ ID NO: 13;
(n) the sense strand comprises a nucleobase sequence according to SEQ ID NO: 110 and the antisense strand comprises a nucleobase sequence according to SEQ ID NO: 14;
(o) the sense strand comprises a nucleobase sequence according to SEQ ID NO: 111 and the antisense strand comprises a nucleobase sequence according to SEQ ID NO: 15;
(p) the sense strand comprises a nucleobase sequence according to SEQ ID NO: 112 and the antisense strand comprises a nucleobase sequence according to SEQ ID NO: 16;
(q) the sense strand comprises a nucleobase sequence according to SEQ ID NO: 113 and the antisense strand comprises a nucleobase sequence according to SEQ ID NO: 17;
(r) the sense strand comprises a nucleobase sequence according to SEQ ID NO: 114 and the antisense strand comprises a nucleobase sequence according to SEQ ID NO: 18;
(s) the sense strand comprises a nucleobase sequence according to SEQ ID NO: 115 and the antisense strand comprises a nucleobase sequence according to SEQ ID NO: 19;
(t) the sense strand comprises a nucleobase sequence according to SEQ ID NO: 116 and the antisense strand comprises a nucleobase sequence according to SEQ ID NO: 20;
(u) the sense strand comprises a nucleobase sequence according to SEQ ID NO: 117 and the antisense strand comprises a nucleobase sequence according to SEQ ID NO: 21;
(v) the sense strand comprises a nucleobase sequence according to SEQ ID NO: 118 and the antisense strand comprises a nucleobase sequence according to SEQ ID NO: 22;
(w) the sense strand comprises a nucleobase sequence according to SEQ ID NO: 119 and the antisense strand comprises a nucleobase sequence according to SEQ ID NO: 23;
(x) the sense strand comprises a nucleobase sequence according to SEQ ID NO: 120 and the antisense strand comprises a nucleobase sequence according to SEQ ID NO: 24;
(y) the sense strand comprises a nucleobase sequence according to SEQ ID NO: 121 and the antisense strand comprises a nucleobase sequence according to SEQ ID NO: 25;
(z) the sense strand comprises a nucleobase sequence according to SEQ ID NO: 122 and the antisense strand comprises a nucleobase sequence according to SEQ ID NO: 26;
(aa) the sense strand comprises a nucleobase sequence according to SEQ ID NO: 123 and the antisense strand comprises a nucleobase sequence according to SEQ ID NO: 27;
(bb) the sense strand comprises a nucleobase sequence according to SEQ ID NO: 124 and the antisense strand comprises a nucleobase sequence according to SEQ ID NO: 28;
(cc) the sense strand comprises a nucleobase sequence according to SEQ ID NO: 125 and the antisense strand comprises a nucleobase sequence according to SEQ ID NO: 29;
(dd) the sense strand comprises a nucleobase sequence according to SEQ ID NO: 126 and the antisense strand comprises a nucleobase sequence according to SEQ ID NO: 30;
(ee) the sense strand comprises a nucleobase sequence according to SEQ ID NO: 127 and the antisense strand comprises a nucleobase sequence according to SEQ ID NO: 31;
(ff) the sense strand comprises a nucleobase sequence according to SEQ ID NO: 128 and the antisense strand comprises a nucleobase sequence according to SEQ ID NO: 32;
(gg) the sense strand comprises a nucleobase sequence according to SEQ ID NO: 129 and the antisense strand comprises a nucleobase sequence according to SEQ ID NO: 33;
(hh) the sense strand comprises a nucleobase sequence according to SEQ ID NO: 130 and the antisense strand comprises a nucleobase sequence according to SEQ ID NO: 34;
(ii) the sense strand comprises a nucleobase sequence according to SEQ ID NO: 131 and the antisense strand comprises a nucleobase sequence according to SEQ ID NO: 35;
(jj) the sense strand comprises a nucleobase sequence according to SEQ ID NO: 132 and the antisense strand comprises a nucleobase sequence according to SEQ ID NO: 36;
(kk) the sense strand comprises a nucleobase sequence according to SEQ ID NO: 133 and the antisense strand comprises a nucleobase sequence according to SEQ ID NO: 37;
(ll) the sense strand comprises a nucleobase sequence according to SEQ ID NO: 134 and the antisense strand comprises a nucleobase sequence according to SEQ ID NO: 38;
(mm) the sense strand comprises a nucleobase sequence according to SEQ ID NO: 135 and the antisense strand comprises a nucleobase sequence according to SEQ ID NO: 39;
(nn) the sense strand comprises a nucleobase sequence according to SEQ ID NO: 136 and the antisense strand comprises a nucleobase sequence according to SEQ ID NO: 40;
(oo) the sense strand comprises a nucleobase sequence according to SEQ ID NO: 137 and the antisense strand comprises a nucleobase sequence according to SEQ ID NO: 41;
(pp) the sense strand comprises a nucleobase sequence according to SEQ ID NO: 138 and the antisense strand comprises a nucleobase sequence according to SEQ ID NO: 42;
(qq) the sense strand comprises a nucleobase sequence according to SEQ ID NO: 139 and the antisense strand comprises a nucleobase sequence according to SEQ ID NO: 43;
(rr) the sense strand comprises a nucleobase sequence according to SEQ ID NO: 140 and the antisense strand comprises a nucleobase sequence according to SEQ ID NO: 44;
(ss) the sense strand comprises a nucleobase sequence according to SEQ ID NO: 141 and the antisense strand comprises a nucleobase sequence according to SEQ ID NO: 45;
(tt) the sense strand comprises a nucleobase sequence according to SEQ ID NO: 142 and the antisense strand comprises a nucleobase sequence according to SEQ ID NO: 46;
(uu) the sense strand comprises a nucleobase sequence according to SEQ ID NO: 143 and the antisense strand comprises a nucleobase sequence according to SEQ ID NO: 47;
(vv) the sense strand comprises a nucleobase sequence according to SEQ ID NO: 144 and the antisense strand comprises a nucleobase sequence according to SEQ ID NO: 48;
(ww) the sense strand comprises a nucleobase sequence according to SEQ ID NO: 145 and the antisense strand comprises a nucleobase sequence according to SEQ ID NO: 49;
(xx) the sense strand comprises a nucleobase sequence according to SEQ ID NO: 146 and the antisense strand comprises a nucleobase sequence according to SEQ ID NO: 50;
(yy) the sense strand comprises a nucleobase sequence according to SEQ ID NO: 147 and the antisense strand comprises a nucleobase sequence according to SEQ ID NO: 51;
(zz) the sense strand comprises a nucleobase sequence according to SEQ ID NO: 148 and the antisense strand comprises a nucleobase sequence according to SEQ ID NO: 52;
(aaa) the sense strand comprises a nucleobase sequence according to SEQ ID NO: 149 and the antisense strand comprises a nucleobase sequence according to SEQ ID NO: 53;
(bbb) the sense strand comprises a nucleobase sequence according to SEQ ID NO: 150 and the antisense strand comprises a nucleobase sequence according to SEQ ID NO: 54;
(ccc) the sense strand comprises a nucleobase sequence according to SEQ ID NO: 151 and the antisense strand comprises a nucleobase sequence according to SEQ ID NO: 55;
(ddd) the sense strand comprises a nucleobase sequence according to SEQ ID NO: 152 and the antisense strand comprises a nucleobase sequence according to SEQ ID NO: 56;
(eee) the sense strand comprises a nucleobase sequence according to SEQ ID NO: 153 and the antisense strand comprises a nucleobase sequence according to SEQ ID NO: 57;
(fff) the sense strand comprises a nucleobase sequence according to SEQ ID NO: 154 and the antisense strand comprises a nucleobase sequence according to SEQ ID NO: 58;
(ggg) the sense strand comprises a nucleobase sequence according to SEQ ID NO: 155 and the antisense strand comprises a nucleobase sequence according to SEQ ID NO: 59;
(hhh) the sense strand comprises a nucleobase sequence according to SEQ ID NO: 156 and the antisense strand comprises a nucleobase sequence according to SEQ ID NO: 60;
(iii) the sense strand comprises a nucleobase sequence according to SEQ ID NO: 157 and the antisense strand comprises a nucleobase sequence according to SEQ ID NO: 61;
(jjj) the sense strand comprises a nucleobase sequence according to SEQ ID NO: 158 and the antisense strand comprises a nucleobase sequence according to SEQ ID NO: 62;
(kkk) the sense strand comprises a nucleobase sequence according to SEQ ID NO: 159 and the antisense strand comprises a nucleobase sequence according to SEQ ID NO: 63;
(lll) the sense strand comprises a nucleobase sequence according to SEQ ID NO: 160 and the antisense strand comprises a nucleobase sequence according to SEQ ID NO: 64;
(mmm) the sense strand comprises a nucleobase sequence according to SEQ ID NO: 161 and the antisense strand comprises a nucleobase sequence according to SEQ ID NO: 65;
(nnn) the sense strand comprises a nucleobase sequence according to SEQ ID NO: 162 and the antisense strand comprises a nucleobase sequence according to SEQ ID NO: 66;
(ooo) the sense strand comprises a nucleobase sequence according to SEQ ID NO: 163 and the antisense strand comprises a nucleobase sequence according to SEQ ID NO: 67;
(ppp) the sense strand comprises a nucleobase sequence according to SEQ ID NO: 164 and the antisense strand comprises a nucleobase sequence according to SEQ ID NO: 68;
(qqq) the sense strand comprises a nucleobase sequence according to SEQ ID NO: 165 and the antisense strand comprises a nucleobase sequence according to SEQ ID NO: 69;
(rrr) the sense strand comprises a nucleobase sequence according to SEQ ID NO: 166 and the antisense strand comprises a nucleobase sequence according to SEQ ID NO: 70;
(sss) the sense strand comprises a nucleobase sequence according to SEQ ID NO: 167 and the antisense strand comprises a nucleobase sequence according to SEQ ID NO: 71;
(ttt) the sense strand comprises a nucleobase sequence according to SEQ ID NO: 168 and the antisense strand comprises a nucleobase sequence according to SEQ ID NO: 72;
(uuu) the sense strand comprises a nucleobase sequence according to SEQ ID NO: 169 and the antisense strand comprises a nucleobase sequence according to SEQ ID NO: 73;
(vvv) the sense strand comprises a nucleobase sequence according to SEQ ID NO: 170 and the antisense strand comprises a nucleobase sequence according to SEQ ID NO: 74;
(www) the sense strand comprises a nucleobase sequence according to SEQ ID NO: 171 and the antisense strand comprises a nucleobase sequence according to SEQ ID NO: 75;
(xxx) the sense strand comprises a nucleobase sequence according to SEQ ID NO: 172 and the antisense strand comprises a nucleobase sequence according to SEQ ID NO: 76;
(yyy) the sense strand comprises a nucleobase sequence according to SEQ ID NO: 173 and the antisense strand comprises a nucleobase sequence according to SEQ ID NO: 77;
(zzz) the sense strand comprises a nucleobase sequence according to SEQ ID NO: 174 and the antisense strand comprises a nucleobase sequence according to SEQ ID NO: 78;
(aaaa) the sense strand comprises a nucleobase sequence according to SEQ ID NO: 175 and the antisense strand comprises a nucleobase sequence according to SEQ ID NO: 79;
(bbbb) the sense strand comprises a nucleobase sequence according to SEQ ID NO: 176 and the antisense strand comprises a nucleobase sequence according to SEQ ID NO: 80;
(cccc) the sense strand comprises a nucleobase sequence according to SEQ ID NO: 177 and the antisense strand comprises a nucleobase sequence according to SEQ ID NO: 81;
(dddd) the sense strand comprises a nucleobase sequence according to SEQ ID NO: 178 and the antisense strand comprises a nucleobase sequence according to SEQ ID NO: 82;
(eeee) the sense strand comprises a nucleobase sequence according to SEQ ID NO: 179 and the antisense strand comprises a nucleobase sequence according to SEQ ID NO: 83;
(ffff) the sense strand comprises a nucleobase sequence according to SEQ ID NO: 180 and the antisense strand comprises a nucleobase sequence according to SEQ ID NO: 84;
(gggg) the sense strand comprises a nucleobase sequence according to SEQ ID NO: 181 and the antisense strand comprises a nucleobase sequence according to SEQ ID NO: 85;
(hhhh) the sense strand comprises a nucleobase sequence according to SEQ ID NO: 182 and the antisense strand comprises a nucleobase sequence according to SEQ ID NO: 86;
(iiii) the sense strand comprises a nucleobase sequence according to SEQ ID NO: 183 and the antisense strand comprises a nucleobase sequence according to SEQ ID NO: 87;
(jjjj) the sense strand comprises a nucleobase sequence according to SEQ ID NO: 184 and the antisense strand comprises a nucleobase sequence according to SEQ ID NO: 88;
(kkkk) the sense strand comprises a nucleobase sequence according to SEQ ID NO: 185 and the antisense strand comprises a nucleobase sequence according to SEQ ID NO: 89;
(llll) the sense strand comprises a nucleobase sequence according to SEQ ID NO: 186 and the antisense strand comprises a nucleobase sequence according to SEQ ID NO: 90;
(mmmm) the sense strand comprises a nucleobase sequence according to SEQ ID NO: 187 and the antisense strand comprises a nucleobase sequence according to SEQ ID NO: 91;
(nnnn) the sense strand comprises a nucleobase sequence according to SEQ ID NO: 188 and the antisense strand comprises a nucleobase sequence according to SEQ ID NO: 92;
(oooo) the sense strand comprises a nucleobase sequence according to SEQ ID NO: 189 and the antisense strand comprises a nucleobase sequence according to SEQ ID NO: 93;
(pppp) the sense strand comprises a nucleobase sequence according to SEQ ID NO: 190 and the antisense strand comprises a nucleobase sequence according to SEQ ID NO: 94;
(qqqq) the sense strand comprises a nucleobase sequence according to SEQ ID NO: 191 and the antisense strand comprises a nucleobase sequence according to SEQ ID NO: 95; or
(rrrr) the sense strand comprises a nucleobase sequence according to SEQ ID NO: 192 and the antisense strand comprises a nucleobase sequence according to SEQ ID NO: 96.

In some implementations, (a) the sense strand comprises a nucleobase sequence according to SEQ ID NO: 112 and the antisense strand comprises a nucleobase sequence according to SEQ ID NO: 16;
(b) the sense strand comprises a nucleobase sequence according to SEQ ID NO: 131 and the antisense strand comprises a nucleobase sequence according to SEQ ID NO: 35;
(c) the sense strand comprises a nucleobase sequence according to SEQ ID NO: 148 and the antisense strand comprises a nucleobase sequence according to SEQ ID NO: 52;
(d) the sense strand comprises a nucleobase sequence according to SEQ ID NO: 180 and the antisense strand comprises a nucleobase sequence according to SEQ ID NO: 84;
(e) the sense strand comprises a nucleobase sequence according to SEQ ID NO: 182 and the antisense strand comprises a nucleobase sequence according to SEQ ID NO: 86;
(f) the sense strand comprises a nucleobase sequence according to SEQ ID NO: 185 and the antisense strand comprises a nucleobase sequence according to SEQ ID NO: 89; or
(g) the sense strand comprises a nucleobase sequence according to SEQ ID NO: 186 and the antisense strand comprises a nucleobase sequence according to SEQ ID NO: 90.

In some implementations, (a) the sense strand comprises a modified nucleotide sequence according to SEQ ID NO: 289 and the antisense strand comprises a modified nucleotide sequence according to SEQ ID NO: 193;
(b) the sense strand comprises a modified nucleotide sequence according to SEQ ID NO: 290 and the antisense strand comprises a modified nucleotide sequence according to SEQ ID NO: 194;
(c) the sense strand comprises a modified nucleotide sequence according to SEQ ID NO: 291 and the antisense strand comprises a modified nucleotide sequence according to SEQ ID NO: 195;
(d) the sense strand comprises a modified nucleotide sequence according to SEQ ID NO: 292 and the antisense strand comprises a modified nucleotide sequence according to SEQ ID NO: 196;
(e) the sense strand comprises a modified nucleotide sequence according to SEQ ID NO: 293 and the antisense strand comprises a modified nucleotide sequence according to SEQ ID NO: 197;
(f) the sense strand comprises a modified nucleotide sequence according to SEQ ID NO: 294 and the antisense strand comprises a modified nucleotide sequence according to SEQ ID NO: 198;
(g) the sense strand comprises a modified nucleotide sequence according to SEQ ID NO: 295 and the antisense strand comprises a modified nucleotide sequence according to SEQ ID NO: 199;
(h) the sense strand comprises a modified nucleotide sequence according to SEQ ID NO: 296 and the antisense strand comprises a modified nucleotide sequence according to SEQ ID NO: 200;
(i) the sense strand comprises a modified nucleotide sequence according to SEQ ID NO: 297 and the antisense strand comprises a modified nucleotide sequence according to SEQ ID NO: 201;
(j) the sense strand comprises a modified nucleotide sequence according to SEQ ID NO: 298 and the antisense strand comprises a modified nucleotide sequence according to SEQ ID NO: 202;
(k) the sense strand comprises a modified nucleotide sequence according to SEQ ID NO: 299 and the antisense strand comprises a modified nucleotide sequence according to SEQ ID NO: 203;
(l) the sense strand comprises a modified nucleotide sequence according to SEQ ID NO: 300 and the antisense strand comprises a modified nucleotide sequence according to SEQ ID NO: 204;
(m) the sense strand comprises a modified nucleotide sequence according to SEQ ID NO: 301 and the antisense strand comprises a modified nucleotide sequence according to SEQ ID NO: 205;
(n) the sense strand comprises a modified nucleotide sequence according to SEQ ID NO: 302 and the antisense strand comprises a modified nucleotide sequence according to SEQ ID NO: 206;
(o) the sense strand comprises a modified nucleotide sequence according to SEQ ID NO: 303 and the antisense strand comprises a modified nucleotide sequence according to SEQ ID NO: 207;
(p) the sense strand comprises a modified nucleotide sequence according to SEQ ID NO: 304 and the antisense strand comprises a modified nucleotide sequence according to SEQ ID NO: 208;
(q) the sense strand comprises a modified nucleotide sequence according to SEQ ID NO: 305 and the antisense strand comprises a modified nucleotide sequence according to SEQ ID NO: 209;
(r) the sense strand comprises a modified nucleotide sequence according to SEQ ID NO: 306 and the antisense strand comprises a modified nucleotide sequence according to SEQ ID NO: 210;
(s) the sense strand comprises a modified nucleotide sequence according to SEQ ID NO: 307 and the antisense strand comprises a modified nucleotide sequence according to SEQ ID NO: 211;
(t) the sense strand comprises a modified nucleotide sequence according to SEQ ID NO: 308 and the antisense strand comprises a modified nucleotide sequence according to SEQ ID NO: 212;
(u) the sense strand comprises a modified nucleotide sequence according to SEQ ID NO: 309 and the antisense strand comprises a modified nucleotide sequence according to SEQ ID NO: 213;
(v) the sense strand comprises a modified nucleotide sequence according to SEQ ID NO: 310 and the antisense strand comprises a modified nucleotide sequence according to SEQ ID NO: 214;
(w) the sense strand comprises a modified nucleotide sequence according to SEQ ID NO: 311 and the antisense strand comprises a modified nucleotide sequence according to SEQ ID NO: 215;
(x) the sense strand comprises a modified nucleotide sequence according to SEQ ID NO: 312 and the antisense strand comprises a modified nucleotide sequence according to SEQ ID NO: 216;
(y) the sense strand comprises a modified nucleotide sequence according to SEQ ID NO: 313 and the antisense strand comprises a modified nucleotide sequence according to SEQ ID NO: 217;
(z) the sense strand comprises a modified nucleotide sequence according to SEQ ID NO: 314 and the antisense strand comprises a modified nucleotide sequence according to SEQ ID NO: 218;
(aa) the sense strand comprises a modified nucleotide sequence according to SEQ ID NO: 315 and the antisense strand comprises a modified nucleotide sequence according to SEQ ID NO: 219;
(bb) the sense strand comprises a modified nucleotide sequence according to SEQ ID NO: 316 and the antisense strand comprises a modified nucleotide sequence according to SEQ ID NO: 220;
(cc) the sense strand comprises a modified nucleotide sequence according to SEQ ID NO: 317 and the antisense strand comprises a modified nucleotide sequence according to SEQ ID NO: 221;
(dd) the sense strand comprises a modified nucleotide sequence according to SEQ ID NO: 318 and the antisense strand comprises a modified nucleotide sequence according to SEQ ID NO: 222;
(ee) the sense strand comprises a modified nucleotide sequence according to SEQ ID NO: 319 and the antisense strand comprises a modified nucleotide sequence according to SEQ ID NO: 223;
(ff) the sense strand comprises a modified nucleotide sequence according to SEQ ID NO: 320 and the antisense strand comprises a modified nucleotide sequence according to SEQ ID NO: 224;
(gg) the sense strand comprises a modified nucleotide sequence according to SEQ ID NO: 321 and the antisense strand comprises a modified nucleotide sequence according to SEQ ID NO: 225;
(hh) the sense strand comprises a modified nucleotide sequence according to SEQ ID NO: 322 and the antisense strand comprises a modified nucleotide sequence according to SEQ ID NO: 226;
(ii) the sense strand comprises a modified nucleotide sequence according to SEQ ID NO: 323 and the antisense strand comprises a modified nucleotide sequence according to SEQ ID NO: 227;
(jj) the sense strand comprises a modified nucleotide sequence according to SEQ ID NO: 324 and the antisense strand comprises a modified nucleotide sequence according to SEQ ID NO: 228;
(kk) the sense strand comprises a modified nucleotide sequence according to SEQ ID NO: 325 and the antisense strand comprises a modified nucleotide sequence according to SEQ ID NO: 229;
(ll) the sense strand comprises a modified nucleotide sequence according to SEQ ID NO: 326 and the antisense strand comprises a modified nucleotide sequence according to SEQ ID NO: 230;
(mm) the sense strand comprises a modified nucleotide sequence according to SEQ ID NO: 327 and the antisense strand comprises a modified nucleotide sequence according to SEQ ID NO: 231;
(nn) the sense strand comprises a modified nucleotide sequence according to SEQ ID NO: 328 and the antisense strand comprises a modified nucleotide sequence according to SEQ ID NO: 232;
(oo) the sense strand comprises a modified nucleotide sequence according to SEQ ID NO: 329 and the antisense strand comprises a modified nucleotide sequence according to SEQ ID NO: 233;
(pp) the sense strand comprises a modified nucleotide sequence according to SEQ ID NO: 330 and the antisense strand comprises a modified nucleotide sequence according to SEQ ID NO: 234;
(qq) the sense strand comprises a modified nucleotide sequence according to SEQ ID NO: 331 and the antisense strand comprises a modified nucleotide sequence according to SEQ ID NO: 235;
(rr) the sense strand comprises a modified nucleotide sequence according to SEQ ID NO: 332 and the antisense strand comprises a modified nucleotide sequence according to SEQ ID NO: 236;
(ss) the sense strand comprises a modified nucleotide sequence according to SEQ ID NO: 333 and the antisense strand comprises a modified nucleotide sequence according to SEQ ID NO: 237;
(tt) the sense strand comprises a modified nucleotide sequence according to SEQ ID NO: 334 and the antisense strand comprises a modified nucleotide sequence according to SEQ ID NO: 238;
(uu) the sense strand comprises a modified nucleotide sequence according to SEQ ID NO: 335 and the antisense strand comprises a modified nucleotide sequence according to SEQ ID NO: 239;
(vv) the sense strand comprises a modified nucleotide sequence according to SEQ ID NO: 336 and the antisense strand comprises a modified nucleotide sequence according to SEQ ID NO: 240;
(ww) the sense strand comprises a modified nucleotide sequence according to SEQ ID NO: 337 and the antisense strand comprises a modified nucleotide sequence according to SEQ ID NO: 241;
(xx) the sense strand comprises a modified nucleotide sequence according to SEQ ID NO: 338 and the antisense strand comprises a modified nucleotide sequence according to SEQ ID NO: 242;
(yy) the sense strand comprises a modified nucleotide sequence according to SEQ ID NO: 339 and the antisense strand comprises a modified nucleotide sequence according to SEQ ID NO: 243;
(zz) the sense strand comprises a modified nucleotide sequence according to SEQ ID NO: 340 and the antisense strand comprises a modified nucleotide sequence according to SEQ ID NO: 244;
(aaa) the sense strand comprises a modified nucleotide sequence according to SEQ ID NO: 341 and the antisense strand comprises a modified nucleotide sequence according to SEQ ID NO: 245;
(bbb) the sense strand comprises a modified nucleotide sequence according to SEQ ID NO: 342 and the antisense strand comprises a modified nucleotide sequence according to SEQ ID NO: 246;
(ccc) the sense strand comprises a modified nucleotide sequence according to SEQ ID NO: 343 and the antisense strand comprises a modified nucleotide sequence according to SEQ ID NO: 247;
(ddd) the sense strand comprises a modified nucleotide sequence according to SEQ ID NO: 344 and the antisense strand comprises a modified nucleotide sequence according to SEQ ID NO: 248;
(eee) the sense strand comprises a modified nucleotide sequence according to SEQ ID NO: 345 and the antisense strand comprises a modified nucleotide sequence according to SEQ ID NO: 249;
(fff) the sense strand comprises a modified nucleotide sequence according to SEQ ID NO: 346 and the antisense strand comprises a modified nucleotide sequence according to SEQ ID NO: 250;
(ggg) the sense strand comprises a modified nucleotide sequence according to SEQ ID NO: 347 and the antisense strand comprises a modified nucleotide sequence according to SEQ ID NO: 251;
(hhh) the sense strand comprises a modified nucleotide sequence according to SEQ ID NO: 348 and the antisense strand comprises a modified nucleotide sequence according to SEQ ID NO: 252;
(iii) the sense strand comprises a modified nucleotide sequence according to SEQ ID NO: 349 and the antisense strand comprises a modified nucleotide sequence according to SEQ ID NO: 253;
(jjj) the sense strand comprises a modified nucleotide sequence according to SEQ ID NO: 350 and the antisense strand comprises a modified nucleotide sequence according to SEQ ID NO: 254;
(kkk) the sense strand comprises a modified nucleotide sequence according to SEQ ID NO: 351 and the antisense strand comprises a modified nucleotide sequence according to SEQ ID NO: 255;
(lll) the sense strand comprises a modified nucleotide sequence according to SEQ ID NO: 352 and the antisense strand comprises a modified nucleotide sequence according to SEQ ID NO: 256;
(mmm) the sense strand comprises a modified nucleotide sequence according to SEQ ID NO: 353 and the antisense strand comprises a modified nucleotide sequence according to SEQ ID NO: 257;
(nnn) the sense strand comprises a modified nucleotide sequence according to SEQ ID NO: 354 and the antisense strand comprises a modified nucleotide sequence according to SEQ ID NO: 258;
(ooo) the sense strand comprises a modified nucleotide sequence according to SEQ ID NO: 355 and the antisense strand comprises a modified nucleotide sequence according to SEQ ID NO: 259;
(ppp) the sense strand comprises a modified nucleotide sequence according to SEQ ID NO: 356 and the antisense strand comprises a modified nucleotide sequence according to SEQ ID NO: 260;
(qqq) the sense strand comprises a modified nucleotide sequence according to SEQ ID NO: 357 and the antisense strand comprises a modified nucleotide sequence according to SEQ ID NO: 261;
(rrr) the sense strand comprises a modified nucleotide sequence according to SEQ ID NO: 358 and the antisense strand comprises a modified nucleotide sequence according to SEQ ID NO: 262;
(sss) the sense strand comprises a modified nucleotide sequence according to SEQ ID NO: 359 and the antisense strand comprises a modified nucleotide sequence according to SEQ ID NO: 263;
(ttt) the sense strand comprises a modified nucleotide sequence according to SEQ ID NO: 360 and the antisense strand comprises a modified nucleotide sequence according to SEQ ID NO: 264;
(uuu) the sense strand comprises a modified nucleotide sequence according to SEQ ID NO: 361 and the antisense strand comprises a modified nucleotide sequence according to SEQ ID NO: 265;
(vvv) the sense strand comprises a modified nucleotide sequence according to SEQ ID NO: 362 and the antisense strand comprises a modified nucleotide sequence according to SEQ ID NO: 266;
(www) the sense strand comprises a modified nucleotide sequence according to SEQ ID NO: 363 and the antisense strand comprises a modified nucleotide sequence according to SEQ ID NO: 267;
(xxx) the sense strand comprises a modified nucleotide sequence according to SEQ ID NO: 364 and the antisense strand comprises a modified nucleotide sequence according to SEQ ID NO: 268;
(yyy) the sense strand comprises a modified nucleotide sequence according to SEQ ID NO: 365 and the antisense strand comprises a modified nucleotide sequence according to SEQ ID NO: 269;
(zzz) the sense strand comprises a modified nucleotide sequence according to SEQ ID NO: 366 and the antisense strand comprises a modified nucleotide sequence according to SEQ ID NO: 270;
(aaaa) the sense strand comprises a modified nucleotide sequence according to SEQ ID NO: 367 and the antisense strand comprises a modified nucleotide sequence according to SEQ ID NO: 271;
(bbbb) the sense strand comprises a modified nucleotide sequence according to SEQ ID NO: 368 and the antisense strand comprises a modified nucleotide sequence according to SEQ ID NO: 272;
(cccc) the sense strand comprises a modified nucleotide sequence according to SEQ ID NO: 369 and the antisense strand comprises a modified nucleotide sequence according to SEQ ID NO: 273;
(dddd) the sense strand comprises a modified nucleotide sequence according to SEQ ID NO: 370 and the antisense strand comprises a modified nucleotide sequence according to SEQ ID NO: 274;
(eeee) the sense strand comprises a modified nucleotide sequence according to SEQ ID NO: 371 and the antisense strand comprises a modified nucleotide sequence according to SEQ ID NO: 275;
(ffff) the sense strand comprises a modified nucleotide sequence according to SEQ ID NO: 372 and the antisense strand comprises a modified nucleotide sequence according to SEQ ID NO: 276;
(gggg) the sense strand comprises a modified nucleotide sequence according to SEQ ID NO: 373 and the antisense strand comprises a modified nucleotide sequence according to SEQ ID NO: 277;
(hhhh) the sense strand comprises a modified nucleotide sequence according to SEQ ID NO: 374 and the antisense strand comprises a modified nucleotide sequence according to SEQ ID NO: 278;
(iiii) the sense strand comprises a modified nucleotide sequence according to SEQ ID NO: 375 and the antisense strand comprises a modified nucleotide sequence according to SEQ ID NO: 279;
(jjjj) the sense strand comprises a modified nucleotide sequence according to SEQ ID NO: 376 and the antisense strand comprises a modified nucleotide sequence according to SEQ ID NO: 280;
(kkkk) the sense strand comprises a modified nucleotide sequence according to SEQ ID NO: 377 and the antisense strand comprises a modified nucleotide sequence according to SEQ ID NO: 281;
(llll) the sense strand comprises a modified nucleotide sequence according to SEQ ID NO: 378 and the antisense strand comprises a modified nucleotide sequence according to SEQ ID NO: 282;
(mmmm) the sense strand comprises a modified nucleotide sequence according to SEQ ID NO: 379 and the antisense strand comprises a modified nucleotide sequence according to SEQ ID NO: 283;
(nnnn) the sense strand comprises a modified nucleotide sequence according to SEQ ID NO: 380 and the antisense strand comprises a modified nucleotide sequence according to SEQ ID NO: 284;
(oooo) the sense strand comprises a modified nucleotide sequence according to SEQ ID NO: 381 and the antisense strand comprises a modified nucleotide sequence according to SEQ ID NO: 285;
(pppp) the sense strand comprises a modified nucleotide sequence according to SEQ ID NO: 382 and the antisense strand comprises a modified nucleotide sequence according to SEQ ID NO: 286;
(qqqq) the sense strand comprises a modified nucleotide sequence according to SEQ ID NO: 383 and the antisense strand comprises a modified nucleotide sequence according to SEQ ID NO: 287;
(rrrr) the sense strand comprises a modified nucleotide sequence according to SEQ ID NO: 384 and the antisense strand comprises a modified nucleotide sequence according to SEQ ID NO: 288;
(ssss) the sense strand comprises a modified nucleotide sequence according to SEQ ID NO: 385 and the antisense strand comprises a modified nucleotide sequence according to SEQ ID NO: 193;
(tttt) the sense strand comprises a modified nucleotide sequence according to SEQ ID NO: 386 and the antisense strand comprises a modified nucleotide sequence according to SEQ ID NO: 194;
(uuuu) the sense strand comprises a modified nucleotide sequence according to SEQ ID NO: 387 and the antisense strand comprises a modified nucleotide sequence according to SEQ ID NO: 195;
(vvvv) the sense strand comprises a modified nucleotide sequence according to SEQ ID NO: 388 and the antisense strand comprises a modified nucleotide sequence according to SEQ ID NO: 196;
(wwww) the sense strand comprises a modified nucleotide sequence according to SEQ ID NO: 389 and the antisense strand comprises a modified nucleotide sequence according to SEQ ID NO: 197;
(xxxx) the sense strand comprises a modified nucleotide sequence according to SEQ ID NO: 390 and the antisense strand comprises a modified nucleotide sequence according to SEQ ID NO: 198;
(yyyy) the sense strand comprises a modified nucleotide sequence according to SEQ ID NO: 391 and the antisense strand comprises a modified nucleotide sequence according to SEQ ID NO: 199;
(zzzz) the sense strand comprises a modified nucleotide sequence according to SEQ ID NO: 392 and the antisense strand comprises a modified nucleotide sequence according to SEQ ID NO: 200;
(aaaaa) the sense strand comprises a modified nucleotide sequence according to SEQ ID NO: 393 and the antisense strand comprises a modified nucleotide sequence according to SEQ ID NO: 201;
(bbbbb) the sense strand comprises a modified nucleotide sequence according to SEQ ID NO: 394 and the antisense strand comprises a modified nucleotide sequence according to SEQ ID NO: 202;
(ccccc) the sense strand comprises a modified nucleotide sequence according to SEQ ID NO: 395 and the antisense strand comprises a modified nucleotide sequence according to SEQ ID NO: 203;
(ddddd) the sense strand comprises a modified nucleotide sequence according to SEQ ID NO: 396 and the antisense strand comprises a modified nucleotide sequence according to SEQ ID NO: 204;
(eeeee) the sense strand comprises a modified nucleotide sequence according to SEQ ID NO: 397 and the antisense strand comprises a modified nucleotide sequence according to SEQ ID NO: 205;
(fffff) the sense strand comprises a modified nucleotide sequence according to SEQ ID NO: 398 and the antisense strand comprises a modified nucleotide sequence according to SEQ ID NO: 206;
(ggggg) the sense strand comprises a modified nucleotide sequence according to SEQ ID NO: 399 and the antisense strand comprises a modified nucleotide sequence according to SEQ ID NO: 207;
(hhhhh) the sense strand comprises a modified nucleotide sequence according to SEQ ID NO: 400 and the antisense strand comprises a modified nucleotide sequence according to SEQ ID NO: 208;
(iiiii) the sense strand comprises a modified nucleotide sequence according to SEQ ID NO: 401 and the antisense strand comprises a modified nucleotide sequence according to SEQ ID NO: 209;
(jjjjj) the sense strand comprises a modified nucleotide sequence according to SEQ ID NO: 402 and the antisense strand comprises a modified nucleotide sequence according to SEQ ID NO: 210;
(kkkkk) the sense strand comprises a modified nucleotide sequence according to SEQ ID NO: 403 and the antisense strand comprises a modified nucleotide sequence according to SEQ ID NO: 211;
(lllll) the sense strand comprises a modified nucleotide sequence according to SEQ ID NO: 404 and the antisense strand comprises a modified nucleotide sequence according to SEQ ID NO: 212;
(mmmmm) the sense strand comprises a modified nucleotide sequence according to SEQ ID NO: 405 and the antisense strand comprises a modified nucleotide sequence according to SEQ ID NO: 213;
(nnnnn) the sense strand comprises a modified nucleotide sequence according to SEQ ID NO: 406 and the antisense strand comprises a modified nucleotide sequence according to SEQ ID NO: 214;
(ooooo) the sense strand comprises a modified nucleotide sequence according to SEQ ID NO: 407 and the antisense strand comprises a modified nucleotide sequence according to SEQ ID NO: 215;
(ppppp) the sense strand comprises a modified nucleotide sequence according to SEQ ID NO: 408 and the antisense strand comprises a modified nucleotide sequence according to SEQ ID NO: 216;
(qqqqq) the sense strand comprises a modified nucleotide sequence according to SEQ ID NO: 409 and the antisense strand comprises a modified nucleotide sequence according to SEQ ID NO: 217;
(rrrrr) the sense strand comprises a modified nucleotide sequence according to SEQ ID NO: 410 and the antisense strand comprises a modified nucleotide sequence according to SEQ ID NO: 218;
(sssss) the sense strand comprises a modified nucleotide sequence according to SEQ ID NO: 411 and the antisense strand comprises a modified nucleotide sequence according to SEQ ID NO: 219;
(ttttt) the sense strand comprises a modified nucleotide sequence according to SEQ ID NO: 412 and the antisense strand comprises a modified nucleotide sequence according to SEQ ID NO: 220;
(uuuuu) the sense strand comprises a modified nucleotide sequence according to SEQ ID NO: 413 and the antisense strand comprises a modified nucleotide sequence according to SEQ ID NO: 221;
(vvvvv) the sense strand comprises a modified nucleotide sequence according to SEQ ID NO: 414 and the antisense strand comprises a modified nucleotide sequence according to SEQ ID NO: 222;
(wwwww) the sense strand comprises a modified nucleotide sequence according to SEQ ID NO: 415 and the antisense strand comprises a modified nucleotide sequence according to SEQ ID NO: 223;
(xxxxx) the sense strand comprises a modified nucleotide sequence according to SEQ ID NO: 416 and the antisense strand comprises a modified nucleotide sequence according to SEQ ID NO: 224;
(yyyyy) the sense strand comprises a modified nucleotide sequence according to SEQ ID NO: 417 and the antisense strand comprises a modified nucleotide sequence according to SEQ ID NO: 225;
(zzzzz) the sense strand comprises a modified nucleotide sequence according to SEQ ID NO: 418 and the antisense strand comprises a modified nucleotide sequence according to SEQ ID NO: 226;
(aaaaaa) the sense strand comprises a modified nucleotide sequence according to SEQ ID NO: 419 and the antisense strand comprises a modified nucleotide sequence according to SEQ ID NO: 227;
(bbbbbb) the sense strand comprises a modified nucleotide sequence according to SEQ ID NO: 420 and the antisense strand comprises a modified nucleotide sequence according to SEQ ID NO: 228;
(cccccc) the sense strand comprises a modified nucleotide sequence according to SEQ ID NO: 421 and the antisense strand comprises a modified nucleotide sequence according to SEQ ID NO: 229;
(dddddd) the sense strand comprises a modified nucleotide sequence according to SEQ ID NO: 422 and the antisense strand comprises a modified nucleotide sequence according to SEQ ID NO: 230;
(eeeeee) the sense strand comprises a modified nucleotide sequence according to SEQ ID NO: 423 and the antisense strand comprises a modified nucleotide sequence according to SEQ ID NO: 231;
(ffffff) the sense strand comprises a modified nucleotide sequence according to SEQ ID NO: 424 and the antisense strand comprises a modified nucleotide sequence according to SEQ ID NO: 232;
(gggggg) the sense strand comprises a modified nucleotide sequence according to SEQ ID NO: 425 and the antisense strand comprises a modified nucleotide sequence according to SEQ ID NO: 233;
(hhhhhh) the sense strand comprises a modified nucleotide sequence according to SEQ ID NO: 426 and the antisense strand comprises a modified nucleotide sequence according to SEQ ID NO: 234;
(iiiiii) the sense strand comprises a modified nucleotide sequence according to SEQ ID NO: 427 and the antisense strand comprises a modified nucleotide sequence according to SEQ ID NO: 235;
(jjjjjj) the sense strand comprises a modified nucleotide sequence according to SEQ ID NO: 428 and the antisense strand comprises a modified nucleotide sequence according to SEQ ID NO: 236;
(kkkkkk) the sense strand comprises a modified nucleotide sequence according to SEQ ID NO: 429 and the antisense strand comprises a modified nucleotide sequence according to SEQ ID NO: 237;
(llllll) the sense strand comprises a modified nucleotide sequence according to SEQ ID NO: 430 and the antisense strand comprises a modified nucleotide sequence according to SEQ ID NO: 238;
(mmmmmm) the sense strand comprises a modified nucleotide sequence according to SEQ ID NO: 431 and the antisense strand comprises a modified nucleotide sequence according to SEQ ID NO: 239;
(nnnnnn) the sense strand comprises a modified nucleotide sequence according to SEQ ID NO: 432 and the antisense strand comprises a modified nucleotide sequence according to SEQ ID NO: 240;
(oooooo) the sense strand comprises a modified nucleotide sequence according to SEQ ID NO: 433 and the antisense strand comprises a modified nucleotide sequence according to SEQ ID NO: 241;
(pppppp) the sense strand comprises a modified nucleotide sequence according to SEQ ID NO: 434 and the antisense strand comprises a modified nucleotide sequence according to SEQ ID NO: 242;
(qqqqqq) the sense strand comprises a modified nucleotide sequence according to SEQ ID NO: 435 and the antisense strand comprises a modified nucleotide sequence according to SEQ ID NO: 243;
(rrrrrr) the sense strand comprises a modified nucleotide sequence according to SEQ ID NO: 436 and the antisense strand comprises a modified nucleotide sequence according to SEQ ID NO: 244;
(ssssss) the sense strand comprises a modified nucleotide sequence according to SEQ ID NO: 437 and the antisense strand comprises a modified nucleotide sequence according to SEQ ID NO: 245;
(tttttt) the sense strand comprises a modified nucleotide sequence according to SEQ ID NO: 438 and the antisense strand comprises a modified nucleotide sequence according to SEQ ID NO: 246;
(uuuuuu) the sense strand comprises a modified nucleotide sequence according to SEQ ID NO: 439 and the antisense strand comprises a modified nucleotide sequence according to SEQ ID NO: 247;
(vvvvvv) the sense strand comprises a modified nucleotide sequence according to SEQ ID NO: 440 and the antisense strand comprises a modified nucleotide sequence according to SEQ ID NO: 248;
(wwwwww) the sense strand comprises a modified nucleotide sequence according to SEQ ID NO: 441 and the antisense strand comprises a modified nucleotide sequence according to SEQ ID NO: 249;
(xxxxxx) the sense strand comprises a modified nucleotide sequence according to SEQ ID NO: 442 and the antisense strand comprises a modified nucleotide sequence according to SEQ ID NO: 250;
(yyyyyy) the sense strand comprises a modified nucleotide sequence according to SEQ ID NO: 443 and the antisense strand comprises a modified nucleotide sequence according to SEQ ID NO: 251;
(zzzzzz) the sense strand comprises a modified nucleotide sequence according to SEQ ID NO: 444 and the antisense strand comprises a modified nucleotide sequence according to SEQ ID NO: 252;
(aaaaaaa) the sense strand comprises a modified nucleotide sequence according to SEQ ID NO: 445 and the antisense strand comprises a modified nucleotide sequence according to SEQ ID NO: 253;
(bbbbbbb) the sense strand comprises a modified nucleotide sequence according to SEQ ID NO: 446 and the antisense strand comprises a modified nucleotide sequence according to SEQ ID NO: 254;
(ccccccc) the sense strand comprises a modified nucleotide sequence according to SEQ ID NO: 447 and the antisense strand comprises a modified nucleotide sequence according to SEQ ID NO: 255;
(ddddddd) the sense strand comprises a modified nucleotide sequence according to SEQ ID NO: 448 and the antisense strand comprises a modified nucleotide sequence according to SEQ ID NO: 256;
(eeeeeee) the sense strand comprises a modified nucleotide sequence according to SEQ ID NO: 449 and the antisense strand comprises a modified nucleotide sequence according to SEQ ID NO: 257;
(fffffff) the sense strand comprises a modified nucleotide sequence according to SEQ ID NO: 450 and the antisense strand comprises a modified nucleotide sequence according to SEQ ID NO: 258;
(ggggggg) the sense strand comprises a modified nucleotide sequence according to SEQ ID NO: 451 and the antisense strand comprises a modified nucleotide sequence according to SEQ ID NO: 259;
(hhhhhhh) the sense strand comprises a modified nucleotide sequence according to SEQ ID NO: 452 and the antisense strand comprises a modified nucleotide sequence according to SEQ ID NO: 260;
(iiiiii) the sense strand comprises a modified nucleotide sequence according to SEQ ID NO: 453 and the antisense strand comprises a modified nucleotide sequence according to SEQ ID NO: 261;
(jjjjjjj) the sense strand comprises a modified nucleotide sequence according to SEQ ID NO: 454 and the antisense strand comprises a modified nucleotide sequence according to SEQ ID NO: 262;
(kkkkkkk) the sense strand comprises a modified nucleotide sequence according to SEQ ID NO: 455 and the antisense strand comprises a modified nucleotide sequence according to SEQ ID NO: 263;
(lllllll) the sense strand comprises a modified nucleotide sequence according to SEQ ID NO: 456 and the antisense strand comprises a modified nucleotide sequence according to SEQ ID NO: 264;
(mmmmmmm) the sense strand comprises a modified nucleotide sequence according to SEQ ID NO: 457 and the antisense strand comprises a modified nucleotide sequence according to SEQ ID NO: 265;
(nnnnnnn) the sense strand comprises a modified nucleotide sequence according to SEQ ID NO: 458 and the antisense strand comprises a modified nucleotide sequence according to SEQ ID NO: 266;
(ooooooo) the sense strand comprises a modified nucleotide sequence according to SEQ ID NO: 459 and the antisense strand comprises a modified nucleotide sequence according to SEQ ID NO: 267;
(ppppppp) the sense strand comprises a modified nucleotide sequence according to SEQ ID NO: 460 and the antisense strand comprises a modified nucleotide sequence according to SEQ ID NO: 268;
(qqqqqqq) the sense strand comprises a modified nucleotide sequence according to SEQ ID NO: 461 and the antisense strand comprises a modified nucleotide sequence according to SEQ ID NO: 269;
(rrrrrrrr) the sense strand comprises a modified nucleotide sequence according to SEQ ID NO: 462 and the antisense strand comprises a modified nucleotide sequence according to SEQ ID NO: 270;
(sssssss) the sense strand comprises a modified nucleotide sequence according to SEQ ID NO: 463 and the antisense strand comprises a modified nucleotide sequence according to SEQ ID NO: 271;
(ttttttt) the sense strand comprises a modified nucleotide sequence according to SEQ ID NO: 464 and the antisense strand comprises a modified nucleotide sequence according to SEQ ID NO: 272;
(uuuuuuu) the sense strand comprises a modified nucleotide sequence according to SEQ ID NO: 465 and the antisense strand comprises a modified nucleotide sequence according to SEQ ID NO: 273;
(vvvvvvv) the sense strand comprises a modified nucleotide sequence according to SEQ ID NO: 466 and the antisense strand comprises a modified nucleotide sequence according to SEQ ID NO: 274;
(wwwwwww) the sense strand comprises a modified nucleotide sequence according to SEQ ID NO: 467 and the antisense strand comprises a modified nucleotide sequence according to SEQ ID NO: 275;
(xxxxxxx) the sense strand comprises a modified nucleotide sequence according to SEQ ID NO: 468 and the antisense strand comprises a modified nucleotide sequence according to SEQ ID NO: 276;
(yyyyyyy) the sense strand comprises a modified nucleotide sequence according to SEQ ID NO: 469 and the antisense strand comprises a modified nucleotide sequence according to SEQ ID NO: 277;
(zzzzzzz) the sense strand comprises a modified nucleotide sequence according to SEQ ID NO: 470 and the antisense strand comprises a modified nucleotide sequence according to SEQ ID NO: 278;
(aaaaaaaa) the sense strand comprises a modified nucleotide sequence according to SEQ ID NO: 471 and the antisense strand comprises a modified nucleotide sequence according to SEQ ID NO: 279;
(bbbbbbbb) the sense strand comprises a modified nucleotide sequence according to SEQ ID NO: 472 and the antisense strand comprises a modified nucleotide sequence according to SEQ ID NO: 280;
(cccccccc) the sense strand comprises a modified nucleotide sequence according to SEQ ID NO: 473 and the antisense strand comprises a modified nucleotide sequence according to SEQ ID NO: 281;
(dddddddd) the sense strand comprises a modified nucleotide sequence according to SEQ ID NO: 474 and the antisense strand comprises a modified nucleotide sequence according to SEQ ID NO: 282;
(eeeeeeee) the sense strand comprises a modified nucleotide sequence according to SEQ ID NO: 475 and the antisense strand comprises a modified nucleotide sequence according to SEQ ID NO: 283;
(ffffffff) the sense strand comprises a modified nucleotide sequence according to SEQ ID NO: 476 and the antisense strand comprises a modified nucleotide sequence according to SEQ ID NO: 284;
(gggggggg) the sense strand comprises a modified nucleotide sequence according to SEQ ID NO: 477 and the antisense strand comprises a modified nucleotide sequence according to SEQ ID NO: 285;
(hhhhhhhh) the sense strand comprises a modified nucleotide sequence according to SEQ ID NO: 478 and the antisense strand comprises a modified nucleotide sequence according to SEQ ID NO: 286;
(iiiiiii) the sense strand comprises a modified nucleotide sequence according to SEQ ID NO: 479 and the antisense strand comprises a modified nucleotide sequence according to SEQ ID NO: 287; or
(jjjjjjjj) the sense strand comprises a modified nucleotide sequence according to SEQ ID NO: 480 and the antisense strand comprises a modified nucleotide sequence according to SEQ ID NO: 288.

In some implementations, (a) the sense strand comprises a modified nucleotide sequence according to SEQ ID NO: 400 and the antisense strand comprises a modified nucleotide sequence according to SEQ ID NO: 208; (b) the sense strand comprises a modified nucleotide sequence according to SEQ ID NO: 419 and the antisense strand comprises a modified nucleotide sequence according to SEQ ID NO: 227; (c) the sense strand comprises a modified nucleotide sequence according to SEQ ID NO: 436 and the antisense strand comprises a modified nucleotide sequence according to SEQ ID NO: 244; (d) the sense strand comprises a modified nucleotide sequence according to SEQ ID NO: 468 and the antisense strand comprises a modified nucleotide sequence according to SEQ ID NO: 276; (e) the sense strand comprises a modified nucleotide sequence according to SEQ ID NO: 470 and the antisense strand comprises a modified nucleotide sequence according to SEQ ID NO: 278; (f) the sense strand comprises a modified nucleotide sequence according to SEQ ID NO: 473 and the antisense strand comprises a modified nucleotide sequence according to SEQ ID NO: 281; or (g) the sense strand comprises a modified nucleotide sequence according to SEQ ID NO: 474 and the antisense strand comprises a modified nucleotide sequence according to SEQ ID NO: 282.

In some implementations, the RNAi agent comprises a targeting ligand comprising a structure according to Formula I:

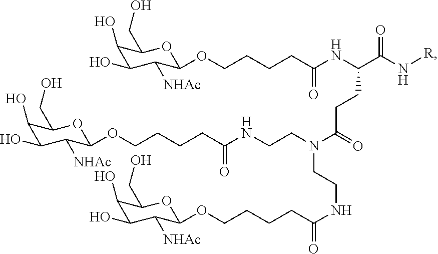

wherein R is a linker conjugated to the wherein the targeting ligand is conjugated to the 3′ end of the sense strand, wherein R comprises a structure according to Formula III:

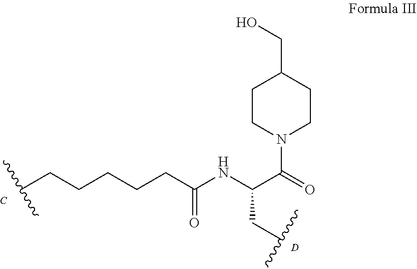

and wherein connection point C of Formula III is conjugated to Formula I, and connection point D of Formula III is conjugated to the 3′ end of the sense strand;
or wherein the RNAi agent comprises a targeting ligand comprising a structure according to Formula V:

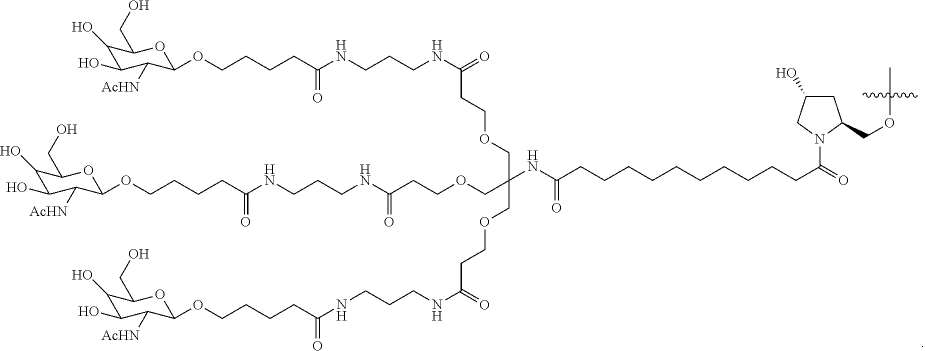 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some implementations, the sense strand comprises a 3′ overhang of 1-5 nucleotides in length. In some implementations, the sense strand comprises a 5′ overhang of 1-5 nucleotides in length. In some implementations, the antisense strand comprises a 3′ overhang of 1-5 nucleotides in length. In some implementations, the antisense strand comprises a 5′ overhang of 1-5 nucleotides in length. In some implementations, the sense strand or the antisense strand comprises at least one modified nucleotide or at least one modified internucleoside linkage. In some implementations, the sense strand and or the antisense strand comprises a modified internucleoside linkage at a first, second, penultimate, or final internucleoside linkage position.

In some implementations, the at least one modified nucleotide or at least one modified internucleoside linkage comprises a 2′-O-methyl modified nucleotide, a nucleotide comprising a 5′-phosphorothioate group, a 2′-deoxy-2′-fluoro modified nucleotide, a 2′-deoxy-modified nucleotide, a locked nucleotide, an abasic nucleotide, deoxythymidine, an inverted deoxythymidine, a 2′-amino-modified nucleotide, a 2′-alkyl-modified nucleotide, a morpholino nucleotide, a phosphoramidate, or a non-natural base comprising nucleotide.

In some implementations, the targeting ligand is conjugated to the sense strand. In some implementations, the targeting ligand is conjugated to the 3′ end of the sense strand. In some implementations, the targeting ligand is conjugated to the 5′ end of the sense strand. In some implementations, the targeting ligand is conjugated to the antisense strand. In some implementations, the targeting ligand is conjugated to the 3′ end of the antisense strand. In some implementations, the targeting ligand is conjugated to the 5′ end of the antisense strand.

In some implementations, the targeting ligand comprises a galactose trimer. In some implementations, the targeting ligand comprises N-acetylgalactosamine (GalNAc). In some implementations, the targeting ligand comprises a structure according to Formula I:

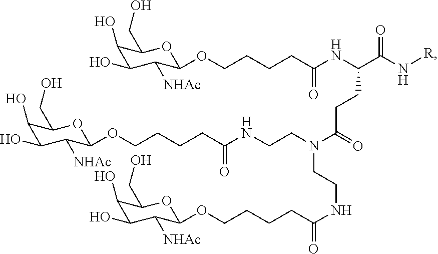

wherein R is:
(i) the sense strand or the antisense strand; or
(ii) a linker conjugated to the sense strand or the antisense strand.

In some implementations, R is a linker conjugated to the sense strand or the antisense strand having a structure according to Formula III:

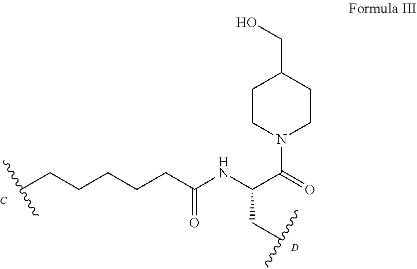

wherein:
(i) connection point C of Formula III is conjugated to Formula I, and connection point D of Formula III is conjugated to the sense strand or the antisense strand; or
(ii) connection point C of Formula III is conjugated to Formula I, and connection point D of Formula III is conjugated to a phosphate group which is conjugated to the sense strand or the antisense strand.

In some implementations, the RNAi agent comprises a targeting ligand and linker according to Formula V:

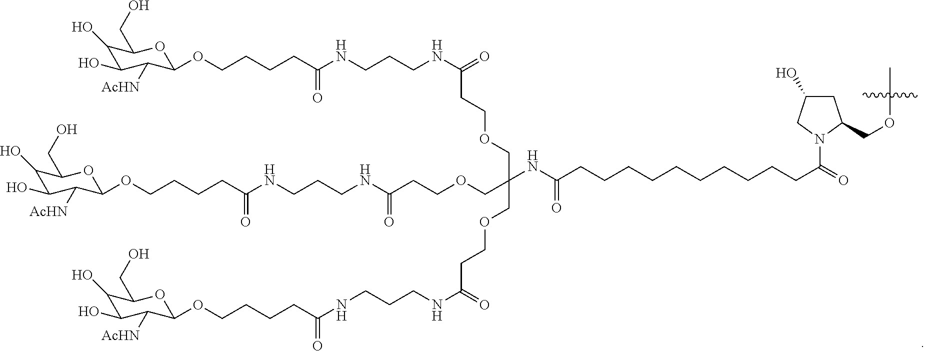 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some implementations, Formula III or the phosphate group is conjugated to the 3′ end or the 5′ end of the sense strand or the antisense strand. In some implementations, Formula III or the phosphate group is conjugated to an internal nucleotide or nucleoside base of the sense strand or the antisense strand.

In some implementations, the RNAi agent is an siRNA molecule. In some implementations, the RNAi agent is an shRNA molecule.

Described herein is a modified oligonucleotide consisting of 12 to 30 or 12 to 50 linked nucleosides, wherein the modified oligonucleotide has a nucleobase sequence comprising at least 12, at least 13, at least 14, at least 15, at least 16, at least 17, at least 18, at least 19, at least 20, at least 21, at least 22, at least 23, at least 24, or at least 25 contiguous nucleobases complementary to an equal length portion of SEQ ID NO: 481, and wherein the modified oligonucleotide comprises at least one synthetically modified internucleoside linkage or at least one synthetically modified sugar-phosphate backbone.

In some implementations, the modified oligonucleotide comprises a synthetically modified internucleoside linkage at a first, second, penultimate, or final internucleoside linkage position. In some implementations, the modified oligonucleotide comprises a 2′-O-methyl modified nucleotide, a nucleotide comprising a 5′-phosphorothioate group, a 2′-deoxy-2′-fluoro modified nucleotide, a 2′-deoxy-modified nucleotide, a locked nucleotide, an abasic nucleotide, deoxythymidine, an inverted deoxythymidine, a 2′-amino-modified nucleotide, a 2′-alkyl-modified nucleotide, a morpholino nucleotide, a phosphoramidate, or a non-natural base comprising nucleotide.

In some implementations, the modified oligonucleotide or a subsequence thereof differs by 0, 1, 2, 3, or 4 nucleotides from an equal length portion of any one of SEQ ID NOS: 193-288, wherein the subsequence is at least 14, 15, 16, 17, 18, 19, 20, 21, 22, or 23 bases in length. In some implementations, the modified oligonucleotide comprises, in order, nucleotides 1-23 of any one of SEQ ID NOS: 193-288. In some implementations, the modified oligonucleotide comprises a modified nucleotide sequence according to any one of SEQ ID NOS: 193-288. In some implementations, the modified oligonucleotide is conjugated to a targeting ligand that targets a hepatocyte. In some implementations, the modified oligonucleotide is conjugated to the targeting ligand conjugated to its 3′ end. In some implementations, the modified oligonucleotide is conjugated to the targeting ligand conjugated to its 5′ end.

In some implementations, the targeting ligand comprises a galactose trimer. In some implementations, the targeting ligand comprises N-acetylgalactosamine (GalNAc). In some implementations, the targeting ligand comprises a structure according to Formula I:

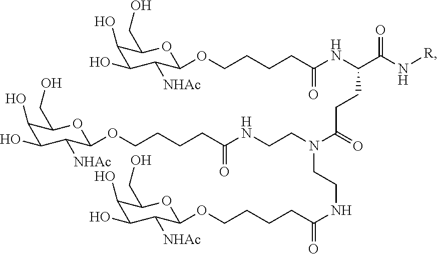

wherein R is:
(i) the modified oligonucleotide; or
(ii) a linker conjugated to the modified oligonucleotide.

In some implementations, R is a linker conjugated to the modified oligonucleotide having a structure according to Formula III:

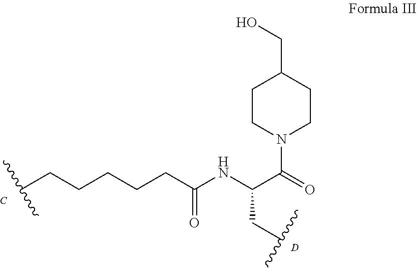

wherein:
(i) connection point C of Formula III is conjugated to Formula I, and connection point D of Formula III is conjugated to the modified oligonucleotide; or
(ii) connection point C of Formula III is conjugated to Formula I, and connection point D of Formula III is conjugated to a phosphate group which is conjugated to the modified oligonucleotide.

In some implementations, Formula III or the phosphate group is conjugated to the 3′ end or the 5′ end of the modified oligonucleotide. In some implementations, Formula III or the phosphate group is conjugated to an internal nucleotide or nucleoside base of the modified oligonucleotide. In some implementations, the modified oligonucleotide comprises a targeting ligand and linker according to Formula V:

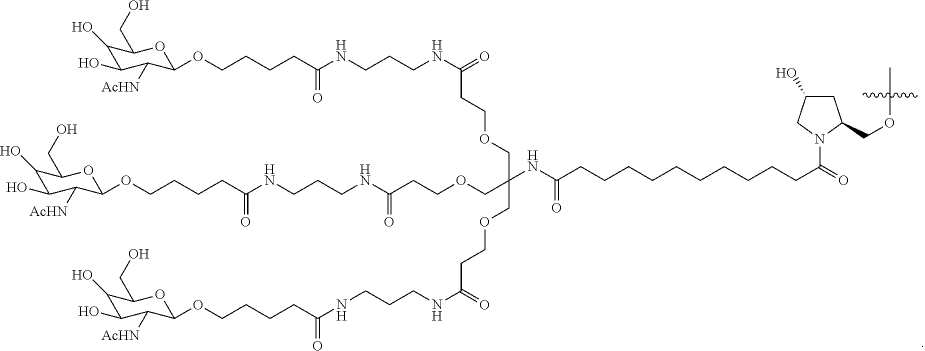 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some implementations, the modified oligonucleotide is an siRNA molecule. In some implementations, the modified oligonucleotide is an shRNA molecule.

Described herein is an oligomeric duplex comprising: (a) a first modified oligonucleotide consisting of 12 to 30 or 12 to 50 linked nucleosides, wherein a nucleobase sequence of the first modified oligonucleotide comprises at least 12, at least 13, at least 14, at least 15, at least 16, at least 17, at least 18, at least 19, at least 20, at least 21, at least 22, at least 23, at least 24, or at least 25 contiguous nucleobases that are at least 80%, 81%, 82%, 83%, 84%, 85%, 86%, 87%, 88%, 89%, 90%, 91%, 92%, 93%, 94%, 95%, 96%, 97%, 98%, 99%, or 100% complementary to an equal length portion of SEQ ID NO: 481; and (b) a second modified oligonucleotide consisting of 12 to 30 or 12 to 50 linked nucleosides, wherein a nucleobase sequence of the second modified oligonucleotide comprises a complementary region of at least 8 nucleobases that is at least 90% complementary to the nucleobase sequence of an equal length portion of the first modified oligonucleotide.

In some implementations, the first modified oligonucleotide and/or second modified oligonucleotide comprises at least one synthetically modified internucleoside linkage or at least one synthetically modified sugar-phosphate backbone. In some implementations, the first modified oligonucleotide and/or the second comprises a 2′-O-methyl modified nucleotide, a nucleotide comprising a 5′-phosphorothioate group, a 2′-deoxy-2′-fluoro modified nucleotide, a 2′-deoxy-modified nucleotide, a locked nucleotide, an abasic nucleotide, deoxythymidine, an inverted deoxythymidine, a 2′-amino-modified nucleotide, a 2′-alkyl-modified nucleotide, a morpholino nucleotide, a phosphoramidate, or a non-natural base comprising nucleotide.

In some implementations, the first modified oligonucleotide or a subsequence thereof differs by 0, 1, 2, 3, or 4 nucleotides from an equal length portion of any one of SEQ ID NOS: 193-288, wherein the subsequence is at least 14, 15, 16, 17, 18, 19, 20, 21, 22, or 23 bases in length. In some implementations, the first modified oligonucleotide comprises, in order, nucleotides 1-23 of any one of SEQ ID NOS: 193-288. In some implementations, the first modified oligonucleotide comprises a modified nucleotide sequence according to any one of SEQ ID NOS: 193-288. In some implementations, the first modified oligonucleotide comprises a modified nucleotide sequence according to any one of SEQ ID NOS: 200, 203, 206, 208, 227, 228, 230, 231, 233, 234, 244, 269, 276, 278, 279, 280, 281, 282, and 285.

In some implementations, the second modified oligonucleotide or subsequence thereof differs by 0, 1, 2, 3, or 4 nucleotides from an equal length portion of any one of SEQ ID NOS: 289-384 and 385-480, wherein the subsequence is at least 14, 15, 16, 17, 18, 19, 20, or 21 bases in length. In some implementations, the second modified oligonucleotide comprises, in order, nucleotides 1-21 of any one of SEQ ID NOS: 289-384 and 385-480. In some implementations, the second modified oligonucleotide comprises a modified nucleotide sequence according to any one of SEQ ID NOS: 289-384 and 385-480. In some implementations, the second modified oligonucleotide comprises a modified nucleotide sequence according to any one of SEQ ID NOS: 104, 107, 110, 112, 131, 132, 134, 135, 137, 138, 148, 173, 180, 182, 183, 184, 185, 186, 189, 296, 299, 302, 304, 323, 324, 326, 327, 329, 330, 340, 365, 372, 374, 375, 376, 377, 378, 381, 392, 395, 398, 400, 419, 420, 422, 423, 425 426, 436, 461, 468, 470, 471, 472, 473, 474, and 477.

In some implementations, (a) the second modified oligonucleotide comprises a modified nucleotide sequence according to SEQ ID NO: 289 and the first modified oligonucleotide comprises a modified nucleotide sequence according to SEQ ID NO: 193;
(b) the second modified oligonucleotide comprises a modified nucleotide sequence according to SEQ ID NO: 290 and the first modified oligonucleotide comprises a modified nucleotide sequence according to SEQ ID NO: 194;
(c) the second modified oligonucleotide comprises a modified nucleotide sequence according to SEQ ID NO: 291 and the first modified oligonucleotide comprises a modified nucleotide sequence according to SEQ ID NO: 195;
(d) the second modified oligonucleotide comprises a modified nucleotide sequence according to SEQ ID NO: 292 and the first modified oligonucleotide comprises a modified nucleotide sequence according to SEQ ID NO: 196;
(e) the second modified oligonucleotide comprises a modified nucleotide sequence according to SEQ ID NO: 293 and the first modified oligonucleotide comprises a modified nucleotide sequence according to SEQ ID NO: 197;
(f) the second modified oligonucleotide comprises a modified nucleotide sequence according to SEQ ID NO: 294 and the first modified oligonucleotide comprises a modified nucleotide sequence according to SEQ ID NO: 198;
(g) the second modified oligonucleotide comprises a modified nucleotide sequence according to SEQ ID NO: 295 and the first modified oligonucleotide comprises a modified nucleotide sequence according to SEQ ID NO: 199;
(h) the second modified oligonucleotide comprises a modified nucleotide sequence according to SEQ ID NO: 296 and the first modified oligonucleotide comprises a modified nucleotide sequence according to SEQ ID NO: 200;
(i) the second modified oligonucleotide comprises a modified nucleotide sequence according to SEQ ID NO: 297 and the first modified oligonucleotide comprises a modified nucleotide sequence according to SEQ ID NO: 201;
(j) the second modified oligonucleotide comprises a modified nucleotide sequence according to SEQ ID NO: 298 and the first modified oligonucleotide comprises a modified nucleotide sequence according to SEQ ID NO: 202;
(k) the second modified oligonucleotide comprises a modified nucleotide sequence according to SEQ ID NO: 299 and the first modified oligonucleotide comprises a modified nucleotide sequence according to SEQ ID NO: 203;
(l) the second modified oligonucleotide comprises a modified nucleotide sequence according to SEQ ID NO: 300 and the first modified oligonucleotide comprises a modified nucleotide sequence according to SEQ ID NO: 204;
(m) the second modified oligonucleotide comprises a modified nucleotide sequence according to SEQ ID NO: 301 and the first modified oligonucleotide comprises a modified nucleotide sequence according to SEQ ID NO: 205;
(n) the second modified oligonucleotide comprises a modified nucleotide sequence according to SEQ ID NO: 302 and the first modified oligonucleotide comprises a modified nucleotide sequence according to SEQ ID NO: 206;
(o) the second modified oligonucleotide comprises a modified nucleotide sequence according to SEQ ID NO: 303 and the first modified oligonucleotide comprises a modified nucleotide sequence according to SEQ ID NO: 207;
(p) the second modified oligonucleotide comprises a modified nucleotide sequence according to SEQ ID NO: 304 and the first modified oligonucleotide comprises a modified nucleotide sequence according to SEQ ID NO: 208;
(q) the second modified oligonucleotide comprises a modified nucleotide sequence according to SEQ ID NO: 305 and the first modified oligonucleotide comprises a modified nucleotide sequence according to SEQ ID NO: 209;
(r) the second modified oligonucleotide comprises a modified nucleotide sequence according to SEQ ID NO: 306 and the first modified oligonucleotide comprises a modified nucleotide sequence according to SEQ ID NO: 210;
(s) the second modified oligonucleotide comprises a modified nucleotide sequence according to SEQ ID NO: 307 and the first modified oligonucleotide comprises a modified nucleotide sequence according to SEQ ID NO: 211;
(t) the second modified oligonucleotide comprises a modified nucleotide sequence according to SEQ ID NO: 308 and the first modified oligonucleotide comprises a modified nucleotide sequence according to SEQ ID NO: 212;
(u) the second modified oligonucleotide comprises a modified nucleotide sequence according to SEQ ID NO: 309 and the first modified oligonucleotide comprises a modified nucleotide sequence according to SEQ ID NO: 213;
(v) the second modified oligonucleotide comprises a modified nucleotide sequence according to SEQ ID NO: 310 and the first modified oligonucleotide comprises a modified nucleotide sequence according to SEQ ID NO: 214;
(w) the second modified oligonucleotide comprises a modified nucleotide sequence according to SEQ ID NO: 311 and the first modified oligonucleotide comprises a modified nucleotide sequence according to SEQ ID NO: 215;
(x) the second modified oligonucleotide comprises a modified nucleotide sequence according to SEQ ID NO: 312 and the first modified oligonucleotide comprises a modified nucleotide sequence according to SEQ ID NO: 216;
(y) the second modified oligonucleotide comprises a modified nucleotide sequence according to SEQ ID NO: 313 and the first modified oligonucleotide comprises a modified nucleotide sequence according to SEQ ID NO: 217;
(z) the second modified oligonucleotide comprises a modified nucleotide sequence according to SEQ ID NO: 314 and the first modified oligonucleotide comprises a modified nucleotide sequence according to SEQ ID NO: 218;
(aa) the second modified oligonucleotide comprises a modified nucleotide sequence according to SEQ ID NO: 315 and the first modified oligonucleotide comprises a modified nucleotide sequence according to SEQ ID NO: 219;
(bb) the second modified oligonucleotide comprises a modified nucleotide sequence according to SEQ ID NO: 316 and the first modified oligonucleotide comprises a modified nucleotide sequence according to SEQ ID NO: 220;
(cc) the second modified oligonucleotide comprises a modified nucleotide sequence according to SEQ ID NO: 317 and the first modified oligonucleotide comprises a modified nucleotide sequence according to SEQ ID NO: 221;
(dd) the second modified oligonucleotide comprises a modified nucleotide sequence according to SEQ ID NO: 318 and the first modified oligonucleotide comprises a modified nucleotide sequence according to SEQ ID NO: 222;
(ee) the second modified oligonucleotide comprises a modified nucleotide sequence according to SEQ ID NO: 319 and the first modified oligonucleotide comprises a modified nucleotide sequence according to SEQ ID NO: 223;
(ff) the second modified oligonucleotide comprises a modified nucleotide sequence according to SEQ ID NO: 320 and the first modified oligonucleotide comprises a modified nucleotide sequence according to SEQ ID NO: 224;
(gg) the second modified oligonucleotide comprises a modified nucleotide sequence according to SEQ ID NO: 321 and the first modified oligonucleotide comprises a modified nucleotide sequence according to SEQ ID NO: 225;
(hh) the second modified oligonucleotide comprises a modified nucleotide sequence according to SEQ ID NO: 322 and the first modified oligonucleotide comprises a modified nucleotide sequence according to SEQ ID NO: 226;
(ii) the second modified oligonucleotide comprises a modified nucleotide sequence according to SEQ ID NO: 323 and the first modified oligonucleotide comprises a modified nucleotide sequence according to SEQ ID NO: 227;
(jj) the second modified oligonucleotide comprises a modified nucleotide sequence according to SEQ ID NO: 324 and the first modified oligonucleotide comprises a modified nucleotide sequence according to SEQ ID NO: 228;
(kk) the second modified oligonucleotide comprises a modified nucleotide sequence according to SEQ ID NO: 325 and the first modified oligonucleotide comprises a modified nucleotide sequence according to SEQ ID NO: 229;
(ll) the second modified oligonucleotide comprises a modified nucleotide sequence according to SEQ ID NO: 326 and the first modified oligonucleotide comprises a modified nucleotide sequence according to SEQ ID NO: 230;
(mm) the second modified oligonucleotide comprises a modified nucleotide sequence according to SEQ ID NO: 327 and the first modified oligonucleotide comprises a modified nucleotide sequence according to SEQ ID NO: 231;
(nn) the second modified oligonucleotide comprises a modified nucleotide sequence according to SEQ ID NO: 328 and the first modified oligonucleotide comprises a modified nucleotide sequence according to SEQ ID NO: 232;
(oo) the second modified oligonucleotide comprises a modified nucleotide sequence according to SEQ ID NO: 329 and the first modified oligonucleotide comprises a modified nucleotide sequence according to SEQ ID NO: 233;
(pp) the second modified oligonucleotide comprises a modified nucleotide sequence according to SEQ ID NO: 330 and the first modified oligonucleotide comprises a modified nucleotide sequence according to SEQ ID NO: 234;
(qq) the second modified oligonucleotide comprises a modified nucleotide sequence according to SEQ ID NO: 331 and the first modified oligonucleotide comprises a modified nucleotide sequence according to SEQ ID NO: 235;
(rr) the second modified oligonucleotide comprises a modified nucleotide sequence according to SEQ ID NO: 332 and the first modified oligonucleotide comprises a modified nucleotide sequence according to SEQ ID NO: 236;
(ss) the second modified oligonucleotide comprises a modified nucleotide sequence according to SEQ ID NO: 333 and the first modified oligonucleotide comprises a modified nucleotide sequence according to SEQ ID NO: 237;
(tt) the second modified oligonucleotide comprises a modified nucleotide sequence according to SEQ ID NO: 334 and the first modified oligonucleotide comprises a modified nucleotide sequence according to SEQ ID NO: 238;
(uu) the second modified oligonucleotide comprises a modified nucleotide sequence according to SEQ ID NO: 335 and the first modified oligonucleotide comprises a modified nucleotide sequence according to SEQ ID NO: 239;
(vv) the second modified oligonucleotide comprises a modified nucleotide sequence according to SEQ ID NO: 336 and the first modified oligonucleotide comprises a modified nucleotide sequence according to SEQ ID NO: 240;
(ww) the second modified oligonucleotide comprises a modified nucleotide sequence according to SEQ ID NO: 337 and the first modified oligonucleotide comprises a modified nucleotide sequence according to SEQ ID NO: 241;
(xx) the second modified oligonucleotide comprises a modified nucleotide sequence according to SEQ ID NO: 338 and the first modified oligonucleotide comprises a modified nucleotide sequence according to SEQ ID NO: 242;
(yy) the second modified oligonucleotide comprises a modified nucleotide sequence according to SEQ ID NO: 339 and the first modified oligonucleotide comprises a modified nucleotide sequence according to SEQ ID NO: 243;
(zz) the second modified oligonucleotide comprises a modified nucleotide sequence according to SEQ ID NO: 340 and the first modified oligonucleotide comprises a modified nucleotide sequence according to SEQ ID NO: 244;
(aaa) the second modified oligonucleotide comprises a modified nucleotide sequence according to SEQ ID NO: 341 and the first modified oligonucleotide comprises a modified nucleotide sequence according to SEQ ID NO: 245;
(bbb) the second modified oligonucleotide comprises a modified nucleotide sequence according to SEQ ID NO: 342 and the first modified oligonucleotide comprises a modified nucleotide sequence according to SEQ ID NO: 246;
(ccc) the second modified oligonucleotide comprises a modified nucleotide sequence according to SEQ ID NO: 343 and the first modified oligonucleotide comprises a modified nucleotide sequence according to SEQ ID NO: 247;
(ddd) the second modified oligonucleotide comprises a modified nucleotide sequence according to SEQ ID NO: 344 and the first modified oligonucleotide comprises a modified nucleotide sequence according to SEQ ID NO: 248;
(eee) the second modified oligonucleotide comprises a modified nucleotide sequence according to SEQ ID NO: 345 and the first modified oligonucleotide comprises a modified nucleotide sequence according to SEQ ID NO: 249;
(fff) the second modified oligonucleotide comprises a modified nucleotide sequence according to SEQ ID NO: 346 and the first modified oligonucleotide comprises a modified nucleotide sequence according to SEQ ID NO: 250;
(ggg) the second modified oligonucleotide comprises a modified nucleotide sequence according to SEQ ID NO: 347 and the first modified oligonucleotide comprises a modified nucleotide sequence according to SEQ ID NO: 251;
(hhh) the second modified oligonucleotide comprises a modified nucleotide sequence according to SEQ ID NO: 348 and the first modified oligonucleotide comprises a modified nucleotide sequence according to SEQ ID NO: 252;
(iii) the second modified oligonucleotide comprises a modified nucleotide sequence according to SEQ ID NO: 349 and the first modified oligonucleotide comprises a modified nucleotide sequence according to SEQ ID NO: 253;
(jjj) the second modified oligonucleotide comprises a modified nucleotide sequence according to SEQ ID NO: 350 and the first modified oligonucleotide comprises a modified nucleotide sequence according to SEQ ID NO: 254;
(kkk) the second modified oligonucleotide comprises a modified nucleotide sequence according to SEQ ID NO: 351 and the first modified oligonucleotide comprises a modified nucleotide sequence according to SEQ ID NO: 255;
(lll) the second modified oligonucleotide comprises a modified nucleotide sequence according to SEQ ID NO: 352 and the first modified oligonucleotide comprises a modified nucleotide sequence according to SEQ ID NO: 256;
(mmm) the second modified oligonucleotide comprises a modified nucleotide sequence according to SEQ ID NO: 353 and the first modified oligonucleotide comprises a modified nucleotide sequence according to SEQ ID NO: 257;
(nnn) the second modified oligonucleotide comprises a modified nucleotide sequence according to SEQ ID NO: 354 and the first modified oligonucleotide comprises a modified nucleotide sequence according to SEQ ID NO: 258;
(ooo) the second modified oligonucleotide comprises a modified nucleotide sequence according to SEQ ID NO: 355 and the first modified oligonucleotide comprises a modified nucleotide sequence according to SEQ ID NO: 259;
(ppp) the second modified oligonucleotide comprises a modified nucleotide sequence according to SEQ ID NO: 356 and the first modified oligonucleotide comprises a modified nucleotide sequence according to SEQ ID NO: 260;
(qqq) the second modified oligonucleotide comprises a modified nucleotide sequence according to SEQ ID NO: 357 and the first modified oligonucleotide comprises a modified nucleotide sequence according to SEQ ID NO: 261;
(rrr) the second modified oligonucleotide comprises a modified nucleotide sequence according to SEQ ID NO: 358 and the first modified oligonucleotide comprises a modified nucleotide sequence according to SEQ ID NO: 262;
(sss) the second modified oligonucleotide comprises a modified nucleotide sequence according to SEQ ID NO: 359 and the first modified oligonucleotide comprises a modified nucleotide sequence according to SEQ ID NO: 263;
(ttt) the second modified oligonucleotide comprises a modified nucleotide sequence according to SEQ ID NO: 360 and the first modified oligonucleotide comprises a modified nucleotide sequence according to SEQ ID NO: 264;
(uuu) the second modified oligonucleotide comprises a modified nucleotide sequence according to SEQ ID NO: 361 and the first modified oligonucleotide comprises a modified nucleotide sequence according to SEQ ID NO: 265;
(vvv) the second modified oligonucleotide comprises a modified nucleotide sequence according to SEQ ID NO: 362 and the first modified oligonucleotide comprises a modified nucleotide sequence according to SEQ ID NO: 266;
(www) the second modified oligonucleotide comprises a modified nucleotide sequence according to SEQ ID NO: 363 and the first modified oligonucleotide comprises a modified nucleotide sequence according to SEQ ID NO: 267;
(xxx) the second modified oligonucleotide comprises a modified nucleotide sequence according to SEQ ID NO: 364 and the first modified oligonucleotide comprises a modified nucleotide sequence according to SEQ ID NO: 268;
(yyy) the second modified oligonucleotide comprises a modified nucleotide sequence according to SEQ ID NO: 365 and the first modified oligonucleotide comprises a modified nucleotide sequence according to SEQ ID NO: 269;
(zzz) the second modified oligonucleotide comprises a modified nucleotide sequence according to SEQ ID NO: 366 and the first modified oligonucleotide comprises a modified nucleotide sequence according to SEQ ID NO: 270;
(aaaa) the second modified oligonucleotide comprises a modified nucleotide sequence according to SEQ ID NO: 367 and the first modified oligonucleotide comprises a modified nucleotide sequence according to SEQ ID NO: 271;
(bbbb) the second modified oligonucleotide comprises a modified nucleotide sequence according to SEQ ID NO: 368 and the first modified oligonucleotide comprises a modified nucleotide sequence according to SEQ ID NO: 272;
(cccc) the second modified oligonucleotide comprises a modified nucleotide sequence according to SEQ ID NO: 369 and the first modified oligonucleotide comprises a modified nucleotide sequence according to SEQ ID NO: 273;
(dddd) the second modified oligonucleotide comprises a modified nucleotide sequence according to SEQ ID NO: 370 and the first modified oligonucleotide comprises a modified nucleotide sequence according to SEQ ID NO: 274;
(eeee) the second modified oligonucleotide comprises a modified nucleotide sequence according to SEQ ID NO: 371 and the first modified oligonucleotide comprises a modified nucleotide sequence according to SEQ ID NO: 275;
(ffff) the second modified oligonucleotide comprises a modified nucleotide sequence according to SEQ ID NO: 372 and the first modified oligonucleotide comprises a modified nucleotide sequence according to SEQ ID NO: 276;
(gggg) the second modified oligonucleotide comprises a modified nucleotide sequence according to SEQ ID NO: 373 and the first modified oligonucleotide comprises a modified nucleotide sequence according to SEQ ID NO: 277;
(hhhh) the second modified oligonucleotide comprises a modified nucleotide sequence according to SEQ ID NO: 374 and the first modified oligonucleotide comprises a modified nucleotide sequence according to SEQ ID NO: 278;
(iiii) the second modified oligonucleotide comprises a modified nucleotide sequence according to SEQ ID NO: 375 and the first modified oligonucleotide comprises a modified nucleotide sequence according to SEQ ID NO: 279;
(jjjj) the second modified oligonucleotide comprises a modified nucleotide sequence according to SEQ ID NO: 376 and the first modified oligonucleotide comprises a modified nucleotide sequence according to SEQ ID NO: 280;
(kkkk) the second modified oligonucleotide comprises a modified nucleotide sequence according to SEQ ID NO: 377 and the first modified oligonucleotide comprises a modified nucleotide sequence according to SEQ ID NO: 281;
(llll) the second modified oligonucleotide comprises a modified nucleotide sequence according to SEQ ID NO: 378 and the first modified oligonucleotide comprises a modified nucleotide sequence according to SEQ ID NO: 282;
(mmmm) the second modified oligonucleotide comprises a modified nucleotide sequence according to SEQ ID NO: 379 and the first modified oligonucleotide comprises a modified nucleotide sequence according to SEQ ID NO: 283;
(nnnn) the second modified oligonucleotide comprises a modified nucleotide sequence according to SEQ ID NO: 380 and the first modified oligonucleotide comprises a modified nucleotide sequence according to SEQ ID NO: 284;
(oooo) the second modified oligonucleotide comprises a modified nucleotide sequence according to SEQ ID NO: 381 and the first modified oligonucleotide comprises a modified nucleotide sequence according to SEQ ID NO: 285;
(pppp) the second modified oligonucleotide comprises a modified nucleotide sequence according to SEQ ID NO: 382 and the first modified oligonucleotide comprises a modified nucleotide sequence according to SEQ ID NO: 286;
(qqqq) the second modified oligonucleotide comprises a modified nucleotide sequence according to SEQ ID NO: 383 and the first modified oligonucleotide comprises a modified nucleotide sequence according to SEQ ID NO: 287;
(rrrr) the second modified oligonucleotide comprises a modified nucleotide sequence according to SEQ ID NO: 384 and the first modified oligonucleotide comprises a modified nucleotide sequence according to SEQ ID NO: 288
(ssss) the sense strand comprises a modified nucleotide sequence according to SEQ ID NO: 385 and the antisense strand comprises a modified nucleotide sequence according to SEQ ID NO: 193;
(tttt) the sense strand comprises a modified nucleotide sequence according to SEQ ID NO: 386 and the antisense strand comprises a modified nucleotide sequence according to SEQ ID NO: 194;
(uuuu) the sense strand comprises a modified nucleotide sequence according to SEQ ID NO: 387 and the antisense strand comprises a modified nucleotide sequence according to SEQ ID NO: 195;
(vvvv) the sense strand comprises a modified nucleotide sequence according to SEQ ID NO: 388 and the antisense strand comprises a modified nucleotide sequence according to SEQ ID NO: 196;
(wwww) the sense strand comprises a modified nucleotide sequence according to SEQ ID NO: 389 and the antisense strand comprises a modified nucleotide sequence according to SEQ ID NO: 197;
(xxxx) the sense strand comprises a modified nucleotide sequence according to SEQ ID NO: 390 and the antisense strand comprises a modified nucleotide sequence according to SEQ ID NO: 198;
(yyyy) the sense strand comprises a modified nucleotide sequence according to SEQ ID NO: 391 and the antisense strand comprises a modified nucleotide sequence according to SEQ ID NO: 199;
(zzzz) the sense strand comprises a modified nucleotide sequence according to SEQ ID NO: 392 and the antisense strand comprises a modified nucleotide sequence according to SEQ ID NO: 200;
(aaaaa) the sense strand comprises a modified nucleotide sequence according to SEQ ID NO: 393 and the antisense strand comprises a modified nucleotide sequence according to SEQ ID NO: 201;
(bbbbb) the sense strand comprises a modified nucleotide sequence according to SEQ ID NO: 394 and the antisense strand comprises a modified nucleotide sequence according to SEQ ID NO: 202;
(ccccc) the sense strand comprises a modified nucleotide sequence according to SEQ ID NO: 395 and the antisense strand comprises a modified nucleotide sequence according to SEQ ID NO: 203;
(ddddd) the sense strand comprises a modified nucleotide sequence according to SEQ ID NO: 396 and the antisense strand comprises a modified nucleotide sequence according to SEQ ID NO: 204;
(eeeee) the sense strand comprises a modified nucleotide sequence according to SEQ ID NO: 397 and the antisense strand comprises a modified nucleotide sequence according to SEQ ID NO: 205;
(fffff) the sense strand comprises a modified nucleotide sequence according to SEQ ID NO: 398 and the antisense strand comprises a modified nucleotide sequence according to SEQ ID NO: 206;
(ggggg) the sense strand comprises a modified nucleotide sequence according to SEQ ID NO: 399 and the antisense strand comprises a modified nucleotide sequence according to SEQ ID NO: 207;
(hhhhh) the sense strand comprises a modified nucleotide sequence according to SEQ ID NO: 400 and the antisense strand comprises a modified nucleotide sequence according to SEQ ID NO: 208;
(iiiii) the sense strand comprises a modified nucleotide sequence according to SEQ ID NO: 401 and the antisense strand comprises a modified nucleotide sequence according to SEQ ID NO: 209;
(jjjjj) the sense strand comprises a modified nucleotide sequence according to SEQ ID NO: 402 and the antisense strand comprises a modified nucleotide sequence according to SEQ ID NO: 210;
(kkkkk) the sense strand comprises a modified nucleotide sequence according to SEQ ID NO: 403 and the antisense strand comprises a modified nucleotide sequence according to SEQ ID NO: 211;
(lllll) the sense strand comprises a modified nucleotide sequence according to SEQ ID NO: 404 and the antisense strand comprises a modified nucleotide sequence according to SEQ ID NO: 212;
(mmmmm) the sense strand comprises a modified nucleotide sequence according to SEQ ID NO: 405 and the antisense strand comprises a modified nucleotide sequence according to SEQ ID NO: 213;
(nnnnn) the sense strand comprises a modified nucleotide sequence according to SEQ ID NO: 406 and the antisense strand comprises a modified nucleotide sequence according to SEQ ID NO: 214;
(ooooo) the sense strand comprises a modified nucleotide sequence according to SEQ ID NO: 407 and the antisense strand comprises a modified nucleotide sequence according to SEQ ID NO: 215;
(ppppp) the sense strand comprises a modified nucleotide sequence according to SEQ ID NO: 408 and the antisense strand comprises a modified nucleotide sequence according to SEQ ID NO: 216;
(qqqqq) the sense strand comprises a modified nucleotide sequence according to SEQ ID NO: 409 and the antisense strand comprises a modified nucleotide sequence according to SEQ ID NO: 217;
(rrrrr) the sense strand comprises a modified nucleotide sequence according to SEQ ID NO: 410 and the antisense strand comprises a modified nucleotide sequence according to SEQ ID NO: 218;
(sssss) the sense strand comprises a modified nucleotide sequence according to SEQ ID NO: 411 and the antisense strand comprises a modified nucleotide sequence according to SEQ ID NO: 219;
(ttttt) the sense strand comprises a modified nucleotide sequence according to SEQ ID NO: 412 and the antisense strand comprises a modified nucleotide sequence according to SEQ ID NO: 220;
(uuuuu) the sense strand comprises a modified nucleotide sequence according to SEQ ID NO: 413 and the antisense strand comprises a modified nucleotide sequence according to SEQ ID NO: 221;
(vvvvv) the sense strand comprises a modified nucleotide sequence according to SEQ ID NO: 414 and the antisense strand comprises a modified nucleotide sequence according to SEQ ID NO: 222;
(wwwww) the sense strand comprises a modified nucleotide sequence according to SEQ ID NO: 415 and the antisense strand comprises a modified nucleotide sequence according to SEQ ID NO: 223;
(xxxxx) the sense strand comprises a modified nucleotide sequence according to SEQ ID NO: 416 and the antisense strand comprises a modified nucleotide sequence according to SEQ ID NO: 224;
(yyyyy) the sense strand comprises a modified nucleotide sequence according to SEQ ID NO: 417 and the antisense strand comprises a modified nucleotide sequence according to SEQ ID NO: 225;
(zzzzz) the sense strand comprises a modified nucleotide sequence according to SEQ ID NO: 418 and the antisense strand comprises a modified nucleotide sequence according to SEQ ID NO: 226;
(aaaaaa) the sense strand comprises a modified nucleotide sequence according to SEQ ID NO: 419 and the antisense strand comprises a modified nucleotide sequence according to SEQ ID NO: 227;
(bbbbbb) the sense strand comprises a modified nucleotide sequence according to SEQ ID NO: 420 and the antisense strand comprises a modified nucleotide sequence according to SEQ ID NO: 228;
(cccccc) the sense strand comprises a modified nucleotide sequence according to SEQ ID NO: 421 and the antisense strand comprises a modified nucleotide sequence according to SEQ ID NO: 229;
(dddddd) the sense strand comprises a modified nucleotide sequence according to SEQ ID NO: 422 and the antisense strand comprises a modified nucleotide sequence according to SEQ ID NO: 230;
(eeeeee) the sense strand comprises a modified nucleotide sequence according to SEQ ID NO: 423 and the antisense strand comprises a modified nucleotide sequence according to SEQ ID NO: 231;
(ffffff) the sense strand comprises a modified nucleotide sequence according to SEQ ID NO: 424 and the antisense strand comprises a modified nucleotide sequence according to SEQ ID NO: 232;
(gggggg) the sense strand comprises a modified nucleotide sequence according to SEQ ID NO: 425 and the antisense strand comprises a modified nucleotide sequence according to SEQ ID NO: 233;
(hhhhhh) the sense strand comprises a modified nucleotide sequence according to SEQ ID NO: 426 and the antisense strand comprises a modified nucleotide sequence according to SEQ ID NO: 234;
(iiiiii) the sense strand comprises a modified nucleotide sequence according to SEQ ID NO: 427 and the antisense strand comprises a modified nucleotide sequence according to SEQ ID NO: 235;
(jjjjjj) the sense strand comprises a modified nucleotide sequence according to SEQ ID NO: 428 and the antisense strand comprises a modified nucleotide sequence according to SEQ ID NO: 236;
(kkkkkk) the sense strand comprises a modified nucleotide sequence according to SEQ ID NO: 429 and the antisense strand comprises a modified nucleotide sequence according to SEQ ID NO: 237;
(llllll) the sense strand comprises a modified nucleotide sequence according to SEQ ID NO: 430 and the antisense strand comprises a modified nucleotide sequence according to SEQ ID NO: 238;
(mmmmmm) the sense strand comprises a modified nucleotide sequence according to SEQ ID NO: 431 and the antisense strand comprises a modified nucleotide sequence according to SEQ ID NO: 239;
(nnnnnn) the sense strand comprises a modified nucleotide sequence according to SEQ ID NO: 432 and the antisense strand comprises a modified nucleotide sequence according to SEQ ID NO: 240;
(oooooo) the sense strand comprises a modified nucleotide sequence according to SEQ ID NO: 433 and the antisense strand comprises a modified nucleotide sequence according to SEQ ID NO: 241;
(pppppp) the sense strand comprises a modified nucleotide sequence according to SEQ ID NO: 434 and the antisense strand comprises a modified nucleotide sequence according to SEQ ID NO: 242;
(qqqqqq) the sense strand comprises a modified nucleotide sequence according to SEQ ID NO: 435 and the antisense strand comprises a modified nucleotide sequence according to SEQ ID NO: 243;
(rrrrrr) the sense strand comprises a modified nucleotide sequence according to SEQ ID NO: 436 and the antisense strand comprises a modified nucleotide sequence according to SEQ ID NO: 244;
(ssssss) the sense strand comprises a modified nucleotide sequence according to SEQ ID NO: 437 and the antisense strand comprises a modified nucleotide sequence according to SEQ ID NO: 245;
(tttttt) the sense strand comprises a modified nucleotide sequence according to SEQ ID NO: 438 and the antisense strand comprises a modified nucleotide sequence according to SEQ ID NO: 246;
(uuuuuu) the sense strand comprises a modified nucleotide sequence according to SEQ ID NO: 439 and the antisense strand comprises a modified nucleotide sequence according to SEQ ID NO: 247;
(vvvvvv) the sense strand comprises a modified nucleotide sequence according to SEQ ID NO: 440 and the antisense strand comprises a modified nucleotide sequence according to SEQ ID NO: 248;
(wwwwww) the sense strand comprises a modified nucleotide sequence according to SEQ ID NO: 441 and the antisense strand comprises a modified nucleotide sequence according to SEQ ID NO: 249;
(xxxxxx) the sense strand comprises a modified nucleotide sequence according to SEQ ID NO: 442 and the antisense strand comprises a modified nucleotide sequence according to SEQ ID NO: 250;
(yyyyyy) the sense strand comprises a modified nucleotide sequence according to SEQ ID NO: 443 and the antisense strand comprises a modified nucleotide sequence according to SEQ ID NO: 251;
(zzzzzz) the sense strand comprises a modified nucleotide sequence according to SEQ ID NO: 444 and the antisense strand comprises a modified nucleotide sequence according to SEQ ID NO: 252;
(aaaaaaa) the sense strand comprises a modified nucleotide sequence according to SEQ ID NO: 445 and the antisense strand comprises a modified nucleotide sequence according to SEQ ID NO: 253;
(bbbbbbb) the sense strand comprises a modified nucleotide sequence according to SEQ ID NO: 446 and the antisense strand comprises a modified nucleotide sequence according to SEQ ID NO: 254;
(ccccccc) the sense strand comprises a modified nucleotide sequence according to SEQ ID NO: 447 and the antisense strand comprises a modified nucleotide sequence according to SEQ ID NO: 255;
(ddddddd) the sense strand comprises a modified nucleotide sequence according to SEQ ID NO: 448 and the antisense strand comprises a modified nucleotide sequence according to SEQ ID NO: 256;
(eeeeeee) the sense strand comprises a modified nucleotide sequence according to SEQ ID NO: 449 and the antisense strand comprises a modified nucleotide sequence according to SEQ ID NO: 257;
(fffffff) the sense strand comprises a modified nucleotide sequence according to SEQ ID NO: 450 and the antisense strand comprises a modified nucleotide sequence according to SEQ ID NO: 258;
(ggggggg) the sense strand comprises a modified nucleotide sequence according to SEQ ID NO: 451 and the antisense strand comprises a modified nucleotide sequence according to SEQ ID NO: 259;
(hhhhhhh) the sense strand comprises a modified nucleotide sequence according to SEQ ID NO: 452 and the antisense strand comprises a modified nucleotide sequence according to SEQ ID NO: 260;
(iiiiii) the sense strand comprises a modified nucleotide sequence according to SEQ ID NO: 453 and the antisense strand comprises a modified nucleotide sequence according to SEQ ID NO: 261;
(jjjjjjj) the sense strand comprises a modified nucleotide sequence according to SEQ ID NO: 454 and the antisense strand comprises a modified nucleotide sequence according to SEQ ID NO: 262;
(kkkkkkk) the sense strand comprises a modified nucleotide sequence according to SEQ ID NO: 455 and the antisense strand comprises a modified nucleotide sequence according to SEQ ID NO: 263;
(lllllll) the sense strand comprises a modified nucleotide sequence according to SEQ ID NO: 456 and the antisense strand comprises a modified nucleotide sequence according to SEQ ID NO: 264;
(mmmmmmm) the sense strand comprises a modified nucleotide sequence according to SEQ ID NO: 457 and the antisense strand comprises a modified nucleotide sequence according to SEQ ID NO: 265;
(nnnnnnn) the sense strand comprises a modified nucleotide sequence according to SEQ ID NO: 458 and the antisense strand comprises a modified nucleotide sequence according to SEQ ID NO: 266;
(ooooooo) the sense strand comprises a modified nucleotide sequence according to SEQ ID NO: 459 and the antisense strand comprises a modified nucleotide sequence according to SEQ ID NO: 267;
(ppppppp) the sense strand comprises a modified nucleotide sequence according to SEQ ID NO: 460 and the antisense strand comprises a modified nucleotide sequence according to SEQ ID NO: 268;
(qqqqqqq) the sense strand comprises a modified nucleotide sequence according to SEQ ID NO: 461 and the antisense strand comprises a modified nucleotide sequence according to SEQ ID NO: 269;
(rrrrrrrr) the sense strand comprises a modified nucleotide sequence according to SEQ ID NO: 462 and the antisense strand comprises a modified nucleotide sequence according to SEQ ID NO: 270;
(sssssss) the sense strand comprises a modified nucleotide sequence according to SEQ ID NO: 463 and the antisense strand comprises a modified nucleotide sequence according to SEQ ID NO: 271;
(ttttttt) the sense strand comprises a modified nucleotide sequence according to SEQ ID NO: 464 and the antisense strand comprises a modified nucleotide sequence according to SEQ ID NO: 272;
(uuuuuuu) the sense strand comprises a modified nucleotide sequence according to SEQ ID NO: 465 and the antisense strand comprises a modified nucleotide sequence according to SEQ ID NO: 273;
(vvvvvvv) the sense strand comprises a modified nucleotide sequence according to SEQ ID NO: 466 and the antisense strand comprises a modified nucleotide sequence according to SEQ ID NO: 274;
(wwwwwww) the sense strand comprises a modified nucleotide sequence according to SEQ ID NO: 467 and the antisense strand comprises a modified nucleotide sequence according to SEQ ID NO: 275;
(xxxxxxx) the sense strand comprises a modified nucleotide sequence according to SEQ ID NO: 468 and the antisense strand comprises a modified nucleotide sequence according to SEQ ID NO: 276;
(yyyyyyy) the sense strand comprises a modified nucleotide sequence according to SEQ ID NO: 469 and the antisense strand comprises a modified nucleotide sequence according to SEQ ID NO: 277;
(zzzzzzz) the sense strand comprises a modified nucleotide sequence according to SEQ ID NO: 470 and the antisense strand comprises a modified nucleotide sequence according to SEQ ID NO: 278;
(aaaaaaaa) the sense strand comprises a modified nucleotide sequence according to SEQ ID NO: 471 and the antisense strand comprises a modified nucleotide sequence according to SEQ ID NO: 279;
(bbbbbbbb) the sense strand comprises a modified nucleotide sequence according to SEQ ID NO: 472 and the antisense strand comprises a modified nucleotide sequence according to SEQ ID NO: 280;
(cccccccc) the sense strand comprises a modified nucleotide sequence according to SEQ ID NO: 473 and the antisense strand comprises a modified nucleotide sequence according to SEQ ID NO: 281;
(dddddddd) the sense strand comprises a modified nucleotide sequence according to SEQ ID NO: 474 and the antisense strand comprises a modified nucleotide sequence according to SEQ ID NO: 282;
(eeeeeeee) the sense strand comprises a modified nucleotide sequence according to SEQ ID NO: 475 and the antisense strand comprises a modified nucleotide sequence according to SEQ ID NO: 283;
(ffffffff) the sense strand comprises a modified nucleotide sequence according to SEQ ID NO: 476 and the antisense strand comprises a modified nucleotide sequence according to SEQ ID NO: 284;
(gggggggg) the sense strand comprises a modified nucleotide sequence according to SEQ ID NO: 477 and the antisense strand comprises a modified nucleotide sequence according to SEQ ID NO: 285;
(hhhhhhhh) the sense strand comprises a modified nucleotide sequence according to SEQ ID NO: 478 and the antisense strand comprises a modified nucleotide sequence according to SEQ ID NO: 286;
(iiiiiii) the sense strand comprises a modified nucleotide sequence according to SEQ ID NO: 479 and the antisense strand comprises a modified nucleotide sequence according to SEQ ID NO: 287; or
(jjjjjjjj) the sense strand comprises a modified nucleotide sequence according to SEQ ID NO: 480 and the antisense strand comprises a modified nucleotide sequence according to SEQ ID NO: 288.

In some implementations, the first modified oligonucleotide and/or second modified oligonucleotide comprises a targeting ligand. In some implementations, the first modified oligonucleotide comprises a targeting ligand conjugated to its 3′ end. In some implementations, the first modified oligonucleotide comprises a targeting ligand conjugated to its 5′ end. In some implementations, the second modified oligonucleotide comprises a targeting ligand conjugated to its 3′ end. In some implementations, the second modified oligonucleotide comprises a targeting ligand conjugated to its 5′ end.

In some implementations, the targeting ligand targets a hepatocyte, and optionally wherein the targeting ligand comprises a galactose trimer. In some implementations, the targeting ligand comprises N-acetylgalactosamine (GalNAc). In some implementations, the targeting ligand comprises a structure according to Formula I:

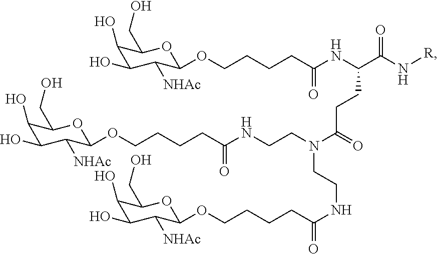

wherein R is:
(i) the first modified oligonucleotide or the second modified oligonucleotide; or
(ii) a linker conjugated to the first modified oligonucleotide or the second modified oligonucleotide.

In some implementations, R is a linker conjugated to the first modified oligonucleotide or the second modified oligonucleotide having a structure according to Formula III:

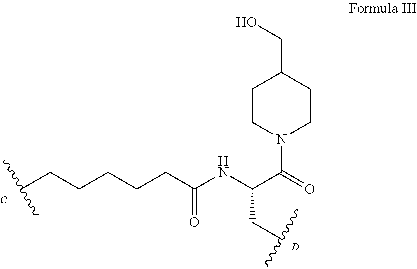

wherein:
(i) connection point C of Formula III is conjugated to Formula I, and connection point D of Formula III is conjugated to the first modified oligonucleotide or the second modified oligonucleotide; or
(ii) connection point C of Formula III is conjugated to Formula I, and connection point D of Formula III is conjugated to a phosphate group which is conjugated to the first modified oligonucleotide or the second modified oligonucleotide.

In some implementations, Formula III or the phosphate group is conjugated to the 3′ end or the 5′ end of the first modified oligonucleotide or the second modified oligonucleotide. In some implementations, Formula III or the phosphate group is conjugated to an internal nucleotide or nucleoside base of the first modified oligonucleotide or the second modified oligonucleotide. In some implementations, the first modified oligonucleotide or the second modified oligonucleotide comprises a targeting ligand and linker according to Formula V:

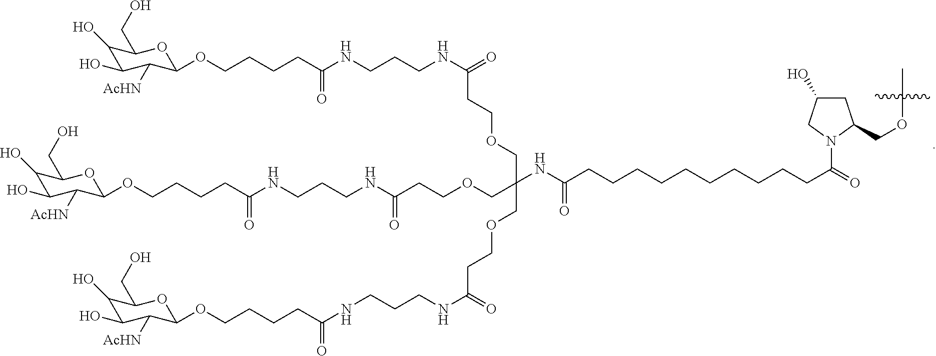 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some implementations, the oligomeric duplex is an siRNA molecule. In some implementations, the oligomeric duplex is an shRNA molecule.

Described herein is a pharmaceutical composition comprising the RNAi agent described herein, the modified oligonucleotide described herein, or the oligomeric duplex of described herein, and a pharmaceutically acceptable carrier.

Described herein is a lipid nanoparticle comprising the RNAi agent described herein, the modified oligonucleotide described herein, the oligomeric duplex described herein, or the pharmaceutical composition described herein.

Described herein is a method of making a pharmaceutical composition, comprising combining the RNAi agent described herein, the modified oligonucleotide described herein, or the oligomeric duplex described herein, with a delivery vehicle selected from the group consisting of a phosphate-buffered saline (PBS), lipid, a nanoparticle, a polymer, a liposome, a micelle, a dynamic polyconjugate (DPC), and antibody-oligo conjugate.

Described herein is a method of treating metabolic dysfunction-associated steatotic liver disease (MASLD) or metabolic dysfunction-associated steatotic liver (MASL) in a subject, comprising: administering to the subject an effective amount of a liver-targeted modified oligonucleotide, a liver-targeted oligomeric duplex, or a liver-targeted RNAi agent that inhibits expression of glucokinase (GCK).

Described herein is a use of a liver-targeted modified oligonucleotide, a liver-targeted oligomeric duplex, or a liver-targeted RNAi agent that inhibits expression of glucokinase (GCK) for the manufacture of a medicament for the treatment of metabolic dysfunction-associated steatotic liver disease (MASLD) or metabolic dysfunction-associated steatotic liver (MASL) in a subject.

Described herein is a method of inhibiting expression of glucokinase (GCK) in a subject, comprising: administering to the subject an effective amount of a liver-targeted modified oligonucleotide, a liver-targeted oligomeric duplex, or a liver-targeted RNAi agent that inhibits expression of GCK.

Described herein is a use of a liver-targeted modified oligonucleotide, a liver-targeted oligomeric duplex, or a liver-targeted RNAi agent in the manufacture of a medicament for inhibiting expression of glucokinase (GCK) in a hepatocyte of a subject.

Described herein is a method of controlling or reducing liver fat accumulation in a subject, comprising: administering to the subject an effective amount of a liver-targeted modified oligonucleotide, a liver-targeted oligomeric duplex, or a liver-targeted RNAi agent that inhibits expression of glucokinase (GCK).

Described herein is a use of a liver-targeted modified oligonucleotide, a liver-targeted oligomeric duplex, or a liver-targeted RNAi agent that inhibits expression of glucokinase (GCK) in the manufacture of a medicament for controlling or reducing liver fat accumulation in a subject.

In some implementations, the method treats metabolic dysfunction-associated steatotic liver disease (MASLD) or metabolic dysfunction-associated steatohepatitis (MASH) in the subject. In some implementations, the method treats nonalcoholic fatty liver disease (NAFLD) or nonalcoholic steatohepatitis (NASH) in the subject. In some implementations, the liver-targeted modified oligonucleotide is the modified oligonucleotide described herein. In some implementations, the liver-targeted oligomeric duplex is the oligomeric duplex described herein.

In some implementations, the RNAi agent comprises an antisense strand and a sense strand that at least partially complements the antisense strand, wherein: (a) the antisense strand comprises at least 14 contiguous bases from any one of SEQ ID NOS: 1-96; and/or (b) the sense strand comprises at least 14 contiguous bases from any one of SEQ ID NOS: 97-192. In some implementations, one or more bases of the antisense stand and/or the sense strand are modified.

In some implementations, (a) the antisense strand comprises at least 14 contiguous bases from any one of SEQ ID NOS: 193-288; and/or (b) the sense strand comprises at least 14 contiguous bases from any one of SEQ ID NOS: 289-384 and 385-480. In some implementations, the RNAi agent comprises an antisense strand and a sense strand that at least partially complements the antisense strand, and a targeting ligand conjugated to the antisense strand or the sense strand, wherein the targeting ligand targets a hepatocyte.

In some implementations, the antisense strand comprises at least 14 contiguous bases that complement a sequence in an mRNA molecule that encodes GCK (SEQ ID NO: 481). In some implementations, the antisense strand and the sense strand form a duplex of at least 14 bases in length. In some implementations, the antisense strand and the sense strand form a duplex of between 15 and 30 bases in length. In some implementations, the antisense strand or a subsequence thereof differs by 0, 1, 2, 3, or 4 nucleotides from an equal length portion of any one of SEQ ID NOS: 1-96 and 193-288, wherein the subsequence is at least 14, 15, 16, 17, 18, 19, 20, 21, 22, or 23 bases in length. In some implementations, the antisense strand comprises, in order, nucleotides 1-23 of any one of SEQ ID NOS: 1-96 and 193-288.

In some implementations, the antisense strand comprises at least 14 contiguous bases from any one of SEQ ID NOS: 1-96 and 193-288. In some implementations, the antisense strand comprises, in order, nucleobases 1-14 of any one of SEQ ID NOS: 1-96 and 193-288. In some implementations, the antisense strand comprises at least 14 contiguous bases from any one of SEQ ID NOS: 8, 11, 14, 16, 35, 36, 38, 39, 41, 42, 52, 77, 84, 86, 87, 88, 89, 90, 93, 200, 203, 206, 208, 227, 228, 230, 231, 233, 234, 244, 269, 276, 278, 279, 280, 281, 282, and 285. In some implementations, the sense strand or a subsequence thereof differs by 0, 1, 2, 3, or 4 nucleotides from an equal length portion of any one of SEQ ID NOS: 97-192, 289-384, and 385-480, wherein the subsequence is at least 14, 15, 16, 17, 18, 19, 20, or 21 bases in length. In some implementations, the sense strand comprises, in order, nucleotides 1-21 of any one of SEQ ID NOS: 97-192, 289-384, and 385-480.

In some implementations, the sense strand comprises at least 14 contiguous bases from any one of SEQ ID NOS: 97-192, 289-384, and 385-480. In some implementations, the sense strand comprises, in order, nucleobases 1-14 of any one of SEQ ID NOS: 97-192, 289-384, and 385-480. In some implementations, the sense strand comprises at least 14 contiguous bases from any one of SEQ ID NOS: 104, 107, 110, 112, 131, 132, 134, 135, 137, 138, 148, 173, 180, 182, 183, 184, 185, 186, 189, 296, 299, 302, 304, 323, 324, 326, 327, 329, 330, 340, 365, 372, 374, 375, 376, 377, 378, 381, 392, 395, 398, 400, 419, 420, 422, 423, 425 426, 436, 461, 468, 470, 471, 472, 473, 474, and 477.

In some implementations, the antisense strand comprises at least 16 contiguous bases that complement a sequence in an mRNA molecule that encodes GCK (SEQ ID NO: 481). In some implementations, the antisense strand and the sense strand form a duplex of at least 16 bases in length. In some implementations, the antisense strand comprises at least 16 contiguous bases from any one of SEQ ID NOS: 1-96 and 193-288. In some implementations, the antisense strand comprises at least 16 contiguous bases from any one of SEQ ID NOS: 8, 11, 14, 16, 35, 36, 38, 39, 41, 42, 52, 77, 84, 86, 87, 88, 89, 90, 93, 200, 203, 206, 208, 227, 228, 230, 231, 233, 234, 244, 269, 276, 278, 279, 280, 281, 282, and 285.

In some implementations, the antisense strand comprises, in order, nucleobases 1-16, 1-17, 1-18, 1-19, 1-20, 1-21, 1-22, 1-23, 2-17, 2-18, 2-19, 2-20, 2-21, 2-22, 2-23, 3-18, 3-19, 3-20, 3-21, 3-22, 3-23, 4-19, 4-20, 4-21, 4-22, 4-23, 5-21, 5-22, 5-23, 6-20, 6-21, 6-22, 6-23, 7-22, 7-23, or 8-23 of any one of SEQ ID NOS: 1-96 and 193-288. In some implementations, the antisense strand comprises a nucleotide sequence according to any one of SEQ ID NOS: 1-96. In some implementations, one or more nucleotides of the antisense strand are modified.

In some implementations, the antisense strand comprises a nucleotide sequence according to any one of SEQ ID NO S: 8, 11, 14, 16, 35, 36, 38, 39, 41, 42, 52, 77, 84, 86, 87, 88, 89, 90, 93. In some implementations, one or more nucleotides of the antisense strand are modified. In some implementations, the antisense strand has a modification pattern according to nsNfsnnnNfnnnnnnnNfnNfnnnnnsnsn. In some implementations, the antisense strand comprises a modified nucleotide sequence according to any one of SEQ ID NOS: 193-288. In some implementations, the antisense strand comprises a modified nucleotide sequence according to any one of SEQ ID NOS: 200, 203, 206, 208, 227, 228, 230, 231, 233, 234, 244, 269, 276, 278, 279, 280, 281, 282, and 285.

In some implementations, the sense strand comprises at least 16 contiguous bases from any one of SEQ ID NOS: 97-192, 289-384, and 385-480. In some implementations, the sense strand comprises at least 16 contiguous bases from any one of SEQ ID NOS: 104, 107, 110, 112, 131, 132, 134, 135, 137, 138, 148, 173, 180, 182, 183, 184, 185, 186, 189, 296, 299, 302, 304, 323, 324, 326, 327, 329, 330, 340, 365, 372, 374, 375, 376, 377, 378, 381, 392, 395, 398, 400, 419, 420, 422, 423, 425 426, 436, 461, 468, 470, 471, 472, 473, 474, and 477.

In some implementations, the sense strand comprises, in order, nucleobases 1-16, 1-17, 1-18, 1-19, 1-20, 1-21, 1-22, 1-23, 2-17, 2-18, 2-19, 2-20, 2-21, 2-22, 2-23, 3-18, 3-19, 3-20, 3-21, 3-22, 3-23, 4-19, 4-20, 4-21, 4-22, 4-23, 5-21, 5-22, 5-23, 6-20, 6-21, 6-22, 6-23, 7-22, 7-23, or 8-23 of any one of SEQ ID NOS: 97-192, 289-384, and 385-480. In some implementations, the sense strand comprises a nucleobase sequence according to any one of SEQ ID NOS: 97-192. In some implementations, one or more nucleotides of the sense strand are modified. In some implementations, the sense strand comprises a nucleobase sequence according to any one of SEQ ID NOS: 104, 107, 110, 112, 131, 132, 134, 135, 137, 138, 148, 173, 180, 182, 183, 184, 185, 186, and 189. In some implementations, one or more nucleotides of the sense strand are modified. In some implementations, the sense strand has a modification pattern according to nsnsnnnnNfnNfNfNfnnnnnnnnsnsn. In some implementations, the sense strand has a modification pattern according to nsnsnnnnNfnNfNfNfnnnnnnnnnn.

In some implementations, the sense strand comprises a modified nucleotide sequence according to any one of SEQ ID NOS: 289-384 and 385-480. In some implementations, the sense strand comprises a modified nucleotide sequence according to any one of SEQ ID NOS: 296, 299, 302, 304, 323, 324, 326, 327, 329, 330, 340, 365, 372, 374, 375, 376, 377, 378, 381, 392, 395, 398, 400, 419, 420, 422, 423, 425 426, 436, 461, 468, 470, 471, 472, 473, 474, and 477.

In some implementations, (a) the sense strand comprises a nucleobase sequence according to SEQ ID NO: 97 and the antisense strand comprises a nucleobase sequence according to SEQ ID NO: 1;
(b) the sense strand comprises a nucleobase sequence according to SEQ ID NO: 98 and the antisense strand comprises a nucleobase sequence according to SEQ ID NO: 2;
(c) the sense strand comprises a nucleobase sequence according to SEQ ID NO: 99 and the antisense strand comprises a nucleobase sequence according to SEQ ID NO: 3;
(d) the sense strand comprises a nucleobase sequence according to SEQ ID NO: 100 and the antisense strand comprises a nucleobase sequence according to SEQ ID NO: 4;
(e) the sense strand comprises a nucleobase sequence according to SEQ ID NO: 101 and the antisense strand comprises a nucleobase sequence according to SEQ ID NO: 5;
(f) the sense strand comprises a nucleobase sequence according to SEQ ID NO: 102 and the antisense strand comprises a nucleobase sequence according to SEQ ID NO: 6;
(g) the sense strand comprises a nucleobase sequence according to SEQ ID NO: 103 and the antisense strand comprises a nucleobase sequence according to SEQ ID NO: 7;
(h) the sense strand comprises a nucleobase sequence according to SEQ ID NO: 104 and the antisense strand comprises a nucleobase sequence according to SEQ ID NO: 8;
(i) the sense strand comprises a nucleobase sequence according to SEQ ID NO: 105 and the antisense strand comprises a nucleobase sequence according to SEQ ID NO: 9;
(j) the sense strand comprises a nucleobase sequence according to SEQ ID NO: 106 and the antisense strand comprises a nucleobase sequence according to SEQ ID NO: 10;
(k) the sense strand comprises a nucleobase sequence according to SEQ ID NO: 107 and the antisense strand comprises a nucleobase sequence according to SEQ ID NO: 11;
(l) the sense strand comprises a nucleobase sequence according to SEQ ID NO: 108 and the antisense strand comprises a nucleobase sequence according to SEQ ID NO: 12;
(m) the sense strand comprises a nucleobase sequence according to SEQ ID NO: 109 and the antisense strand comprises a nucleobase sequence according to SEQ ID NO: 13;
(n) the sense strand comprises a nucleobase sequence according to SEQ ID NO: 110 and the antisense strand comprises a nucleobase sequence according to SEQ ID NO: 14;
(o) the sense strand comprises a nucleobase sequence according to SEQ ID NO: 111 and the antisense strand comprises a nucleobase sequence according to SEQ ID NO: 15;
(p) the sense strand comprises a nucleobase sequence according to SEQ ID NO: 112 and the antisense strand comprises a nucleobase sequence according to SEQ ID NO: 16;
(q) the sense strand comprises a nucleobase sequence according to SEQ ID NO: 113 and the antisense strand comprises a nucleobase sequence according to SEQ ID NO: 17;
(r) the sense strand comprises a nucleobase sequence according to SEQ ID NO: 114 and the antisense strand comprises a nucleobase sequence according to SEQ ID NO: 18;
(s) the sense strand comprises a nucleobase sequence according to SEQ ID NO: 115 and the antisense strand comprises a nucleobase sequence according to SEQ ID NO: 19;
(t) the sense strand comprises a nucleobase sequence according to SEQ ID NO: 116 and the antisense strand comprises a nucleobase sequence according to SEQ ID NO: 20;
(u) the sense strand comprises a nucleobase sequence according to SEQ ID NO: 117 and the antisense strand comprises a nucleobase sequence according to SEQ ID NO: 21;
(v) the sense strand comprises a nucleobase sequence according to SEQ ID NO: 118 and the antisense strand comprises a nucleobase sequence according to SEQ ID NO: 22;
(w) the sense strand comprises a nucleobase sequence according to SEQ ID NO: 119 and the antisense strand comprises a nucleobase sequence according to SEQ ID NO: 23;
(x) the sense strand comprises a nucleobase sequence according to SEQ ID NO: 120 and the antisense strand comprises a nucleobase sequence according to SEQ ID NO: 24;
(y) the sense strand comprises a nucleobase sequence according to SEQ ID NO: 121 and the antisense strand comprises a nucleobase sequence according to SEQ ID NO: 25;
(z) the sense strand comprises a nucleobase sequence according to SEQ ID NO: 122 and the antisense strand comprises a nucleobase sequence according to SEQ ID NO: 26;
(aa) the sense strand comprises a nucleobase sequence according to SEQ ID NO: 123 and the antisense strand comprises a nucleobase sequence according to SEQ ID NO: 27;
(bb) the sense strand comprises a nucleobase sequence according to SEQ ID NO: 124 and the antisense strand comprises a nucleobase sequence according to SEQ ID NO: 28;
(cc) the sense strand comprises a nucleobase sequence according to SEQ ID NO: 125 and the antisense strand comprises a nucleobase sequence according to SEQ ID NO: 29;
(dd) the sense strand comprises a nucleobase sequence according to SEQ ID NO: 126 and the antisense strand comprises a nucleobase sequence according to SEQ ID NO: 30;
(ee) the sense strand comprises a nucleobase sequence according to SEQ ID NO: 127 and the antisense strand comprises a nucleobase sequence according to SEQ ID NO: 31;
(ff) the sense strand comprises a nucleobase sequence according to SEQ ID NO: 128 and the antisense strand comprises a nucleobase sequence according to SEQ ID NO: 32;
(gg) the sense strand comprises a nucleobase sequence according to SEQ ID NO: 129 and the antisense strand comprises a nucleobase sequence according to SEQ ID NO: 33;
(hh) the sense strand comprises a nucleobase sequence according to SEQ ID NO: 130 and the antisense strand comprises a nucleobase sequence according to SEQ ID NO: 34;
(ii) the sense strand comprises a nucleobase sequence according to SEQ ID NO: 131 and the antisense strand comprises a nucleobase sequence according to SEQ ID NO: 35;
(jj) the sense strand comprises a nucleobase sequence according to SEQ ID NO: 132 and the antisense strand comprises a nucleobase sequence according to SEQ ID NO: 36;
(kk) the sense strand comprises a nucleobase sequence according to SEQ ID NO: 133 and the antisense strand comprises a nucleobase sequence according to SEQ ID NO: 37;
(ll) the sense strand comprises a nucleobase sequence according to SEQ ID NO: 134 and the antisense strand comprises a nucleobase sequence according to SEQ ID NO: 38;
(mm) the sense strand comprises a nucleobase sequence according to SEQ ID NO: 135 and the antisense strand comprises a nucleobase sequence according to SEQ ID NO: 39;
(nn) the sense strand comprises a nucleobase sequence according to SEQ ID NO: 136 and the antisense strand comprises a nucleobase sequence according to SEQ ID NO: 40;
(oo) the sense strand comprises a nucleobase sequence according to SEQ ID NO: 137 and the antisense strand comprises a nucleobase sequence according to SEQ ID NO: 41;
(pp) the sense strand comprises a nucleobase sequence according to SEQ ID NO: 138 and the antisense strand comprises a nucleobase sequence according to SEQ ID NO: 42;
(qq) the sense strand comprises a nucleobase sequence according to SEQ ID NO: 139 and the antisense strand comprises a nucleobase sequence according to SEQ ID NO: 43;
(rr) the sense strand comprises a nucleobase sequence according to SEQ ID NO: 140 and the antisense strand comprises a nucleobase sequence according to SEQ ID NO: 44;
(ss) the sense strand comprises a nucleobase sequence according to SEQ ID NO: 141 and the antisense strand comprises a nucleobase sequence according to SEQ ID NO: 45;
(tt) the sense strand comprises a nucleobase sequence according to SEQ ID NO: 142 and the antisense strand comprises a nucleobase sequence according to SEQ ID NO: 46;
(uu) the sense strand comprises a nucleobase sequence according to SEQ ID NO: 143 and the antisense strand comprises a nucleobase sequence according to SEQ ID NO: 47;
(vv) the sense strand comprises a nucleobase sequence according to SEQ ID NO: 144 and the antisense strand comprises a nucleobase sequence according to SEQ ID NO: 48;
(ww) the sense strand comprises a nucleobase sequence according to SEQ ID NO: 145 and the antisense strand comprises a nucleobase sequence according to SEQ ID NO: 49;
(xx) the sense strand comprises a nucleobase sequence according to SEQ ID NO: 146 and the antisense strand comprises a nucleobase sequence according to SEQ ID NO: 50;
(yy) the sense strand comprises a nucleobase sequence according to SEQ ID NO: 147 and the antisense strand comprises a nucleobase sequence according to SEQ ID NO: 51;
(zz) the sense strand comprises a nucleobase sequence according to SEQ ID NO: 148 and the antisense strand comprises a nucleobase sequence according to SEQ ID NO: 52;
(aaa) the sense strand comprises a nucleobase sequence according to SEQ ID NO: 149 and the antisense strand comprises a nucleobase sequence according to SEQ ID NO: 53;
(bbb) the sense strand comprises a nucleobase sequence according to SEQ ID NO: 150 and the antisense strand comprises a nucleobase sequence according to SEQ ID NO: 54;
(ccc) the sense strand comprises a nucleobase sequence according to SEQ ID NO: 151 and the antisense strand comprises a nucleobase sequence according to SEQ ID NO: 55;
(ddd) the sense strand comprises a nucleobase sequence according to SEQ ID NO: 152 and the antisense strand comprises a nucleobase sequence according to SEQ ID NO: 56;
(eee) the sense strand comprises a nucleobase sequence according to SEQ ID NO: 153 and the antisense strand comprises a nucleobase sequence according to SEQ ID NO: 57;
(fff) the sense strand comprises a nucleobase sequence according to SEQ ID NO: 154 and the antisense strand comprises a nucleobase sequence according to SEQ ID NO: 58;
(ggg) the sense strand comprises a nucleobase sequence according to SEQ ID NO: 155 and the antisense strand comprises a nucleobase sequence according to SEQ ID NO: 59;
(hhh) the sense strand comprises a nucleobase sequence according to SEQ ID NO: 156 and the antisense strand comprises a nucleobase sequence according to SEQ ID NO: 60;
(iii) the sense strand comprises a nucleobase sequence according to SEQ ID NO: 157 and the antisense strand comprises a nucleobase sequence according to SEQ ID NO: 61;
(jjj) the sense strand comprises a nucleobase sequence according to SEQ ID NO: 158 and the antisense strand comprises a nucleobase sequence according to SEQ ID NO: 62;
(kkk) the sense strand comprises a nucleobase sequence according to SEQ ID NO: 159 and the antisense strand comprises a nucleobase sequence according to SEQ ID NO: 63;
(lll) the sense strand comprises a nucleobase sequence according to SEQ ID NO: 160 and the antisense strand comprises a nucleobase sequence according to SEQ ID NO: 64;
(mmm) the sense strand comprises a nucleobase sequence according to SEQ ID NO: 161 and the antisense strand comprises a nucleobase sequence according to SEQ ID NO: 65;
(nnn) the sense strand comprises a nucleobase sequence according to SEQ ID NO: 162 and the antisense strand comprises a nucleobase sequence according to SEQ ID NO: 66;
(ooo) the sense strand comprises a nucleobase sequence according to SEQ ID NO: 163 and the antisense strand comprises a nucleobase sequence according to SEQ ID NO: 67;
(ppp) the sense strand comprises a nucleobase sequence according to SEQ ID NO: 164 and the antisense strand comprises a nucleobase sequence according to SEQ ID NO: 68;
(qqq) the sense strand comprises a nucleobase sequence according to SEQ ID NO: 165 and the antisense strand comprises a nucleobase sequence according to SEQ ID NO: 69;
(rrr) the sense strand comprises a nucleobase sequence according to SEQ ID NO: 166 and the antisense strand comprises a nucleobase sequence according to SEQ ID NO: 70;
(sss) the sense strand comprises a nucleobase sequence according to SEQ ID NO: 167 and the antisense strand comprises a nucleobase sequence according to SEQ ID NO: 71;
(ttt) the sense strand comprises a nucleobase sequence according to SEQ ID NO: 168 and the antisense strand comprises a nucleobase sequence according to SEQ ID NO: 72;
(uuu) the sense strand comprises a nucleobase sequence according to SEQ ID NO: 169 and the antisense strand comprises a nucleobase sequence according to SEQ ID NO: 73;
(vvv) the sense strand comprises a nucleobase sequence according to SEQ ID NO: 170 and the antisense strand comprises a nucleobase sequence according to SEQ ID NO: 74;
(www) the sense strand comprises a nucleobase sequence according to SEQ ID NO: 171 and the antisense strand comprises a nucleobase sequence according to SEQ ID NO: 75;
(xxx) the sense strand comprises a nucleobase sequence according to SEQ ID NO: 172 and the antisense strand comprises a nucleobase sequence according to SEQ ID NO: 76;
(yyy) the sense strand comprises a nucleobase sequence according to SEQ ID NO: 173 and the antisense strand comprises a nucleobase sequence according to SEQ ID NO: 77;
(zzz) the sense strand comprises a nucleobase sequence according to SEQ ID NO: 174 and the antisense strand comprises a nucleobase sequence according to SEQ ID NO: 78;
(aaaa) the sense strand comprises a nucleobase sequence according to SEQ ID NO: 175 and the antisense strand comprises a nucleobase sequence according to SEQ ID NO: 79;
(bbbb) the sense strand comprises a nucleobase sequence according to SEQ ID NO: 176 and the antisense strand comprises a nucleobase sequence according to SEQ ID NO: 80;
(cccc) the sense strand comprises a nucleobase sequence according to SEQ ID NO: 177 and the antisense strand comprises a nucleobase sequence according to SEQ ID NO: 81;
(dddd) the sense strand comprises a nucleobase sequence according to SEQ ID NO: 178 and the antisense strand comprises a nucleobase sequence according to SEQ ID NO: 82;
(eeee) the sense strand comprises a nucleobase sequence according to SEQ ID NO: 179 and the antisense strand comprises a nucleobase sequence according to SEQ ID NO: 83;
(ffff) the sense strand comprises a nucleobase sequence according to SEQ ID NO: 180 and the antisense strand comprises a nucleobase sequence according to SEQ ID NO: 84;
(gggg) the sense strand comprises a nucleobase sequence according to SEQ ID NO: 181 and the antisense strand comprises a nucleobase sequence according to SEQ ID NO: 85;
(hhhh) the sense strand comprises a nucleobase sequence according to SEQ ID NO: 182 and the antisense strand comprises a nucleobase sequence according to SEQ ID NO: 86;
(iiii) the sense strand comprises a nucleobase sequence according to SEQ ID NO: 183 and the antisense strand comprises a nucleobase sequence according to SEQ ID NO: 87;
(jjjj) the sense strand comprises a nucleobase sequence according to SEQ ID NO: 184 and the antisense strand comprises a nucleobase sequence according to SEQ ID NO: 88;
(kkkk) the sense strand comprises a nucleobase sequence according to SEQ ID NO: 185 and the antisense strand comprises a nucleobase sequence according to SEQ ID NO: 89;
(llll) the sense strand comprises a nucleobase sequence according to SEQ ID NO: 186 and the antisense strand comprises a nucleobase sequence according to SEQ ID NO: 90;
(mmmm) the sense strand comprises a nucleobase sequence according to SEQ ID NO: 187 and the antisense strand comprises a nucleobase sequence according to SEQ ID NO: 91;
(nnnn) the sense strand comprises a nucleobase sequence according to SEQ ID NO: 188 and the antisense strand comprises a nucleobase sequence according to SEQ ID NO: 92;
(oooo) the sense strand comprises a nucleobase sequence according to SEQ ID NO: 189 and the antisense strand comprises a nucleobase sequence according to SEQ ID NO: 93;
(pppp) the sense strand comprises a nucleobase sequence according to SEQ ID NO: 190 and the antisense strand comprises a nucleobase sequence according to SEQ ID NO: 94;
(qqqq) the sense strand comprises a nucleobase sequence according to SEQ ID NO: 191 and the antisense strand comprises a nucleobase sequence according to SEQ ID NO: 95; or
(rrrr) the sense strand comprises a nucleobase sequence according to SEQ ID NO: 192 and the antisense strand comprises a nucleobase sequence according to SEQ ID NO: 96.

In some implementations, (a) the sense strand comprises a nucleobase sequence according to SEQ ID NO: 104 and the antisense strand comprises a nucleobase sequence according to SEQ ID NO: 8;
(b) the sense strand comprises a nucleobase sequence according to SEQ ID NO: 107 and the antisense strand comprises a nucleobase sequence according to SEQ ID NO: 11;
(c) the sense strand comprises a nucleobase sequence according to SEQ ID NO: 110 and the antisense strand comprises a nucleobase sequence according to SEQ ID NO: 14;
(d) the sense strand comprises a nucleobase sequence according to SEQ ID NO: 112 and the antisense strand comprises a nucleobase sequence according to SEQ ID NO: 16;
(e) the sense strand comprises a nucleobase sequence according to SEQ ID NO: 131 and the antisense strand comprises a nucleobase sequence according to SEQ ID NO: 35;
(f) the sense strand comprises a nucleobase sequence according to SEQ ID NO: 132 and the antisense strand comprises a nucleobase sequence according to SEQ ID NO: 36;
(g) the sense strand comprises a nucleobase sequence according to SEQ ID NO: 134 and the antisense strand comprises a nucleobase sequence according to SEQ ID NO: 38;
(h) the sense strand comprises a nucleobase sequence according to SEQ ID NO: 135 and the antisense strand comprises a nucleobase sequence according to SEQ ID NO: 39;
(i) the sense strand comprises a nucleobase sequence according to SEQ ID NO: 137 and the antisense strand comprises a nucleobase sequence according to SEQ ID NO: 41;
(j) the sense strand comprises a nucleobase sequence according to SEQ ID NO: 138 and the antisense strand comprises a nucleobase sequence according to SEQ ID NO: 42;
(k) the sense strand comprises a nucleobase sequence according to SEQ ID NO: 148 and the antisense strand comprises a nucleobase sequence according to SEQ ID NO: 52;
(l) the sense strand comprises a nucleobase sequence according to SEQ ID NO: 173 and the antisense strand comprises a nucleobase sequence according to SEQ ID NO: 77.
(m) the sense strand comprises a nucleobase sequence according to SEQ ID NO: 180 and the antisense strand comprises a nucleobase sequence according to SEQ ID NO: 84;
(n) the sense strand comprises a nucleobase sequence according to SEQ ID NO: 182 and the antisense strand comprises a nucleobase sequence according to SEQ ID NO: 86;
(o) the sense strand comprises a nucleobase sequence according to SEQ ID NO: 183 and the antisense strand comprises a nucleobase sequence according to SEQ ID NO: 87;
(p) the sense strand comprises a nucleobase sequence according to SEQ ID NO: 184 and the antisense strand comprises a nucleobase sequence according to SEQ ID NO: 88;
(q) the sense strand comprises a nucleobase sequence according to SEQ ID NO: 185 and the antisense strand comprises a nucleobase sequence according to SEQ ID NO: 89;
(r) the sense strand comprises a nucleobase sequence according to SEQ ID NO: 186 and the antisense strand comprises a nucleobase sequence according to SEQ ID NO: 90; or(s) the sense strand comprises a nucleobase sequence according to SEQ ID NO: 189 and the antisense strand comprises a nucleobase sequence according to SEQ ID NO: 93.

In some implementations, (a) the sense strand comprises a modified nucleotide sequence according to SEQ ID NO: 289 and the antisense strand comprises a modified nucleotide sequence according to SEQ ID NO: 193;
(b) the sense strand comprises a modified nucleotide sequence according to SEQ ID NO: 290 and the antisense strand comprises a modified nucleotide sequence according to SEQ ID NO: 194;
(c) the sense strand comprises a modified nucleotide sequence according to SEQ ID NO: 291 and the antisense strand comprises a modified nucleotide sequence according to SEQ ID NO: 195;
(d) the sense strand comprises a modified nucleotide sequence according to SEQ ID NO: 292 and the antisense strand comprises a modified nucleotide sequence according to SEQ ID NO: 196;
(e) the sense strand comprises a modified nucleotide sequence according to SEQ ID NO: 293 and the antisense strand comprises a modified nucleotide sequence according to SEQ ID NO: 197;
(f) the sense strand comprises a modified nucleotide sequence according to SEQ ID NO: 294 and the antisense strand comprises a modified nucleotide sequence according to SEQ ID NO: 198;
(g) the sense strand comprises a modified nucleotide sequence according to SEQ ID NO: 295 and the antisense strand comprises a modified nucleotide sequence according to SEQ ID NO: 199;
(h) the sense strand comprises a modified nucleotide sequence according to SEQ ID NO: 296 and the antisense strand comprises a modified nucleotide sequence according to SEQ ID NO: 200;
(i) the sense strand comprises a modified nucleotide sequence according to SEQ ID NO: 297 and the antisense strand comprises a modified nucleotide sequence according to SEQ ID NO: 201;
(j) the sense strand comprises a modified nucleotide sequence according to SEQ ID NO: 298 and the antisense strand comprises a modified nucleotide sequence according to SEQ ID NO: 202;
(k) the sense strand comprises a modified nucleotide sequence according to SEQ ID NO: 299 and the antisense strand comprises a modified nucleotide sequence according to SEQ ID NO: 203;
(l) the sense strand comprises a modified nucleotide sequence according to SEQ ID NO: 300 and the antisense strand comprises a modified nucleotide sequence according to SEQ ID NO: 204;
(m) the sense strand comprises a modified nucleotide sequence according to SEQ ID NO: 301 and the antisense strand comprises a modified nucleotide sequence according to SEQ ID NO: 205;
(n) the sense strand comprises a modified nucleotide sequence according to SEQ ID NO: 302 and the antisense strand comprises a modified nucleotide sequence according to SEQ ID NO: 206;
(o) the sense strand comprises a modified nucleotide sequence according to SEQ ID NO: 303 and the antisense strand comprises a modified nucleotide sequence according to SEQ ID NO: 207;
(p) the sense strand comprises a modified nucleotide sequence according to SEQ ID NO: 304 and the antisense strand comprises a modified nucleotide sequence according to SEQ ID NO: 208;
(q) the sense strand comprises a modified nucleotide sequence according to SEQ ID NO: 305 and the antisense strand comprises a modified nucleotide sequence according to SEQ ID NO: 209;
(r) the sense strand comprises a modified nucleotide sequence according to SEQ ID NO: 306 and the antisense strand comprises a modified nucleotide sequence according to SEQ ID NO: 210;
(s) the sense strand comprises a modified nucleotide sequence according to SEQ ID NO: 307 and the antisense strand comprises a modified nucleotide sequence according to SEQ ID NO: 211;
(t) the sense strand comprises a modified nucleotide sequence according to SEQ ID NO: 308 and the antisense strand comprises a modified nucleotide sequence according to SEQ ID NO: 212;
(u) the sense strand comprises a modified nucleotide sequence according to SEQ ID NO: 309 and the antisense strand comprises a modified nucleotide sequence according to SEQ ID NO: 213;
(v) the sense strand comprises a modified nucleotide sequence according to SEQ ID NO: 310 and the antisense strand comprises a modified nucleotide sequence according to SEQ ID NO: 214;
(w) the sense strand comprises a modified nucleotide sequence according to SEQ ID NO: 311 and the antisense strand comprises a modified nucleotide sequence according to SEQ ID NO: 215;
(x) the sense strand comprises a modified nucleotide sequence according to SEQ ID NO: 312 and the antisense strand comprises a modified nucleotide sequence according to SEQ ID NO: 216;
(y) the sense strand comprises a modified nucleotide sequence according to SEQ ID NO: 313 and the antisense strand comprises a modified nucleotide sequence according to SEQ ID NO: 217;
(z) the sense strand comprises a modified nucleotide sequence according to SEQ ID NO: 314 and the antisense strand comprises a modified nucleotide sequence according to SEQ ID NO: 218;
(aa) the sense strand comprises a modified nucleotide sequence according to SEQ ID NO: 315 and the antisense strand comprises a modified nucleotide sequence according to SEQ ID NO: 219;
(bb) the sense strand comprises a modified nucleotide sequence according to SEQ ID NO: 316 and the antisense strand comprises a modified nucleotide sequence according to SEQ ID NO: 220;
(cc) the sense strand comprises a modified nucleotide sequence according to SEQ ID NO: 317 and the antisense strand comprises a modified nucleotide sequence according to SEQ ID NO: 221;
(dd) the sense strand comprises a modified nucleotide sequence according to SEQ ID NO: 318 and the antisense strand comprises a modified nucleotide sequence according to SEQ ID NO: 222;
(ee) the sense strand comprises a modified nucleotide sequence according to SEQ ID NO: 319 and the antisense strand comprises a modified nucleotide sequence according to SEQ ID NO: 223;
(ff) the sense strand comprises a modified nucleotide sequence according to SEQ ID NO: 320 and the antisense strand comprises a modified nucleotide sequence according to SEQ ID NO: 224;
(gg) the sense strand comprises a modified nucleotide sequence according to SEQ ID NO: 321 and the antisense strand comprises a modified nucleotide sequence according to SEQ ID NO: 225;
(hh) the sense strand comprises a modified nucleotide sequence according to SEQ ID NO: 322 and the antisense strand comprises a modified nucleotide sequence according to SEQ ID NO: 226;
(ii) the sense strand comprises a modified nucleotide sequence according to SEQ ID NO: 323 and the antisense strand comprises a modified nucleotide sequence according to SEQ ID NO: 227;
(jj) the sense strand comprises a modified nucleotide sequence according to SEQ ID NO: 324 and the antisense strand comprises a modified nucleotide sequence according to SEQ ID NO: 228;
(kk) the sense strand comprises a modified nucleotide sequence according to SEQ ID NO: 325 and the antisense strand comprises a modified nucleotide sequence according to SEQ ID NO: 229;
(ll) the sense strand comprises a modified nucleotide sequence according to SEQ ID NO: 326 and the antisense strand comprises a modified nucleotide sequence according to SEQ ID NO: 230;
(mm) the sense strand comprises a modified nucleotide sequence according to SEQ ID NO: 327 and the antisense strand comprises a modified nucleotide sequence according to SEQ ID NO: 231;
(nn) the sense strand comprises a modified nucleotide sequence according to SEQ ID NO: 328 and the antisense strand comprises a modified nucleotide sequence according to SEQ ID NO: 232;
(oo) the sense strand comprises a modified nucleotide sequence according to SEQ ID NO: 329 and the antisense strand comprises a modified nucleotide sequence according to SEQ ID NO: 233;
(pp) the sense strand comprises a modified nucleotide sequence according to SEQ ID NO: 330 and the antisense strand comprises a modified nucleotide sequence according to SEQ ID NO: 234;
(qq) the sense strand comprises a modified nucleotide sequence according to SEQ ID NO: 331 and the antisense strand comprises a modified nucleotide sequence according to SEQ ID NO: 235;
(rr) the sense strand comprises a modified nucleotide sequence according to SEQ ID NO: 332 and the antisense strand comprises a modified nucleotide sequence according to SEQ ID NO: 236;
(ss) the sense strand comprises a modified nucleotide sequence according to SEQ ID NO: 333 and the antisense strand comprises a modified nucleotide sequence according to SEQ ID NO: 237;
(tt) the sense strand comprises a modified nucleotide sequence according to SEQ ID NO: 334 and the antisense strand comprises a modified nucleotide sequence according to SEQ ID NO: 238;
(uu) the sense strand comprises a modified nucleotide sequence according to SEQ ID NO: 335 and the antisense strand comprises a modified nucleotide sequence according to SEQ ID NO: 239;
(vv) the sense strand comprises a modified nucleotide sequence according to SEQ ID NO: 336 and the antisense strand comprises a modified nucleotide sequence according to SEQ ID NO: 240;
(ww) the sense strand comprises a modified nucleotide sequence according to SEQ ID NO: 337 and the antisense strand comprises a modified nucleotide sequence according to SEQ ID NO: 241;
(xx) the sense strand comprises a modified nucleotide sequence according to SEQ ID NO: 338 and the antisense strand comprises a modified nucleotide sequence according to SEQ ID NO: 242;
(yy) the sense strand comprises a modified nucleotide sequence according to SEQ ID NO: 339 and the antisense strand comprises a modified nucleotide sequence according to SEQ ID NO: 243;
(zz) the sense strand comprises a modified nucleotide sequence according to SEQ ID NO: 340 and the antisense strand comprises a modified nucleotide sequence according to SEQ ID NO: 244;
(aaa) the sense strand comprises a modified nucleotide sequence according to SEQ ID NO: 341 and the antisense strand comprises a modified nucleotide sequence according to SEQ ID NO: 245;
(bbb) the sense strand comprises a modified nucleotide sequence according to SEQ ID NO: 342 and the antisense strand comprises a modified nucleotide sequence according to SEQ ID NO: 246;
(ccc) the sense strand comprises a modified nucleotide sequence according to SEQ ID NO: 343 and the antisense strand comprises a modified nucleotide sequence according to SEQ ID NO: 247;
(ddd) the sense strand comprises a modified nucleotide sequence according to SEQ ID NO: 344 and the antisense strand comprises a modified nucleotide sequence according to SEQ ID NO: 248;
(eee) the sense strand comprises a modified nucleotide sequence according to SEQ ID NO: 345 and the antisense strand comprises a modified nucleotide sequence according to SEQ ID NO: 249;
(fff) the sense strand comprises a modified nucleotide sequence according to SEQ ID NO: 346 and the antisense strand comprises a modified nucleotide sequence according to SEQ ID NO: 250;
(ggg) the sense strand comprises a modified nucleotide sequence according to SEQ ID NO: 347 and the antisense strand comprises a modified nucleotide sequence according to SEQ ID NO: 251;
(hhh) the sense strand comprises a modified nucleotide sequence according to SEQ ID NO: 348 and the antisense strand comprises a modified nucleotide sequence according to SEQ ID NO: 252;
(iii) the sense strand comprises a modified nucleotide sequence according to SEQ ID NO: 349 and the antisense strand comprises a modified nucleotide sequence according to SEQ ID NO: 253;
(jjj) the sense strand comprises a modified nucleotide sequence according to SEQ ID NO: 350 and the antisense strand comprises a modified nucleotide sequence according to SEQ ID NO: 254;
(kkk) the sense strand comprises a modified nucleotide sequence according to SEQ ID NO: 351 and the antisense strand comprises a modified nucleotide sequence according to SEQ ID NO: 255;
(lll) the sense strand comprises a modified nucleotide sequence according to SEQ ID NO: 352 and the antisense strand comprises a modified nucleotide sequence according to SEQ ID NO: 256;
(mmm) the sense strand comprises a modified nucleotide sequence according to SEQ ID NO: 353 and the antisense strand comprises a modified nucleotide sequence according to SEQ ID NO: 257;
(nnn) the sense strand comprises a modified nucleotide sequence according to SEQ ID NO: 354 and the antisense strand comprises a modified nucleotide sequence according to SEQ ID NO: 258;
(ooo) the sense strand comprises a modified nucleotide sequence according to SEQ ID NO: 355 and the antisense strand comprises a modified nucleotide sequence according to SEQ ID NO: 259;
(ppp) the sense strand comprises a modified nucleotide sequence according to SEQ ID NO: 356 and the antisense strand comprises a modified nucleotide sequence according to SEQ ID NO: 260;
(qqq) the sense strand comprises a modified nucleotide sequence according to SEQ ID NO: 357 and the antisense strand comprises a modified nucleotide sequence according to SEQ ID NO: 261;
(rrr) the sense strand comprises a modified nucleotide sequence according to SEQ ID NO: 358 and the antisense strand comprises a modified nucleotide sequence according to SEQ ID NO: 262;
(sss) the sense strand comprises a modified nucleotide sequence according to SEQ ID NO: 359 and the antisense strand comprises a modified nucleotide sequence according to SEQ ID NO: 263;
(ttt) the sense strand comprises a modified nucleotide sequence according to SEQ ID NO: 360 and the antisense strand comprises a modified nucleotide sequence according to SEQ ID NO: 264;
(uuu) the sense strand comprises a modified nucleotide sequence according to SEQ ID NO: 361 and the antisense strand comprises a modified nucleotide sequence according to SEQ ID NO: 265;
(vvv) the sense strand comprises a modified nucleotide sequence according to SEQ ID NO: 362 and the antisense strand comprises a modified nucleotide sequence according to SEQ ID NO: 266;
(www) the sense strand comprises a modified nucleotide sequence according to SEQ ID NO: 363 and the antisense strand comprises a modified nucleotide sequence according to SEQ ID NO: 267;
(xxx) the sense strand comprises a modified nucleotide sequence according to SEQ ID NO: 364 and the antisense strand comprises a modified nucleotide sequence according to SEQ ID NO: 268;
(yyy) the sense strand comprises a modified nucleotide sequence according to SEQ ID NO: 365 and the antisense strand comprises a modified nucleotide sequence according to SEQ ID NO: 269;
(zzz) the sense strand comprises a modified nucleotide sequence according to SEQ ID NO: 366 and the antisense strand comprises a modified nucleotide sequence according to SEQ ID NO: 270;
(aaaa) the sense strand comprises a modified nucleotide sequence according to SEQ ID NO: 367 and the antisense strand comprises a modified nucleotide sequence according to SEQ ID NO: 271;
(bbbb) the sense strand comprises a modified nucleotide sequence according to SEQ ID NO: 368 and the antisense strand comprises a modified nucleotide sequence according to SEQ ID NO: 272;
(cccc) the sense strand comprises a modified nucleotide sequence according to SEQ ID NO: 369 and the antisense strand comprises a modified nucleotide sequence according to SEQ ID NO: 273;
(dddd) the sense strand comprises a modified nucleotide sequence according to SEQ ID NO: 370 and the antisense strand comprises a modified nucleotide sequence according to SEQ ID NO: 274;
(eeee) the sense strand comprises a modified nucleotide sequence according to SEQ ID NO: 371 and the antisense strand comprises a modified nucleotide sequence according to SEQ ID NO: 275;
(ffff) the sense strand comprises a modified nucleotide sequence according to SEQ ID NO: 372 and the antisense strand comprises a modified nucleotide sequence according to SEQ ID NO: 276;
(gggg) the sense strand comprises a modified nucleotide sequence according to SEQ ID NO: 373 and the antisense strand comprises a modified nucleotide sequence according to SEQ ID NO: 277;
(hhhh) the sense strand comprises a modified nucleotide sequence according to SEQ ID NO: 374 and the antisense strand comprises a modified nucleotide sequence according to SEQ ID NO: 278;
(iiii) the sense strand comprises a modified nucleotide sequence according to SEQ ID NO: 375 and the antisense strand comprises a modified nucleotide sequence according to SEQ ID NO: 279;
(jjjj) the sense strand comprises a modified nucleotide sequence according to SEQ ID NO: 376 and the antisense strand comprises a modified nucleotide sequence according to SEQ ID NO: 280;
(kkkk) the sense strand comprises a modified nucleotide sequence according to SEQ ID NO: 377 and the antisense strand comprises a modified nucleotide sequence according to SEQ ID NO: 281;
(llll) the sense strand comprises a modified nucleotide sequence according to SEQ ID NO: 378 and the antisense strand comprises a modified nucleotide sequence according to SEQ ID NO: 282;
(mmmm) the sense strand comprises a modified nucleotide sequence according to SEQ ID NO: 379 and the antisense strand comprises a modified nucleotide sequence according to SEQ ID NO: 283;
(nnnn) the sense strand comprises a modified nucleotide sequence according to SEQ ID NO: 380 and the antisense strand comprises a modified nucleotide sequence according to SEQ ID NO: 284;
(oooo) the sense strand comprises a modified nucleotide sequence according to SEQ ID NO: 381 and the antisense strand comprises a modified nucleotide sequence according to SEQ ID NO: 285;
(pppp) the sense strand comprises a modified nucleotide sequence according to SEQ ID NO: 382 and the antisense strand comprises a modified nucleotide sequence according to SEQ ID NO: 286;
(qqqq) the sense strand comprises a modified nucleotide sequence according to SEQ ID NO: 383 and the antisense strand comprises a modified nucleotide sequence according to SEQ ID NO: 287;
(rrrr) the sense strand comprises a modified nucleotide sequence according to SEQ ID NO: 384 and the antisense strand comprises a modified nucleotide sequence according to SEQ ID NO: 288;
(ssss) the sense strand comprises a modified nucleotide sequence according to SEQ ID NO: 385 and the antisense strand comprises a modified nucleotide sequence according to SEQ ID NO: 193;
(tttt) the sense strand comprises a modified nucleotide sequence according to SEQ ID NO: 386 and the antisense strand comprises a modified nucleotide sequence according to SEQ ID NO: 194;
(uuuu) the sense strand comprises a modified nucleotide sequence according to SEQ ID NO: 387 and the antisense strand comprises a modified nucleotide sequence according to SEQ ID NO: 195;
(vvvv) the sense strand comprises a modified nucleotide sequence according to SEQ ID NO: 388 and the antisense strand comprises a modified nucleotide sequence according to SEQ ID NO: 196;
(wwww) the sense strand comprises a modified nucleotide sequence according to SEQ ID NO: 389 and the antisense strand comprises a modified nucleotide sequence according to SEQ ID NO: 197;
(xxxx) the sense strand comprises a modified nucleotide sequence according to SEQ ID NO: 390 and the antisense strand comprises a modified nucleotide sequence according to SEQ ID NO: 198;
(yyyy) the sense strand comprises a modified nucleotide sequence according to SEQ ID NO: 391 and the antisense strand comprises a modified nucleotide sequence according to SEQ ID NO: 199;
(zzzz) the sense strand comprises a modified nucleotide sequence according to SEQ ID NO: 392 and the antisense strand comprises a modified nucleotide sequence according to SEQ ID NO: 200;
(aaaaa) the sense strand comprises a modified nucleotide sequence according to SEQ ID NO: 393 and the antisense strand comprises a modified nucleotide sequence according to SEQ ID NO: 201;
(bbbbb) the sense strand comprises a modified nucleotide sequence according to SEQ ID NO: 394 and the antisense strand comprises a modified nucleotide sequence according to SEQ ID NO: 202;
(ccccc) the sense strand comprises a modified nucleotide sequence according to SEQ ID NO: 395 and the antisense strand comprises a modified nucleotide sequence according to SEQ ID NO: 203;
(ddddd) the sense strand comprises a modified nucleotide sequence according to SEQ ID NO: 396 and the antisense strand comprises a modified nucleotide sequence according to SEQ ID NO: 204;
(eeeee) the sense strand comprises a modified nucleotide sequence according to SEQ ID NO: 397 and the antisense strand comprises a modified nucleotide sequence according to SEQ ID NO: 205;
(fffff) the sense strand comprises a modified nucleotide sequence according to SEQ ID NO: 398 and the antisense strand comprises a modified nucleotide sequence according to SEQ ID NO: 206;
(ggggg) the sense strand comprises a modified nucleotide sequence according to SEQ ID NO: 399 and the antisense strand comprises a modified nucleotide sequence according to SEQ ID NO: 207;
(hhhhh) the sense strand comprises a modified nucleotide sequence according to SEQ ID NO: 400 and the antisense strand comprises a modified nucleotide sequence according to SEQ ID NO: 208;
(iiiii) the sense strand comprises a modified nucleotide sequence according to SEQ ID NO: 401 and the antisense strand comprises a modified nucleotide sequence according to SEQ ID NO: 209;
(jjjjj) the sense strand comprises a modified nucleotide sequence according to SEQ ID NO: 402 and the antisense strand comprises a modified nucleotide sequence according to SEQ ID NO: 210;
(kkkkk) the sense strand comprises a modified nucleotide sequence according to SEQ ID NO: 403 and the antisense strand comprises a modified nucleotide sequence according to SEQ ID NO: 211;
(lllll) the sense strand comprises a modified nucleotide sequence according to SEQ ID NO: 404 and the antisense strand comprises a modified nucleotide sequence according to SEQ ID NO: 212;
(mmmmm) the sense strand comprises a modified nucleotide sequence according to SEQ ID NO: 405 and the antisense strand comprises a modified nucleotide sequence according to SEQ ID NO: 213;
(nnnnn) the sense strand comprises a modified nucleotide sequence according to SEQ ID NO: 406 and the antisense strand comprises a modified nucleotide sequence according to SEQ ID NO: 214;
(ooooo) the sense strand comprises a modified nucleotide sequence according to SEQ ID NO: 407 and the antisense strand comprises a modified nucleotide sequence according to SEQ ID NO: 215;
(ppppp) the sense strand comprises a modified nucleotide sequence according to SEQ ID NO: 408 and the antisense strand comprises a modified nucleotide sequence according to SEQ ID NO: 216;
(qqqqq) the sense strand comprises a modified nucleotide sequence according to SEQ ID NO: 409 and the antisense strand comprises a modified nucleotide sequence according to SEQ ID NO: 217;
(rrrrr) the sense strand comprises a modified nucleotide sequence according to SEQ ID NO: 410 and the antisense strand comprises a modified nucleotide sequence according to SEQ ID NO: 218;
(sssss) the sense strand comprises a modified nucleotide sequence according to SEQ ID NO: 411 and the antisense strand comprises a modified nucleotide sequence according to SEQ ID NO: 219;
(ttttt) the sense strand comprises a modified nucleotide sequence according to SEQ ID NO: 412 and the antisense strand comprises a modified nucleotide sequence according to SEQ ID NO: 220;
(uuuuu) the sense strand comprises a modified nucleotide sequence according to SEQ ID NO: 413 and the antisense strand comprises a modified nucleotide sequence according to SEQ ID NO: 221;
(vvvvv) the sense strand comprises a modified nucleotide sequence according to SEQ ID NO: 414 and the antisense strand comprises a modified nucleotide sequence according to SEQ ID NO: 222;
(wwwww) the sense strand comprises a modified nucleotide sequence according to SEQ ID NO: 415 and the antisense strand comprises a modified nucleotide sequence according to SEQ ID NO: 223;
(xxxxx) the sense strand comprises a modified nucleotide sequence according to SEQ ID NO: 416 and the antisense strand comprises a modified nucleotide sequence according to SEQ ID NO: 224;
(yyyyy) the sense strand comprises a modified nucleotide sequence according to SEQ ID NO: 417 and the antisense strand comprises a modified nucleotide sequence according to SEQ ID NO: 225;
(zzzzz) the sense strand comprises a modified nucleotide sequence according to SEQ ID NO: 418 and the antisense strand comprises a modified nucleotide sequence according to SEQ ID NO: 226;
(aaaaaa) the sense strand comprises a modified nucleotide sequence according to SEQ ID NO: 419 and the antisense strand comprises a modified nucleotide sequence according to SEQ ID NO: 227;
(bbbbbb) the sense strand comprises a modified nucleotide sequence according to SEQ ID NO: 420 and the antisense strand comprises a modified nucleotide sequence according to SEQ ID NO: 228;
(cccccc) the sense strand comprises a modified nucleotide sequence according to SEQ ID NO: 421 and the antisense strand comprises a modified nucleotide sequence according to SEQ ID NO: 229;
(dddddd) the sense strand comprises a modified nucleotide sequence according to SEQ ID NO: 422 and the antisense strand comprises a modified nucleotide sequence according to SEQ ID NO: 230;
(eeeeee) the sense strand comprises a modified nucleotide sequence according to SEQ ID NO: 423 and the antisense strand comprises a modified nucleotide sequence according to SEQ ID NO: 231;
(ffffff) the sense strand comprises a modified nucleotide sequence according to SEQ ID NO: 424 and the antisense strand comprises a modified nucleotide sequence according to SEQ ID NO: 232;
(gggggg) the sense strand comprises a modified nucleotide sequence according to SEQ ID NO: 425 and the antisense strand comprises a modified nucleotide sequence according to SEQ ID NO: 233;
(hhhhhh) the sense strand comprises a modified nucleotide sequence according to SEQ ID NO: 426 and the antisense strand comprises a modified nucleotide sequence according to SEQ ID NO: 234;
(iiiiii) the sense strand comprises a modified nucleotide sequence according to SEQ ID NO: 427 and the antisense strand comprises a modified nucleotide sequence according to SEQ ID NO: 235;
(jjjjjj) the sense strand comprises a modified nucleotide sequence according to SEQ ID NO: 428 and the antisense strand comprises a modified nucleotide sequence according to SEQ ID NO: 236;
(kkkkkk) the sense strand comprises a modified nucleotide sequence according to SEQ ID NO: 429 and the antisense strand comprises a modified nucleotide sequence according to SEQ ID NO: 237;
(llllll) the sense strand comprises a modified nucleotide sequence according to SEQ ID NO: 430 and the antisense strand comprises a modified nucleotide sequence according to SEQ ID NO: 238;
(mmmmmm) the sense strand comprises a modified nucleotide sequence according to SEQ ID NO: 431 and the antisense strand comprises a modified nucleotide sequence according to SEQ ID NO: 239;
(nnnnnn) the sense strand comprises a modified nucleotide sequence according to SEQ ID NO: 432 and the antisense strand comprises a modified nucleotide sequence according to SEQ ID NO: 240;
(oooooo) the sense strand comprises a modified nucleotide sequence according to SEQ ID NO: 433 and the antisense strand comprises a modified nucleotide sequence according to SEQ ID NO: 241;
(pppppp) the sense strand comprises a modified nucleotide sequence according to SEQ ID NO: 434 and the antisense strand comprises a modified nucleotide sequence according to SEQ ID NO: 242;
(qqqqqq) the sense strand comprises a modified nucleotide sequence according to SEQ ID NO: 435 and the antisense strand comprises a modified nucleotide sequence according to SEQ ID NO: 243;
(rrrrrr) the sense strand comprises a modified nucleotide sequence according to SEQ ID NO: 436 and the antisense strand comprises a modified nucleotide sequence according to SEQ ID NO: 244;
(ssssss) the sense strand comprises a modified nucleotide sequence according to SEQ ID NO: 437 and the antisense strand comprises a modified nucleotide sequence according to SEQ ID NO: 245;
(tttttt) the sense strand comprises a modified nucleotide sequence according to SEQ ID NO: 438 and the antisense strand comprises a modified nucleotide sequence according to SEQ ID NO: 246;
(uuuuuu) the sense strand comprises a modified nucleotide sequence according to SEQ ID NO: 439 and the antisense strand comprises a modified nucleotide sequence according to SEQ ID NO: 247;
(vvvvvv) the sense strand comprises a modified nucleotide sequence according to SEQ ID NO: 440 and the antisense strand comprises a modified nucleotide sequence according to SEQ ID NO: 248;
(wwwwww) the sense strand comprises a modified nucleotide sequence according to SEQ ID NO: 441 and the antisense strand comprises a modified nucleotide sequence according to SEQ ID NO: 249;
(xxxxxx) the sense strand comprises a modified nucleotide sequence according to SEQ ID NO: 442 and the antisense strand comprises a modified nucleotide sequence according to SEQ ID NO: 250;
(yyyyyy) the sense strand comprises a modified nucleotide sequence according to SEQ ID NO: 443 and the antisense strand comprises a modified nucleotide sequence according to SEQ ID NO: 251;
(zzzzzz) the sense strand comprises a modified nucleotide sequence according to SEQ ID NO: 444 and the antisense strand comprises a modified nucleotide sequence according to SEQ ID NO: 252;
(aaaaaaa) the sense strand comprises a modified nucleotide sequence according to SEQ ID NO: 445 and the antisense strand comprises a modified nucleotide sequence according to SEQ ID NO: 253;
(bbbbbbb) the sense strand comprises a modified nucleotide sequence according to SEQ ID NO: 446 and the antisense strand comprises a modified nucleotide sequence according to SEQ ID NO: 254;
(ccccccc) the sense strand comprises a modified nucleotide sequence according to SEQ ID NO: 447 and the antisense strand comprises a modified nucleotide sequence according to SEQ ID NO: 255;
(ddddddd) the sense strand comprises a modified nucleotide sequence according to SEQ ID NO: 448 and the antisense strand comprises a modified nucleotide sequence according to SEQ ID NO: 256;
(eeeeeee) the sense strand comprises a modified nucleotide sequence according to SEQ ID NO: 449 and the antisense strand comprises a modified nucleotide sequence according to SEQ ID NO: 257;
(fffffff) the sense strand comprises a modified nucleotide sequence according to SEQ ID NO: 450 and the antisense strand comprises a modified nucleotide sequence according to SEQ ID NO: 258;
(ggggggg) the sense strand comprises a modified nucleotide sequence according to SEQ ID NO: 451 and the antisense strand comprises a modified nucleotide sequence according to SEQ ID NO: 259;
(hhhhhhh) the sense strand comprises a modified nucleotide sequence according to SEQ ID NO: 452 and the antisense strand comprises a modified nucleotide sequence according to SEQ ID NO: 260;
(iiiiii) the sense strand comprises a modified nucleotide sequence according to SEQ ID NO: 453 and the antisense strand comprises a modified nucleotide sequence according to SEQ ID NO: 261;
(jjjjjjj) the sense strand comprises a modified nucleotide sequence according to SEQ ID NO: 454 and the antisense strand comprises a modified nucleotide sequence according to SEQ ID NO: 262;
(kkkkkkk) the sense strand comprises a modified nucleotide sequence according to SEQ ID NO: 455 and the antisense strand comprises a modified nucleotide sequence according to SEQ ID NO: 263;
(lllllll) the sense strand comprises a modified nucleotide sequence according to SEQ ID NO: 456 and the antisense strand comprises a modified nucleotide sequence according to SEQ ID NO: 264;
(mmmmmmm) the sense strand comprises a modified nucleotide sequence according to SEQ ID NO: 457 and the antisense strand comprises a modified nucleotide sequence according to SEQ ID NO: 265;
(nnnnnnn) the sense strand comprises a modified nucleotide sequence according to SEQ ID NO: 458 and the antisense strand comprises a modified nucleotide sequence according to SEQ ID NO: 266;
(ooooooo) the sense strand comprises a modified nucleotide sequence according to SEQ ID NO: 459 and the antisense strand comprises a modified nucleotide sequence according to SEQ ID NO: 267;
(ppppppp) the sense strand comprises a modified nucleotide sequence according to SEQ ID NO: 460 and the antisense strand comprises a modified nucleotide sequence according to SEQ ID NO: 268;
(qqqqqqq) the sense strand comprises a modified nucleotide sequence according to SEQ ID NO: 461 and the antisense strand comprises a modified nucleotide sequence according to SEQ ID NO: 269;
(rrrrrrrr) the sense strand comprises a modified nucleotide sequence according to SEQ ID NO: 462 and the antisense strand comprises a modified nucleotide sequence according to SEQ ID NO: 270;
(sssssss) the sense strand comprises a modified nucleotide sequence according to SEQ ID NO: 463 and the antisense strand comprises a modified nucleotide sequence according to SEQ ID NO: 271;
(ttttttt) the sense strand comprises a modified nucleotide sequence according to SEQ ID NO: 464 and the antisense strand comprises a modified nucleotide sequence according to SEQ ID NO: 272;
(uuuuuuu) the sense strand comprises a modified nucleotide sequence according to SEQ ID NO: 465 and the antisense strand comprises a modified nucleotide sequence according to SEQ ID NO: 273;
(vvvvvvv) the sense strand comprises a modified nucleotide sequence according to SEQ ID NO: 466 and the antisense strand comprises a modified nucleotide sequence according to SEQ ID NO: 274;
(wwwwwww) the sense strand comprises a modified nucleotide sequence according to SEQ ID NO: 467 and the antisense strand comprises a modified nucleotide sequence according to SEQ ID NO: 275;
(xxxxxxx) the sense strand comprises a modified nucleotide sequence according to SEQ ID NO: 468 and the antisense strand comprises a modified nucleotide sequence according to SEQ ID NO: 276;
(yyyyyyy) the sense strand comprises a modified nucleotide sequence according to SEQ ID NO: 469 and the antisense strand comprises a modified nucleotide sequence according to SEQ ID NO: 277;
(zzzzzzz) the sense strand comprises a modified nucleotide sequence according to SEQ ID NO: 470 and the antisense strand comprises a modified nucleotide sequence according to SEQ ID NO: 278;
(aaaaaaaa) the sense strand comprises a modified nucleotide sequence according to SEQ ID NO: 471 and the antisense strand comprises a modified nucleotide sequence according to SEQ ID NO: 279;
(bbbbbbbb) the sense strand comprises a modified nucleotide sequence according to SEQ ID NO: 472 and the antisense strand comprises a modified nucleotide sequence according to SEQ ID NO: 280;
(cccccccc) the sense strand comprises a modified nucleotide sequence according to SEQ ID NO: 473 and the antisense strand comprises a modified nucleotide sequence according to SEQ ID NO: 281;
(dddddddd) the sense strand comprises a modified nucleotide sequence according to SEQ ID NO: 474 and the antisense strand comprises a modified nucleotide sequence according to SEQ ID NO: 282;
(eeeeeeee) the sense strand comprises a modified nucleotide sequence according to SEQ ID NO: 475 and the antisense strand comprises a modified nucleotide sequence according to SEQ ID NO: 283;
(ffffffff) the sense strand comprises a modified nucleotide sequence according to SEQ ID NO: 476 and the antisense strand comprises a modified nucleotide sequence according to SEQ ID NO: 284;
(gggggggg) the sense strand comprises a modified nucleotide sequence according to SEQ ID NO: 477 and the antisense strand comprises a modified nucleotide sequence according to SEQ ID NO: 285;
(hhhhhhhh) the sense strand comprises a modified nucleotide sequence according to SEQ ID NO: 478 and the antisense strand comprises a modified nucleotide sequence according to SEQ ID NO: 286;
(iiiiiii) the sense strand comprises a modified nucleotide sequence according to SEQ ID NO: 479 and the antisense strand comprises a modified nucleotide sequence according to SEQ ID NO: 287; or
(jjjjjjjj) the sense strand comprises a modified nucleotide sequence according to SEQ ID NO: 480 and the antisense strand comprises a modified nucleotide sequence according to SEQ ID NO: 288.

In some implementations, (a) the sense strand comprises a modified nucleobase sequence according to SEQ ID NO: 296 and the antisense strand comprises a modified nucleobase sequence according to SEQ ID NO: 200;
(b) the sense strand comprises a modified nucleobase sequence according to SEQ ID NO: 299 and the antisense strand comprises a modified nucleobase sequence according to SEQ ID NO: 203;
(c) the sense strand comprises a modified nucleobase sequence according to SEQ ID NO: 302 and the antisense strand comprises a modified nucleobase sequence according to SEQ ID NO: 206;
(d) the sense strand comprises a modified nucleobase sequence according to SEQ ID NO: 304 and the antisense strand comprises a modified nucleobase sequence according to SEQ ID NO: 208;
(e) the sense strand comprises a modified nucleobase sequence according to SEQ ID NO: 323 and the antisense strand comprises a modified nucleobase sequence according to SEQ ID NO: 227;
(f) the sense strand comprises a modified nucleobase sequence according to SEQ ID NO: 324 and the antisense strand comprises a modified nucleobase sequence according to SEQ ID NO: 228;
(g) the sense strand comprises a modified nucleobase sequence according to SEQ ID NO: 326 and the antisense strand comprises a modified nucleobase sequence according to SEQ ID NO: 230;
(h) the sense strand comprises a modified nucleobase sequence according to SEQ ID NO: 327 and the antisense strand comprises a modified nucleobase sequence according to SEQ ID NO: 231;
(i) the sense strand comprises a modified nucleobase sequence according to SEQ ID NO: 329 and the antisense strand comprises a modified nucleobase sequence according to SEQ ID NO: 233;
(j) the sense strand comprises a modified nucleobase sequence according to SEQ ID NO: 330 and the antisense strand comprises a modified nucleobase sequence according to SEQ ID NO: 234;
(k) the sense strand comprises a modified nucleobase sequence according to SEQ ID NO: 340 and the antisense strand comprises a modified nucleobase sequence according to SEQ ID NO: 244;
(l) the sense strand comprises a modified nucleobase sequence according to SEQ ID NO: 365 and the antisense strand comprises a modified nucleobase sequence according to SEQ ID NO: 269;
(m) the sense strand comprises a modified nucleobase sequence according to SEQ ID NO: 372 and the antisense strand comprises a modified nucleobase sequence according to SEQ ID NO: 276;
(n) the sense strand comprises a modified nucleobase sequence according to SEQ ID NO: 374 and the antisense strand comprises a modified nucleobase sequence according to SEQ ID NO: 278;
(o) the sense strand comprises a modified nucleobase sequence according to SEQ ID NO: 375 and the antisense strand comprises a modified nucleobase sequence according to SEQ ID NO: 279;
(p) the sense strand comprises a modified nucleobase sequence according to SEQ ID NO: 376 and the antisense strand comprises a modified nucleobase sequence according to SEQ ID NO: 280;
(q) the sense strand comprises a modified nucleobase sequence according to SEQ ID NO: 377 and the antisense strand comprises a modified nucleobase sequence according to SEQ ID NO: 281;
(r) the sense strand comprises a modified nucleobase sequence according to SEQ ID NO: 378 and the antisense strand comprises a modified nucleobase sequence according to SEQ ID NO: 282;
(s) the sense strand comprises a modified nucleobase sequence according to SEQ ID NO: 381 and the antisense strand comprises a modified nucleobase sequence according to SEQ ID NO: 285;
(t) the sense strand comprises a modified nucleobase sequence according to SEQ ID NO: 392 and the antisense strand comprises a modified nucleobase sequence according to SEQ ID NO: 200;
(u) the sense strand comprises a modified nucleobase sequence according to SEQ ID NO: 395 and the antisense strand comprises a modified nucleobase sequence according to SEQ ID NO: 203;
(v) the sense strand comprises a modified nucleobase sequence according to SEQ ID NO: 398 and the antisense strand comprises a modified nucleobase sequence according to SEQ ID NO: 206;
(w) the sense strand comprises a modified nucleobase sequence according to SEQ ID NO: 400 and the antisense strand comprises a modified nucleobase sequence according to SEQ ID NO: 208;
(x) the sense strand comprises a modified nucleobase sequence according to SEQ ID NO: 419 and the antisense strand comprises a modified nucleobase sequence according to SEQ ID NO: 227;
(y) the sense strand comprises a modified nucleobase sequence according to SEQ ID NO: 420 and the antisense strand comprises a modified nucleobase sequence according to SEQ ID NO: 228;
(z) the sense strand comprises a modified nucleobase sequence according to SEQ ID NO: 422 and the antisense strand comprises a modified nucleobase sequence according to SEQ ID NO: 230;
(aa) the sense strand comprises a modified nucleobase sequence according to SEQ ID NO: 423 and the antisense strand comprises a modified nucleobase sequence according to SEQ ID NO: 231;
(bb) the sense strand comprises a modified nucleobase sequence according to SEQ ID NO: 425 and the antisense strand comprises a modified nucleobase sequence according to SEQ ID NO: 233;
(cc) the sense strand comprises a modified nucleobase sequence according to SEQ ID NO: 426 and the antisense strand comprises a modified nucleobase sequence according to SEQ ID NO: 234;
(dd) the sense strand comprises a modified nucleobase sequence according to SEQ ID NO: 436 and the antisense strand comprises a modified nucleobase sequence according to SEQ ID NO: 244;
(ee) the sense strand comprises a modified nucleobase sequence according to SEQ ID NO: 461 and the antisense strand comprises a modified nucleobase sequence according to SEQ ID NO: 269;
(ff) the sense strand comprises a modified nucleobase sequence according to SEQ ID NO: 468 and the antisense strand comprises a modified nucleobase sequence according to SEQ ID NO: 276;
(gg) the sense strand comprises a modified nucleobase sequence according to SEQ ID NO: 470 and the antisense strand comprises a modified nucleobase sequence according to SEQ ID NO: 278;
(hh) the sense strand comprises a modified nucleobase sequence according to SEQ ID NO: 471 and the antisense strand comprises a modified nucleobase sequence according to SEQ ID NO: 279;
(ii) the sense strand comprises a modified nucleobase sequence according to SEQ ID NO: 472 and the antisense strand comprises a modified nucleobase sequence according to SEQ ID NO: 280;
(jj) the sense strand comprises a modified nucleobase sequence according to SEQ ID NO: 473 and the antisense strand comprises a modified nucleobase sequence according to SEQ ID NO: 281;
(kk) the sense strand comprises a modified nucleobase sequence according to SEQ ID NO: 474 and the antisense strand comprises a modified nucleobase sequence according to SEQ ID NO: 282; or
(ll) the sense strand comprises a modified nucleobase sequence according to SEQ ID NO: 477 and the antisense strand comprises a modified nucleobase sequence according to SEQ ID NO: 285.

In some implementations, the RNAi agent comprises a targeting ligand comprising a structure according to Formula I:

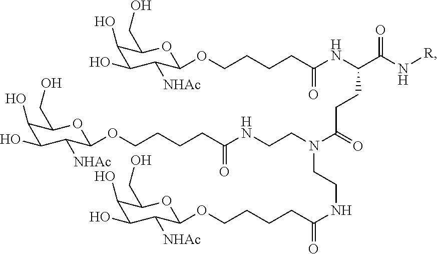

wherein R is a linker conjugated to the wherein the targeting ligand is conjugated to the 3′ end of the sense strand, wherein R comprises a structure according to Formula III:

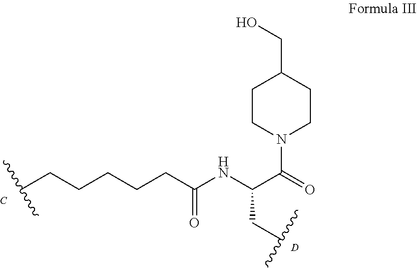

and wherein connection point C of Formula III is conjugated to Formula I, and connection point D of Formula III is conjugated to the 3′ end of the sense strand.

In some implementations, the sense strand comprises a 3′ overhang of 1-5 nucleotides in length. In some implementations, the sense strand comprises a 5′ overhang of 1-5 nucleotides in length. In some implementations, the antisense strand comprises a 3′ overhang of 1-5 nucleotides in length. In some implementations, the antisense strand comprises a 5′ overhang of 1-5 nucleotides in length. In some implementations, the sense strand or the antisense strand comprises at least one modified nucleotide or at least one modified internucleoside linkage. In some implementations, the sense strand and or the antisense strand comprises a modified internucleoside linkage at a first, second, penultimate, or final internucleoside linkage position.

In some implementations, the at least one modified nucleotide or at least one modified internucleoside linkage comprises a 2′-O-methyl modified nucleotide, a nucleotide comprising a 5′-phosphorothioate group, a 2′-deoxy-2′-fluoro modified nucleotide, a 2′-deoxy-modified nucleotide, a locked nucleotide, an abasic nucleotide, deoxythymidine, an inverted deoxythymidine, a 2′-amino-modified nucleotide, a 2′-alkyl-modified nucleotide, a morpholino nucleotide, a phosphoramidate, or a non-natural base comprising nucleotide.

In some implementations, the targeting ligand is conjugated to the sense strand. In some implementations, the targeting ligand is conjugated to the 3′ end of the sense strand. In some implementations, the targeting ligand is conjugated to the 5′ end of the sense strand. In some implementations, the targeting ligand is conjugated to the antisense strand. In some implementations, the targeting ligand is conjugated to the 3′ end of the antisense strand. In some implementations, the targeting ligand is conjugated to the 5′ end of the antisense strand.

In some implementations, the targeting ligand comprises a galactose trimer. In some implementations, the targeting ligand comprises N-acetylgalactosamine (GalNAc). In some implementations, the targeting ligand comprises a structure according to Formula I:

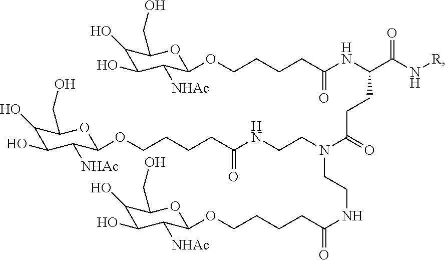

wherein R is:
(i) the sense strand or the antisense strand; or
(ii) a linker conjugated to the sense strand or the antisense strand.

In some implementations, R is a linker conjugated to the sense strand or the antisense strand having a structure according to Formula III:

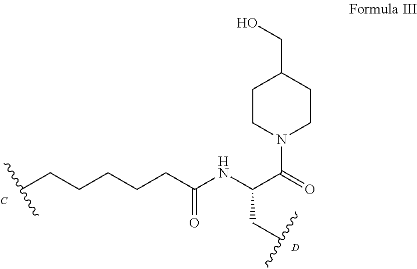

wherein:
(i) connection point C of Formula III is conjugated to Formula I, and connection point D of Formula III is conjugated to the sense strand or the antisense strand; or
(ii) connection point C of Formula III is conjugated to Formula I, and connection point D of Formula III is conjugated to a phosphate group which is conjugated to the sense strand or the antisense strand.

In some implementations, Formula III or the phosphate group is conjugated to the 3′ end or the 5′ end of the sense strand or the antisense strand. In some implementations, Formula III or the phosphate group is conjugated to an internal nucleotide or nucleoside base of the sense strand or the antisense strand. In some implementations, the RNAi agent comprises a targeting ligand and linker according to Formula V:

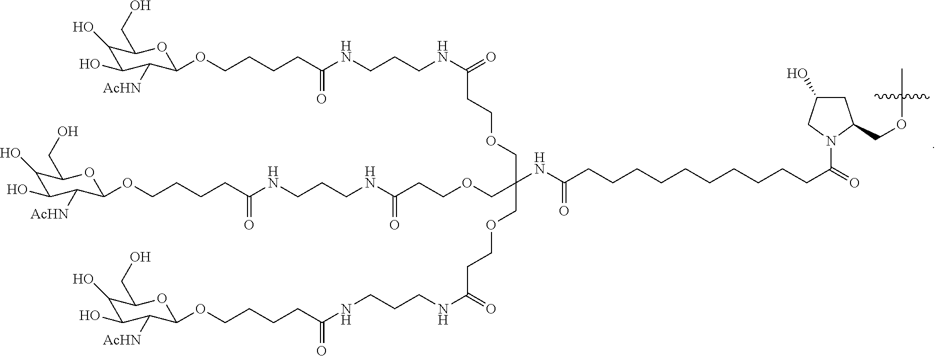 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some implementations, the RNAi agent is an siRNA molecule. In some implementations, the RNAi agent is an shRNA molecule.

Described herein is a method of producing the RNAi agent described herein, comprising annealing together the sense strand and the antisense strand. In some implementations, the method comprises synthesizing the antisense strand and/or the sense strand.

### BRIEF DESCRIPTION OF THE DRAWINGS

FIGS. 1A-1E show dose dependent response curves of exemplary GCK siRNA agents inhibiting GCK mRNA. FIG. 1A shows a dose dependent response curve of exemplary GCK siRNA agents in CaSki cells. FIGS. 1B and 1C show dose dependent response curves of exemplary GalNAc conjugated-GCK siRNA agents in non-human primate primary hepatocytes (NHPPH). FIGS. 1D and 1E show dose dependent response curves of exemplary GalNAc conjugated-GCK siRNA agents in constitutively expressing GCK HepG2 cells (HepG2-GCK).

FIGS. 2A and 2B show a percent GCK mRNA expression (FIG. 2A) and a percent GCK protein expression (FIG. 2B) in harvested liver tissue from mice on a high fat diet treated with exemplary GCK siRNA agents as compared to mice on a high fat diet treated with vehicle control (PBS), as described in Example 6.

FIG. 3 shows the percent change in hepatic triglycerides in mice on a high fat diet treated with exemplary GCK siRNA agents as compared to mice on a high fat diet administered vehicle control, as described in Example 6.

### DETAILED DESCRIPTION

Provided herein are inhibitory nucleic acids (e.g., RNAi agents) to block gene expression in a highly conserved regulatory mechanism known as RNA interference. In particular, the disclosed RNAi agents inhibit the expression of glucokinase (GCK). RNAi agents comprise short interfering RNA (siRNA) and short hairpin RNA (shRNA). The interfering RNA can be assembled from two separate oligonucleotides, where one strand is the sense strand and the other is the antisense strand, herein the antisense and sense strands are self-complementary; the antisense strand comprises nucleotide sequence that is complementary to a nucleotide sequence in a target nucleic acid molecule or a portion thereof and the sense strand comprises nucleotide sequence corresponding to the target nucleic acid sequence or a portion thereof. In some embodiments, the nucleic acid targeted by the RNAi may produce an unwanted protein wherein its inhibition would result in a therapeutic benefit.

Glucokinase (GCK) is an enzyme responsible for the conversion of glucose into glucose-6 phosphate. This is the rate-limiting step in glucose metabolism. GCK is found in mammalian cells of the liver, pancreas, gut, and brain. GCK catalyzes the first reaction of the glycolytic pathway and acts as the primary glucose sensor in the body. Activating mutations in glucokinase cause too much insulin secretion (hyperinsulinism), whereas inactivating mutations cause too little insulin release and varying degrees of diabetes (Ashcroft, et al., Trends Endocrinol Metab. 2023 February; 34(2):119-130). Heterozygous mutations in the glucokinase (GCK) gene produce two distinct diseases, MODY type 2 (MODY 2) and persistent hyperinsulinemia of infancy (Capuano M. et al., (2012) PLoS ON E 7(6): e38906.). Targeting GCK may represent a strategy for restoring lipid metabolic balance.

Provided herein are RNAi agents and compositions designed to target and inhibit the production of GCK expression. Also described herein are methods of using the RNAi agents or compositions thereof in treating or ameliorating metabolic dysfunction-associated steatotic liver disease (MASLD). Also described herein are methods of using the RNAi agents or compositions thereof in treating or ameliorating metabolic dysfunction-associated steatohepatitis (MASH).

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

It is understood that aspects and variations of the invention described herein encompass “comprising,” “consisting,” and/or “consisting essentially of” aspects and variations. The term “including” encompasses “comprising,” “consisting,” and “consisting essentially of” unless otherwise specified.

The term “controlling” as used herein, in reference to a liver fat accumulation in a subject, is understood to mean reducing an increase in an amount of liver fat accumulation compared to an untreated subject. Controlling can include preventing an increase in an amount of liver fat.

As used herein, the term “nucleic acid” refers to a polymer or oligomer of nucleotides. Nucleic acids are also referred as “ribonucleic acids” when the sugar moiety of the nucleotides is D-ribose and as “deoxyribonucleic acids” when the sugar moiety is 2-deoxy-D-ribose.

As used herein, the term “nucleotide” usually refers to a compound consisting of a nucleoside esterified to a monophosphate, polyphosphate, or phosphate-derivative group via the hydroxyl group of the 5-carbon of the sugar moiety. Nucleotides are also referred as “ribonucleotides” when the sugar moiety is D-ribose and as “deoxyribonucleotides” when the sugar moiety is 2-deoxy-D-ribose.

As used herein, the term “nucleoside” refers to a glycosylamine consisting of a nucleobase, such as a purine or pyrimidine, usually linked to a 5-carbon sugar (e.g. D-ribose or 2-deoxy-D-ribose) via a β-glycosidic linkage. Nucleosides are also referred as “ribonucleosides” when the sugar moiety is D-ribose and as “deoxyribonucleosides” when the sugar moiety is 2-deoxy-D-ribose.

As used herein, the term “nucleobase”, refers to a compound containing a nitrogen atom that has the chemical properties of a base. N on-limiting examples of nucleobases are compounds comprising pyridine, purine, or pyrimidine moieties, including, but not limited to adenine, guanine, hypoxanthine, thymine, cytosine, and uracil.

As used herein, the term “nucleotide sequences” encompasses nucleotide sequences with no modified nucleotides and nucleotide sequences with modified nucleotides. The latter are referred to as “modified nucleotide sequences” or simply “modified sequences.”

As used herein, the term “modified nucleotide” is a nucleotide other than a ribonucleotide (2′-hydroxyl nucleotide).

As used herein, the following notations are used to indicate modified nucleotides, targeting groups, and linking groups. As the person of ordinary skill in the art would readily understand, unless otherwise indicated by the sequence, that when present in an oligonucleotide, the monomers are mutually linked by 5′-3′-phosphodiester bonds:
A=adenosine-3′-phosphate;
C=cytidine-3′-phosphate;
G=guanosine-3′-phosphate;
U=uridine-3′-phosphate
n=any 2′-OMe modified nucleotide
a=2′-O-methyladenosine-3′-phosphate
as=2′-O-methyladenosine-3′-phosphorothioate
c=2′-O-methylcytidine-3′-phosphate
cs=2′-O-methylcytidine-3′-phosphorothioate
g=2′-O-methylguanosine-3′-phosphate
gs=2′-O-methylguanosine-3′-phosphorothioate
t=2′-O-methyl-5-methyluridine-3′-phosphate
ts=2′-O-methyl-5-methyluridine-3′-phosphorothioate
u=2′-O-methyluridine-3′-phosphate
us=2′-O-methyluridine-3′-phosphorothioate
Nf=any 2′-fluoro modified nucleotide
Nfs=any 2′-fluoro modified nucleotide with 3′-phosporothioate
Af=2′-fluoroadenosine-3′-phosphate
Afs=2′-fluoroadenosine-3′-phosporothioate
Cf=2′-fluorocytidine-3′-phosphate
Cfs=2′-fluorocytidine-3′-phosphorothioate
Gf=2′-fluoroguanosine-3′-phosphate
Gfs=2′-fluoroguanosine-3′-phosphorothioate
Tf=2′-fluoro-5′-methyluridine-3′-phosphate
Tfs=2′-fluoro-5′-methyluridine-3′-phosphorothioate
Uf=2′-fluorouridine-3′-phosphate
Ufs=2′-fluorouridine-3′-phosphorothioate
dN=any 2′-deoxyribonucleotide
dT=2′-deoxythymidine-3′-phosphate
NUNA=2′, 3′-seco nucleotide mimics (unlocked nucleobase analogs)
NLNA=locked nucleotide
NfANA=2′-F-Arabino nucleotide
NM=2′-methoxyethyl nucleotide
AM=2′-methoxyethyladenosine-3′-phosphate
AMs=2′-methoxyethyladenosine-3′-phosphorothioate
TM=2′-methoxyethylthymidine-3′-phosphate
TMs=2′-methoxyethylthymidine-3′-phosphorothioate
R=ribitol
(invdN)=any inverted deoxyribonucleotide (3′-3′ linked nucleotide)
(invAb)=inverted (3′-3′ linked) abasic deoxyribonucleotide, see Table 4
(invAb)s=inverted (3′-3′ linked) abasic deoxyribonucleotide-5′-phosphorothioate, see Table 4
(invn)=any inverted 2′-OMe nucleotide (3′-3′ linked nucleotide)
s=phosphorothioate linkage
vpdN=vinyl phosphonate deoxyribonucleotide
(5Me-Nf)=5′-Me, 2′-fluoro nucleotide
cPrp=cyclopropyl phosphonate, see Table 4
epTcPr=see Table 4
epTM=see Table 4

As used herein, an “RNAi agent” means a composition that contains an RNA or RNA-like (e.g., chemically modified RNA) oligonucleotide molecule that is capable of degrading or inhibiting translation of messenger RNA (mRNA) transcripts of a target mRNA in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short interfering RNAs (siRNAs), double-stranded RNAs (dsRNA), micro RNAs (miRNAs), short hairpin RNAs (shRNA), and dicer substrates. The antisense strand of the RNAi agents described herein is at least partially complementary to the mRNA being targeted.

As used herein, and unless otherwise indicated, the term “complementary,” when used to describe a first nucleotide sequence (e.g., RNAi agent sense strand or targeted mRNA) in relation to a second nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or similar conditions in vitro)) and form a duplex or double helical structure under certain conditions with an oligonucleotide or polynucleotide including the second nucleotide sequence. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af are complementary to U (or T) and identical to A for the purposes of determining identity or complementarity.

As used herein, “partially complementary” means that in a hybridized pair of nucleobase sequences, at least 70%, but not all, of the bases in a contiguous sequence of a first polynucleotide will hybridize with the same number of bases in a contiguous sequence of a second polynucleotide.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing recurrence of disease, alleviation of symptoms, diminishment of any direct or indirect pathological consequences of the disease, decreasing the rate of disease progression, amelioration or palliation of the disease state, and remission or improved prognosis. The terms do not require complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as metabolic dysfunction-associated steatotic liver disease (MASLD) and/or metabolic dysfunction-associated steatohepatitis (MASH)). This delay can be of varying lengths of time, depending on the history of the disease and/or subject being treated. As sufficient or significant delay can, in effect, encompass prevention, in that the subject does not develop the disease.

“Preventing,” as used herein, includes providing prophylaxis with respect to the occurrence or recurrence of a disease in a subject that may be predisposed to the disease but has not yet been diagnosed with the disease. In some embodiments, the provided RNAi agent and compositions are used to delay development of a disease or to slow the progression of a disease.

An “effective amount” of an agent, e.g., a pharmaceutical formulation, RNAi agent, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation, RNAi agent, or composition refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject. In some embodiments, the provided methods involve administering the RNAi agent and/or compositions at effective amounts, e.g., therapeutically effective amounts.

As used herein, a “subject” or an “individual” is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject is human.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. W here the stated range includes one or both of the limits, ranges excluding either or both of those included limits are also included in the claimed subject matter. This applies regardless of the breadth of the range.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### II. RNAi AGENTS

Provided herein are inhibitory nucleic acids produced to inhibit the expression of glucokinase (GCK). In some embodiments, the inhibitory nucleic acids described herein include modified oligonucleotides, oligomeric duplexes, antisense oligonucleotides (ASOs), ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), and other oligomeric compounds or oligonucleotide mimetics which hybridize to at least a portion of (GCK) and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. In a specific embodiment, the inhibitory nucleic acid is an interference RNA (RNAi).

In some embodiments, the inhibitory agent is a modified oligonucleotide. In some embodiments, the modified oligonucleotide consists of 10 to 50 linked nucleosides, 10 to 30 linked nucleosides, 12 to 30 linked nucleosides, 15 to 50 linked nucleosides, 20 to 50 linked nucleosides, or 12 to 50 linked nucleosides. In an exemplary embodiment, the modified oligonucleotide consists of between 12 to 30 or 12 to 50 linked nucleosides. In some embodiments, the modified oligonucleotide includes at least one synthetically modified internucleoside linkage. In some embodiments, the modified oligonucleotide includes at least one synthetically modified sugar-phosphate backbone. In some embodiments, the modified oligonucleotide comprises a nucleobase sequence including at least 12, at least 13, at least 14, at least 15, at least 16, at least 17, at least 18, at least 19, at least 20, at least 21, at least 22, at least 23, at least 24, or at least 25 contiguous nucleobases complementary to an equal length portion of SEQ ID NO: 481 (GCK). In some embodiments, the modified oligonucleotide is a modified antisense strand. In some embodiments, the modified oligonucleotide is conjugated to a targeting ligand that targets a hepatocyte.

In some embodiments, the inhibitory agent is an oligomeric duplex. In some embodiments, the oligomeric duplex comprises a first modified oligonucleotide and a second modified oligonucleotide. In some embodiments, the first modified oligonucleotide consists of 10 to 50 linked nucleosides, 10 to 30 linked nucleosides, 12 to 30 linked nucleosides, 15 to 50 linked nucleosides, 20 to 50 linked nucleosides, or 12 to 50 linked nucleosides. In an exemplary embodiment, the first modified oligonucleotide consists of between 12 to 30 or 12 to 50 linked nucleosides. In some embodiments, the first modified oligonucleotide comprises a nucleobase sequence including at least 12, at least 13, at least 14, at least 15, at least 16, at least 17, at least 18, at least 19, at least 20, at least 21, at least 22, at least 23, at least 24, or at least 25 contiguous nucleobases complementary to an equal length portion of SEQ ID NO: 481 (GCK). In some embodiments, the first modified oligonucleotide is a modified antisense strand. In some embodiments, the second modified oligonucleotide consists of 10 to 50 linked nucleosides, 10 to 30 linked nucleosides, 12 to 30 linked nucleosides, 15 to 50 linked nucleosides, 20 to 50 linked nucleosides, or 12 to 50 linked nucleosides. In an exemplary embodiment, the second modified oligonucleotide consists of between 12 to 30 or 12 to 50 linked nucleosides. In some embodiments, the second modified oligonucleotide comprises a complementary region of at least 8 nucleobases that is at least 90% complementary (e.g., at least 91%, 92%, 93%, 94%, 95%, 96%, 97%, 98%, 99%, or 100% complementary) to the nucleobase sequence of an equal length portion of the first modified oligonucleotide. In some embodiments, the second modified oligonucleotide is a modified sense strand. In some embodiments, the first and/or second modified oligonucleotide is conjugated to a targeting ligand that targets a hepatocyte.

In some embodiments, the inhibitory agent is an RNAi agent. In some embodiments, the RNAi agent comprises an antisense strand and a sense strand. In some embodiments, the antisense strand of the RNAi agent is 10 to 40, 10 to 30, 13 to 30, or 13 to 26 nucleotides in length. In some embodiments, the antisense strand of the RNAi agent is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In various implementations, the length of the antisense strand is in a range between any two numbers selected from 10-40. Namely, the length of the antisense strand is in the range of, e.g., 10-11, 10-12, 10-13, 10-14, 10-15, 10-16, 10-17, 10-18, 10-19, 10-20, 10-21, 10-22, 10-23, 10-24, 10-25, 10-26, 10-27, 10-28, 10-29, 10-30, 10-31, 10-32, 10-33, 10-34, 10-35, 10-36, 10-37, 10-38, 10-39, 10-40, 11-12, 11-13, 11-14, 11-15, 11-16, 11-17, 11-18, 11-19, 11-20, 11-21, 11-22, 11-23, 11-24, 11-25, 11-26, 11-27, 11-28, 11-29, 11-30, 11-31, 11-32, 11-33, 11-34, 11-35, 11-36, 11-37, 11-38, 11-39, 11-40, 12-13, 12-14, 12-15, 12-16, 12-17, 12-18, 12-19, 12-20, 12-21, 12-22, 12-23, 12-24, 12-25, 12-26, 12-27, 12-28, 12-29, 12-30, 12-31, 12-32, 12-33, 12-34, 12-35, 12-36, 12-37, 12-38, 12-39, 12-40, 13-14, 13-15, 13-16, 13-17, 13-18, 13-19, 13-20, 13-21, 13-22, 13-23, 13-24, 13-25, 13-26, 13-27, 13-28, 13-29, 13-30, 13-31, 13-32, 13-33, 13-34, 13-35, 13-36, 13-37, 13-38, 13-39, 13-40, 14-15, 14-16, 14-17, 14-18, 14-19, 14-20, 14-21, 14-22, 14-23, 14-24, 14-25, 14-26, 14-27, 14-28, 14-29, 14-30, 14-31, 14-32, 14-33, 14-34, 14-35, 14-36, 14-37, 14-38, 14-39, 14-40, 15-16, 15-17, 15-18, 15-19, 15-20, 15-21, 15-22, 15-23, 15-24, 15-25, 15-26, 15-27, 15-28, 15-29, 15-30, 15-31, 15-32, 15-33, 15-34, 15-35, 15-36, 15-37, 15-38, 15-39, 15-40, 16-17, 16-18, 16-19, 16-20, 16-21, 16-22, 16-23, 16-24, 16-25, 16-26, 16-27, 16-28, 16-29, 16-30, 16-31, 16-32, 16-33, 16-34, 16-35, 16-36, 16-37, 16-38, 16-39, 16-40, 17-18, 17-19, 17-20, 17-21, 17-22, 17-23, 17-24, 17-25, 17-26, 17-27, 17-28, 17-29, 17-30, 17-31, 17-32, 17-33, 17-34, 17-35, 17-36, 17-37, 17-38, 17-39, 17-40, 18-19, 18-20, 18-21, 18-22, 18-23, 18-24, 18-25, 18-26, 18-27, 18-28, 18-29, 18-30, 18-31, 18-32, 18-33, 18-34, 18-35, 18-36, 18-37, 18-38, 18-39, 18-40, 19-20, 19-21, 19-22, 19-23, 19-24, 19-25, 19-26, 19-27, 19-28, 19-29, 19-30, 19-31, 19-32, 19-33, 19-34, 19-35, 19-36, 19-37, 19-38, 19-39, 19-40, 20-21, 20-22, 20-23, 20-24, 20-25, 20-26, 20-27, 20-28, 20-29, 20-30, 20-31, 20-32, 20-33, 20-34, 20-35, 20-36, 20-37, 20-38, 20-39, 20-40, 21-22, 21-23, 21-24, 21-25, 21-26, 21-27, 21-28, 21-29, 21-30, 21-31, 21-32, 21-33, 21-34, 21-35, 21-36, 21-37, 21-38, 21-39, 21-40, 22-23, 22-24, 22-25, 22-26, 22-27, 22-28, 22-29, 22-30, 22-31, 22-32, 22-33, 22-34, 22-35, 22-36, 22-37, 22-38, 22-39, 22-40, 23-24, 23-25, 23-26, 23-27, 23-28, 23-29, 23-30, 23-31, 23-32, 23-33, 23-34, 23-35, 23-36, 23-37, 23-38, 23-39, 23-40, 24-25, 24-26, 24-27, 24-28, 24-29, 24-30, 24-31, 24-32, 24-33, 24-34, 24-35, 24-36, 24-37, 24-38, 24-39, 24-40, 25-26, 25-27, 25-28, 25-29, 25-30, 25-31, 25-32, 25-33, 25-34, 25-35, 25-36, 25-37, 25-38, 25-39, 25-40, 26-27, 26-28, 26-29, 26-30, 26-31, 26-32, 26-33, 26-34, 26-35, 26-36, 26-37, 26-38, 26-39, 26-40, 27-28, 27-29, 27-30, 27-31, 27-32, 27-33, 27-34, 27-35, 27-36, 27-37, 27-38, 27-39, 27-40, 28-29, 28-30, 28-31, 28-32, 28-33, 28-34, 28-35, 28-36, 28-37, 28-38, 28-39, 28-40, 29-30, 29-31, 29-32, 29-33, 29-34, 29-35, 29-36, 29-37, 29-38, 29-39, 29-40, 30-31, 30-32, 30-33, 30-34, 30-35, 30-36, 30-37, 30-38, 30-39, 30-40, 31-32, 31-33, 31-34, 31-35, 31-36, 31-37, 31-38, 31-39, 31-40, 32-33, 32-34, 32-35, 32-36, 32-37, 32-38, 32-39, 32-40, 33-34, 33-35, 33-36, 33-37, 33-38, 33-39, 33-40, 34-35, 34-36, 34-37, 34-38, 34-39, 34-40, 35-36, 35-37, 35-38, 35-39, 35-40, 36-37, 36-38, 36-39, 36-40, 37-38, 37-39, 37-40, 38-39, 38-40, 39-40 or nucleotides. In some embodiments, the antisense strand of the RNAi agent is between 1 to 10 nucleotides, 5 to 15 nucleotides, 10 to 20 nucleotides, 15-25 nucleotides, 20-30 nucleotides, 25-35 nucleotides, or 30 to 40 nucleotides in length. In a specific embodiment, the antisense strand of the RNAi agent is between 20-25 nucleotides in length.

In some embodiments, the sense strand of the RNAi agent is 10 to 40, 10 to 30, 13 to 30, or 13 to 26 nucleotides in length. In some embodiments, the sense strand of the RNAi agent is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In various implementations, the length of the sense strand is in a range between any two numbers selected from 10-40. Namely, the length of the sense strand is in the range of, e.g., 10-11, 10-12, 10-13, 10-14, 10-15, 10-16, 10-17, 10-18, 10-19, 10-20, 10-21, 10-22, 10-23, 10-24, 10-25, 10-26, 10-27, 10-28, 10-29, 10-30, 10-31, 10-32, 10-33, 10-34, 10-35, 10-36, 10-37, 10-38, 10-39, 10-40, 11-12, 11-13, 11-14, 11-15, 11-16, 11-17, 11-18, 11-19, 11-20, 11-21, 11-22, 11-23, 11-24, 11-25, 11-26, 11-27, 11-28, 11-29, 11-30, 11-31, 11-32, 11-33, 11-34, 11-35, 11-36, 11-37, 11-38, 11-39, 11-40, 12-13, 12-14, 12-15, 12-16, 12-17, 12-18, 12-19, 12-20, 12-21, 12-22, 12-23, 12-24, 12-25, 12-26, 12-27, 12-28, 12-29, 12-30, 12-31, 12-32, 12-33, 12-34, 12-35, 12-36, 12-37, 12-38, 12-39, 12-40, 13-14, 13-15, 13-16, 13-17, 13-18, 13-19, 13-20, 13-21, 13-22, 13-23, 13-24, 13-25, 13-26, 13-27, 13-28, 13-29, 13-30, 13-31, 13-32, 13-33, 13-34, 13-35, 13-36, 13-37, 13-38, 13-39, 13-40, 14-15, 14-16, 14-17, 14-18, 14-19, 14-20, 14-21, 14-22, 14-23, 14-24, 14-25, 14-26, 14-27, 14-28, 14-29, 14-30, 14-31, 14-32, 14-33, 14-34, 14-35, 14-36, 14-37, 14-38, 14-39, 14-40, 15-16, 15-17, 15-18, 15-19, 15-20, 15-21, 15-22, 15-23, 15-24, 15-25, 15-26, 15-27, 15-28, 15-29, 15-30, 15-31, 15-32, 15-33, 15-34, 15-35, 15-36, 15-37, 15-38, 15-39, 15-40, 16-17, 16-18, 16-19, 16-20, 16-21, 16-22, 16-23, 16-24, 16-25, 16-26, 16-27, 16-28, 16-29, 16-30, 16-31, 16-32, 16-33, 16-34, 16-35, 16-36, 16-37, 16-38, 16-39, 16-40, 17-18, 17-19, 17-20, 17-21, 17-22, 17-23, 17-24, 17-25, 17-26, 17-27, 17-28, 17-29, 17-30, 17-31, 17-32, 17-33, 17-34, 17-35, 17-36, 17-37, 17-38, 17-39, 17-40, 18-19, 18-20, 18-21, 18-22, 18-23, 18-24, 18-25, 18-26, 18-27, 18-28, 18-29, 18-30, 18-31, 18-32, 18-33, 18-34, 18-35, 18-36, 18-37, 18-38, 18-39, 18-40, 19-20, 19-21, 19-22, 19-23, 19-24, 19-25, 19-26, 19-27, 19-28, 19-29, 19-30, 19-31, 19-32, 19-33, 19-34, 19-35, 19-36, 19-37, 19-38, 19-39, 19-40, 20-21, 20-22, 20-23, 20-24, 20-25, 20-26, 20-27, 20-28, 20-29, 20-30, 20-31, 20-32, 20-33, 20-34, 20-35, 20-36, 20-37, 20-38, 20-39, 20-40, 21-22, 21-23, 21-24, 21-25, 21-26, 21-27, 21-28, 21-29, 21-30, 21-31, 21-32, 21-33, 21-34, 21-35, 21-36, 21-37, 21-38, 21-39, 21-40, 22-23, 22-24, 22-25, 22-26, 22-27, 22-28, 22-29, 22-30, 22-31, 22-32, 22-33, 22-34, 22-35, 22-36, 22-37, 22-38, 22-39, 22-40, 23-24, 23-25, 23-26, 23-27, 23-28, 23-29, 23-30, 23-31, 23-32, 23-33, 23-34, 23-35, 23-36, 23-37, 23-38, 23-39, 23-40, 24-25, 24-26, 24-27, 24-28, 24-29, 24-30, 24-31, 24-32, 24-33, 24-34, 24-35, 24-36, 24-37, 24-38, 24-39, 24-40, 25-26, 25-27, 25-28, 25-29, 25-30, 25-31, 25-32, 25-33, 25-34, 25-35, 25-36, 25-37, 25-38, 25-39, 25-40, 26-27, 26-28, 26-29, 26-30, 26-31, 26-32, 26-33, 26-34, 26-35, 26-36, 26-37, 26-38, 26-39, 26-40, 27-28, 27-29, 27-30, 27-31, 27-32, 27-33, 27-34, 27-35, 27-36, 27-37, 27-38, 27-39, 27-40, 28-29, 28-30, 28-31, 28-32, 28-33, 28-34, 28-35, 28-36, 28-37, 28-38, 28-39, 28-40, 29-30, 29-31, 29-32, 29-33, 29-34, 29-35, 29-36, 29-37, 29-38, 29-39, 29-40, 30-31, 30-32, 30-33, 30-34, 30-35, 30-36, 30-37, 30-38, 30-39, 30-40, 31-32, 31-33, 31-34, 31-35, 31-36, 31-37, 31-38, 31-39, 31-40, 32-33, 32-34, 32-35, 32-36, 32-37, 32-38, 32-39, 32-40, 33-34, 33-35, 33-36, 33-37, 33-38, 33-39, 33-40, 34-35, 34-36, 34-37, 34-38, 34-39, 34-40, 35-36, 35-37, 35-38, 35-39, 35-40, 36-37, 36-38, 36-39, 36-40, 37-38, 37-39, 37-40, 38-39, 38-40, 39-40 or nucleotides In some embodiments, the sense strand of the RNAi agent is between 1 to 10 nucleotides, 5 to 15 nucleotides, 10 to 20 nucleotides, 15-25 nucleotides, 20-30 nucleotides, 25-35 nucleotides, or 30 to 40 nucleotides in length. In a specific embodiment, the antisense strand of the RNAi agent is between 20-25 nucleotides in length.

In some embodiments, the RNAi agent comprises an antisense strand with a 3′ overhang of nucleotides. In some embodiments, the antisense strand 3′ overhang is 1, 2, 3, 4, 5, 6, 7, 8, 9, or 10 nucleotides in length. In some embodiments, the antisense strand comprises a 3′ overhang of 1-10 nucleotides in length, 1-9 nucleotides in length, 1-8 nucleotides in length, 1-7 nucleotides in length, 1-6 nucleotides in length, 1-5 nucleotides in length, 1-4 nucleotides in length, 1-3 nucleotides in length, 1 or 2 nucleotides in length, or 1 nucleotide in length. In a specific embodiment, the RNAi agent comprises an antisense strand comprising a 3′ overhang of 1-5 nucleotides in length.

In some embodiments, the RNAi agent comprises an antisense strand with a 5′ overhang of nucleotides. In some embodiments, the antisense strand 5′ overhang is 1, 2, 3, 4, 5, 6, 7, 8, 9, or 10 nucleotides in length. In some embodiments, the antisense strand comprises a 5′ overhang of 1-10 nucleotides in length, 1-9 nucleotides in length, 1-8 nucleotides in length, 1-7 nucleotides in length, 1-6 nucleotides in length, 1-5 nucleotides in length, 1-4 nucleotides in length, 1-3 nucleotides in length, 1 or 2 nucleotides in length, or 1 nucleotide in length. In a specific embodiment, the RNAi agent comprises an antisense strand comprising a 5′ overhang of 1-5 nucleotides in length.

In some embodiments, the RNAi agent comprises a sense strand with a 3′ overhang of nucleotides. In some embodiments, the sense strand 3′ overhang is 1, 2, 3, 4, 5, 6, 7, 8, 9, or 10 nucleotides in length. In some embodiments, the sense strand comprises a 3′ overhang of 1-10 nucleotides in length, 1-9 nucleotides in length, 1-8 nucleotides in length, 1-7 nucleotides in length, 1-6 nucleotides in length, 1-5 nucleotides in length, 1-4 nucleotides in length, 1-3 nucleotides in length, 1 or 2 nucleotides in length, or 1 nucleotide in length. In a specific embodiment, the RNAi agent comprises a sense strand comprising a 3′ overhang of 1-5 nucleotides in length.

In some embodiments, the RNAi agent comprises a sense strand with a 5′ overhang of nucleotides. In some embodiments, the sense strand 5′ overhang is 1, 2, 3, 4, 5, 6, 7, 8, 9, or 10 nucleotides in length. In some embodiments, the sense strand comprises a 5′ overhang of 1-10 nucleotides in length, 1-9 nucleotides in length, 1-8 nucleotides in length, 1-7 nucleotides in length, 1-6 nucleotides in length, 1-5 nucleotides in length, 1-4 nucleotides in length, 1-3 nucleotides in length, 1 or 2 nucleotides in length, or 1 nucleotide in length. In a specific embodiment, the RNAi agent comprises a sense strand comprising a 5′ overhang of 1-5 nucleotides in length.

In some embodiments, the 3′ overhang of the sense and/or the antisense strand comprises uracil and/or comprises nucleotides complementary to a sequence in a pregenomic RNA and/or mRNA molecule that encodes GCK. In some embodiments, the 5′ overhang of the sense and/or the antisense strand comprises uracil and/or comprises nucleotides complementary to a sequence in a pregenomic RNA and/or mRNA molecule that encodes GCK.

In some embodiments, the 3′ overhang of the sense and/or the antisense strand comprises uracil and/or comprises nucleotides partially complementary to a sequence in a pregenomic RNA and/or mRNA molecule that encodes GCK. In some embodiments, the 5′ overhang of the sense and/or the antisense strand comprises uracil and/or comprises nucleotides partially complementary to a sequence in a pregenomic RNA and/or mRNA molecule that encodes GCK.

In some embodiments, the antisense strand of the RNAi agent comprises nucleobases that complement a sequence in an mRNA molecule that encodes GCK. In some embodiments, the mRNA molecule that encodes GCK comprises a nucleobase sequence according to SEQ ID NO: 481. In some embodiments, the antisense strand comprises at least 5, 6, 7, 8, 9, 10, 11, 12, 13, 14, 15, 16, 17, 18, 19, 20, 21, 22, 23, 24, 25, 26, 27, 28, 29, or 30 contiguous bases that complement a sequence in a GCK mRNA molecule. In some embodiments, the antisense strand comprises at least 14 contiguous bases that complement a sequence in an GCK mRNA molecule. In some embodiments, the antisense strand comprises at least 15 contiguous bases that complement a sequence in a GCK mRNA molecule. In some embodiments, the antisense strand comprises at least 16 contiguous bases that complement a sequence in a GCK mRNA molecule. In some embodiments, the antisense strand comprises at least 17 contiguous bases that complement a sequence in a GCK mRNA molecule.

In some embodiments, the antisense strand of the RNAi agent comprises a uracil nucleobase at the 5′ terminus or the sense strand of the RNAi agent comprises an adenine nucleobase at the 3′ terminus. In some embodiments, the antisense strand of the RNAi agent comprises a uracil nucleobase at the 5′ terminus and the sense strand of the RNAi agent comprises an adenine nucleobase at the 3′ terminus. The addition of an extra uracil nucleobase at the 5′ terminus of the antisense strand and/or an extra adenine nucleobase at the 3′ terminus of the sense strand can optimize performance of the RNAi agent by promoting the correct strand uptake by the RNA-induced silencing complex (RISC), enhancing duplex stability, protecting against degradation, and minimizing off-target effects. These modifications may help ensure that the designed siRNA is efficient and effective in gene silencing. The antisense strand sequences provided herein may comprise an extra uracil nucleobase at the 5′ terminus, and/or the sense strand sequences provided herein may comprise an extra adenine nucleobase at the 3′ terminus. In some embodiments, the antisense strand comprises, in order, nucleotides 1-23 of any one of SEQ ID NOS: 1-96 and 193-288. In some embodiments, the sense strand comprises, nucleotides 1-20 of any one of SEQ ID NOS: 97-192, 289-384, and 385-480.

In some embodiments, the antisense strand and the sense strand of the RNAi agent are self-complementary (i.e., each strand comprises a nucleotide sequence that is complementary to nucleotide sequence of the other strand) or at least partially complementary. In this instance, the antisense strand and sense strand will form a duplex (i.e., double stranded structure) that does not comprise overhangs. In some embodiments, the RNAi agent comprises a duplex structure. In some embodiments, the RNAi agent comprises a duplex length of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides long. In some embodiments, the RNAi agent comprises a duplex of between 5 to 25 nucleotides long, 10 to 30 nucleotides long, 15 to 35 nucleotides long, 20 to 40 nucleotides long, 10 to 20 nucleotides long, 15 to 25 nucleotides long, 20 to 30 nucleotides long, 25 to 35 nucleotides long, 30 to 40 nucleotides long, 5 to 20 nucleotides long, 10 to 25 nucleotides long, 15 to 30 nucleotides long, 20 to 35 nucleotides long, or 25 to 40 nucleotides long. In an exemplary embodiment, the RNAi agent comprises a duplex of between 15 to 30 nucleotides long. In some embodiments, the RNAi agent comprises a duplex of 14 nucleotides in length, a duplex of 15 nucleotides in length, 16 nucleotides in length, 17 nucleotides in length, 18 nucleotides in length, 19 nucleotides in length, or 20 nucleotides in length. In a specific embodiment, the RNAi agent comprises a duplex of 16 nucleotides in length.

In some embodiments, the RNAi agent comprises a duplex region of any length that permits specific degradation of a desired target mRNA (i.e., GCK) through RISC, and may range from about 9 to 36 nucleotides in length, e.g., about 10-30, 10-35, 14-30, 14-35, 15-30, 15-35, 24-30, or 19-23 nucleotides in length. In some embodiments, the RNAi agent comprises a duplex region of about 9, 10, 11, 12, 13, 14, 15, 16, 17, 18, 19, 20, 21, 22, 23, 24, 25, 26, 27, 28, 29, 30, 31, 32, 33, 34, 35, or 36 nucleotides in length, such as about 14-30, 14-35, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. Ranges and lengths intermediate to the above recited example ranges and lengths are also contemplated to be part of the invention.

TABLE 1
GCK RNA i agent antisense strand sequences.

| | | Modified | |
|---|---|---|---|
| Antisense | SEQ | Antisense | SEQ |
| Sequence | ID | Sequence | ID |
| (5′ → 3′) | NO: | (5′ → 3′) | NO: |
| CCGTCTCATCACCTTCTTCAGGT | 1 | csCfsgucUfcaucac | 193 |
| | | cUfuCfuucagsgsu | |
| ATCCGTCTCATCACCTTCTTCAG | 2 | asUfsccgUfcucauc | 194 |
| | | aCfcUfucuucsasg | |
| ATCTTCACACTGGCCTCTTCATG | 3 | asUfscuuCfacacug | 195 |
| | | gCfcUfcuucasusg | |
| GCATCTTCACACTGGCCTCTTCA | 4 | gsCfsaucUfucacac | 196 |
| | | uGfgCfcucuuscsa | |
| GAGCGCACGTAGGTGGGCAGCAT | 5 | gsAfsgcgCfacguag | 197 |
| | | gUfgGfgcagcsasu | |
| CTCACCTTCTCCCACCTTCACCA | 6 | csUfscacCfuucucc | 198 |
| | | cAfcCfuucacscsa | |
| GGTGTTTGGTCTTCACGCTCCAC | 7 | gsGfsuguUfuggucu | 199 |
| | | uCfaCfgcuccsasc | |
| ATCTGGTGTTTGGTCTTCACGCT | 8 | asUfscugGfuguuug | 200 |
| | | gUfcUfucacgscsu | |
| ACATCTGGTGTTTGGTCTTCACG | 9 | asCfsaucUfgguguu | 201 |
| | | uGfgUfcuucascsg | |
| TACATCTGGTGTTTGGTCTTCAC | 10 | usAfscauCfuggugu | 202 |
| | | uUfgGfucuucsasc | |
| AGTACATCTGGTGTTTGGTCTTC | 11 | asGfsuacAfucuggu | 203 |
| | | gUfuUfggucususc | |
| GCATCTCAGCAGTGCCGGTCATG | 12 | gsCfsaucUfcagcag | 204 |
| | | uGfcCfggucasusg | |
| AGCATCTCAGCAGTGCCGGTCAT | 13 | asGfscauCfucagca | 205 |
| | | gUfgCfcggucsasu | |
| TCGAAGAGCATCTCAGCAGTGCC | 14 | usCfsgaaGfagcauc | 206 |
| | | uCfaGfcagugscsc | |
| GTCGAAGAGCATCTCAGCAGTGC | 15 | gsUfscgaAfgagcau | 207 |
| | | cUfcAfgcagusgsc | |
| ATGTAGTCGAAGAGCATCTCAGC | 16 | asUfsguaGfucgaag | 208 |
| | | aGfcAfucucasgsc | |
| TCATCTGATGCTTGTCCAGGAAG | 17 | usCfsaucUfgaugcu | 209 |
| | | uGfuCfcaggasasg | |
| GATGCCCTTATCGATGTCTTCGT | 18 | gsAfsugcCfcuuauc | 210 |
| | | gAfuGfucuucsgsu | |
| AGGATGCCCTTATCGATGTCTTC | 19 | asGfsgauGfcccuua | 211 |
| | | uCfgAfugucususc | |
| GTTGAGAAGGATGCCCTTATCGA | 20 | gsUfsugaGfaaggau | 212 |
| | | gCfcCfuuaucsgsa | |
| TCCAGTTGAGAAGGATGCCCTTA | 21 | usCfscagUfugagaa | 213 |
| | | gGfaUfgcccususa | |
| TGGTCCAGTTGAGAAGGATGCCC | 22 | usGfsgucCfaguuga | 214 |
| | | gAfaGfgaugcscsc | |
| CATTGTTCCCTTCTGCTCCTGAG | 23 | csAfsuugUfucccuu | 215 |
| | | cUfgCfuccugsasg | |
| GACATTGTTCCCTTCTGCTCCTG | 24 | gsAfscauUfguuccc | 216 |
| | | uUfcUfgcuccsusg | |
| CGACATTGTTCCCTTCTGCTCCT | 25 | csGfsacaUfuguucc | 217 |
| | | cUfuCfugcucscsu | |
| ACGACATTGTTCCCTTCTGCTCC | 26 | asCfsgacAfuuguuc | 218 |
| | | cCfuUfcugcuscsc | |
| CCACGACATTGTTCCCTTCTGCT | 27 | csCfsacgAfcauugu | 219 |
| | | uCfcCfuucugscsu | |
| CCCACGACATTGTTCCCTTCTGC | 28 | csCfscacGfacauug | 220 |
| | | uUfcCfcuucusgsc | |
| ACCATTGCCACCACATCCATTTC | 29 | asCfscauUfgccacc | 221 |
| | | aCfaUfccauususc | |
| ATTCACCATTGCCACCACATCCA | 30 | asUfsucaCfcauugc | 222 |
| | | cAfcCfacaucscsa | |
| CATTCACCATTGCCACCACATCC | 31 | csAfsuucAfccauug | 223 |
| | | cCfaCfcacauscsc | |
| TCATTCACCATTGCCACCACATC | 32 | usCfsauuCfaccauu | 224 |
| | | gCfcAfccacasusc | |
| GTGTCATTCACCATTGCCACCAC | 33 | gsUfsgucAfuucacc | 225 |
| | | aUfuGfccaccsasc | |
| ACCGTGTCATTCACCATTGCCAC | 34 | asCfscguGfucauuc | 226 |
| | | aCfcAfuugccsasc | |
| CACCGTGTCATTCACCATTGCCA | 35 | csAfsccgUfgucauu | 227 |
| | | cAfcCfauugcscsa | |
| GGCCACCGTGTCATTCACCATTG | 36 | gsGfsccaCfcguguc | 228 |
| | | aUfuCfaccaususg | |
| TGGCCACCGTGTCATTCACCATT | 37 | usGfsgccAfccgugu | 229 |
| | | cAfuUfcaccasusu | |
| AGTAGCAGGAGATCATCGTGGCC | 38 | asGfsuagCfaggaga | 230 |
| | | uCfaUfcguggscsc | |
| TAGTAGCAGGAGATCATCGTGGC | 39 | usAfsguaGfcaggag | 231 |
| | | aUfcAfucgugsgsc | |
| GTAGTAGCAGGAGATCATCGTGG | 40 | gsUfsaguAfgcagga | 232 |
| | | gAfuCfaucgusgsg | |
| CGTAGTAGCAGGAGATCATCGTG | 41 | csGfsuagUfagcagg | 233 |
| | | aGfaUfcaucgsusg | |
| TCGTAGTAGCAGGAGATCATCGT | 42 | usCfsguaGfuagcag | 234 |
| | | gAfgAfucaucsgsu | |
| CTTCGTAGTAGCAGGAGATCATC | 43 | csUfsucgUfaguagc | 235 |
| | | aGfgAfgaucasusc | |
| TCTTCGTAGTAGCAGGAGATCAT | 44 | usCfsuucGfuaguag | 236 |
| | | cAfgGfagaucsasu | |
| CACTGATGGTCTTCGTAGTAGCA | 45 | csAfscugAfuggucu | 237 |
| | | uCfgUfaguagscsa | |
| GCACTGATGGTCTTCGTAGTAGC | 46 | gsCfsacuGfaugguc | 238 |
| | | uUfcGfuaguasgsc | |
| CTCGCACTGATGGTCTTCGTAGT | 47 | csUfscgcAfcugaug | 239 |
| | | gUfcUfucguasgsu | |
| GACCTCGCACTGATGGTCTTCGT | 48 | gsAfsccuCfgcacug | 240 |
| | | aUfgGfucuucsgsu | |
| CCGACCTCGCACTGATGGTCTTC | 49 | csCfsgacCfucgcac | 241 |
| | | uGfaUfggucususc | |
| TGCCGACCTCGCACTGATGGTCT | 50 | usGfsccgAfccucgc | 242 |
| | | aCfuGfaugguscsu | |
| CATGCCGACCTCGCACTGATGGT | 51 | csAfsugcCfgaccuc | 243 |
| | | gCfaCfugaugsgsu | |
| ATCATGCCGACCTCGCACTGATG | 52 | asUfscauGfccgacc | 244 |
| | | uCfgCfacugasusg | |
| CCACATTCTGCATCTCCTCCATG | 53 | csCfsacaUfucugca | 245 |
| | | uCfuCfcuccasusg | |
| AGGCGGTCATACTCCAGCAGGAA | 54 | asGfsgcgGfucauac | 246 |
| | | uCfcAfgcaggsasa | |
| TCCACCAGGCGGTCATACTCCAG | 55 | usCfscacCfaggcgg | 247 |
| | | uCfaUfacuccsasg | |
| GTCCACCAGGCGGTCATACTCCA | 56 | gsUfsccaCfcaggcg | 248 |
| | | gUfcAfuacucscsa | |
| CGTCCACCAGGCGGTCATACTCC | 57 | csGfsuccAfccaggc | 249 |
| | | gGfuCfauacuscsc | |
| TCGTCCACCAGGCGGTCATACTC | 58 | usCfsgucCfaccagg | 250 |
| | | cGfgUfcauacsusc | |
| CTCGTCCACCAGGCGGTCATACT | 59 | csUfscguCfcaccag | 251 |
| | | gCfgGfucauascsu | |
| GAGCTTCTCATACAGCTGCTGAC | 60 | gsAfsgcuUfoucaua | 252 |
| | | cAfgCfugcugsasc | |
| CCTATGAGCTTCTCATACAGCTG | 61 | csCfsuauGfagcuuc | 253 |
| | | uCfaUfacagcsusg | |
| CACCTATGAGCTTCTCATACAGC | 62 | csAfsccuAfugagcu | 254 |
| | | uCfuCfauacasgsc | |
| CCACCTATGAGCTTCTCATACAG | 63 | csCfsaccUfaugagc | 255 |
| | | uUfcUfcauacsasg | |
| GCCACCTATGAGCTTCTCATACA | 64 | gsCfscacCfuaugag | 256 |
| | | cUfuCfucauascsa | |
| TGCCACCTATGAGCTTCTCATAC | 65 | usGfsccaCfcuauga | 257 |
| | | gCfuUfcucausasc | |
| GTACTTGCCACCTATGAGCTTCT | 66 | gsUfsacuUfgccacc | 258 |
| | | uAfuGfagcuuscsu | |
| TGTACTTGCCACCTATGAGCTTC | 67 | usGfsuacUfugccac | 259 |
| | | cUfaUfgagcususc | |
| CCATGTACTTGCCACCTATGAGC | 68 | csCfsaugUfacuugc | 260 |
| | | cAfcCfuaugasgsc | |
| TCGCCCATGTACTTGCCACCTAT | 69 | usCfsgccCfauguac | 261 |
| | | uUfgCfcaccusasu | |
| GCTCGCCCATGTACTTGCCACCT | 70 | gsCfsucgCfccaugu | 262 |
| | | aCfuUfgccacscsu | |
| CAGCTCGCCCATGTACTTGCCAC | 71 | csAfsgcuCfgcccau | 263 |
| | | gUfaCfuugccsasc | |
| TCGTCCACGAGCCTGAGCAGCAC | 72 | usCfsgucCfacgagc | 264 |
| | | cUfgAfgcagcsasc | |
| TTCGTCCACGAGCCTGAGCAGCA | 73 | usUfscguCfcacgag | 265 |
| | | cCfuGfagcagscsa | |
| TTGTAGATCTGCTTGCGGTCGCC | 74 | usUfsguaGfaucugc | 266 |
| | | uUfgCfggucgscsc | |
| TCAGGATGTTGTAGATCTGCTTG | 75 | usCfsaggAfuguugu | 267 |
| | | aGfaUfcugcususg | |
| CGTGCTCAGGATGTTGTAGATCT | 76 | csGfsugcUfcaggau | 268 |
| | | gUfuGfuagauscsu | |
| AGCGTGCTCAGGATGTTGTAGAT | 77 | asGfscguGfcucagg | 269 |
| | | aUfgUfuguagsasu | |
| CACGCCCACAGTGATGCGCATTA | 78 | csAfscgcCfcacagu | 270 |
| | | gAfuGfcgcaususa | |
| AGCCATCCACGCCCACAGTGATG | 79 | asGfsccaUfccacgc | 271 |
| | | cCfaCfagugasusg | |
| GAGCCATCCACGCCCACAGTGAT | 80 | gsAfsgccAfuccacg | 272 |
| | | cCfcAfcagugsasu | |
| GGAGCCATCCACGCCCACAGTGA | 81 | gsGfsagcCfauccac | 273 |
| | | gCfcCfacagusgsa | |
| ACACGGAGCCATCCACGCCCACA | 82 | asCfsacgGfagccau | 274 |
| | | cCfaCfgcccascsa | |
| GTACACGGAGCCATCCACGCCCA | 83 | gsUfsacaCfggagcc | 275 |
| | | aUfcCfacgccscsa | |
| TGTACACGGAGCCATCCACGCCC | 84 | usGfsuacAfcggagc | 276 |
| | | cAfuCfcacgcscsc | |
| AGCTTGTACACGGAGCCATCCAC | 85 | asGfscuuGfuacacg | 277 |
| | | gAfgCfcauccsasc | |
| GTGATCTCGCAGCTGGGCGTCAG | 86 | gsUfsgauCfucgcag | 278 |
| | | cUfgGfgcgucsasg | |
| GACTCGATGAAGGTGATCTCGCA | 87 | gsAfscucGfaugaag | 279 |
| | | gUfgAfucucgscsa | |
| TCCGACTCGATGAAGGTGATCTC | 88 | usCfscgaCfucgaug | 280 |
| | | aAfgGfugaucsusc | |
| CTCCGACTCGATGAAGGTGATCT | 89 | csUfsccgAfcucgau | 281 |
| | | gAfaGfgugauscsu | |
| CTGACAGTGTGGAAATGCATCAA | 90 | csUfsgacAfgugugg | 282 |
| | | aAfaUfgcaucsasa | |
| CCCACACAGGATGAGTTCCCAGG | 91 | csCfscacAfcaggau | 283 |
| | | gAfgUfucccasgsg | |
| TGGTCAAGCTGTTGGAGCTGCCT | 92 | usGfsgucAfagcugu | 284 |
| | | uGfgAfgcugcscsu | |
| TAGGTCTGGTCAAGCTGTTGGAG | 93 | usAfsgguCfugguca | 285 |
| | | aGfcUfguuggsasg | |
| GTCCATCCCAGAATCACAAGCCA | 94 | gsUfsccaUfcccaga | 286 |
| | | aUfcAfcaagcscsa | |
| GGTCCATCCCAGAATCACAAGCC | 95 | gsGfsuccAfucccag | 287 |
| | | aAfuCfacaagscsc | |
| GGGTTTCTTCCTGAGCCAGCGCT | 96 | gsGfsguuUfcuuccu | 288 |
| | | gAfgCfcagcgscsu | |

TABLE 2
GCK RNA i agent sense strand sequences.

| | | | | Modified | |
|---|---|---|---|---|---|
| | | | | Sense | |
| | | | | Sequence | |
| | | | | with 3′ | |
| | | Modified | | conjugated | |
| Sense | SEQ | Sense | SEQ | targeting | SEQ |
| Sequence | ID | Sequence | ID | moiety | ID |
| (5′ → 3′) | NO: | (5′ → 3′) | NO: | (5′ → 3′) | NO: |
| CTGAAGAAGGT | 97 | csusgaagAf | 289 | csusgaagAfaGfG | 385 |
| GATGAGACGG | | aGfGfUfgau | | fUfgaugagacgg | |
| | | gagacsgsg | | | |
| GAAGAAGGTGA | 98 | gsasagaaGf | 290 | gsasagaaGfgUfG | 386 |
| TGAGACGGAT | | gUfGfAfuga | | fAfugagacggau | |
| | | gacggsasu | | | |
| TGAAGAGGCCA | 99 | usgsaagaGf | 291 | usgsaagaGfgCfC | 387 |
| GTGTGAAGAT | | gCfCfAfgug | | fAfgugugaagau | |
| | | ugaagsasu | | | |
| AAGAGGCCAGT | 100 | asasgaggCf | 292 | asasgaggCfcAfG | 388 |
| GTGAAGATGC | | cAfGfUfgug | | fUfgugaagaugc | |
| | | aagausgsc | | | |
| GCTGCCCACCT | 101 | gscsugccCf | 293 | gscsugccCfaCfC | 389 |
| ACGTGCGCTC | | aCfCfUfacg | | fUfacgugcgcuc | |
| | | ugcgcsusc | | | |
| GTGAAGGTGGG | 102 | gsusgaagGf | 294 | gsusgaagGfuGfG | 390 |
| AGAAGGTGAG | | uGfGfGfaga | | fGfagaaggugag | |
| | | aggugsasg | | | |
| GGAGCGTGAAG | 103 | gsgsagcgUf | 295 | gsgsagcgUfgAfA | 391 |
| ACCAAACACC | | gAfAfGfacc | | fGfaccaaacacc | |
| | | aaacascsc | | | |
| CGTGAAGACCA | 104 | csgsugaaGf | 296 | csgsugaaGfaCfC | 392 |
| AACACCAGAT | | aCfCfAfaac | | fAfaacaccagau | |
| | | accagsasu | | | |
| TGAAGACCAAA | 105 | usgsaagaCf | 297 | usgsaagaCfcAfA | 393 |
| CACCAGATGT | | cAfAfAfcac | | fAfcaccagaugu | |
| | | cagausgsu | | | |
| GAAGACCAAAC | 106 | gsasagacCf | 298 | gsasagacCfaAfA | 394 |
| ACCAGATGTA | | aAfAfCfacc | | fCfaccagaugua | |
| | | agaugsusa | | | |
| AGACCAAACAC | 107 | asgsaccaAf | 299 | asgsaccaAfaCfA | 395 |
| CAGATGTACT | | aCfAfCfcag | | fCfcagauguacu | |
| | | auguascsu | | | |
| TGACCGGCACT | 108 | usgsaccgGf | 300 | usgsaccgGfcAfC | 396 |
| GCTGAGATGC | | cAfCfUfgcu | | fUfgcugagaugc | |
| | | gagausgsc | | | |
| GACCGGCACTG | 109 | gsasccggCf | 301 | gsasccggCfaCfU | 397 |
| CTGAGATGCT | | aCfUfGfcug | | fGfcugagaugcu | |
| | | agaugscsu | | | |
| CACTGCTGAGA | 110 | csascugcUf | 302 | csascugcUfgAfG | 398 |
| TGCTCTTCGA | | gAfGfAfugc | | fAfugcucuucga | |
| | | ucuucsgsa | | | |
| ACTGCTGAGAT | 111 | ascsugcuGf | 303 | ascsugcuGfaGfA | 399 |
| GCTCTTCGAC | | aGfAfUfgcu | | fUfgcucuucgac | |
| | | cuucgsasc | | | |
| TGAGATGCTCT | 112 | usgsagauGf | 304 | usgsagauGfcUfC | 400 |
| TCGACTACAT | | cUfCfUfucg | | fUfucgacuacau | |
| | | acuacsasu | | | |
| TCCTGGACAAG | 113 | uscscuggAf | 305 | uscscuggAfcAfA | 401 |
| CATCAGATGA | | cAfAfGfcau | | fGfcaucagauga | |
| | | cagausgsa | | | |
| GAAGACATCGA | 114 | gsasagacAf | 306 | gsasagacAfuCfG | 402 |
| TAAGGGCATC | | uCfGfAfuaa | | fAfuaagggcauc | |
| | | gggcasusc | | | |
| AGACATCGATA | 115 | asgsacauCf | 307 | asgsacauCfgAfU | 403 |
| AGGGCATCCT | | gAfUfAfagg | | fAfagggcauccu | |
| | | gcaucscsu | | | |
| GATAAGGGCAT | 116 | gsasuaagGf | 308 | gsasuaagGfgCfA | 404 |
| CCTTCTCAAC | | gCfAfUfccu | | fUfccuucucaac | |
| | | ucucasasc | | | |
| AGGGCATCCTT | 117 | asgsggcaUf | 309 | asgsggcaUfcCfU | 405 |
| CTCAACTGGA | | cCfUfUfcuc | | fUfcucaacugga | |
| | | aacugsgsa | | | |
| GCATCCTTCTC | 118 | gscsauccUf | 310 | gscsauccUfuCfU | 406 |
| AACTGGACCA | | uCfUfCfaac | | fCfaacuggacca | |
| | | uggacscsa | | | |
| CAGGAGCAGAA | 119 | csasggagCf | 311 | csasggagCfaGfA | 407 |
| GGGAACAATG | | aGfAfAfggg | | fAfgggaacaaug | |
| | | aacaasusg | | | |
| GGAGCAGAAGG | 120 | gsgsagcaGf | 312 | gsgsagcaGfaAfG | 408 |
| GAACAATGTC | | aAfGfGfgaa | | fGfgaacaauguc | |
| | | caaugsusc | | | |
| GAGCAGAAGGG | 121 | gsasgcagAf | 313 | gsasgcagAfaGfG | 409 |
| AACAATGTCG | | aGfGfGfaac | | fGfaacaaugucg | |
| | | aauguscsg | | | |
| AGCAGAAGGGA | 122 | asgscagaAf | 314 | asgscagaAfgGfG | 410 |
| ACAATGTCGT | | gGfGfAfaca | | fAfacaaugucgu | |
| | | augucsgsu | | | |
| CAGAAGGGAAC | 123 | csasgaagGf | 315 | csasgaagGfgAfA | 411 |
| AATGTCGTGG | | gAfAfCfaau | | fCfaaugucgugg | |
| | | gucgusgsg | | | |
| AGAAGGGAACA | 124 | asgsaaggGf | 316 | asgsaaggGfaAfC | 412 |
| ATGTCGTGGG | | aAfCfAfaug | | fAfaugucguggg | |
| | | ucgugsgsg | | | |
| AATGGATGTGG | 125 | asasuggaUf | 317 | asasuggaUfgUfG | 413 |
| TGGCAATGGT | | gUfGfGfugg | | fGfuggcaauggu | |
| | | caaugsgsu | | | |
| GATGTGGTGGC | 126 | gsasugugGf | 318 | gsasugugGfuGfG | 414 |
| AATGGTGAAT | | uGfGfCfaau | | fCfaauggugaau | |
| | | ggugasasu | | | |
| ATGTGGTGGCA | 127 | asusguggUf | 319 | asusguggUfgGfC | 415 |
| ATGGTGAATG | | gGfCfAfaug | | fAfauggugaaug | |
| | | gugaasusg | | | |
| TGTGGTGGCAA | 128 | usgsugguGf | 320 | usgsugguGfgCfA | 416 |
| TGGTGAATGA | | gCfAfAfugg | | fAfuggugaauga | |
| | | ugaausgsa | | | |
| GGTGGCAATGG | 129 | gsgsuggcAf | 321 | gsgsuggcAfaUfG | 417 |
| TGAATGACAC | | aUfGfGfuga | | fGfugaaugacac | |
| | | augacsasc | | | |
| GGCAATGGTGA | 130 | gsgscaauGf | 322 | gsgscaauGfgUfG | 418 |
| ATGACACGGT | | gUfGfAfaug | | fAfaugacacggu | |
| | | acacgsgsu | | | |
| GCAATGGTGAA | 131 | gscsaaugGf | 323 | gscsaaugGfuGfA | 419 |
| TGACACGGTG | | uGfAfAfuga | | fAfugacacggug | |
| | | cacggsusg | | | |
| ATGGTGAATGA | 132 | asusggugAf | 324 | asusggugAfaUfG | 420 |
| CACGGTGGCC | | aUfGfAfcac | | fAfcacgguggcc | |
| | | gguggscsc | | | |
| TGGTGAATGAC | 133 | usgsgugaAf | 325 | usgsgugaAfuGfA | 421 |
| ACGGTGGCCA | | uGfAfCfacg | | fCfacgguggcca | |
| | | guggcscsa | | | |
| CCACGATGATC | 134 | cscsacgaUf | 326 | cscsacgaUfgAfU | 422 |
| TCCTGCTACT | | gAfUfCfucc | | fCfuccugcuacu | |
| | | ugcuascsu | | | |
| CACGATGATCT | 135 | csascgauGf | 327 | csascgauGfaUfC | 423 |
| CCTGCTACTA | | aUfCfUfccu | | fUfccugcuacua | |
| | | gcuacsusa | | | |
| ACGATGATCTC | 136 | ascsgaugAf | 328 | ascsgaugAfuCfU | 424 |
| CTGCTACTAC | | uCfUfCfcug | | fCfcugcuacuac | |
| | | cuacusasc | | | |
| CGATGATCTCC | 137 | csgsaugaUf | 329 | csgsaugaUfcUfC | 425 |
| TGCTACTACG | | cUfCfCfugc | | fCfugcuacuacg | |
| | | uacuascsg | | | |
| GATGATCTCCT | 138 | gsasugauCf | 330 | gsasugauCfuCfC | 426 |
| GCTACTACGA | | uCfCfUfgcu | | fUfgcuacuacga | |
| | | acuacsgsa | | | |
| TGATCTCCTGC | 139 | usgsaucuCf | 331 | usgsaucuCfcUfG | 427 |
| TACTACGAAG | | cUfGfCfuac | | fCfuacuacgaag | |
| | | uacgasasg | | | |
| GATCTCCTGCT | 140 | gsasucucCf | 332 | gsasucucCfuGfC | 428 |
| ACTACGAAGA | | uGfCfUfacu | | fUfacuacgaaga | |
| | | acgaasgsa | | | |
| CTACTACGAAG | 141 | csusacuaCf | 333 | csusacuaCfgAfA | 429 |
| ACCATCAGTG | | gAfAfGfacc | | fGfaccaucagug | |
| | | aucagsusg | | | |
| TACTACGAAGA | 142 | usascuacGf | 334 | usascuacGfaAfG | 430 |
| CCATCAGTGC | | aAfGfAfcca | | fAfccaucagugc | |
| | | ucagusgsc | | | |
| TACGAAGACCA | 143 | usascgaaGf | 335 | usascgaaGfaCfC | 431 |
| TCAGTGCGAG | | aCfCffuca | | fAfucagugcgag | |
| | | Agugcgsasg | | | |
| GAAGACCATCA | 144 | gsasagacCf | 336 | gsasagacCfaUfC | 432 |
| GTGCGAGGTC | | aUfCfAfgug | | fAfgugcgagguc | |
| | | cgaggsusc | | | |
| AGACCATCAGT | 145 | asgsaccaUf | 337 | asgsaccaUfcAfG | 433 |
| GCGAGGTCGG | | cAfGfUfgcg | | fUfgcgaggucgg | |
| | | aggucsgsg | | | |
| ACCATCAGTGC | 146 | ascscaucAf | 338 | ascscaucAfgUfG | 434 |
| GAGGTCGGCA | | gUfGfCfgag | | fCfgaggucggca | |
| | | gucggscsa | | | |
| CATCAGTGCGA | 147 | csasucagUf | 339 | csasucagUfgCfG | 435 |
| GGTCGGCATG | | gCfGfAfggu | | fAfggucggcaug | |
| | | cggcasusg | | | |
| TCAGTGCGAGG | 148 | uscsagugCf | 340 | uscsagugCfgAfG | 436 |
| TCGGCATGAT | | gAfGfGfucg | | fGfucggcaugau | |
| | | gcaugsasu | | | |
| TGGAGGAGATG | 149 | usgsgaggAf | 341 | usgsgaggAfgAfU | 437 |
| CAGAATGTGG | | gAfUfGfcag | | fGfcagaaugugg | |
| | | aaugusgsg | | | |
| CCTGCTGGAGT | 150 | cscsugcuGf | 342 | cscsugcuGfgAfG | 438 |
| ATGACCGCCT | | gAfGfUfaug | | fUfaugaccgccu | |
| | | accgcscsu | | | |
| GGAGTATGACC | 151 | gsgsaguaUf | 343 | gsgsaguaUfgAfC | 439 |
| GCCTGGTGGA | | gAfCfCfgcc | | fCfgccuggugga | |
| | | uggugsgsa | | | |
| GAGTATGACCG | 152 | gsasguauGf | 344 | gsasguauGfaCfC | 440 |
| CCTGGTGGAC | | aCfCfGfccu | | fGfccugguggac | |
| | | gguggsasc | | | |
| AGTATGACCGC | 153 | asgsuaugAf | 345 | asgsuaugAfcCfG | 441 |
| CTGGTGGACG | | cCfGfCfcug | | fCfcugguggacg | |
| | | guggascsg | | | |
| GTATGACCGCC | 154 | gsusaugaCf | 346 | gsusaugaCfcGfC | 442 |
| TGGTGGACGA | | cGfCfCfugg | | fCfugguggacga | |
| | | uggacsgsa | | | |
| TATGACCGCCT | 155 | usasugacCf | 347 | usasugacCfgCfC | 443 |
| GGTGGACGAG | | gCfCfUfggu | | fUfgguggacgag | |
| | | ggacgsasg | | | |
| CAGCAGCTGTA | 156 | csasgcagCf | 348 | csasgcagCfuGfU | 444 |
| TGAGAAGCTC | | uGfUfAfuga | | fAfugagaagcuc | |
| | | gaagcsusc | | | |
| GCTGTATGAGA | 157 | gscsuguaUf | 349 | gscsuguaUfgAfG | 445 |
| AGCTCATAGG | | gAfGfAfagc | | fAfagcucauagg | |
| | | ucauasgsg | | | |
| TGTATGAGAAG | 158 | usgsuaugAf | 350 | usgsuaugAfgAfA | 446 |
| CTCATAGGTG | | gAfAfGfcuc | | fGfcucauaggug | |
| | | auaggsusg | | | |
| GTATGAGAAGC | 159 | gsusaugaGf | 351 | gsusaugaGfaAfG | 447 |
| TCATAGGTGG | | aAfGfCfuca | | fCfucauaggugg | |
| | | uaggusgsg | | | |
| TATGAGAAGCT | 160 | usasugagAf | 352 | usasugagAfaGfC | 448 |
| CATAGGTGGC | | aGfCfUfcau | | fUfcauagguggc | |
| | | aggugsgsc | | | |
| ATGAGAAGCTC | 161 | asusgagaAf | 353 | asusgagaAfgCfU | 449 |
| ATAGGTGGCA | | gCfUfCfaua | | fCfauagguggca | |
| | | gguggscsa | | | |
| AAGCTCATAGG | 162 | asasgcucAf | 354 | asasgcucAfuAfG | 450 |
| TGGCAAGTAC | | uAfGfGfugg | | fGfuggcaaguac | |
| | | caagusasc | | | |
| AGCTCATAGGT | 163 | asgscucaUf | 355 | asgscucaUfaGfG | 451 |
| GGCAAGTACA | | aGfGfUfggc | | fUfggcaaguaca | |
| | | aaguascsa | | | |
| TCATAGGTGGC | 164 | uscsauagGf | 356 | uscsauagGfuGfG | 452 |
| AAGTACATGG | | uGfGfCfaag | | fCfaaguacaugg | |
| | | uacausgsg | | | |
| AGGTGGCAAGT | 165 | asgsguggCf | 357 | asgsguggCfaAfG | 453 |
| ACATGGGCGA | | aAfGfUfaca | | fUfacaugggcga | |
| | | ugggcsgsa | | | |
| GTGGCAAGTAC | 166 | gsusggcaAf | 358 | gsusggcaAfgUfA | 454 |
| ATGGGCGAGC | | gUfAfCfaug | | fCfaugggcgagc | |
| | | ggcgasgsc | | | |
| GGCAAGTACAT | 167 | gsgscaagUf | 359 | gsgscaagUfaCfA | 455 |
| GGGCGAGCTG | | aCfAfUfggg | | fUfgggcgagcug | |
| | | cgagcsusg | | | |
| GCTGCTCAGGC | 168 | gscsugcuCf | 360 | gscsugcuCfaGfG | 456 |
| TCGTGGACGA | | aGfGfCfucg | | fCfucguggacga | |
| | | uggacsgsa | | | |
| CTGCTCAGGCT | 169 | csusgcucAf | 361 | csusgcucAfgGfC | 457 |
| CGTGGACGAA | | gGfCfUfcgu | | fUfcguggacgaa | |
| | | ggacgsasa | | | |
| CGACCGCAAGC | 170 | csgsaccgCf | 362 | csgsaccgCfaAfG | 458 |
| AGATCTACAA | | aAfGfCfaga | | fCfagaucuacaa | |
| | | ucuacsasa | | | |
| AGCAGATCTAC | 171 | asgscagaUf | 363 | asgscagaUfcUfA | 459 |
| AACATCCTGA | | cUfAfCfaac | | fCfaacauccuga | |
| | | auccusgsa | | | |
| ATCTACAACAT | 172 | asuscuacAf | 364 | asuscuacAfaCfA | 460 |
| CCTGAGCACG | | aCfAfUfccu | | fUfccugagcacg | |
| | | gagcascsg | | | |
| CTACAACATCC | 173 | csusacaaCf | 365 | csusacaaCfaUfC | 461 |
| TGAGCACGCT | | aUfCfCfuga | | fCfugagcacgcu | |
| | | gcacgscsu | | | |
| ATGCGCATCAC | 174 | asusgcgcAf | 366 | asusgcgcAfuCfA | 462 |
| TGTGGGCGTG | | uCfAfCfugu | | fCfugugggcgug | |
| | | gggcgsusg | | | |
| TCACTGTGGGC | 175 | uscsacugUf | 367 | uscsacugUfgGfG | 463 |
| GTGGATGGCT | | gGfGfCfgug | | fCfguggauggcu | |
| | | gauggscsu | | | |
| CACTGTGGGCG | 176 | csascuguGf | 368 | csascuguGfgGfC | 464 |
| TGGATGGCTC | | gGfCfGfugg | | fGfuggauggcuc | |
| | | auggcsusc | | | |
| ACTGTGGGCGT | 177 | ascsugugGf | 369 | ascsugugGfgCfG | 465 |
| GGATGGCTCC | | gCfGfUfgga | | fUfggauggcucc | |
| | | uggcuscsc | | | |
| TGGGCGTGGAT | 178 | usgsggcgUf | 370 | usgsggcgUfgGfA | 466 |
| GGCTCCGTGT | | gGfAfUfggc | | fUfggcuccgugu | |
| | | uccgusgsu | | | |
| GGCGTGGATGG | 179 | gsgscgugGf | 371 | gsgscgugGfaUfG | 467 |
| CTCCGTGTAC | | aUfGfGfcuc | | fGfcuccguguac | |
| | | cgugusasc | | | |
| GCGTGGATGGC | 180 | gscsguggAf | 372 | gscsguggAfuGfG | 468 |
| TCCGTGTACA | | uGfGfCfucc | | fCfuccguguaca | |
| | | guguascsa | | | |
| GGATGGCTCCG | 181 | gsgsauggCf | 373 | gsgsauggCfuCfC | 469 |
| TGTACAAGCT | | uCfCfGfugu | | fGfuguacaagcu | |
| | | acaagscsu | | | |
| GACGCCCAGCT | 182 | gsascgccCf | 374 | gsascgccCfaGfC | 470 |
| GCGAGATCAC | | aGfCfUfgcg | | fUfgcgagaucac | |
| | | agaucsasc | | | |
| CGAGATCACCT | 183 | csgsagauCf | 375 | csgsagauCfaCfC | 471 |
| TCATCGAGTC | | aCfCfUfuca | | fUfucaucgaguc | |
| | | ucgagsusc | | | |
| GATCACCTTCA | 184 | gsasucacCf | 376 | gsasucacCfuUfC | 472 |
| TCGAGTCGGA | | uUfCfAfucg | | fAfucgagucgga | |
| | | agucgsgsa | | | |
| ATCACCTTCAT | 185 | asuscaccUf | 377 | asuscaccUfuCfA | 473 |
| CGAGTCGGAG | | uCfAfUfcga | | fUfcgagucggag | |
| | | gucggsasg | | | |
| GATGCATTTCC | 186 | gsasugcaUf | 378 | gsasugcaUfuUfC | 474 |
| ACACTGTCAG | | uUfCfCfaca | | fCfacacugucag | |
| | | cugucsasg | | | |
| TGGGAACTCAT | 187 | usgsggaaCf | 379 | usgsggaaCfuCfA | 475 |
| CCTGTGTGGG | | uCfAfUfccu | | fUfccuguguggg | |
| | | gugugsgsg | | | |
| GCAGCTCCAAC | 188 | gscsagcuCf | 380 | gscsagcuCfcAfA | 476 |
| AGCTTGACCA | | cAfAfCfagc | | fCfagcuugacca | |
| | | uugacscsa | | | |
| CCAACAGCTTG | 189 | cscsaacaGf | 381 | cscsaacaGfcUfU | 477 |
| ACCAGACCTA | | cUfUfGfacc | | fGfaccagaccua | |
| | | agaccsusa | | | |
| GCTTGTGATTC | 190 | gscsuuguGf | 382 | gscsuuguGfaUfU | 478 |
| TGGGATGGAC | | aUfUfCfugg | | fCfugggauggac | |
| | | gauggsasc | | | |
| CTTGTGATTCT | 191 | csusugugAf | 383 | csusugugAfuUfC | 479 |
| GGGATGGACC | | uUfCfUfggg | | fUfgggauggacc | |
| | | auggascsc | | | |
| CGCTGGCTCAG | 192 | csgscuggCf | 384 | csgscuggCfuCfA | 480 |
| GAAGAAACCC | | uCfAfGfgaa | | fGfgaagaaaccc | |
| | | gaaacscsc | | | |

In some embodiments, the antisense strand of the RNAi agent comprises a nucleotide sequence of any of the sequences in Table 1. In some embodiments, the antisense strand comprises a nucleotide sequence set forth in any of SEQ ID NOs: 1-96 or 193-288. In some embodiments, the antisense comprises a sequence that has at least at or about 80%, at or about 81%, at or about 82%, at or about 83%, at or about 84%, at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98%, or at or about 99% sequence identity to the sequences set forth in any of SEQ ID NOs: 1-96 or 193-288. In some embodiments, the antisense strand or a subsequence thereof of the RNAi agent differs by 0, 1, 2, 3, or 4 nucleotides from an equal length portion of any of the antisense strand sequences set forth in any of SEQ ID NOs: 1-96 or 193-288, wherein the subsequence is at least 14, 15, 16, 17, 18, 19, 20, 21, 22, or 23 bases in length.

In some embodiments, the antisense strand of the RNAi agent comprises at least 10, 11, 12, 13, 14, 15, 16, 17, 18, 19, 20, 21, 22, or 23 contiguous bases from any one of SEQ ID NOs: 1-96 or 193-288. In some embodiments, the antisense strand comprises, in order, nucleobases 1-16, 1-17, 1-18, 1-19, 1-20, 1-21, 1-22, 1-23, 2-17, 2-18, 2-19, 2-20, 2-21, 2-22, 2 23, 3-18, 4-19, 4-20, 4-21, 4-22, 4-23, 5-21, 5-22, 5-23, 6-20, 6-21, 6-22, 6-23, 7-22, 7-23, or 8-23 of any one of SEQ ID NOS: 1-96 or 193-288.

In some embodiments, the sense strand of the RNAi agent comprises a nucleotide sequence of any of the sequences in Table 2. In some embodiments, the sense strand comprises a nucleotide sequence set forth in any of SEQ ID NOs: 97-192, 289-384, or 385-480. In some embodiments, the sense strand comprises a sequence that has at least at or about 80%, at or about 81%, at or about 82%, at or about 83%, at or about 84%, at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98%, or at or about 99% sequence identity to the sequences set forth in any of SEQ ID NOs: 97-192, 289-384, or 385-480. In some embodiments, the sense strand or a subsequences thereof of the RNAi agent differs by 0, 1, 2, 3, or 4 nucleotides from an equal length portion of any of the sense strand sequences set forth in any of SEQ ID NOs: 97-192, 289-384, or 385-480, wherein the subsequence is at least 14, 15, 16, 17, 18, 19, 20, or 21 bases in length.

In some embodiments, the sense strand of the RNAi agent comprises at least 10, 11, 12, 13, 14, 15, 16, 17, 18, 19, or 20 contiguous bases from any one of SEQ ID NOS: 97-192, 289-384, or 385-480. In some embodiments, the sense strand comprises, in order, nucleobases 1-16, 1-17, 1-18, 1-19, 1-20, 1-21, 1-22, 1-23, 2-17, 2-18, 2-19, 2-20, 2-21, 2-22, 2 23, 3-18, 4-19, 4-20, 4-21, 4-22, 4-23, 5-21, 5-22, 5-23, 6-20, 6-21, 6-22, 6-23, 7-22, 7-23, or 8-23 of any one of SEQ ID NOS: 97-192, 289-384, or 385-480.

Among the RNAi agents provided herein, the RNAi agent comprises an antisense strand comprising a nucleotide sequence having at least at or about 80%, at or about 81%, at or about 82%, at or about 83%, at or about 84%, at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98%, or at or about 99% sequence identity to the sequences set forth in any of SEQ ID NOs: 1-96 and a sense strand comprising a nucleotide sequence having at least at or about 80%, at or about 81%, at or about 82%, at or about 83%, at or about 84%, at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98%, or at or about 99% sequence identity to the sequences set forth in any of SEQ ID NOs: 97-192.

In some embodiments, the antisense strand of the RNAi agent comprises a nucleotide sequence of any of SEQ ID NO: 1-96 and the sense strand of the RNAi agent comprises a nucleotide sequence of any of SEQ ID NO: 97-192. In some embodiments, the antisense and the sense strands of the RNAi agent comprise the nucleotide sequences of SEQ ID NOs: 1 and 97, respectively; SEQ ID NOs: 2 and 98, respectively; SEQ ID NOs: 3 and 99, respectively; SEQ ID NOs: 4 and 100, respectively; SEQ ID NOs: 5 and 101, respectively; SEQ ID NOs: 6 and 102, respectively; SEQ ID NOs: 7 and 103, respectively; SEQ ID NOs: 8 and 104, respectively; SEQ ID NOs: 9 and 105, respectively; SEQ ID NOs: 10 and 106, respectively; SEQ ID NOs: 11 and 107, respectively; SEQ ID NOs: 12 and 108, respectively; SEQ ID NOs: 13 and 109, respectively; SEQ ID NOs: 14 and 110, respectively; SEQ ID NOs: 15 and 111, respectively; SEQ ID NOs: 16 and 112, respectively; SEQ ID NOs: 17 and 113, respectively; SEQ ID NOs: 18 and 114, respectively; SEQ ID NOs: 19 and 115, respectively; or SEQ ID NOs: 20 and 116, respectively, or any antisense or sense strand that has at least 80% sequence identity to any of the above sequences, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, the antisense and the sense strands of the RNAi agent comprise the nucleotide sequences of SEQ ID NOs: 21 and 117, respectively; SEQ ID NOs: 22 and 118, respectively; SEQ ID NOs: 23 and 119, respectively; SEQ ID NOs: 24 and 120, respectively; SEQ ID NOs: 25 and 121, respectively; SEQ ID NOs: 26 and 122, respectively; SEQ ID NOs: 27 and 123, respectively; SEQ ID NOs: 28 and 124, respectively; SEQ ID NOs: 29 and 125, respectively; SEQ ID NOs: 30 and 126, respectively; SEQ ID NOs: 31 and 127, respectively; SEQ ID NOs: 32 and 128, respectively; SEQ ID NOs: 33 and 129, respectively; SEQ ID NOs: 34 and 130, respectively; SEQ ID NOs: 35 and 131, respectively; SEQ ID NOs: 36 and 132, respectively; SEQ ID NOs: 37 and 133, respectively; SEQ ID NOs: 38 and 134, respectively; SEQ ID NOs: 39 and 135, respectively; or SEQ ID NOs: 40 and 136, respectively, or any antisense or sense strand that has at least 80% sequence identity to any of the above sequences, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, the antisense and the sense strands of the RNAi agent comprise the nucleotide sequences of SEQ ID NOs: 41 and 137, respectively; SEQ ID NOs: 42 and 138, respectively; SEQ ID NOs: 43 and 139, respectively; SEQ ID NOs: 44 and 140, respectively; SEQ ID NOs: 45 and 141, respectively; SEQ ID NOs: 46 and 142, respectively; SEQ ID NOs: 47 and 143, respectively; SEQ ID NOs: 48 and 144, respectively; SEQ ID NOs: 49 and 145, respectively; SEQ ID NOs: 50 and 146, respectively; SEQ ID NOs: 51 and 147, respectively; SEQ ID NOs: 52 and 148, respectively; SEQ ID NOs: 53 and 149, respectively; SEQ ID NOs: 54 and 150, respectively; SEQ ID NOs: 55 and 151, respectively; SEQ ID NOs: 56 and 152, respectively; SEQ ID NOs: 57 and 153, respectively; SEQ ID NOs: 58 and 154, respectively; SEQ ID NOs: 59 and 155, respectively; or SEQ ID NOs: 60 and 156, respectively, or any antisense or sense strand that has at least 80% sequence identity to any of the above sequences, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, the antisense and the sense strands of the RNAi agent comprise the nucleotide sequences of SEQ ID NOs: 61 and 157, respectively; SEQ ID NOs: 62 and 158, respectively; SEQ ID NOs: 63 and 159, respectively; SEQ ID NOs: 64 and 160, respectively; SEQ ID NOs: 65 and 161, respectively; SEQ ID NOs: 66 and 162, respectively; SEQ ID NOs: 67 and 163, respectively; SEQ ID NOs: 68 and 164, respectively; SEQ ID NOs: 69 and 165, respectively; SEQ ID NOs: 70 and 166, respectively; SEQ ID NOs: 71 and 167, respectively; SEQ ID NOs: 72 and 168, respectively; SEQ ID NOs: 73 and 169, respectively; SEQ ID NOs: 74 and 170, respectively; SEQ ID NOs: 75 and 171, respectively; SEQ ID NOs: 76 and 172, respectively; SEQ ID NOs: 77 and 173, respectively; SEQ ID NOs: 78 and 174, respectively; SEQ ID NOs: 79 and 175, respectively; or SEQ ID NOs: 80 and 176, respectively, or any antisense or sense strand that has at least 80% sequence identity to any of the above sequences, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, the antisense and the sense strands of the RNAi agent comprise the nucleotide sequences of SEQ ID NOs: 81 and 177, respectively; SEQ ID NOs: 82 and 178, respectively; SEQ ID NOs: 83 and 179, respectively; SEQ ID NOs: 84 and 180, respectively; SEQ ID NOs: 85 and 181, respectively; SEQ ID NOs: 86 and 182, respectively; SEQ ID NOs: 87 and 183, respectively; SEQ ID NOs: 88 and 184, respectively; SEQ ID NOs: 89 and 185, respectively; SEQ ID NOs: 90 and 186, respectively; SEQ ID NOs: 91 and 187, respectively; SEQ ID NOs: 92 and 188, respectively; SEQ ID NOs: 93 and 189, respectively; SEQ ID NOs: 94 and 190, respectively; SEQ ID NOs: 95 and 191, respectively; or SEQ ID NOs: 96 and 192, respectively, or any antisense or sense strand that has at least 80% sequence identity to any of the above sequences, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, the RNAi agent is prepared or provided as a salt, mixed salt, or a free-acid. In some embodiments, the RNAi agent is prepared as a sodium salt. Such forms are within the scope of the embodiments disclosed herein.

In some embodiments, the GCK RNAi agent comprises an antisense strand and a sense strand having the nucleotide sequences of any of the antisense strand/sense strand duplexes of Table 3. In some embodiments, the GCK RNAi agent comprises an antisense strand and a sense strand having the nucleotide sequences of any of the antisense strand/sense strand duplexes of Table 3, and further comprises an asialoglycoprotein receptor ligand targeting group. In some embodiments, the GCK RNAi agent comprises any of the duplexes of Table 3.

TABLE 3
Examples of GCK RNAi agent duplexes.

| | | | | | Modified Sense |
|---|---|---|---|---|---|
| | | | Modified | Modified | Strand with 3′ |
| | Antisense | Sense | Antisense | Sense | conjugated |
| | Strand | Strand | Strand | Strand | targeting |
| Duplex | SEQ | SEQ | SEQ | SEQ | moiety SEQ |
| ID NO: | ID NO: | ID NO: | ID NO: | ID NO: | ID NO: |
| 572 | 1 | 97 | 193 | 289 | 385 |
| 574 | 2 | 98 | 194 | 290 | 386 |
| 634 | 3 | 99 | 195 | 291 | 387 |
| 636 | 4 | 100 | 196 | 292 | 388 |
| 655 | 5 | 101 | 197 | 293 | 389 |
| 749 | 6 | 102 | 198 | 294 | 390 |
| 780 | 7 | 103 | 199 | 295 | 391 |
| 784 | 8 | 104 | 200 | 296 | 392 |
| 786 | 9 | 105 | 201 | 297 | 393 |
| 787 | 10 | 106 | 202 | 298 | 394 |
| 789 | 11 | 107 | 203 | 299 | 395 |
| 828 | 12 | 108 | 204 | 300 | 396 |
| 829 | 13 | 109 | 205 | 301 | 397 |
| 835 | 14 | 110 | 206 | 302 | 398 |
| 836 | 15 | 111 | 207 | 303 | 399 |
| 841 | 16 | 112 | 208 | 304 | 400 |
| 882 | 17 | 113 | 209 | 305 | 401 |
| 953 | 18 | 114 | 210 | 306 | 402 |
| 955 | 19 | 115 | 211 | 307 | 403 |
| 962 | 20 | 116 | 212 | 308 | 404 |
| 966 | 21 | 117 | 213 | 309 | 405 |
| 969 | 22 | 118 | 214 | 310 | 406 |
| 1005 | 23 | 119 | 215 | 311 | 407 |
| 1007 | 24 | 120 | 216 | 312 | 408 |
| 1008 | 25 | 121 | 217 | 313 | 409 |
| 1009 | 26 | 122 | 218 | 314 | 410 |
| 1011 | 27 | 123 | 219 | 315 | 411 |
| 1012 | 28 | 124 | 220 | 316 | 412 |
| 1072 | 29 | 125 | 221 | 317 | 413 |
| 1076 | 30 | 126 | 222 | 318 | 414 |
| 1077 | 31 | 127 | 223 | 319 | 415 |
| 1078 | 32 | 128 | 224 | 320 | 416 |
| 1081 | 33 | 129 | 225 | 321 | 417 |
| 1084 | 34 | 130 | 226 | 322 | 418 |
| 1085 | 35 | 131 | 227 | 323 | 419 |
| 1088 | 36 | 132 | 228 | 324 | 420 |
| 1089 | 37 | 133 | 229 | 325 | 421 |
| 1107 | 38 | 134 | 230 | 326 | 422 |
| 1108 | 39 | 135 | 231 | 327 | 423 |
| 1109 | 40 | 136 | 232 | 328 | 424 |
| 1110 | 41 | 137 | 233 | 329 | 425 |
| 1111 | 42 | 138 | 234 | 330 | 426 |
| 1113 | 43 | 139 | 235 | 331 | 427 |
| 1114 | 44 | 140 | 236 | 332 | 428 |
| 1123 | 45 | 141 | 237 | 333 | 429 |
| 1124 | 46 | 142 | 238 | 334 | 430 |
| 1127 | 47 | 143 | 239 | 335 | 431 |
| 1130 | 48 | 144 | 240 | 336 | 432 |
| 1132 | 49 | 145 | 241 | 337 | 433 |
| 1134 | 50 | 146 | 242 | 338 | 434 |
| 1136 | 51 | 147 | 243 | 339 | 435 |
| 1138 | 52 | 148 | 244 | 340 | 436 |
| 1188 | 53 | 149 | 245 | 341 | 437 |
| 1291 | 54 | 150 | 246 | 342 | 438 |
| 1297 | 55 | 151 | 247 | 343 | 439 |
| 1298 | 56 | 152 | 248 | 344 | 440 |
| 1299 | 57 | 153 | 249 | 345 | 441 |
| 1300 | 58 | 154 | 250 | 346 | 442 |
| 1301 | 59 | 155 | 251 | 347 | 443 |
| 1340 | 60 | 156 | 252 | 348 | 444 |
| 1345 | 61 | 157 | 253 | 349 | 445 |
| 1347 | 62 | 158 | 254 | 350 | 446 |
| 1348 | 63 | 159 | 255 | 351 | 447 |
| 1349 | 64 | 160 | 256 | 352 | 448 |
| 1350 | 65 | 161 | 257 | 353 | 449 |
| 1355 | 66 | 162 | 258 | 354 | 450 |
| 1356 | 67 | 163 | 259 | 355 | 451 |
| 1359 | 68 | 164 | 260 | 356 | 452 |
| 1363 | 69 | 165 | 261 | 357 | 453 |
| 1365 | 70 | 166 | 262 | 358 | 454 |
| 1367 | 71 | 167 | 263 | 359 | 455 |
| 1399 | 72 | 168 | 264 | 360 | 456 |
| 1400 | 73 | 169 | 265 | 361 | 457 |
| 1513 | 74 | 170 | 266 | 362 | 458 |
| 1521 | 75 | 171 | 267 | 363 | 459 |
| 1526 | 76 | 172 | 268 | 364 | 460 |
| 1528 | 77 | 173 | 269 | 365 | 461 |
| 1688 | 78 | 174 | 270 | 366 | 462 |
| 1695 | 79 | 175 | 271 | 367 | 463 |
| 1696 | 80 | 176 | 272 | 368 | 464 |
| 1697 | 81 | 177 | 273 | 369 | 465 |
| 1701 | 82 | 178 | 274 | 370 | 466 |
| 1703 | 83 | 179 | 275 | 371 | 467 |
| 1704 | 84 | 180 | 276 | 372 | 468 |
| 1708 | 85 | 181 | 277 | 373 | 469 |
| 1774 | 86 | 182 | 278 | 374 | 470 |
| 1786 | 87 | 183 | 279 | 375 | 471 |
| 1789 | 88 | 184 | 280 | 376 | 472 |
| 1790 | 89 | 185 | 281 | 377 | 473 |
| 2180 | 90 | 186 | 282 | 378 | 474 |
| 2273 | 91 | 187 | 283 | 379 | 475 |
| 2297 | 92 | 188 | 284 | 380 | 476 |
| 2303 | 93 | 189 | 285 | 381 | 477 |
| 2492 | 94 | 190 | 286 | 382 | 478 |
| 2493 | 95 | 191 | 287 | 383 | 479 |
| 2609 | 96 | 192 | 288 | 384 | 480 |

### A. Modified Nucleotides

In some embodiments, the RNAi agent comprise an antisense strand and a sense strand, wherein all or substantially all of the nucleotides in the sense strand are modified. In some embodiments, the RNAi agent comprise an antisense strand and a sense strand, wherein all or substantially all of the nucleotides in the antisense strand are modified. In some embodiments, the RNAi agent comprises an antisense strand that comprises a nucleobase sequence differing by 0, 1, 2, 3 or 4 nucleobases from an equal length portion of any of SEQ ID NOs: 1-96. In some embodiments, the RNAi agent comprises an antisense strand that comprises a nucleobase sequence differing by 1 nucleobase from any of SEQ ID NOs: 1-96. In some embodiments, the RNAi agent comprises a sense strand that comprises a nucleobase sequence differing by 0, 1, 2, 3 or 4 nucleobases from any of SEQ ID NOs: 97-192. In some embodiments, the RNAi agent comprises a sense strand that comprises a nucleobase sequence differing by 1 nucleobase from any of SEQ ID NOs: 97-192.

In some embodiments, the RNAi agent comprises one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides include, but are not limited to, deoxyribonucleotides, nucleotide mimics, abasic nucleotides (represented herein as Ab), 2′-modified nucleotides, 3′ to 3′ linkages (inverted) nucleotides (represented herein as invdN, invN, invn, invAb), nonnatural base-comprising nucleotides, bridged nucleotides, peptide nucleic acids (PNAs), 2′, 3′seco nucleotide mimics (unlocked nucleobase analogues, represented herein as N_{UNA} or NU NA), locked nucleotides (represented herein as N_{LNA} or NLNA), 3′-O-methoxy (2′ internucleoside linked) nucleotides (represented herein as 3′-OMen), 2′-F-Arabino nucleotides (represented herein as NfANA or Nf_{ANA}), 5′-Me, 2′-fluoro nucleotide (represented herein as 5Me-Nf), morpholino nucleotides, vinyl phosphonate deoxyribonucleotides (represented herein as vpdN), vinyl phosphonate containing nucleotides, and cyclopropyl phosphonate containing nucleotides (cPrpN). 2′-modified nucleotides (i.e. a nucleotide with a group other than a hydroxyl group at the 2′ position of the five-membered sugar ring) include, but are not limited to, 2′-O-methyl nucleotides (represented herein as a lower case letter ‘n’ in a nucleotide sequence), 2′-deoxy-2′-fluoro nucleotides (represented herein as Nf, also represented herein as 2′-fluoro nucleotide), 2′-deoxy nucleotides (represented herein as dN), 2′-methoxyethyl (2′-O-2-methoxylethyl) nucleotides (represented herein as NM or 2′-MOE), 2′-amino nucleotides, and 2′-alkyl nucleotides. It is not necessary for all positions in a given compound to be uniformly modified. Conversely, more than one modification may be incorporated in a single RNAi agent or even in a single nucleotide thereof. The RNAi agent sense strands and antisense strands may be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and 0-6 substituted purines, (e.g., 2-aminopropyladenine, 5-propynyluracil, or 5-propynylcytosine), 5-methylcytosine (5-me-C), 5hydroxymethyl cytosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5propynyl uracil, 5propynyl cytosine, 6-azo uracil, 6-azo cytosine, 6-azo thymine, 5-uracil (pseudouracil), 4thiouracil, 8-halo, 8amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7methylguanine and 7-methyladenine, 8-azaguanine and 8-azaadenine, 7-deazaguanine, 7deazaadenine, 3-deazaguanine, and 3-deazaadenine.

In some embodiments, the 3′ end and/or the 5′ end of the sense strand may include additional abasic nucleosides. In some embodiments, an “A” may be added to the 3′ terminal end of the sense strand. In some embodiments, the one or more abasic nucleosides added to the 3′ end or 5′ end of the sense strand may be inverted (invAb). In some embodiments, one or more inverted abasic nucleosides may be inserted between the targeting ligand and the nucleobase sequence of the sense strand of the RNAi agent. In some embodiments, the inclusion of one or more inverted abasic nucleosides at or near the terminal end or terminal ends of the sense strand of an RNAi agent may allow for enhanced activity or other desired properties of an RNAi agent.

In some embodiments, the 3′ end and/or the 5′ end of the antisense strand may include additional abasic nucleosides. In some embodiments, a “U” may be added to the 5′ terminal end of the antisense strand. In some embodiments, the one or more abasic nucleosides added to the 3′ end or 5′ end of the antisense strand may be inverted (invAb). In some embodiments, one or more inverted abasic nucleosides may be inserted between the targeting ligand and the nucleobase sequence of the sense strand of the RNAi agent. In some embodiments, the inclusion of one or more inverted abasic nucleosides at or near the terminal end or terminal ends of the antisense strand of an RNAi agent may allow for enhanced activity or other desired properties of an RNAi agent.

In some embodiments, the modification comprises fluorinated bases. In some embodiments, 1 or more, 2 or more, 3 or more, 4 or more, 5 or more, 6 or more, 7 or more, 8 or more, 9 or more, or 10 or more bases of the sense strand and/or antisense strand of the RNAi agent are fluorinated. In some embodiments, base 1, 2, 3, 4, 5, 6, 7, 8, 9, 10, 11, 12, 13, 14, 15, 16, 17, 18, 19, and/or 20 of the sense strand of the RNAi agent are fluorinated. In some embodiments, bases 7, 9, 10, and 11 of the sense strand of the RNAi agent are fluorinated. In some embodiments, base 1, 2, 3, 4, 5, 6, 7, 8, 9, 10, 11, 12, 13, 14, 15, 16, 17, 18, 19, 20, 21, 22, and/or 23 of the antisense strand of the RNAi agent are fluorinated. In some embodiments, bases 2, 6, 14, and 16 of the antisense strand are fluorinated.

In some embodiments, the modification comprises 2′-O-methyl (2′-OMe) and/or 2′-deoxy-2′-fluoro (2′-F) ribosugar modifications. In some embodiments, the modification comprises those described in Foster, et al., Molecular Therapy. Vol. 26 No 3 M arch 2018 which is herein incorporated by reference. In some embodiments, all or substantially all of the nucleotides of the RNAi agent are modified nucleotides. A s used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides. As used herein, a sense strand wherein substantially all of the nucleotides present are modified nucleotides is a sense strand having two or fewer (i.e., 0, 1, or 2) nucleotides in the sense strand being ribonucleotides. As used herein, an antisense sense strand wherein substantially all of the nucleotides present are modified nucleotides is an antisense strand having two or fewer (i.e., 0, 1, or 2) nucleotides in the sense strand being ribonucleotides. In some embodiments, one or more nucleotides of an RNAi agent is a ribonucleotide.

In some embodiments, the modified antisense strand of the RNAi agent comprises a nucleotide sequence of any of the sequences in Table 1. In some embodiments, the modified sense strand of the RNAi agent comprises a nucleotide sequence of any of the sequences in Table 2.

Among the RNAi agents provided herein, the RNAi agent comprises a modified antisense strand comprising a nucleotide sequence having at least at or about 80%, at or about 81%, at or about 82%, at or about 83%, at or about 84%, at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98%, or at or about 99% sequence identity to the sequences set forth in any of SEQ ID NOs: 193-288 and a modified sense strand comprising a nucleotide sequence having at least at or about 80%, at or about 81%, at or about 82%, at or about 83%, at or about 84%, at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98%, or at or about 99% sequence identity to the sequences set forth in any of SEQ ID NOs: 289-384 or 385-480. Furthermore, among the RNAi agents provided herein, the RNAi agent comprises a modified antisense strand that differs by 1, 2, 3, or 4 nucleotides from an equal length portion of the sequences set forth in any of SEQ ID NOs: 289-384, 385-480, or a subsequence thereof, and a modified sense strand that differs by 1, 2, 3, or 4 nucleotides from an equal length portion of the sequences set forth in any of SEQ ID NOs: 289-384, 385-480, or a subsequence thereof.

In some embodiments, the modified antisense strand of the RNAi agent comprises a nucleotide sequence of any of SEQ ID NO: 193-288 and the modified sense strand of the RNAi agent comprises a nucleotide sequence of any of SEQ ID NO: 289-384 or 385-480. In some embodiments, the modified antisense and the modified sense strands of the RNAi agent comprise the nucleotide sequences of SEQ ID NOs: 193 and 289, respectively; SEQ ID NOs: 194 and 290, respectively; SEQ ID NOs: 195 and 291, respectively; SEQ ID NOs: 196 and 292, respectively; SEQ ID NOs: 197 and 293, respectively; SEQ ID NOs: 198 and 294, respectively; SEQ ID NOs: 199 and 295, respectively; SEQ ID NOs: 200 and 296, respectively; SEQ ID NOs: 201 and 297, respectively; SEQ ID NOs: 202 and 298, respectively; SEQ ID NOs: 203 and 299, respectively; SEQ ID NOs: 204 and 300, respectively; SEQ ID NOs: 205 and 301, respectively; SEQ ID NOs: 206 and 302, respectively; SEQ ID NOs: 207 and 303, respectively; SEQ ID NOs: 208 and 304, respectively; SEQ ID NOs: 209 and 305, respectively; SEQ ID NOs: 210 and 306, respectively; SEQ ID NOs: 211 and 307, respectively; or SEQ ID NOs: 212 and 308, respectively, or any antisense or sense strand that has at least 80% sequence identity to any of the above sequences, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto, or any antisense or sense stand that differs by 1, 2, 3, or 4 nucleotides from an equal length portion of the sequences of SEQ ID NOs: 193 and 289, respectively; SEQ ID NOs: 194 and 290, respectively; SEQ ID NOs: 195 and 291, respectively; SEQ ID NOs: 196 and 292, respectively; SEQ ID NOs: 197 and 293, respectively; SEQ ID NOs: 198 and 294, respectively; SEQ ID NOs: 199 and 295, respectively; SEQ ID NOs: 200 and 296, respectively; SEQ ID NOs: 201 and 297, respectively; SEQ ID NOs: 202 and 298, respectively; SEQ ID NOs: 203 and 299, respectively; SEQ ID NOs: 204 and 300, respectively; SEQ ID NOs: 205 and 301, respectively; SEQ ID NOs: 206 and 302, respectively; SEQ ID NOs: 207 and 303, respectively; SEQ ID NOs: 208 and 304, respectively; SEQ ID NOs: 209 and 305, respectively; SEQ ID NOs: 210 and 306, respectively; SEQ ID NOs: 211 and 307, respectively; or SEQ ID NOs: 212 and 308, respectively.

In some embodiments, the modified antisense and the modified sense strands of the RNAi agent comprise the nucleotide sequences of SEQ ID NOs: 213 and 309, respectively; SEQ ID NOs: 214 and 310, respectively; SEQ ID NOs: 215 and 311, respectively; SEQ ID NOs: 216 and 312, respectively; SEQ ID NOs: 217 and 313, respectively; SEQ ID NOs: 218 and 314, respectively; SEQ ID NOs: 219 and 315, respectively; SEQ ID NOs: 220 and 316, respectively; SEQ ID NOs: 221 and 317, respectively; SEQ ID NOs: 222 and 318, respectively; SEQ ID NOs: 223 and 319, respectively; SEQ ID NOs: 224 and 320, respectively; SEQ ID NOs: 225 and 321, respectively; SEQ ID NOs: 226 and 322, respectively; SEQ ID NOs: 227 and 323, respectively; SEQ ID NOs: 228 and 324, respectively; SEQ ID NOs: 229 and 325, respectively; SEQ ID NOs: 230 and 326, respectively; SEQ ID NOs: 231 and 327, respectively; or SEQ ID NOs: 232 and 328, respectively, or any antisense or sense strand that has at least 80% sequence identity to any of the above sequences, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto, or any antisense or sense stand that differs by 1, 2, 3, or 4 nucleotides from an equal length portion of the sequences of SEQ ID NOs: 213 and 309, respectively; SEQ ID NOs: 214 and 310, respectively; SEQ ID NOs: 215 and 311, respectively; SEQ ID NOs: 216 and 312, respectively; SEQ ID NOs: 217 and 313, respectively; SEQ ID NOs: 218 and 314, respectively; SEQ ID NOs: 219 and 315, respectively; SEQ ID NOs: 220 and 316, respectively; SEQ ID NOs: 221 and 317, respectively; SEQ ID NOs: 222 and 318, respectively; SEQ ID NOs: 223 and 319, respectively; SEQ ID NOs: 224 and 320, respectively; SEQ ID NOs: 225 and 321, respectively; SEQ ID NOs: 226 and 322, respectively; SEQ ID NOs: 227 and 323, respectively; SEQ ID NOs: 228 and 324, respectively; SEQ ID NOs: 229 and 325, respectively; SEQ ID NOs: 230 and 326, respectively; SEQ ID NOs: 231 and 327, respectively; or SEQ ID NOs: 232 and 328, respectively.

In some embodiments, the modified antisense and the modified sense strands of the RNAi agent comprise the nucleotide sequences of SEQ ID NOs: 233 and 329, respectively; SEQ ID NOs: 234 and 330, respectively; SEQ ID NOs: 235 and 331, respectively; SEQ ID NOs: 236 and 332, respectively; SEQ ID NOs: 237 and 333, respectively; SEQ ID NOs: 238 and 334, respectively; SEQ ID NOs: 239 and 335, respectively; SEQ ID NOs: 240 and 336, respectively; SEQ ID NOs: 241 and 337, respectively; SEQ ID NOs: 242 and 338, respectively; SEQ ID NOs: 243 and 339, respectively; SEQ ID NOs: 244 and 340, respectively; SEQ ID NOs: 245 and 341, respectively; SEQ ID NOs: 246 and 342, respectively; SEQ ID NOs: 247 and 343, respectively; SEQ ID NOs: 248 and 344, respectively; SEQ ID NOs: 249 and 345, respectively; SEQ ID NOs: 250 and 346, respectively; SEQ ID NOs: 251 and 347, respectively; or SEQ ID NOs: 252 and 348, respectively, or any antisense or sense strand that has at least 80% sequence identity to any of the above sequences, such as at least 80% sequence identity to any of the above sequences, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto, or any antisense or sense stand that differs by 1, 2, 3, or 4 nucleotides from an equal length portion of the sequences of SEQ ID NOs: 233 and 329, respectively; SEQ ID NOs: 234 and 330, respectively; SEQ ID NOs: 235 and 331, respectively; SEQ ID NOs: 236 and 332, respectively; SEQ ID NOs: 237 and 333, respectively; SEQ ID NOs: 238 and 334, respectively; SEQ ID NOs: 239 and 335, respectively; SEQ ID NOs: 240 and 336, respectively; SEQ ID NOs: 241 and 337, respectively; SEQ ID NOs: 242 and 338, respectively; SEQ ID NOs: 243 and 339, respectively; SEQ ID NOs: 244 and 340, respectively; SEQ ID NOs: 245 and 341, respectively; SEQ ID NOs: 246 and 342, respectively; SEQ ID NOs: 247 and 343, respectively; SEQ ID NOs: 248 and 344, respectively; SEQ ID NOs: 249 and 345, respectively; SEQ ID NOs: 250 and 346, respectively; SEQ ID NOs: 251 and 347, respectively; or SEQ ID NOs: 252 and 348.

In some embodiments, the modified antisense and the modified sense strands of the RNAi agent comprise the nucleotide sequences of SEQ ID NOs: 253 and 349, respectively; SEQ ID NOs: 254 and 350, respectively; SEQ ID NOs: 255 and 351, respectively; SEQ ID NOs: 256 and 352, respectively; SEQ ID NOs: 257 and 353, respectively; SEQ ID NOs: 258 and 354, respectively; SEQ ID NOs: 259 and 355, respectively; SEQ ID NOs: 260 and 356, respectively; SEQ ID NOs: 261 and 357, respectively; SEQ ID NOs: 262 and 358, respectively; SEQ ID NOs: 263 and 359, respectively; SEQ ID NOs: 264 and 360, respectively; SEQ ID NOs: 265 and 361, respectively; SEQ ID NOs: 266 and 362, respectively; SEQ ID NOs: 267 and 363, respectively; SEQ ID NOs: 268 and 364, respectively; SEQ ID NOs: 269 and 365, respectively; SEQ ID NOs: 270 and 366, respectively; SEQ ID NOs: 271 and 367, respectively; or SEQ ID NOs: 272 and 368, respectively, or any antisense or sense strand that has at least 80% sequence identity to any of the above sequences, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto, or any antisense or sense stand that differs by 1, 2, 3, or 4 nucleotides from an equal length portion of the sequences of SEQ ID NOs: 253 and 349, respectively; SEQ ID NOs: 254 and 350, respectively; SEQ ID NOs: 255 and 351, respectively; SEQ ID NOs: 256 and 352, respectively; SEQ ID NOs: 257 and 353, respectively; SEQ ID NOs: 258 and 354, respectively; SEQ ID NOs: 259 and 355, respectively; SEQ ID NOs: 260 and 356, respectively; SEQ ID NOs: 261 and 357, respectively; SEQ ID NOs: 262 and 358, respectively; SEQ ID NOs: 263 and 359, respectively; SEQ ID NOs: 264 and 360, respectively; SEQ ID NOs: 265 and 361, respectively; SEQ ID NOs: 266 and 362, respectively; SEQ ID NOs: 267 and 363, respectively; SEQ ID NOs: 268 and 364, respectively; SEQ ID NOs: 269 and 365, respectively; SEQ ID NOs: 270 and 366, respectively; SEQ ID NOs: 271 and 367, respectively; or SEQ ID NOs: 272 and 368, respectively.

In some embodiments, the modified antisense and the modified sense strands of the RNAi agent comprise the nucleotide sequences of SEQ ID NOs: 273 and 369, respectively; SEQ ID NOs: 274 and 370, respectively; SEQ ID NOs: 275 and 371, respectively; SEQ ID NOs: 276 and 372, respectively; SEQ ID NOs: 277 and 373, respectively; SEQ ID NOs: 278 and 374, respectively; SEQ ID NOs: 279 and 375, respectively; SEQ ID NOs: 280 and 376, respectively; SEQ ID NOs: 281 and 377, respectively; SEQ ID NOs: 282 and 378, respectively; SEQ ID NOs: 283 and 379, respectively; SEQ ID NOs: 284 and 380, respectively; SEQ ID NOs: 285 and 381, respectively; SEQ ID NOs: 286 and 382, respectively; SEQ ID NOs: 287 and 383, respectively; or SEQ ID NOs: 288 and 384, respectively, or any antisense or sense strand that has at least 80% sequence identity to any of the above sequences, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto, or any antisense or sense stand that differs by 1, 2, 3, or 4 nucleotides from an equal length portion of the sequences of SEQ ID NOs: 273 and 369, respectively; SEQ ID NOs: 274 and 370, respectively; SEQ ID NOs: 275 and 371, respectively; SEQ ID NOs: 276 and 372, respectively; SEQ ID NOs: 277 and 373, respectively; SEQ ID NOs: 278 and 374, respectively; SEQ ID NOs: 279 and 375, respectively; SEQ ID NOs: 280 and 376, respectively; SEQ ID NOs: 281 and 377, respectively; SEQ ID NOs: 282 and 378, respectively; SEQ ID NOs: 283 and 379, respectively; SEQ ID NOs: 284 and 380, respectively; SEQ ID NOs: 285 and 381, respectively; SEQ ID NOs: 286 and 382, respectively; SEQ ID NOs: 287 and 383, respectively; or SEQ ID NOs: 288 and 384, respectively.

In some embodiments, the modified antisense and the modified sense strands of the RNAi agent comprise the nucleotide sequences of SEQ ID NOs: 193 and 385, respectively; SEQ ID NOs: 194 and 386, respectively; SEQ ID NOs: 195 and 387, respectively; SEQ ID NOs: 196 and 388, respectively; SEQ ID NOs: 197 and 389, respectively; SEQ ID NOs: 198 and 390, respectively; SEQ ID NOs: 199 and 391, respectively; SEQ ID NOs: 200 and 392, respectively; SEQ ID NOs: 201 and 393, respectively; SEQ ID NOs: 202 and 394, respectively; SEQ ID NOs: 203 and 395, respectively; SEQ ID NOs: 204 and 396, respectively; SEQ ID NOs: 205 and 397, respectively; SEQ ID NOs: 206 and 398, respectively; SEQ ID NOs: 207 and 399, respectively; SEQ ID NOs: 208 and 400, respectively; SEQ ID NOs: 209 and 401, respectively; SEQ ID NOs: 210 and 402, respectively; SEQ ID NOs: 211 and 403, respectively; or SEQ ID NOs: 212 and 404, respectively, or any antisense or sense strand that has at least 80% sequence identity to any of the above sequences, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto, or any antisense or sense stand that differs by 1, 2, 3, or 4 nucleotides from an equal length portion of the sequences of SEQ ID NOs: 193 and 385, respectively; SEQ ID NOs: 194 and 386, respectively; SEQ ID NOs: 195 and 387, respectively; SEQ ID NOs: 196 and 388, respectively; SEQ ID NOs: 197 and 389, respectively; SEQ ID NOs: 198 and 390, respectively; SEQ ID NOs: 199 and 391, respectively; SEQ ID NOs: 200 and 392, respectively; SEQ ID NOs: 201 and 393, respectively; SEQ ID NOs: 202 and 394, respectively; SEQ ID NOs: 203 and 395, respectively; SEQ ID NOs: 204 and 396, respectively; SEQ ID NOs: 205 and 397, respectively; SEQ ID NOs: 206 and 398, respectively; SEQ ID NOs: 207 and 399, respectively; SEQ ID NOs: 208 and 400, respectively; SEQ ID NOs: 209 and 401, respectively; SEQ ID NOs: 210 and 402, respectively; SEQ ID NOs: 211 and 403, respectively; or SEQ ID NOs: 212 and 404, respectively.

In some embodiments, the modified antisense and the modified sense strands of the RNAi agent comprise the nucleotide sequences of SEQ ID NOs: 213 and 405, respectively; SEQ ID NOs: 214 and 406, respectively; SEQ ID NOs: 215 and 407, respectively; SEQ ID NOs: 216 and 408, respectively; SEQ ID NOs: 217 and 409, respectively; SEQ ID NOs: 218 and 410, respectively; SEQ ID NOs: 219 and 411, respectively; SEQ ID NOs: 220 and 412, respectively; SEQ ID NOs: 221 and 413, respectively; SEQ ID NOs: 222 and 414, respectively; SEQ ID NOs: 223 and 415, respectively; SEQ ID NOs: 224 and 416, respectively; SEQ ID NOs: 225 and 417, respectively; SEQ ID NOs: 226 and 418, respectively; SEQ ID NOs: 227 and 419, respectively; SEQ ID NOs: 228 and 420, respectively; SEQ ID NOs: 229 and 421, respectively; SEQ ID NOs: 230 and 422, respectively; SEQ ID NOs: 231 and 423, respectively; or SEQ ID NOs: 232 and 424, respectively, or any antisense or sense strand that has at least 80% sequence identity to any of the above sequences, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto, or any antisense or sense stand that differs by 1, 2, 3, or 4 nucleotides from an equal length portion of the sequences of SEQ ID NOs: 213 and 405, respectively; SEQ ID NOs: 214 and 406, respectively; SEQ ID NOs: 215 and 407, respectively; SEQ ID NOs: 216 and 408, respectively; SEQ ID NOs: 217 and 409, respectively; SEQ ID NOs: 218 and 410, respectively; SEQ ID NOs: 219 and 411, respectively; SEQ ID NOs: 220 and 412, respectively; SEQ ID NOs: 221 and 413, respectively; SEQ ID NOs: 222 and 414, respectively; SEQ ID NOs: 223 and 415, respectively; SEQ ID NOs: 224 and 416, respectively; SEQ ID NOs: 225 and 417, respectively; SEQ ID NOs: 226 and 418, respectively; SEQ ID NOs: 227 and 419, respectively; SEQ ID NOs: 228 and 420, respectively; SEQ ID NOs: 229 and 421, respectively; SEQ ID NOs: 230 and 422, respectively; SEQ ID NOs: 231 and 423, respectively; or SEQ ID NOs: 232 and 424, respectively.

In some embodiments, the modified antisense and the modified sense strands of the RNAi agent comprise the nucleotide sequences of SEQ ID NOs: 233 and 425, respectively; SEQ ID NOs: 234 and 426, respectively; SEQ ID NOs: 235 and 427, respectively; SEQ ID NOs: 236 and 428, respectively; SEQ ID NOs: 237 and 429, respectively; SEQ ID NOs: 238 and 430, respectively; SEQ ID NOs: 239 and 431, respectively; SEQ ID NOs: 240 and 432, respectively; SEQ ID NOs: 241 and 433, respectively; SEQ ID NOs: 242 and 434, respectively; SEQ ID NOs: 243 and 435, respectively; SEQ ID NOs: 244 and 436, respectively; SEQ ID NOs: 245 and 437, respectively; SEQ ID NOs: 246 and 438, respectively; SEQ ID NOs: 247 and 439, respectively; SEQ ID NOs: 248 and 440, respectively; SEQ ID NOs: 249 and 441, respectively; SEQ ID NOs: 250 and 442, respectively; SEQ ID NOs: 251 and 443, respectively; or SEQ ID NOs: 252 and 444, respectively, or any antisense or sense strand that has at least 80% sequence identity to any of the above sequences, such as at least 80% sequence identity to any of the above sequences, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto, or any antisense or sense stand that differs by 1, 2, 3, or 4 nucleotides from an equal length portion of the sequences of SEQ ID NOs: 233 and 425, respectively; SEQ ID NOs: 234 and 426, respectively; SEQ ID NOs: 235 and 427, respectively; SEQ ID NOs: 236 and 428, respectively; SEQ ID NOs: 237 and 429, respectively; SEQ ID NOs: 238 and 430, respectively; SEQ ID NOs: 239 and 431, respectively; SEQ ID NOs: 240 and 432, respectively; SEQ ID NOs: 241 and 433, respectively; SEQ ID NOs: 242 and 434, respectively; SEQ ID NOs: 243 and 435, respectively; SEQ ID NOs: 244 and 436, respectively; SEQ ID NOs: 245 and 437, respectively; SEQ ID NOs: 246 and 438, respectively; SEQ ID NOs: 247 and 439, respectively; SEQ ID NOs: 248 and 440, respectively; SEQ ID NOs: 249 and 441, respectively; SEQ ID NOs: 250 and 442, respectively; SEQ ID NOs: 251 and 443, respectively; or SEQ ID NOs: 252 and 444.

In some embodiments, the modified antisense and the modified sense strands of the RNAi agent comprise the nucleotide sequences of SEQ ID NOs: 253 and 445, respectively; SEQ ID NOs: 254 and 446, respectively; SEQ ID NOs: 255 and 447, respectively; SEQ ID NOs: 256 and 448, respectively; SEQ ID NOs: 257 and 449, respectively; SEQ ID NOs: 258 and 450, respectively; SEQ ID NOs: 259 and 451, respectively; SEQ ID NOs: 260 and 452, respectively; SEQ ID NOs: 261 and 453, respectively; SEQ ID NOs: 262 and 454, respectively; SEQ ID NOs: 263 and 455, respectively; SEQ ID NOs: 264 and 456, respectively; SEQ ID NOs: 265 and 457, respectively; SEQ ID NOs: 266 and 458, respectively; SEQ ID NOs: 267 and 459, respectively; SEQ ID NOs: 268 and 460, respectively; SEQ ID NOs: 269 and 461, respectively; SEQ ID NOs: 270 and 462, respectively; SEQ ID NOs: 271 and 463, respectively; or SEQ ID NOs: 272 and 464, respectively, or any antisense or sense strand that has at least 80% sequence identity to any of the above sequences, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto, or any antisense or sense stand that differs by 1, 2, 3, or 4 nucleotides from an equal length portion of the sequences of SEQ ID NOs: 253 and 445, respectively; SEQ ID NOs: 254 and 446, respectively; SEQ ID NOs: 255 and 447, respectively; SEQ ID NOs: 256 and 448, respectively; SEQ ID NOs: 257 and 449, respectively; SEQ ID NOs: 258 and 450, respectively; SEQ ID NOs: 259 and 451, respectively; SEQ ID NOs: 260 and 452, respectively; SEQ ID NOs: 261 and 453, respectively; SEQ ID NOs: 262 and 454, respectively; SEQ ID NOs: 263 and 455, respectively; SEQ ID NOs: 264 and 456, respectively; SEQ ID NOs: 265 and 457, respectively; SEQ ID NOs: 266 and 458, respectively; SEQ ID NOs: 267 and 459, respectively; SEQ ID NOs: 268 and 460, respectively; SEQ ID NOs: 269 and 461, respectively; SEQ ID NOs: 270 and 462, respectively; SEQ ID NOs: 271 and 463, respectively; or SEQ ID NOs: 272 and 464, respectively.

In some embodiments, the modified antisense and the modified sense strands of the RNAi agent comprise the nucleotide sequences of SEQ ID NOs: 273 and 465, respectively; SEQ ID NOs: 274 and 466, respectively; SEQ ID NOs: 275 and 467, respectively; SEQ ID NOs: 276 and 468, respectively; SEQ ID NOs: 277 and 469, respectively; SEQ ID NOs: 278 and 470, respectively; SEQ ID NOs: 279 and 471, respectively; SEQ ID NOs: 280 and 472, respectively; SEQ ID NOs: 281 and 473, respectively; SEQ ID NOs: 282 and 474, respectively; SEQ ID NOs: 283 and 475, respectively; SEQ ID NOs: 284 and 476, respectively; SEQ ID NOs: 285 and 477, respectively; SEQ ID NOs: 286 and 478, respectively; SEQ ID NOs: 287 and 479, respectively; or SEQ ID NOs: 288 and 480, respectively, or any antisense or sense strand that has at least 80% sequence identity to any of the above sequences, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto, or any antisense or sense stand that differs by 1, 2, 3, or 4 nucleotides from an equal length portion of the sequences of SEQ ID NOs: 273 and 465, respectively; SEQ ID NOs: 274 and 466, respectively; SEQ ID NOs: 275 and 467, respectively; SEQ ID NOs: 276 and 468, respectively; SEQ ID NOs: 277 and 469, respectively; SEQ ID NOs: 278 and 470, respectively; SEQ ID NOs: 279 and 471, respectively; SEQ ID NOs: 280 and 472, respectively; SEQ ID NOs: 281 and 473, respectively; SEQ ID NOs: 282 and 474, respectively; SEQ ID NOs: 283 and 475, respectively; SEQ ID NOs: 284 and 476, respectively; SEQ ID NOs: 285 and 477, respectively; SEQ ID NOs: 286 and 478, respectively; SEQ ID NOs: 287 and 479, respectively; or SEQ ID NOs: 288 and 480, respectively.

In some embodiments, the RNAi agent is prepared or provided as a salt, mixed salt, or a free-acid. In some embodiments, the RNAi agent is prepared as a sodium salt. Such forms are within the scope of the embodiments disclosed herein.

In some embodiments, the GCK RNAi agent comprises a modified antisense strand and a modified sense strand having the nucleotide sequences of any of the modified antisense strand and/or modified sense strand nucleotide sequences of any of the duplexes of Table 3. In some embodiments, the duplexes of shown in Table 3 and further comprises an asialoglycoprotein receptor ligand targeting group. In some embodiments, the GCK RNAi agent comprises any of the duplexes of Table 3.

### B. Modified Internucleoside Linkages and Modified Sugar-Phosphate Backbone

In some embodiments, one or more nucleotides of the RNAi agent are linked by non-standard internucleoside linkages or sugar-phosphate backbones (i.e., modified internucleoside linkages or modified backbones). Although some modifications to standard linkages or backbones can occur naturally, many synthetic modifications in the disclosed oligonucleotides are not found in the disclosed oligonucleotides in nature. The term “synthetically modified” herein refers to modifications not found in nature. In some embodiments, a modified internucleoside linkage is a non-phosphate-containing covalent internucleoside linkage. Modified internucleoside linkages or backbones include, but are not limited to, 5′-phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, the first and/or second internucleoside linkages of the sense strand and/or the first and/or second internuculeoside linkages of the antisense strand are modified (e.g., phosphorothioate) linkages. In some embodiments, the final and/or penultimate internucleoside linkages of the sense strand and/or the final and/or penultimate internucleoside linkages of the antisense strand are modified (e.g., phosphorothioate) linkages.

In some embodiments, a sense strand of the RNAi agent comprises 1, 2, 3, 4, 5, or 6 phosphorothioate linkages, an antisense strand of the RNAi agent comprises 1, 2, 3, 4, 5, or 6 phosphorothioate linkages, or both the sense strand and the antisense strand independently comprise 1, 2, 3, 4, 5, or 6 phosphorothioate linkages. In some embodiments, a sense strand of the RNAi agent comprises 1, 2, 3, or 4 phosphorothioate linkages, an antisense strand of the RNAi agent comprises 1, 2, 3, or 4 phosphorothioate linkages, or both the sense strand and the antisense strand independently comprise 1, 2, 3, or 4 phosphorothioate linkages.

In some embodiments, the RNAi agent sense strand comprises at least two phosphorothioate internucleoside linkages. In some embodiments, the at least two phosphorothioate internucleoside linkages are between the nucleotides at positions 1-3 from the 3′ end of the sense strand. In some embodiments, the at least two phosphorothioate internucleoside linkages are between the nucleotides at positions 1-3, 2-4, 3-5, 4-6, 4-5, or 6-8 from the 5′ end of the sense strand. In some embodiments, the at least two phosphorothioate internucleoside linkages are following bases 1, 2, 19, and 20 of the sense strand. In some embodiments, the RNAi agent antisense strand comprises four phosphorothioate internucleoside linkages. In some embodiments, the four phosphorothioate internucleoside linkages are between the nucleotides at positions 1-3 from the 5′ end of the sense strand and between the nucleotides at positions 19-21, 20-22, 21-23, 22-24, 23-25, or 24-26 from the 5′ end. In some embodiments, the four phosphorothioate internucleoside linkages are following bases 1, 2, 21, and 22 of the antisense strand. In some embodiments, the RNAi agent comprises at least two phosphorothioate internucleoside linkages in the sense strand and three or four phosphorothioate internucleoside linkages in the antisense strand. In some embodiments, the RNAi agent contains one or more modified nucleotides and one or more modified internucleoside linkages. In some embodiments, a 2′-modified nucleoside is combined with modified internucleoside linkage.

Various implementations provide a modified oligonucleotide having 12 to 30 or 12 to 50 linked nucleosides. The modified oligonucleotide includes at least one synthetically modified internucleoside linkage or at least one synthetically modified sugar-phosphate backbone. The modified oligonucleotide has a nucleobase sequence including at least 12, at least 13, at least 14, at least 15, at least 16, at least 17, at least 18, at least 19, at least 20, at least 21, at least 22, at least 23, at least 24, or at least 25 contiguous nucleobases complementary to an equal length portion of SEQ ID NO: 481 (GCK).

The modified oligonucleotides described herein encompass a broad range of oligonucleotide-based therapeutics. These include, but are not limited to, small interfering RNA (siRNA), small activating RNA (saRNA), antisense oligonucleotides (ASO), regulatory oligonucleotides, miRNA mimics and inhibitors, ribozymes, DNAzymes, splice-switching oligonucleotides (SSOs), decoy oligonucleotides, aptamers, DNA triplex-forming oligonucleotides (TFOs), etc.

Small activating RNA (saRNA) are short double-stranded RNA molecules designed to upregulate gene expression by targeting promoter regions or enhancer elements. Modifications similar to those used in siRNA can be applied to saRNA to enhance their stability and targeting efficacy, ensuring prolonged activation of gene expression.

Antisense oligonucleotides (ASO) are single-stranded DNA or RNA sequences designed to bind complementary mRNA transcripts, thereby blocking translation or promoting degradation through RNase H activity. ASOs often include modifications such as phosphorothioate backbones, 2′-O-methyl or 2′-O-methoxyethyl sugar modifications, and incorporation of L NAs to improve resistance to nucleases and increase binding affinity.

MicroRNA (miRNA) mimics are synthetic oligonucleotides designed to mimic endogenous miRNAs—small, non-coding RNA molecules typically 19-25 nucleotides in length that play a critical role in regulating gene expression. miRNA mimics can restore the function of miRNAs that are downregulated in certain diseases such as cancer.

MicroRNA inhibitors (antagomirs) are chemically modified, single-stranded oligonucleotides designed to bind and inhibit endogenous miRNAs. They can be used to upregulate target gene expression by blocking miRNA function. Common modifications include locked nucleic acids (LNA), 2′-O-methyl, and phosphorothioate linkages to enhance stability and binding affinity.

Ribozymes are RNA molecules with catalytic activity, capable of cleaving specific RNA sequences. Engineered ribozymes can be designed to target and degrade pathogenic mRNAs, offering a therapeutic avenue for diseases caused by aberrant gene expression.

DNAzymes are DNA oligonucleotides with catalytic functions similar to ribozymes. DNAzymes can be designed to cleave specific RNA sequences, providing a potential therapeutic strategy for inactivating harmful mRNA transcripts. Chemical modifications such as phosphorothioate backbones can improve their stability and efficacy.

Splice-switching oligonucleotides (SSOs) are designed to modulate pre-mRNA splicing, allowing for the correction of aberrant splicing patterns or the exclusion of deleterious exons. By binding to splicing enhancers or silencers, SSOs can restore the expression of functional proteins. Modified backbones such as phosphorodiamidate morpholino oligomers (PMOs) and 2′-O-methoxyethyl modifications are often used to improve their pharmacological properties.

Decoy oligonucleotides are designed to mimic specific regulatory DNA or RNA sequences, competitively binding to transcription factors, RNA-binding proteins, or other regulatory molecules. This can inhibit the interaction of these regulatory molecules with their natural targets, thereby modulating gene expression. These decoys often incorporate modifications to increase their stability and binding affinity.

A ptamers are short nucleic acid sequences that can fold into specific three-dimensional shapes, allowing them to bind their target molecules with high specificity and affinity, akin to antibodies. A ptamers can be used as therapeutic agents to neutralize proteins, interfere with protein-protein interactions, or deliver drugs to specific cells. Modifications such as polyethylene glycol (PEG) conjugation and nucleotide analogs can enhance their stability and prolong their activity in biological systems.

DNA triplex-forming oligonucleotides (TFOs) bind to the major groove of double-stranded DNA, forming a triple-helix structure. This can be used to modulate gene expression by blocking the binding of transcription factors or other DNA-binding proteins, thereby regulating the transcription of target genes. TFOs often include modifications such as 2′-O-methyl or locked nucleic acids to enhance their stability and binding specificity.

Additionally, the modified oligonucleotides are envisioned to include other emerging oligonucleotide-based therapeutics not enumerated above but have the same recited features. Synthetic oligonucleotides are versatile in treating a wide array of diseases. With various chemical modifications, these oligos can betailored to improve their stability, specificity, and therapeutic efficacy in precision medicine and targeted therapies.

### C. Targeting Groups

In some embodiments, the RNAi agent as provided herein is conjugated to one or more non-nucleotide groups including, but not limited to a targeting group, linking group, delivery polymer, or a delivery vehicle. In some embodiments, the targeting group (e.g. targeting ligand) is conjugated to the RNAi agent. The non-nucleotide group can enhance targeting, delivery or attachment of the RNAi agent. Examples of targeting groups are provided in Table 4. The targeting group (e.g. targeting ligand) can be covalently linked to the 3′ and/or 5′ end of either the sense strand and/or the antisense strand. In some implementations, the targeting group is covalently linked to the 3′ end of the sense strand.

In some embodiments, the RNAi agent includes a non-nucleotide group linked to the 3′ and/or 5′ end of the sense strand and/or the antisense strand. In some embodiments, a non-nucleotide group is linked to the 3′ end of the RNAi agent sense strand. In various embodiments, the non-nucleotide group includes a targeting group or targeting ligand. A non-nucleotide group may be linked directly or indirectly to the RNAi agent via a linker/linking group. In some embodiments, a non-nucleotide group is linked to the RNAi agent via a labile, cleavable, or reversible bond or linker. Exemplary targeting groups (e.g., ligands) are disclosed in U.S. Pat. No. 9,771,591, which is incorporated herein in its entirety. In some embodiments, the RNAi agent includes a non-nucleotide group linked to an internal nucleotide of the sense strand and/or the antisense strand, the internal nucleotide being any nucleotide between the 3′ end and the 5′ end of a strand. In some embodiments, the non-nucleotide group is covalently linked to a sugar or a base of the internal nucleotide.

In some embodiments, a non-nucleotide group enhances the pharmacokinetic or biodistribution properties of the RNAi agent or conjugate to which it is attached to improve cell- or tissue-specific distribution and cell-specific uptake of the conjugate. In some embodiments, a non-nucleotide group enhances endocytosis of the RNAi agent. Such moieties include but are not limited to lipid moieties such as a cholesterol moiety (Letsinger et al., (1989) Proc. Natl. Acid. Sci. USA, 86: 6553-6556), cholic acid (Manoharan et al., (1994) Biorg. Med. Chem. L et., 4:1053-1060), a thioether, e.g., beryl-S-tritylthiol (Manoharan et al., (1992) Ann. N.Y. Acad. Sci., 660:306-309; Manoharan et al., (1993) Biorg. Med. Chem. Let., 3:2765-2770), a thiocholesterol (Oberhauser et al., (1992) Nucl. Acids Res., 20:533-538), an aliphatic chain, e.g., dodecandiol or undecyl residues (Saison-Behmoaras et al., (1991) EMBO J, 10:1111-1118; Kabanov et al., (1990) FEBS Lett., 259:327-330; Svinarchuk et al., (1993) Biochimie, 75:49-54), a phospholipid, e.g., di-hexadecyl-rac-glycerol or triethyl-ammonium 1,2-di-O-hexadecyl-rac-glycero-3-phosphonate (Manoharan et al., (1995) Tetrahedron Lett., 36:3651-3654; Shea et al., (1990) Nucl. Acids Res., 18:3777-3783), a polyamine or a polyethylene glycol chain (Manoharan et al., (1995) Nucleosides & Nucleotides, 14:969-973), or adamantane acetic acid (Manoharan et al., (1995) Tetrahedron Lett., 36:3651-3654), a palmityl moiety (Mishra et al., (1995) Biochim. Biophys. Acta, 1264:229-237), or an octadecylamine or hexylamino-carbonyloxycholesterol moiety (Crooke et al., (1996) J. Pharmacol. Exp. Ther., 277:923-937).

Targeting groups or targeting moieties enhance the pharmacokinetic or biodistribution properties of a conjugate to which they are attached to improve cell-specific distribution and cell-specific uptake of the conjugate. In some embodiments, the targeting group provides an enhanced affinity for a selected target, e.g., molecule, cell or cell type, compartment, e.g., a cellular or organ compartment, tissue, organ or region of the body, as, e.g., compared to a species absent such a targeting group. In some embodiments, the targeting group will not take part in duplex pairing in a duplexed nucleic acid.

Targeting groups can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

A targeting group can be monovalent, divalent, trivalent, tetravalent, or have higher valency. Representative targeting groups include, without limitation, compounds with affinity to cell surface molecule, cell receptor ligands, hapten, antibodies, monoclonal antibodies, antibody fragments, and antibody mimics with affinity to cell surface molecules. In some embodiments, the targeting group is a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Targeting groups can be proteins, e.g., glycoproteins, or peptides, e.g., molecules having a specific affinity for a co-ligand, or antibodies e.g., an antibody, that binds to a specified cell type such as a hepatic cell. Targeting groups can also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose. The ligand can be, for example, a lipopolysaccharide, an activator of p38 MAP kinase, or an activator of NF-κB.

The targeting group can be a substance, e.g., a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a targeting group is linked to the RNAi agent using a linker, such as a PEG linker or one, two, or three abasic and/or ribitol (abasic ribose) groups. In some embodiments, a targeting group comprises a galactose derivative cluster.

The RNAi agent described herein may be synthesized having a reactive group, such as an amine group, at the 5′-terminus. The reactive group may be used to subsequently attach a targeting moiety using methods typical in the art.

In some embodiments, a targeting group comprises an asialoglycoprotein receptor ligand. In some embodiments, an asialoglycoprotein receptor ligand includes or comprises of one or more galactose derivatives. As used herein, the term galactose derivative includes both galactose and derivatives of galactose having affinity for the asialoglycoprotein receptor that is equal to or greater than that of galactose. Galactose derivatives include, but are not limited to, galactose, galactosamine, N-formylgalactosamine, N-acetyl-galactosamine, N propionyl-galactosamine, N-n-butanoyl-galactosamine, and N-iso-butanoylgalactos-amine (see for example: Iobst, S. T. and Drickamer, K. J.B.C. 1996, 271, 6686). Galactose derivatives, and clusters of galactose derivatives, that are useful for in vivo targeting of oligonucleotides and other molecules to the liver are known in the art (see, for example, Baenziger and Fiete, 1980, Cell, 22, 611-620; Connolly et al., 1982, J. Biol. Chem., 257, 939-945). Galactose derivatives have been used to target molecules to hepatocytes in vivo through their binding to the asialoglycoprotein receptor (ASGPr) expressed on the surface of hepatocytes. Binding of ASGPr ligands to the ASGPr(s) facilitates cell-specific targeting to hepatocytes and endocytosis of the molecule into hepatocytes. ASGPr ligands can be monomeric (e.g., having a single galactose derivative) or multimeric (e.g., having multiple galactose derivatives). The galactose derivative or galactose derivative cluster may be attached to the 3′ or 5′ end of the RNAi polynucleotide using methods known in the art. The preparation of targeting groups, such as galactose derivative clusters, is described in, for example, U.S. Patent Publication Nos. US20180064819A 1 and US20170253875A 1, the contents of each of which are incorporated herein in their entirety.

In some embodiments, the RNAi agent are targeted to hepatocytes by using conjugated targeting ligands.

In some embodiments, a galactose derivative cluster comprises a molecule having two to four terminal galactose derivatives. A terminal galactose derivative is attached to a molecule through its C-1 carbon. In some embodiments, the galactose derivative cluster is a galactose derivative trimer (also referred to as tri-antennary galactose derivative or tri-valent galactose derivative). In some embodiments, the galactose derivative cluster comprises N-acetyl-galactosamines. In some embodiments, the galactose derivative cluster comprises three N-acetyl-galactosamines. In some embodiments, the galactose derivative cluster is a galactose derivative tetramer (also referred to as tetra-antennary galactose derivative or tetra-valent galactose derivative). In some embodiments, the galactose derivative cluster comprises four N-acetyl-galactosamines.

In some embodiments, a galactose derivative trimer contains three galactose derivatives, each linked to a central branch point. As used herein, a galactose derivative tetramer contains four galactose derivatives, each linked to a central branch point. The galactose derivatives can be attached to the central branch point through the C-1 carbons of the saccharides. In some embodiments, the galactose derivatives are linked to the branch point via linkers or spacers. In some embodiments, the linker or spacer is a flexible hydrophilic spacer, such as a PEG group (see, for example, U.S. Pat. No. 5,885,968; Biessen et al. J. Med. Chem. 1995 Vol. 39 p. 1538-1546). In some embodiments, the PEG spacer is a PEG₃ spacer. The branch point can be any small molecule which permits attachment of three galactose derivatives and further permits attachment of the branch point to the RNAi agent. A n example of branch point group is a di-lysine or di-glutamate. Attachment of the branch point to the RNAi agent can occur through a linker or spacer. In some embodiments, the linker or spacer comprises a flexible hydrophilic spacer, such as, but not limited to, a PEG spacer. In some embodiments, the linker comprises a rigid linker, such as a cyclic group. In some embodiments, a galactose derivative comprises or consists of N-acetyl-galactosamine. In some embodiments, the galactose derivative cluster is comprised of a galactose derivative tetramer, which can be, for example, an N-acetyl-galactosamine tetramer. In some embodiments, the galactose derivative cluster is comprised of a galactose derivative trimer, which can be, for example, an N-acetyl-galactosamine trimer.

Targeting ligands include, but are not limited to, (PAZ), (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), and (NAG39)s. Other targeting groups, including galactose cluster targeting ligands, are known in the art.

In a particularly useful implementation, the targeting ligand has a structure according to Formula I:

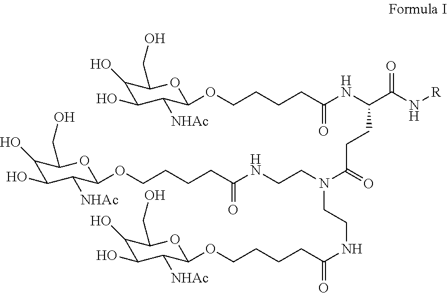

See WO 2022/271806 A 1, which is incorporated herein by reference. Formula I may have modifications or additions or deletions of one or more atoms within Formula I, or the compounds may comprise additional moieties. For example, one or more alkyl chains in Formula I may be extended or shortened, or the compound comprising Formula I may further comprise one or more RNAi agents as described herein. The compounds herein comprising Formula I are useful for delivering one or more described RNAi agents to a cell that has a receptor for one or more N-acetylgalactosamine (GalNAc, also N-GalNAc or galnac) moieties, such as the asialoglycoprotein receptor (ASPGR) that typically binds three GalNAc moieties. Accordingly, the compounds comprising Formula I herein may be used to preferentially bind to liver cells that express ASPGR, thereby facilitating entry of the compounds into liver cells. As ASPGR is also present on adipose tissue, the compounds of Formula I thus may be used to deliver RNAi agents to fat cells that express ASPGR.

In one embodiment of an RNAi agent described herein, R of Formula I is a compound comprising one or more oligonucleotides of an RNAi agent described herein. A linker, for example as further described herein, may be used to link Formula I to the one or oligonucleotides described herein. In a suitable further embodiment, the delivery moiety comprising Formula I delivers the one or more RNAi agents to liver tissue, by binding to the extracellular receptor ASPGR and permitting entry of the RNAi agents into the cells that comprise the liver tissue. The delivery moiety comprising Formula I can be used to deliver RNAi agents for diagnostic or therapeutic purposes.

In one embodiment is a compound comprising Formula I, wherein R is an oligonucleotide (e.g., an oligonucleotide comprising a sense strand and an antisense strand)linked to the remainder of Formula I either directly or through a linker, for decreasing expression of a target transcript. In a further embodiment the compound comprising Formula I, wherein R is an oligonucleotide (e.g., an oligonucleotide comprising a sense strand and an antisense strand) linked to the remainder of Formula I either directly or through a linker, for decreasing expression of a target transcript which further decreases protein expression.

In some embodiments R is conjugated to Formula I via a linker. Suitable linkers are known in the art, examples of which are described herein. In one embodiment, the linker comprises an alkyl chain, suitably C1-10. In a further embodiment, the linker is shown below as Linker 1, Formula II. In another embodiment the linker comprises a piperidine. In a further suitable embodiment, the linker is shown below as Linker 2, Formula III. In some embodiments, R is conjugated to Formula I via Formula III (also referred to herein as “Formula IV”).

### D. Linking Groups

In some embodiments, the RNAi agent as provided herein is conjugated to a linking group. Examples of linking groups are provided in Table 4. The linking group can be covalently linked to the 3′ and/or 5′ end of either the sense strand and/or the antisense strand. Exemplary linking groups are disclosed in U.S. Pat. No. 9,771,591, which is incorporated herein in its entirety.

In some embodiments, a linking group is conjugated to the RNAi agent. The linking group facilitates covalent linkage of the agent to a targeting group or delivery polymer or delivery vehicle. The linking group can be linked to the 3′ or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, include, but are not limited to: reactive groups such a primary amines and alkynes, alkyl groups, abasic nucleosides, ribitol (abasic ribose), and/or PEG groups.

A linker or linking group is a connection between two atoms that links one chemical group (such as an RNAi agent) or segment of interest to another chemical group (such as a targeting group or delivery polymer) or segment of interest via one or more covalent bonds. A labile linkage contains a labile bond. A linkage may optionally include a spacer that increases the distance between the two joined atoms. A spacer may further add flexibility and/or length to the linkage. Spacers may include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description.

In some embodiments, the linker comprises a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, substituted or unsubstituted heterocyclic; where R8 is hydrogen, acyl, aliphatic or substituted aliphatic. In one embodiment, the linker is between about 1-24 atoms, 2-24, 3-24, 4-24, 5-24, 6-24, 6-18, 7-18, 8-18 atoms, 7-17, 8-17, 6-16, 7-17, or 8-16 atoms.

In some embodiments, the linker is a cleavable linker (also referred to herein as a “cleavable linking group” that is sufficiently stable outside the cell, but which upon entry into a target cell is cleaved to release the two parts the linker is holding together. In some embodiments, the cleavable linker is cleaved at least about 10 times, 20, times, 30 times, 40 times, 50 times, 60 times, 70 times, 80 times, 90 times or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In some embodiments, a cleavable linking group is a redox cleavable linking group that is cleaved upon reduction or oxidation. A n example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

In some embodiments, a cleavable linker comprises a phosphate-based cleavable linking group. A phosphate-based cleavable linking group is cleaved by agents that degrade or hydrolyze the phosphate group. A n example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)+O—, —O—P(S)(OH)+O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

In some embodiments, a cleavable linker comprises an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula—C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

In some embodiments, a cleavable linker comprises an ester-based cleavable linking group. A n ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

In some embodiments, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula—NHCHRAC(O)NHCHRBC(O)—, where RA and RB are the R groups of the two adjacent amino acids. These candidates can be evaluated using methods analogous to those described above.

In some embodiments, the RNAi agent nucleotide sequences comprises a 3′ or 5′ targeting ligand and/or linking group. In some embodiments, the RNAi agent nucleotide sequence does not comprise a 3′ or 5′ targeting ligand and/or linking group. In some embodiments, the targeting ligand or linking group may be attached to the 3′ or 5′ terminus of either the sense strand or the antisense strand of the RNAi agent duplex.

In an exemplary embodiment, the linker is shown below as Linker 1, Formula II. In another embodiment the linker comprises a piperidine. In a further suitable embodiment, the linker is shown below as Linker 2, Formula III.

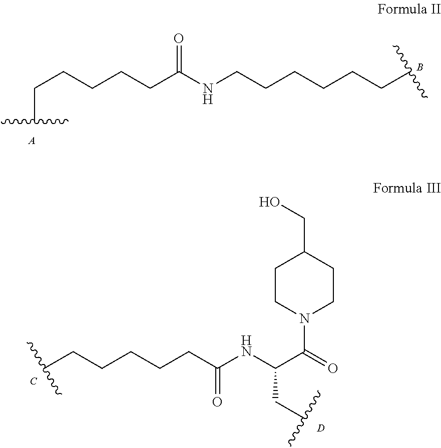

In an embodiment Linker 1 (Formula II), connection point A, or Linker 2 (Formula III), connection point C, is conjugated to Formula I. In an embodiment Linker 1, connection point A, is conjugated to Formula I and connection point B is conjugated to R (e.g., an oligonucleotide). In an embodiment Linker 2, connection point C, is conjugated to Formula I and connection point D is conjugated to R (e.g., an oligonucleotide). In an embodiment Linker 1, connection point A, is conjugated to Formula I and connection point B is conjugated to a phosphate group which is conjugated to R. In an embodiment Linker 2, connection point C, is conjugated to Formula I and connection point D is conjugated to a phosphate group which is conjugated to R. One of skill in the art will recognize that the linker may be on the 5′ or 3′ end of an oligonucleotide, or attached to one of the internal nucleotide or nucleoside bases.

One of skill in the art will also recognize that the linker maybe linked or conjugated to the 5′ or 3′ end of an oligonucleotide. One of skill in the art will also recognize that placement of a delivery moiety, such as the delivery moieties comprising Formula I, whether via a linker or not, on the 5′ end an oligonucleotide may need to overcome potential inefficient loading of Ago2 loading, or other hindrance of the RISC complex activity. For example, for a delivery moiety comprising Formula I linked or directly conjugated to an siRNA comprising a sense and an antisense strand, placement of the delivery moiety at the 5′ end of the antisense strand may create difficulties for Ago2 loading and prevent efficient knockdown. In a suitable embodiment, the one or more oligonucleotides comprise an siRNA comprising a sense and an antisense strand, and the delivery moiety comprising Formula I is present on the 3′ end of the sense strand. In a further embodiment, the delivery moiety comprising Formula I is conjugated to the 3′ end of the sense strand via a linker. In yet a further embodiment the linker comprises a ring structure, suitably a piperidine ring. In yet a further embodiment, the linker comprises Linker 2.

Additional examples of targeting ligands, linking groups, and combinations thereof are provided in Table 4. In some embodiments, the targeting ligand and linking group combination comprises Formula IV. In some embodiments, the targeting ligand and linking group combination comprises Formula V. In some embodiments, the targeting ligand and linking group combination comprises Formula VI.

Table 4. Structures representing various modified nucleotides, targeting groups, linking groups, and combinations of targeting groups and linking groups.

TABLE 4
Structures representing various modified nucleotides, targeting groups, linking
groups, and combinations of targeting groups and linking groups.

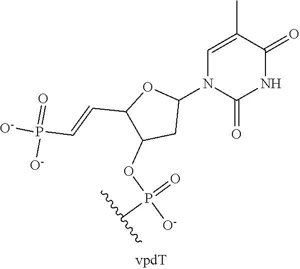 [SMILES: *P(=O)([O-])OC1CC(n2cc(C)c(=O)[nH]c2=O)OC1/C=C/P(=O)([O-])[O-]]

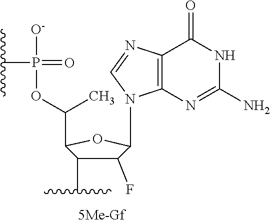 [SMILES: *C1C(F)C(n2cnc3c(=O)[nH]c(N)nc32)OC1C(C)OP(*)(=O)[O-]]

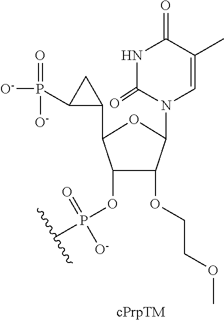 [SMILES: *P(=O)([O-])OC1C(C2CC2P(=O)([O-])[O-])OC(n2cc(C)c(=O)[nH]c2=O)C1OCCOC]

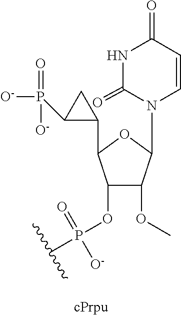 [SMILES: *P(=O)([O-])OC1C(C2CC2P(=O)([O-])[O-])OC(n2ccc(=O)[nH]c2=O)C1OC]

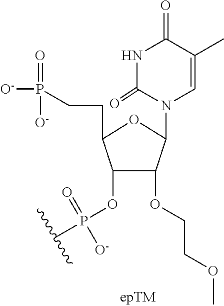 [SMILES: *P(=O)([O-])OC1C(CCP(=O)([O-])[O-])OC(n2cc(C)c(=O)[nH]c2=O)C1OCCOC]

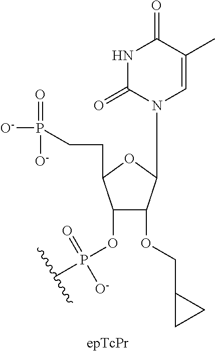 [SMILES: *P(=O)([O-])OC1C(CCP(=O)([O-])[O-])OC(n2cc(C)c(=O)[nH]c2=O)C1OCC1CC1]

When positioned internally on oligonucleotide:

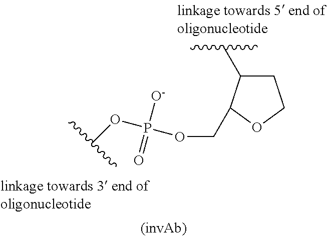 [SMILES: *OP(=O)([O-])OCC1OCCC1*]

When positioned internally on oligonucleotide:

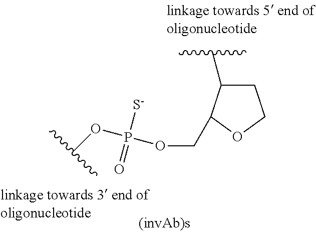 [SMILES: *OP(=O)([S-])OCC1OCCC1*]

When positioned at the 3′ terminal end of oligonucleotide:

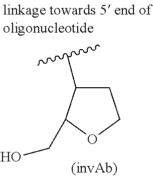

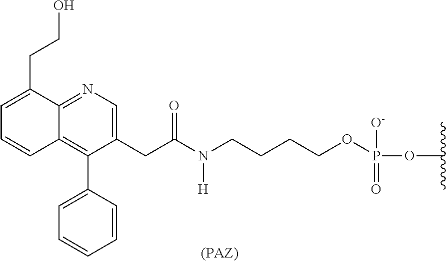

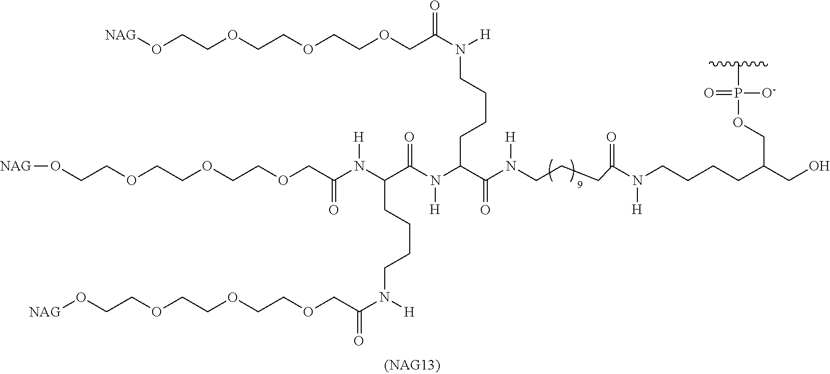

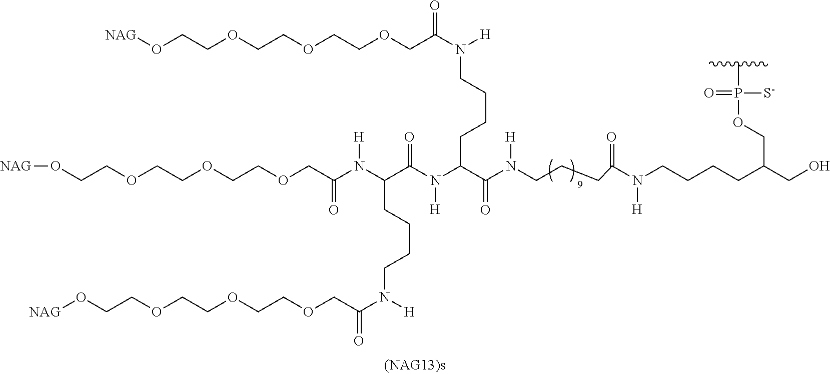

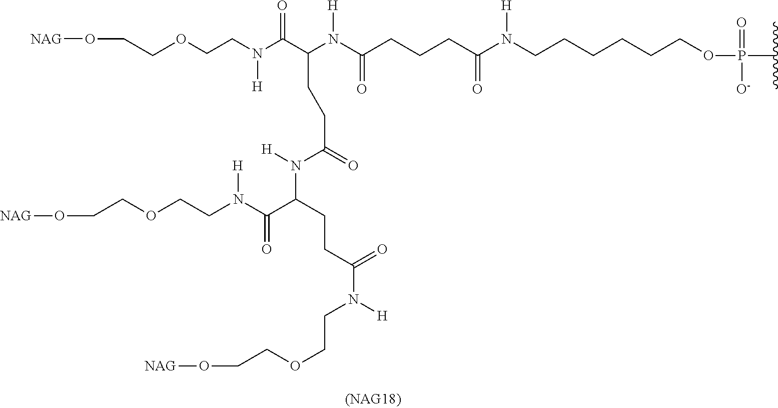

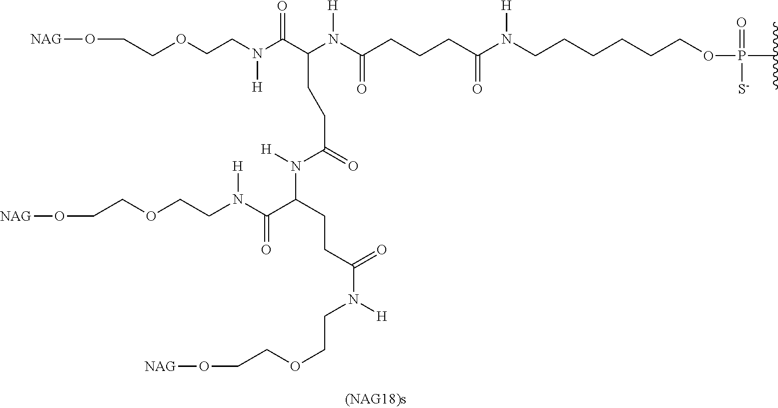

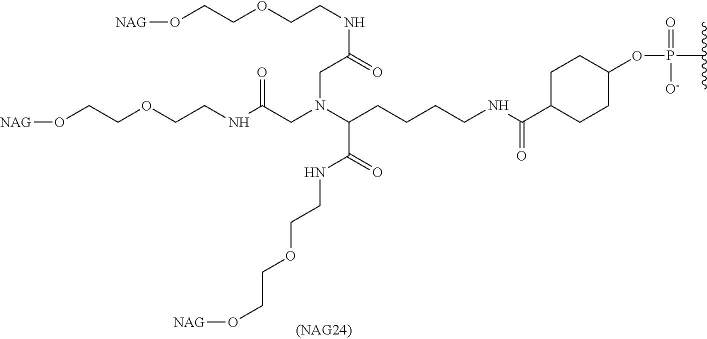

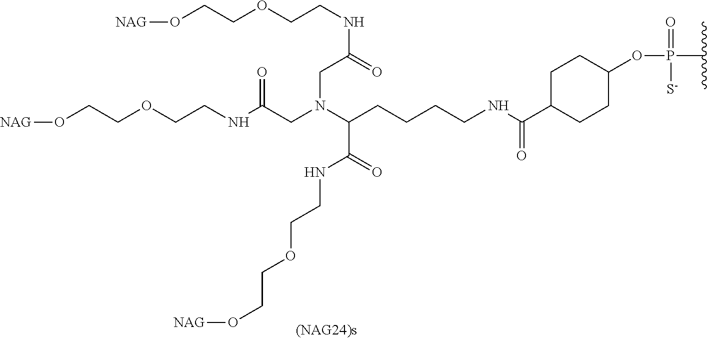

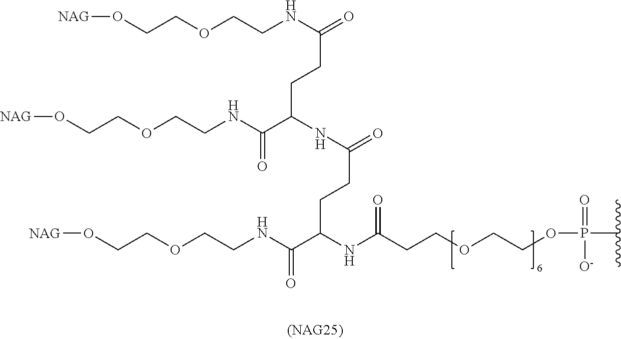

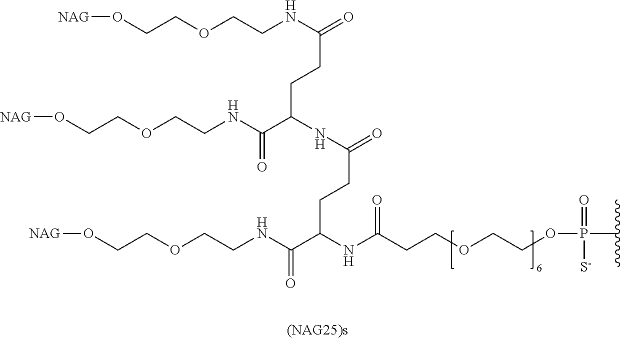

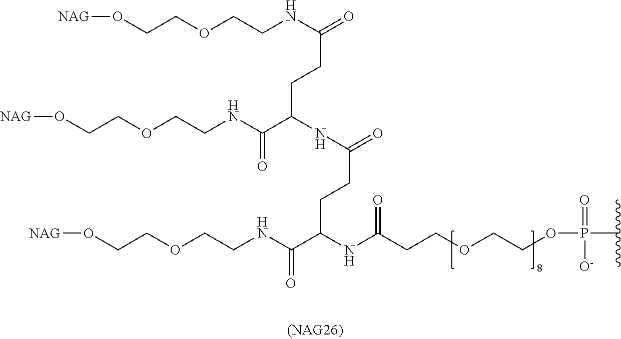

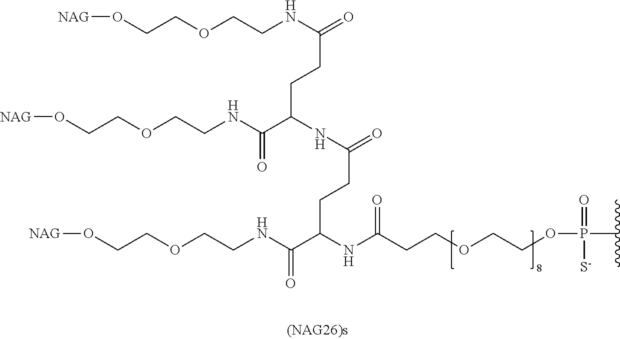

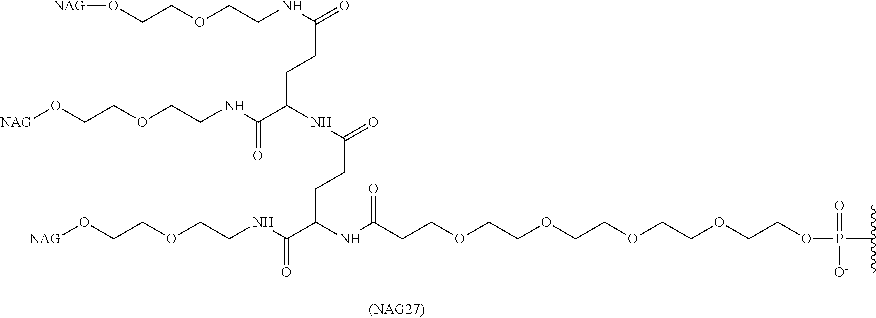

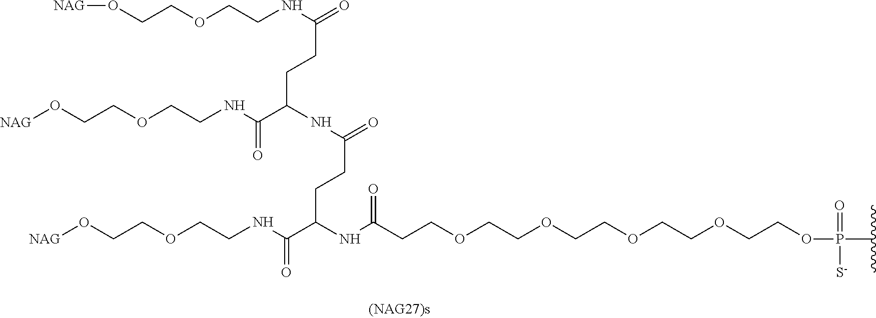

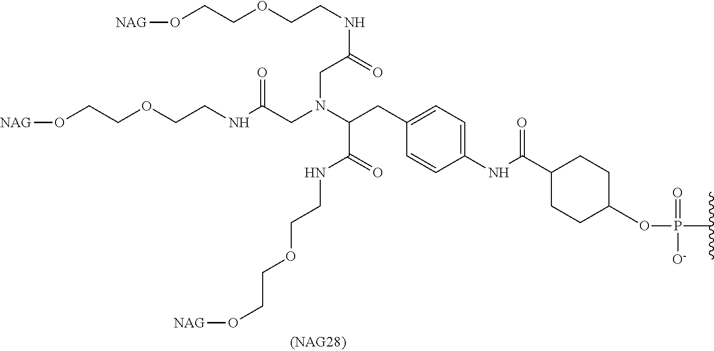

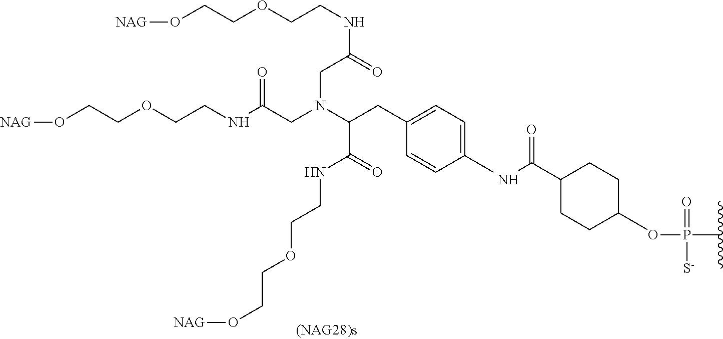

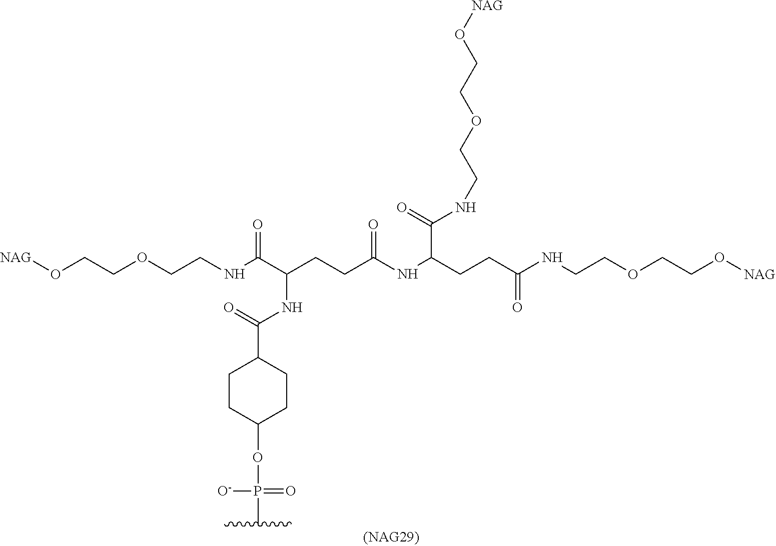

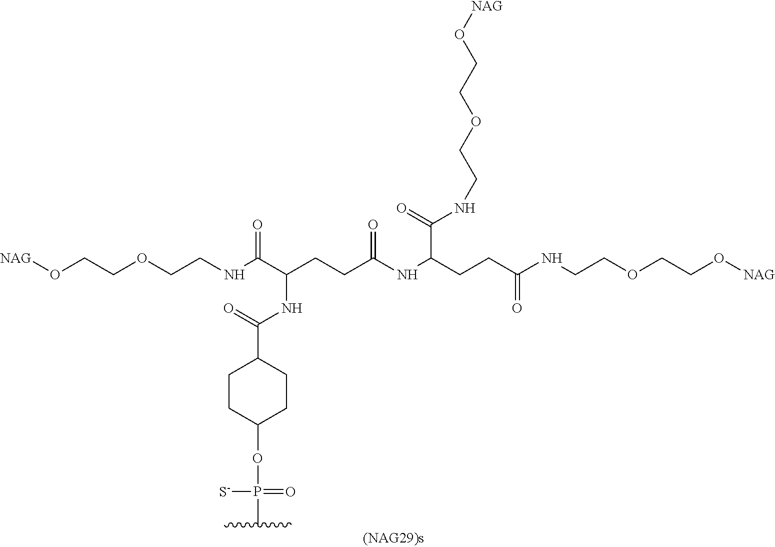

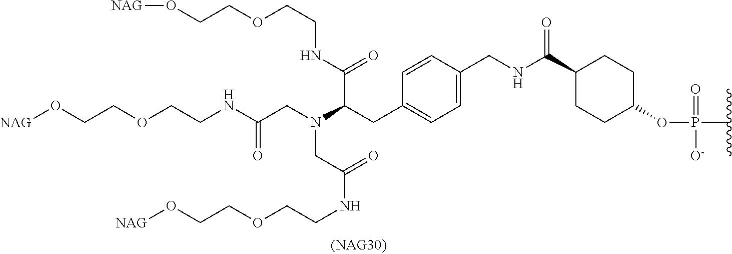

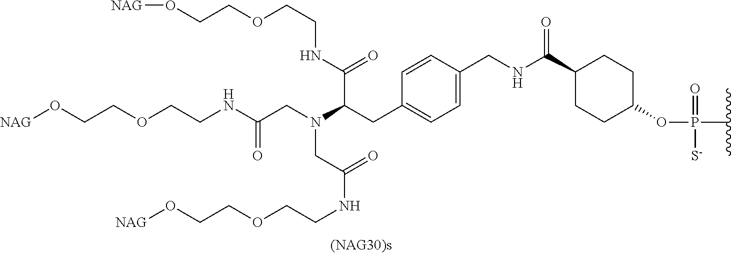

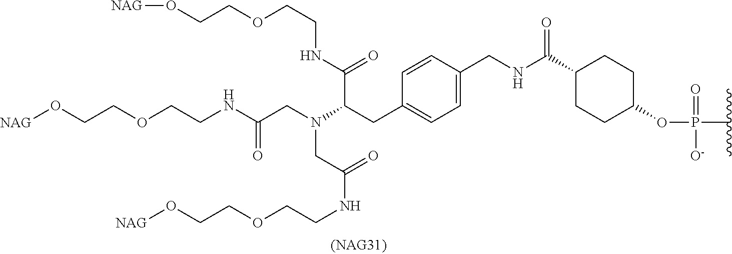

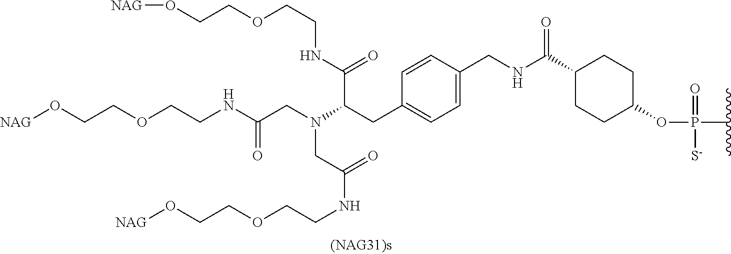

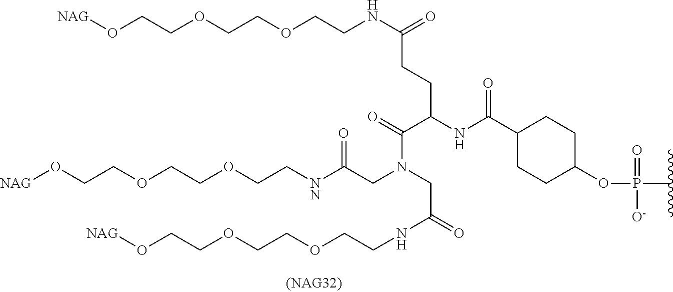

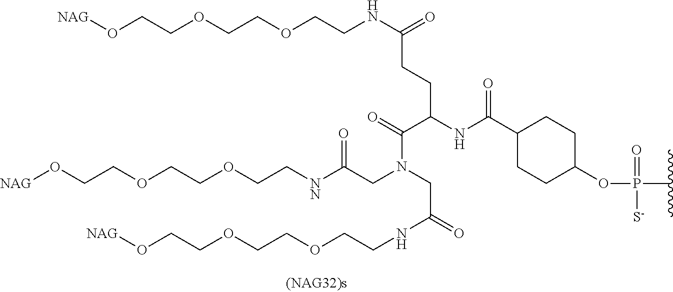

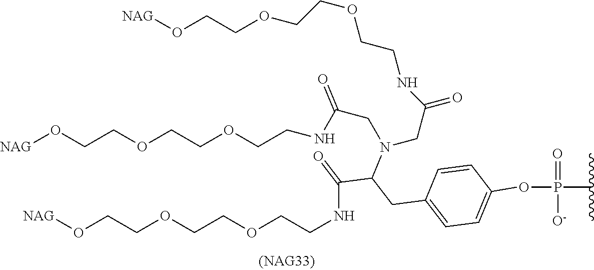

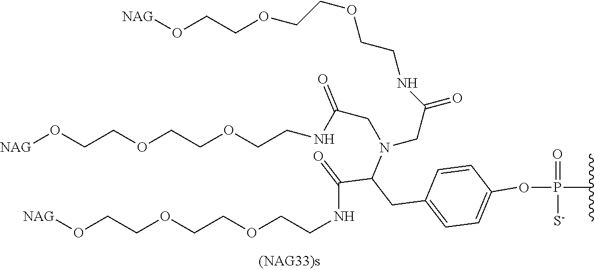

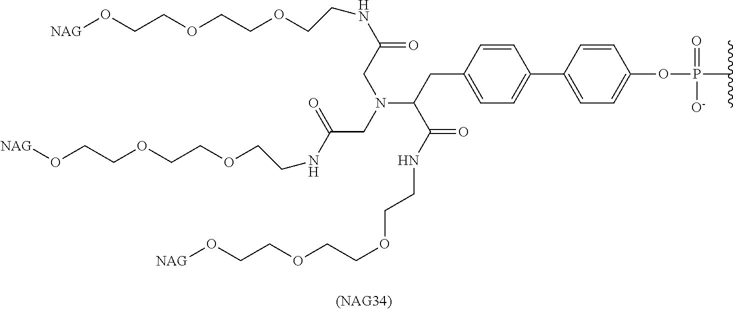

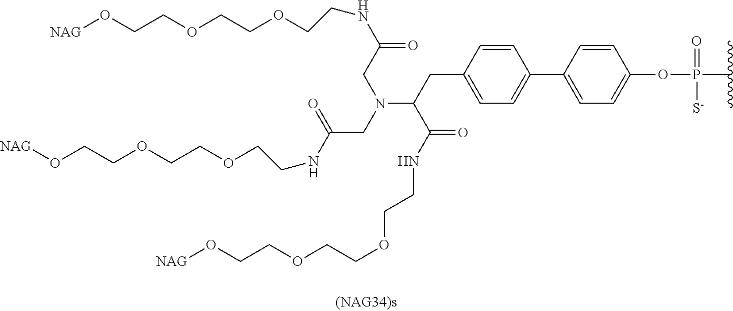

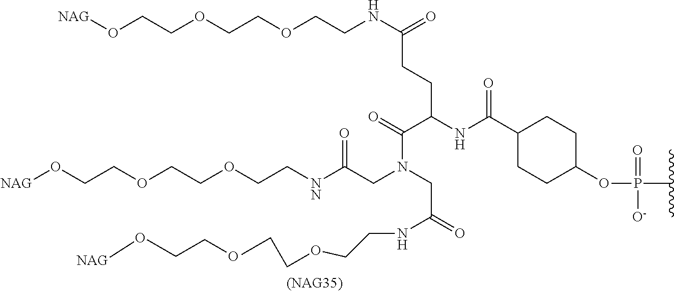

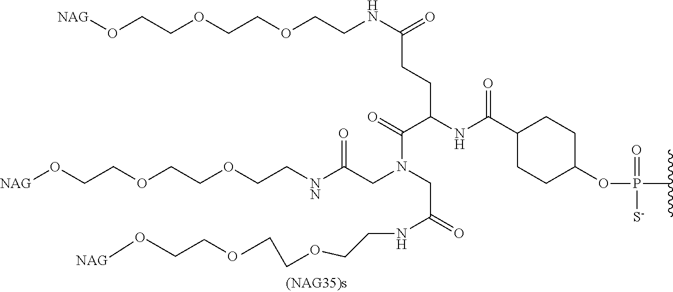

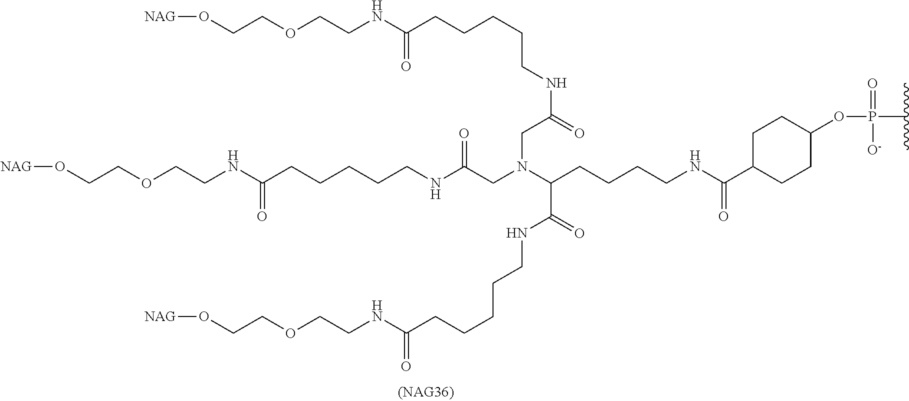

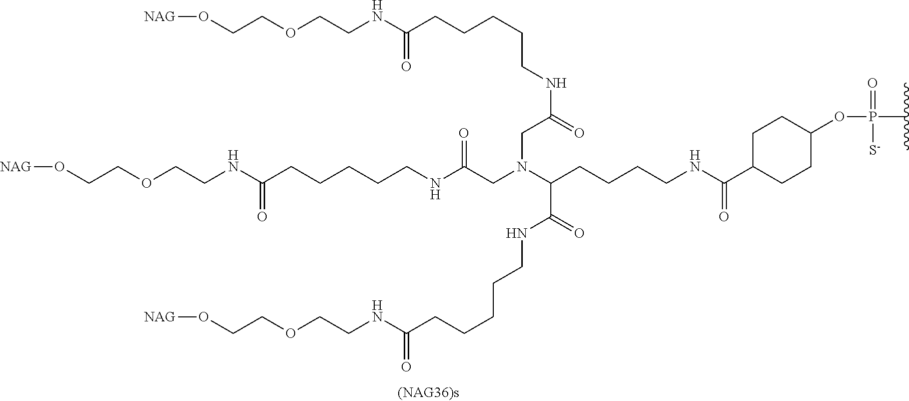

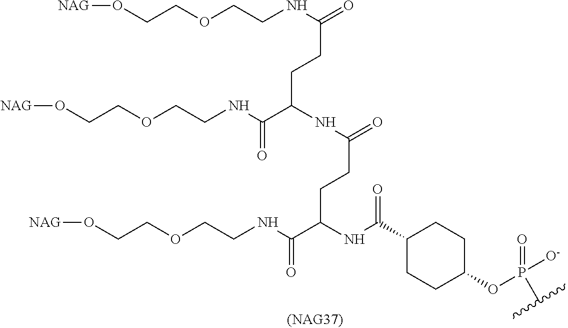

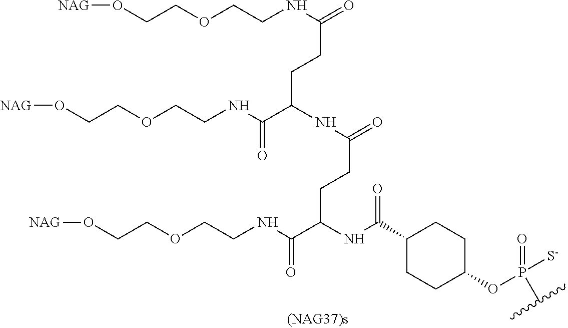

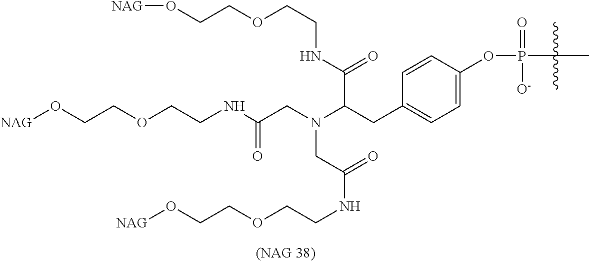

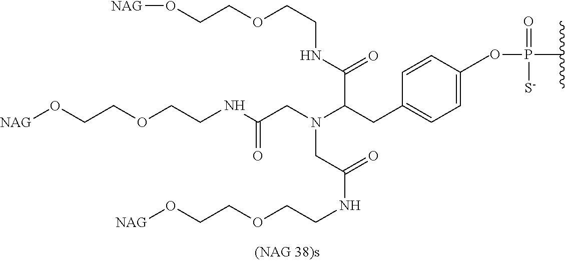

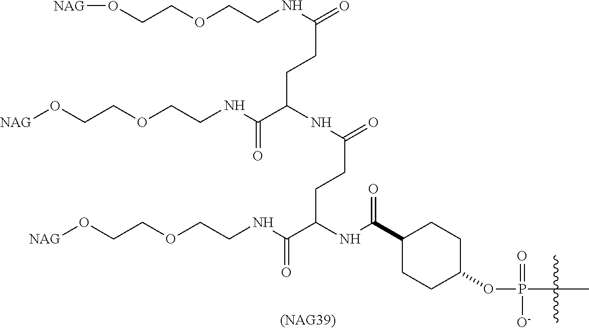

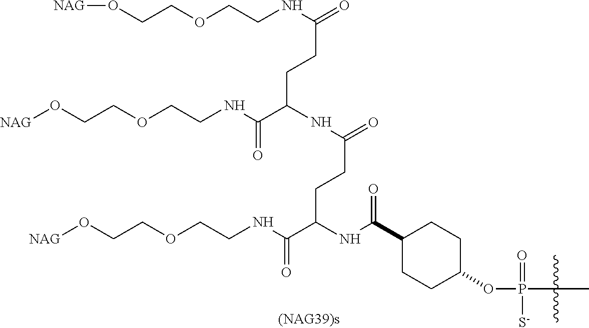

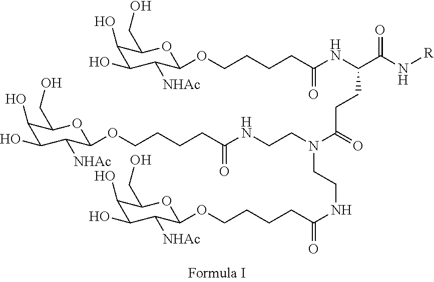

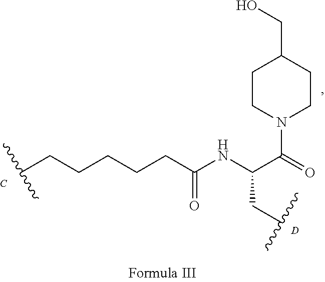

where R is an oligonucleotide conjugated to Formula I via
Formula III
Formula IV

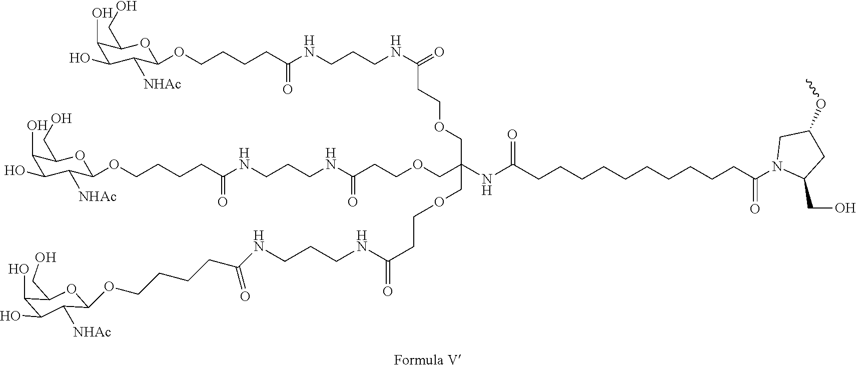

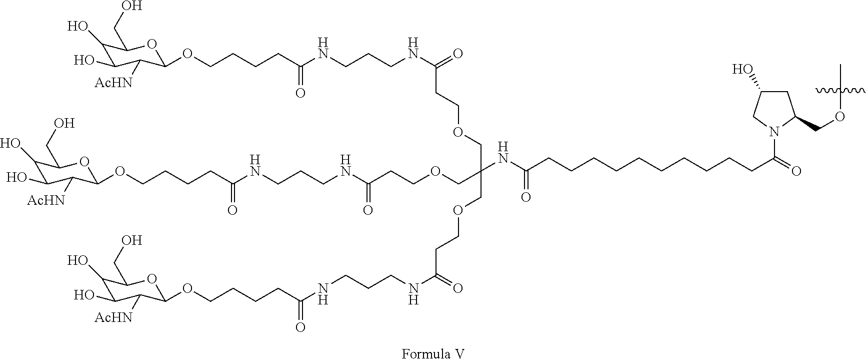 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

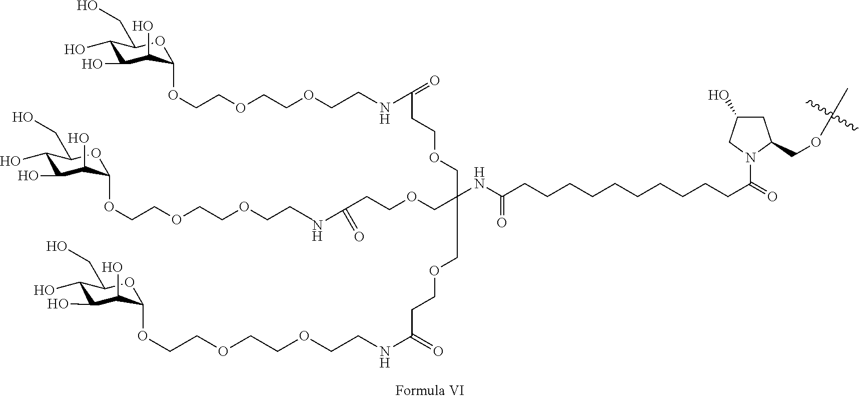

Formula V′ is a precursor of the Formula V as it may exist before it is conjugated to the RNAi agent. In some implementations of making a GalNAc-RNAi conjugate, Formula V′ is bound, e.g., to a support at the squiggle sign. Then the 3′ phosphate of a nucleotide is reacted with the methoxy off the pyrrolidine ring, and then additional nucleotides are added, growing a strand 3′→5′. Finally the GalNAc-RNAi conjugate is hydrolyzed at the site of the squiggle sign of Formula V′, cleaving it off the support, resulting a GalNAc-RNAi conjugate that includes the GalNAc of Formula V.

In some embodiments, the modified sense strand of the RNAi agent is conjugated to the targeting ligand and linking group of Formula IV.

In some embodiments, the RNAi agent has a modified sense strand of SEQ ID NO: 385 conjugated to Formula IV at the 3′ end. In some embodiments, the RNAi agent has a modified antisense strand of SEQ ID NO: 193. In some embodiments, the RNAi agent has a modified sense strand of SEQ ID NO: 385 conjugated to Formula IV at the 3′ end and a modified antisense strand of SEQ ID NO: 193.

In some embodiments, the RNAi agent has a modified sense strand of SEQ ID NO: 386 conjugated to Formula IV at the 3′ end. In some embodiments, the RNAi agent has a modified antisense strand of SEQ ID NO: 194. In some embodiments, the RNAi agent has a modified sense strand of SEQ ID NO: 386 conjugated to Formula IV at the 3′ end and a modified antisense strand of SEQ ID NO: 194.

In some embodiments, the RNAi agent has a modified sense strand of SEQ ID NO: 387 conjugated to Formula IV at the 3′ end. In some embodiments, the RNAi agent has a modified antisense strand of SEQ ID NO: 195. In some embodiments, the RNAi agent has a modified sense strand of SEQ ID NO: 387 conjugated to Formula IV at the 3′ end and a modified antisense strand of SEQ ID NO: 195.

In some embodiments, the RNAi agent has a modified sense strand of SEQ ID NO: 388 conjugated to Formula IV at the 3′ end. In some embodiments, the RNAi agent has a modified antisense strand of SEQ ID NO: 196. In some embodiments, the RNAi agent has a modified sense strand of SEQ ID NO: 388 conjugated to Formula IV at the 3′ end and a modified antisense strand of SEQ ID NO: 196.

In some embodiments, the RNAi agent has a modified sense strand of SEQ ID NO: 389 conjugated to Formula IV at the 3′ end. In some embodiments, the RNAi agent has a modified antisense strand of SEQ ID NO: 197. In some embodiments, the RNAi agent has a modified sense strand of SEQ ID NO: 389 conjugated to Formula IV at the 3′ end and a modified antisense strand of SEQ ID NO: 197.

In some embodiments, the RNAi agent has a modified sense strand of SEQ ID NO: 390 conjugated to Formula IV at the 3′ end. In some embodiments, the RNAi agent has a modified antisense strand of SEQ ID NO: 198. In some embodiments, the RNAi agent has a modified sense strand of SEQ ID NO: 390 conjugated to Formula IV at the 3′ end and a modified antisense strand of SEQ ID NO: 198.

In some embodiments, the RNAi agent has a modified sense strand of SEQ ID NO: 391 conjugated to Formula IV at the 3′ end. In some embodiments, the RNAi agent has a modified antisense strand of SEQ ID NO: 199. In some embodiments, the RNAi agent has a modified sense strand of SEQ ID NO: 391 conjugated to Formula IV at the 3′ end and a modified antisense strand of SEQ ID NO: 199.

In some embodiments, the RNAi agent has a modified sense strand of SEQ ID NO: 392 conjugated to Formula IV at the 3′ end. In some embodiments, the RNAi agent has a modified antisense strand of SEQ ID NO: 200. In some embodiments, the RNAi agent has a modified sense strand of SEQ ID NO: 392 conjugated to Formula IV at the 3′ end and a modified antisense strand of SEQ ID NO: 200.

In some embodiments, the RNAi agent has a modified sense strand of SEQ ID NO: 393 conjugated to Formula IV at the 3′ end. In some embodiments, the RNAi agent has a modified antisense strand of SEQ ID NO: 201. In some embodiments, the RNAi agent has a modified sense strand of SEQ ID NO: 393 conjugated to Formula IV at the 3′ end and a modified antisense strand of SEQ ID NO: 201.

In some embodiments, the RNAi agent has a modified sense strand of SEQ ID NO: 394 conjugated to Formula IV at the 3′ end. In some embodiments, the RNAi agent has a modified antisense strand of SEQ ID NO: 202. In some embodiments, the RNAi agent has a modified sense strand of SEQ ID NO: 394 conjugated to Formula IV at the 3′ end and a modified antisense strand of SEQ ID NO: 202.

In some embodiments, the RNAi agent has a modified sense strand of SEQ ID NO: 395 conjugated to Formula IV at the 3′ end. In some embodiments, the RNAi agent has a modified antisense strand of SEQ ID NO: 203. In some embodiments, the RNAi agent has a modified sense strand of SEQ ID NO: 395 conjugated to Formula IV at the 3′ end and a modified antisense strand of SEQ ID NO: 203.

In some embodiments, the RNAi agent has a modified sense strand of SEQ ID NO: 396 conjugated to Formula IV at the 3′ end. In some embodiments, the RNAi agent has a modified antisense strand of SEQ ID NO: 204. In some embodiments, the RNAi agent has a modified sense strand of SEQ ID NO: 306 conjugated to Formula IV at the 3′ end and a modified antisense strand of SEQ ID NO: 204.

In some embodiments, the RNAi agent has a modified sense strand of SEQ ID NO: 397 conjugated to Formula IV at the 3′ end. In some embodiments, the RNAi agent has a modified antisense strand of SEQ ID NO: 205. In some embodiments, the RNAi agent has a modified sense strand of SEQ ID NO: 397 conjugated to Formula IV at the 3′ end and a modified antisense strand of SEQ ID NO: 205.

In some embodiments, the RNAi agent has a modified sense strand of SEQ ID NO: 398 conjugated to Formula IV at the 3′ end. In some embodiments, the RNAi agent has a modified antisense strand of SEQ ID NO: 206. In some embodiments, the RNAi agent has a modified sense strand of SEQ ID NO: 398 conjugated to Formula IV at the 3′ end and a modified antisense strand of SEQ ID NO: 206.

In some embodiments, the RNAi agent has a modified sense strand of SEQ ID NO: 399 conjugated to Formula IV at the 3′ end. In some embodiments, the RNAi agent has a modified antisense strand of SEQ ID NO: 207. In some embodiments, the RNAi agent has a modified sense strand of SEQ ID NO: 399 conjugated to Formula IV at the 3′ end and a modified antisense strand of SEQ ID NO: 207.

In some embodiments, the RNAi agent has a modified sense strand of SEQ ID NO: 400 conjugated to Formula IV at the 3′ end. In some embodiments, the RNAi agent has a modified antisense strand of SEQ ID NO: 208. In some embodiments, the RNAi agent has a modified sense strand of SEQ ID NO: 400 conjugated to Formula IV at the 3′ end and a modified antisense strand of SEQ ID NO: 208.

In some embodiments, the RNAi agent has a modified sense strand of SEQ ID NO: 401 conjugated to Formula IV at the 3′ end. In some embodiments, the RNAi agent has a modified antisense strand of SEQ ID NO: 209. In some embodiments, the RNAi agent has a modified sense strand of SEQ ID NO: 401 conjugated to Formula IV at the 3′ end and a modified antisense strand of SEQ ID NO: 209.

In some embodiments, the RNAi agent has a modified sense strand of SEQ ID NO: 402 conjugated to Formula IV at the 3′ end. In some embodiments, the RNAi agent has a modified antisense strand of SEQ ID NO: 210. In some embodiments, the RNAi agent has a modified sense strand of SEQ ID NO: 402 conjugated to Formula IV at the 3′ end and a modified antisense strand of SEQ ID NO: 210.

In some embodiments, the RNAi agent has a modified sense strand of SEQ ID NO: 403 conjugated to Formula IV at the 3′ end. In some embodiments, the RNAi agent has a modified antisense strand of SEQ ID NO: 211. In some embodiments, the RNAi agent has a modified sense strand of SEQ ID NO: 403 conjugated to Formula IV at the 3′ end and a modified antisense strand of SEQ ID NO: 211.

In some embodiments, the RNAi agent has a modified sense strand of SEQ ID NO: 404 conjugated to Formula IV at the 3′ end. In some embodiments, the RNAi agent has a modified antisense strand of SEQ ID NO: 212. In some embodiments, the RNAi agent has a modified sense strand of SEQ ID NO: 404 conjugated to Formula IV at the 3′ end and a modified antisense strand of SEQ ID NO: 212.

In some embodiments, the RNAi agent has a modified sense strand of SEQ ID NO: 405 conjugated to Formula IV at the 3′ end. In some embodiments, the RNAi agent has a modified antisense strand of SEQ ID NO: 213. In some embodiments, the RNAi agent has a modified sense strand of SEQ ID NO: 405 conjugated to Formula IV at the 3′ end and a modified antisense strand of SEQ ID NO: 213.

In some embodiments, the RNAi agent has a modified sense strand of SEQ ID NO: 406 conjugated to Formula IV at the 3′ end. In some embodiments, the RNAi agent has a modified antisense strand of SEQ ID NO: 214. In some embodiments, the RNAi agent has a modified sense strand of SEQ ID NO: 406 conjugated to Formula IV at the 3′ end and a modified antisense strand of SEQ ID NO: 214.

In some embodiments, the RNAi agent has a modified sense strand of SEQ ID NO: 407 conjugated to Formula IV at the 3′ end. In some embodiments, the RNAi agent has a modified antisense strand of SEQ ID NO: 215. In some embodiments, the RNAi agent has a modified sense strand of SEQ ID NO: 407 conjugated to Formula IV at the 3′ end and a modified antisense strand of SEQ ID NO: 215.

In some embodiments, the RNAi agent has a modified sense strand of SEQ ID NO: 408 conjugated to Formula IV at the 3′ end. In some embodiments, the RNAi agent has a modified antisense strand of SEQ ID NO: 216. In some embodiments, the RNAi agent has a modified sense strand of SEQ ID NO: 408 conjugated to Formula IV at the 3′ end and a modified antisense strand of SEQ ID NO: 216.

In some embodiments, the RNAi agent has a modified sense strand of SEQ ID NO: 409 conjugated to Formula IV at the 3′ end. In some embodiments, the RNAi agent has a modified antisense strand of SEQ ID NO: 217. In some embodiments, the RNAi agent has a modified sense strand of SEQ ID NO: 409 conjugated to Formula IV at the 3′ end and a modified antisense strand of SEQ ID NO: 217.

In some embodiments, the RNAi agent has a modified sense strand of SEQ ID NO: 410 conjugated to Formula IV at the 3′ end. In some embodiments, the RNAi agent has a modified antisense strand of SEQ ID NO: 218. In some embodiments, the RNAi agent has a modified sense strand of SEQ ID NO: 410 conjugated to Formula IV at the 3′ end and a modified antisense strand of SEQ ID NO: 218.

In some embodiments, the RNAi agent has a modified sense strand of SEQ ID NO: 411 conjugated to Formula IV at the 3′ end. In some embodiments, the RNAi agent has a modified antisense strand of SEQ ID NO: 219. In some embodiments, the RNAi agent has a modified sense strand of SEQ ID NO: 411 conjugated to Formula IV at the 3′ end and a modified antisense strand of SEQ ID NO: 219.

In some embodiments, the RNAi agent has a modified sense strand of SEQ ID NO: 412 conjugated to Formula IV at the 3′ end. In some embodiments, the RNAi agent has a modified antisense strand of SEQ ID NO: 220. In some embodiments, the RNAi agent has a modified sense strand of SEQ ID NO: 412 conjugated to Formula IV at the 3′ end and a modified antisense strand of SEQ ID NO: 220.

In some embodiments, the RNAi agent has a modified sense strand of SEQ ID NO: 413 conjugated to Formula IV at the 3′ end. In some embodiments, the RNAi agent has a modified antisense strand of SEQ ID NO: 221. In some embodiments, the RNAi agent has a modified sense strand of SEQ ID NO: 413 conjugated to Formula IV at the 3′ end and a modified antisense strand of SEQ ID NO: 221.

In some embodiments, the RNAi agent has a modified sense strand of SEQ ID NO: 414 conjugated to Formula IV at the 3′ end. In some embodiments, the RNAi agent has a modified antisense strand of SEQ ID NO: 222. In some embodiments, the RNAi agent has a modified sense strand of SEQ ID NO: 414 conjugated to Formula IV at the 3′ end and a modified antisense strand of SEQ ID NO: 222.

In some embodiments, the RNAi agent has a modified sense strand of SEQ ID NO: 415 conjugated to Formula IV at the 3′ end. In some embodiments, the RNAi agent has a modified antisense strand of SEQ ID NO: 223. In some embodiments, the RNAi agent has a modified sense strand of SEQ ID NO: 415 conjugated to Formula IV at the 3′ end and a modified antisense strand of SEQ ID NO: 223.

In some embodiments, the RNAi agent has a modified sense strand of SEQ ID NO: 416 conjugated to Formula IV at the 3′ end. In some embodiments, the RNAi agent has a modified antisense strand of SEQ ID NO: 224. In some embodiments, the RNAi agent has a modified sense strand of SEQ ID NO: 416 conjugated to Formula IV at the 3′ end and a modified antisense strand of SEQ ID NO: 224.

In some embodiments, the RNAi agent has a modified sense strand of SEQ ID NO: 417 conjugated to Formula IV at the 3′ end. In some embodiments, the RNAi agent has a modified antisense strand of SEQ ID NO: 225. In some embodiments, the RNAi agent has a modified sense strand of SEQ ID NO: 417 conjugated to Formula IV at the 3′ end and a modified antisense strand of SEQ ID NO: 225.

In some embodiments, the RNAi agent has a modified sense strand of SEQ ID NO: 418 conjugated to Formula IV at the 3′ end. In some embodiments, the RNAi agent has a modified antisense strand of SEQ ID NO: 226. In some embodiments, the RNAi agent has a modified sense strand of SEQ ID NO: 418 conjugated to Formula IV at the 3′ end and a modified antisense strand of SEQ ID NO: 226.

In some embodiments, the RNAi agent has a modified sense strand of SEQ ID NO: 419 conjugated to Formula IV at the 3′ end. In some embodiments, the RNAi agent has a modified antisense strand of SEQ ID NO: 227. In some embodiments, the RNAi agent has a modified sense strand of SEQ ID NO: 419 conjugated to Formula IV at the 3′ end and a modified antisense strand of SEQ ID NO: 227.

In some embodiments, the RNAi agent has a modified sense strand of SEQ ID NO: 420 conjugated to Formula IV at the 3′ end. In some embodiments, the RNAi agent has a modified antisense strand of SEQ ID NO: 228. In some embodiments, the RNAi agent has a modified sense strand of SEQ ID NO: 420 conjugated to Formula IV at the 3′ end and a modified antisense strand of SEQ ID NO: 228.

In some embodiments, the RNAi agent has a modified sense strand of SEQ ID NO: 421 conjugated to Formula IV at the 3′ end. In some embodiments, the RNAi agent has a modified antisense strand of SEQ ID NO: 229. In some embodiments, the RNAi agent has a modified sense strand of SEQ ID NO: 421 conjugated to Formula IV at the 3′ end and a modified antisense strand of SEQ ID NO: 229.

In some embodiments, the RNAi agent has a modified sense strand of SEQ ID NO: 422 conjugated to Formula IV at the 3′ end. In some embodiments, the RNAi agent has a modified antisense strand of SEQ ID NO: 230. In some embodiments, the RNAi agent has a modified sense strand of SEQ ID NO: 422 conjugated to Formula IV at the 3′ end and a modified antisense strand of SEQ ID NO: 230.

In some embodiments, the RNAi agent has a modified sense strand of SEQ ID NO: 423 conjugated to Formula IV at the 3′ end. In some embodiments, the RNAi agent has a modified antisense strand of SEQ ID NO: 231. In some embodiments, the RNAi agent has a modified sense strand of SEQ ID NO: 423 conjugated to Formula IV at the 3′ end and a modified antisense strand of SEQ ID NO: 231.

In some embodiments, the RNAi agent has a modified sense strand of SEQ ID NO: 424 conjugated to Formula IV at the 3′ end. In some embodiments, the RNAi agent has a modified antisense strand of SEQ ID NO: 232. In some embodiments, the RNAi agent has a modified sense strand of SEQ ID NO: 424 conjugated to Formula IV at the 3′ end and a modified antisense strand of SEQ ID NO: 232.

In some embodiments, the RNAi agent has a modified sense strand of SEQ ID NO: 425 conjugated to Formula IV at the 3′ end. In some embodiments, the RNAi agent has a modified antisense strand of SEQ ID NO: 233. In some embodiments, the RNAi agent has a modified sense strand of SEQ ID NO: 425 conjugated to Formula IV at the 3′ end and a modified antisense strand of SEQ ID NO: 233.

In some embodiments, the RNAi agent has a modified sense strand of SEQ ID NO: 426 conjugated to Formula IV at the 3′ end. In some embodiments, the RNAi agent has a modified antisense strand of SEQ ID NO: 234. In some embodiments, the RNAi agent has a modified sense strand of SEQ ID NO: 426 conjugated to Formula IV at the 3′ end and a modified antisense strand of SEQ ID NO: 234.

In some embodiments, the RNAi agent has a modified sense strand of SEQ ID NO: 427 conjugated to Formula IV at the 3′ end. In some embodiments, the RNAi agent has a modified antisense strand of SEQ ID NO: 235. In some embodiments, the RNAi agent has a modified sense strand of SEQ ID NO: 427 conjugated to Formula IV at the 3′ end and a modified antisense strand of SEQ ID NO: 235.

In some embodiments, the RNAi agent has a modified sense strand of SEQ ID NO: 428 conjugated to Formula IV at the 3′ end. In some embodiments, the RNAi agent has a modified antisense strand of SEQ ID NO: 236. In some embodiments, the RNAi agent has a modified sense strand of SEQ ID NO: 428 conjugated to Formula IV at the 3′ end and a modified antisense strand of SEQ ID NO: 236.

In some embodiments, the RNAi agent has a modified sense strand of SEQ ID NO: 429 conjugated to Formula IV at the 3′ end. In some embodiments, the RNAi agent has a modified antisense strand of SEQ ID NO: 237. In some embodiments, the RNAi agent has a modified sense strand of SEQ ID NO: 429 conjugated to Formula IV at the 3′ end and a modified antisense strand of SEQ ID NO: 237.

In some embodiments, the RNAi agent has a modified sense strand of SEQ ID NO: 430 conjugated to Formula IV at the 3′ end. In some embodiments, the RNAi agent has a modified antisense strand of SEQ ID NO: 238. In some embodiments, the RNAi agent has a modified sense strand of SEQ ID NO: 430 conjugated to Formula IV at the 3′ end and a modified antisense strand of SEQ ID NO: 238.

In some embodiments, the RNAi agent has a modified sense strand of SEQ ID NO: 431 conjugated to Formula IV at the 3′ end. In some embodiments, the RNAi agent has a modified antisense strand of SEQ ID NO: 239. In some embodiments, the RNAi agent has a modified sense strand of SEQ ID NO: 431 conjugated to Formula IV at the 3′ end and a modified antisense strand of SEQ ID NO: 239.

In some embodiments, the RNAi agent has a modified sense strand of SEQ ID NO: 432 conjugated to Formula IV at the 3′ end. In some embodiments, the RNAi agent has a modified antisense strand of SEQ ID NO: 240. In some embodiments, the RNAi agent has a modified sense strand of SEQ ID NO: 432 conjugated to Formula IV at the 3′ end and a modified antisense strand of SEQ ID NO: 240.

In some embodiments, the RNAi agent has a modified sense strand of SEQ ID NO: 433 conjugated to Formula IV at the 3′ end. In some embodiments, the RNAi agent has a modified antisense strand of SEQ ID NO: 241. In some embodiments, the RNAi agent has a modified sense strand of SEQ ID NO: 433 conjugated to Formula IV at the 3′ end and a modified antisense strand of SEQ ID NO: 241.

In some embodiments, the RNAi agent has a modified sense strand of SEQ ID NO: 434 conjugated to Formula IV at the 3′ end. In some embodiments, the RNAi agent has a modified antisense strand of SEQ ID NO: 242. In some embodiments, the RNAi agent has a modified sense strand of SEQ ID NO: 434 conjugated to Formula IV at the 3′ end and a modified antisense strand of SEQ ID NO: 242.

In some embodiments, the RNAi agent has a modified sense strand of SEQ ID NO: 435 conjugated to Formula IV at the 3′ end. In some embodiments, the RNAi agent has a modified antisense strand of SEQ ID NO: 243. In some embodiments, the RNAi agent has a modified sense strand of SEQ ID NO: 435 conjugated to Formula IV at the 3′ end and a modified antisense strand of SEQ ID NO: 243.

In some embodiments, the RNAi agent has a modified sense strand of SEQ ID NO: 436 conjugated to Formula IV at the 3′ end. In some embodiments, the RNAi agent has a modified antisense strand of SEQ ID NO: 244. In some embodiments, the RNAi agent has a modified sense strand of SEQ ID NO: 436 conjugated to Formula IV at the 3′ end and a modified antisense strand of SEQ ID NO: 244.

In some embodiments, the RNAi agent has a modified sense strand of SEQ ID NO: 437 conjugated to Formula IV at the 3′ end. In some embodiments, the RNAi agent has a modified antisense strand of SEQ ID NO: 245. In some embodiments, the RNAi agent has a modified sense strand of SEQ ID NO: 437 conjugated to Formula IV at the 3′ end and a modified antisense strand of SEQ ID NO: 245.

In some embodiments, the RNAi agent has a modified sense strand of SEQ ID NO: 438 conjugated to Formula IV at the 3′ end. In some embodiments, the RNAi agent has a modified antisense strand of SEQ ID NO: 246. In some embodiments, the RNAi agent has a modified sense strand of SEQ ID NO: 438 conjugated to Formula IV at the 3′ end and a modified antisense strand of SEQ ID NO: 246.

In some embodiments, the RNAi agent has a modified sense strand of SEQ ID NO: 439 conjugated to Formula IV at the 3′ end. In some embodiments, the RNAi agent has a modified antisense strand of SEQ ID NO: 247. In some embodiments, the RNAi agent has a modified sense strand of SEQ ID NO: 439 conjugated to Formula IV at the 3′ end and a modified antisense strand of SEQ ID NO: 247.

In some embodiments, the RNAi agent has a modified sense strand of SEQ ID NO: 440 conjugated to Formula IV at the 3′ end. In some embodiments, the RNAi agent has a modified antisense strand of SEQ ID NO: 248. In some embodiments, the RNAi agent has a modified sense strand of SEQ ID NO: 440 conjugated to Formula IV at the 3′ end and a modified antisense strand of SEQ ID NO: 248.

In some embodiments, the RNAi agent has a modified sense strand of SEQ ID NO: 441 conjugated to Formula IV at the 3′ end. In some embodiments, the RNAi agent has a modified antisense strand of SEQ ID NO: 249. In some embodiments, the RNAi agent has a modified sense strand of SEQ ID NO: 441 conjugated to Formula IV at the 3′ end and a modified antisense strand of SEQ ID NO: 249.

In some embodiments, the RNAi agent has a modified sense strand of SEQ ID NO: 442 conjugated to Formula IV at the 3′ end. In some embodiments, the RNAi agent has a modified antisense strand of SEQ ID NO: 250. In some embodiments, the RNAi agent has a modified sense strand of SEQ ID NO: 442 conjugated to Formula IV at the 3′ end and a modified antisense strand of SEQ ID NO: 250.

In some embodiments, the RNAi agent has a modified sense strand of SEQ ID NO: 443 conjugated to Formula IV at the 3′ end. In some embodiments, the RNAi agent has a modified antisense strand of SEQ ID NO: 251. In some embodiments, the RNAi agent has a modified sense strand of SEQ ID NO: 443 conjugated to Formula IV at the 3′ end and a modified antisense strand of SEQ ID NO: 251.

In some embodiments, the RNAi agent has a modified sense strand of SEQ ID NO: 444 conjugated to Formula IV at the 3′ end. In some embodiments, the RNAi agent has a modified antisense strand of SEQ ID NO: 252. In some embodiments, the RNAi agent has a modified sense strand of SEQ ID NO: 444 conjugated to Formula IV at the 3′ end and a modified antisense strand of SEQ ID NO: 252.

In some embodiments, the RNAi agent has a modified sense strand of SEQ ID NO: 445 conjugated to Formula IV at the 3′ end. In some embodiments, the RNAi agent has a modified antisense strand of SEQ ID NO: 253. In some embodiments, the RNAi agent has a modified sense strand of SEQ ID NO: 445 conjugated to Formula IV at the 3′ end and a modified antisense strand of SEQ ID NO: 253.

In some embodiments, the RNAi agent has a modified sense strand of SEQ ID NO: 446 conjugated to Formula IV at the 3′ end. In some embodiments, the RNAi agent has a modified antisense strand of SEQ ID NO: 254. In some embodiments, the RNAi agent has a modified sense strand of SEQ ID NO: 446 conjugated to Formula IV at the 3′ end and a modified antisense strand of SEQ ID NO: 254.

In some embodiments, the RNAi agent has a modified sense strand of SEQ ID NO: 447 conjugated to Formula IV at the 3′ end. In some embodiments, the RNAi agent has a modified antisense strand of SEQ ID NO: 255. In some embodiments, the RNAi agent has a modified sense strand of SEQ ID NO: 447 conjugated to Formula IV at the 3′ end and a modified antisense strand of SEQ ID NO: 255.

In some embodiments, the RNAi agent has a modified sense strand of SEQ ID NO: 448 conjugated to Formula IV at the 3′ end. In some embodiments, the RNAi agent has a modified antisense strand of SEQ ID NO: 256. In some embodiments, the RNAi agent has a modified sense strand of SEQ ID NO: 448 conjugated to Formula IV at the 3′ end and a modified antisense strand of SEQ ID NO: 256.

In some embodiments, the RNAi agent has a modified sense strand of SEQ ID NO: 449 conjugated to Formula IV at the 3′ end. In some embodiments, the RNAi agent has a modified antisense strand of SEQ ID NO: 257. In some embodiments, the RNAi agent has a modified sense strand of SEQ ID NO: 449 conjugated to Formula IV at the 3′ end and a modified antisense strand of SEQ ID NO: 257.

In some embodiments, the RNAi agent has a modified sense strand of SEQ ID NO: 450 conjugated to Formula IV at the 3′ end. In some embodiments, the RNAi agent has a modified antisense strand of SEQ ID NO: 258. In some embodiments, the RNAi agent has a modified sense strand of SEQ ID NO: 450 conjugated to Formula IV at the 3′ end and a modified antisense strand of SEQ ID NO: 258.

In some embodiments, the RNAi agent has a modified sense strand of SEQ ID NO: 451 conjugated to Formula IV at the 3′ end. In some embodiments, the RNAi agent has a modified antisense strand of SEQ ID NO: 259. In some embodiments, the RNAi agent has a modified sense strand of SEQ ID NO: 451 conjugated to Formula IV at the 3′ end and a modified antisense strand of SEQ ID NO: 259.

In some embodiments, the RNAi agent has a modified sense strand of SEQ ID NO: 452 conjugated to Formula IV at the 3′ end. In some embodiments, the RNAi agent has a modified antisense strand of SEQ ID NO: 260. In some embodiments, the RNAi agent has a modified sense strand of SEQ ID NO: 452 conjugated to Formula IV at the 3′ end and a modified antisense strand of SEQ ID NO: 260.

In some embodiments, the RNAi agent has a modified sense strand of SEQ ID NO: 453 conjugated to Formula IV at the 3′ end. In some embodiments, the RNAi agent has a modified antisense strand of SEQ ID NO: 261. In some embodiments, the RNAi agent has a modified sense strand of SEQ ID NO: 453 conjugated to Formula IV at the 3′ end and a modified antisense strand of SEQ ID NO: 261.

In some embodiments, the RNAi agent has a modified sense strand of SEQ ID NO: 454 conjugated to Formula IV at the 3′ end. In some embodiments, the RNAi agent has a modified antisense strand of SEQ ID NO: 262. In some embodiments, the RNAi agent has a modified sense strand of SEQ ID NO: 454 conjugated to Formula IV at the 3′ end and a modified antisense strand of SEQ ID NO: 262.

In some embodiments, the RNAi agent has a modified sense strand of SEQ ID NO: 455 conjugated to Formula IV at the 3′ end. In some embodiments, the RNAi agent has a modified antisense strand of SEQ ID NO: 263. In some embodiments, the RNAi agent has a modified sense strand of SEQ ID NO: 455 conjugated to Formula IV at the 3′ end and a modified antisense strand of SEQ ID NO: 263.

In some embodiments, the RNAi agent has a modified sense strand of SEQ ID NO: 456 conjugated to Formula IV at the 3′ end. In some embodiments, the RNAi agent has a modified antisense strand of SEQ ID NO: 264. In some embodiments, the RNAi agent has a modified sense strand of SEQ ID NO: 456 conjugated to Formula IV at the 3′ end and a modified antisense strand of SEQ ID NO: 264.

In some embodiments, the RNAi agent has a modified sense strand of SEQ ID NO: 457 conjugated to Formula IV at the 3′ end. In some embodiments, the RNAi agent has a modified antisense strand of SEQ ID NO: 265. In some embodiments, the RNAi agent has a modified sense strand of SEQ ID NO: 457 conjugated to Formula IV at the 3′ end and a modified antisense strand of SEQ ID NO: 265.

In some embodiments, the RNAi agent has a modified sense strand of SEQ ID NO: 458 conjugated to Formula IV at the 3′ end. In some embodiments, the RNAi agent has a modified antisense strand of SEQ ID NO: 266. In some embodiments, the RNAi agent has a modified sense strand of SEQ ID NO: 458 conjugated to Formula IV at the 3′ end and a modified antisense strand of SEQ ID NO: 266.

In some embodiments, the RNAi agent has a modified sense strand of SEQ ID NO: 459 conjugated to Formula IV at the 3′ end. In some embodiments, the RNAi agent has a modified antisense strand of SEQ ID NO: 267. In some embodiments, the RNAi agent has a modified sense strand of SEQ ID NO: 459 conjugated to Formula IV at the 3′ end and a modified antisense strand of SEQ ID NO: 267.

In some embodiments, the RNAi agent has a modified sense strand of SEQ ID NO: 460 conjugated to Formula IV at the 3′ end. In some embodiments, the RNAi agent has a modified antisense strand of SEQ ID NO: 268. In some embodiments, the RNAi agent has a modified sense strand of SEQ ID NO: 460 conjugated to Formula IV at the 3′ end and a modified antisense strand of SEQ ID NO: 268.

In some embodiments, the RNAi agent has a modified sense strand of SEQ ID NO: 461 conjugated to Formula IV at the 3′ end. In some embodiments, the RNAi agent has a modified antisense strand of SEQ ID NO: 269. In some embodiments, the RNAi agent has a modified sense strand of SEQ ID NO: 461 conjugated to Formula IV at the 3′ end and a modified antisense strand of SEQ ID NO: 269.

In some embodiments, the RNAi agent has a modified sense strand of SEQ ID NO: 462 conjugated to Formula IV at the 3′ end. In some embodiments, the RNAi agent has a modified antisense strand of SEQ ID NO: 270. In some embodiments, the RNAi agent has a modified sense strand of SEQ ID NO: 462 conjugated to Formula IV at the 3′ end and a modified antisense strand of SEQ ID NO: 270.

In some embodiments, the RNAi agent has a modified sense strand of SEQ ID NO: 463 conjugated to Formula IV at the 3′ end. In some embodiments, the RNAi agent has a modified antisense strand of SEQ ID NO: 271. In some embodiments, the RNAi agent has a modified sense strand of SEQ ID NO: 463 conjugated to Formula IV at the 3′ end and a modified antisense strand of SEQ ID NO: 271.

In some embodiments, the RNAi agent has a modified sense strand of SEQ ID NO: 464 conjugated to Formula IV at the 3′ end. In some embodiments, the RNAi agent has a modified antisense strand of SEQ ID NO: 272. In some embodiments, the RNAi agent has a modified sense strand of SEQ ID NO: 464 conjugated to Formula IV at the 3′ end and a modified antisense strand of SEQ ID NO: 272.

In some embodiments, the RNAi agent has a modified sense strand of SEQ ID NO: 465 conjugated to Formula IV at the 3′ end. In some embodiments, the RNAi agent has a modified antisense strand of SEQ ID NO: 273. In some embodiments, the RNAi agent has a modified sense strand of SEQ ID NO: 465 conjugated to Formula IV at the 3′ end and a modified antisense strand of SEQ ID NO: 273.

In some embodiments, the RNAi agent has a modified sense strand of SEQ ID NO: 466 conjugated to Formula IV at the 3′ end. In some embodiments, the RNAi agent has a modified antisense strand of SEQ ID NO: 274. In some embodiments, the RNAi agent has a modified sense strand of SEQ ID NO: 466 conjugated to Formula IV at the 3′ end and a modified antisense strand of SEQ ID NO: 274.

In some embodiments, the RNAi agent has a modified sense strand of SEQ ID NO: 467 conjugated to Formula IV at the 3′ end. In some embodiments, the RNAi agent has a modified antisense strand of SEQ ID NO: 275. In some embodiments, the RNAi agent has a modified sense strand of SEQ ID NO: 467 conjugated to Formula IV at the 3′ end and a modified antisense strand of SEQ ID NO: 275.

In some embodiments, the RNAi agent has a modified sense strand of SEQ ID NO: 468 conjugated to Formula IV at the 3′ end. In some embodiments, the RNAi agent has a modified antisense strand of SEQ ID NO: 276. In some embodiments, the RNAi agent has a modified sense strand of SEQ ID NO: 468 conjugated to Formula IV at the 3′ end and a modified antisense strand of SEQ ID NO: 276.

In some embodiments, the RNAi agent has a modified sense strand of SEQ ID NO: 469 conjugated to Formula IV at the 3′ end. In some embodiments, the RNAi agent has a modified antisense strand of SEQ ID NO: 277. In some embodiments, the RNAi agent has a modified sense strand of SEQ ID NO: 469 conjugated to Formula IV at the 3′ end and a modified antisense strand of SEQ ID NO: 277.

In some embodiments, the RNAi agent has a modified sense strand of SEQ ID NO: 470 conjugated to Formula IV at the 3′ end. In some embodiments, the RNAi agent has a modified antisense strand of SEQ ID NO: 278. In some embodiments, the RNAi agent has a modified sense strand of SEQ ID NO: 470 conjugated to Formula IV at the 3′ end and a modified antisense strand of SEQ ID NO: 278.

In some embodiments, the RNAi agent has a modified sense strand of SEQ ID NO: 471 conjugated to Formula IV at the 3′ end. In some embodiments, the RNAi agent has a modified antisense strand of SEQ ID NO: 279. In some embodiments, the RNAi agent has a modified sense strand of SEQ ID NO: 471 conjugated to Formula IV at the 3′ end and a modified antisense strand of SEQ ID NO: 279.

In some embodiments, the RNAi agent has a modified sense strand of SEQ ID NO: 472 conjugated to Formula IV at the 3′ end. In some embodiments, the RNAi agent has a modified antisense strand of SEQ ID NO: 280. In some embodiments, the RNAi agent has a modified sense strand of SEQ ID NO: 472 conjugated to Formula IV at the 3′ end and a modified antisense strand of SEQ ID NO: 280.

In some embodiments, the RNAi agent has a modified sense strand of SEQ ID NO: 473 conjugated to Formula IV at the 3′ end. In some embodiments, the RNAi agent has a modified antisense strand of SEQ ID NO: 281. In some embodiments, the RNAi agent has a modified sense strand of SEQ ID NO: 473 conjugated to Formula IV at the 3′ end and a modified antisense strand of SEQ ID NO: 281.

In some embodiments, the RNAi agent has a modified sense strand of SEQ ID NO: 474 conjugated to Formula IV at the 3′ end. In some embodiments, the RNAi agent has a modified antisense strand of SEQ ID NO: 282. In some embodiments, the RNAi agent has a modified sense strand of SEQ ID NO: 474 conjugated to Formula IV at the 3′ end and a modified antisense strand of SEQ ID NO: 282.

In some embodiments, the RNAi agent has a modified sense strand of SEQ ID NO: 475 conjugated to Formula IV at the 3′ end. In some embodiments, the RNAi agent has a modified antisense strand of SEQ ID NO: 283. In some embodiments, the RNAi agent has a modified sense strand of SEQ ID NO: 475 conjugated to Formula IV at the 3′ end and a modified antisense strand of SEQ ID NO: 283.

In some embodiments, the RNAi agent has a modified sense strand of SEQ ID NO: 476 conjugated to Formula IV at the 3′ end. In some embodiments, the RNAi agent has a modified antisense strand of SEQ ID NO: 284. In some embodiments, the RNAi agent has a modified sense strand of SEQ ID NO: 476 conjugated to Formula IV at the 3′ end and a modified antisense strand of SEQ ID NO: 284.

In some embodiments, the RNAi agent has a modified sense strand of SEQ ID NO: 477 conjugated to Formula IV at the 3′ end. In some embodiments, the RNAi agent has a modified antisense strand of SEQ ID NO: 285. In some embodiments, the RNAi agent has a modified sense strand of SEQ ID NO: 477 conjugated to Formula IV at the 3′ end and a modified antisense strand of SEQ ID NO: 285.

In some embodiments, the RNAi agent has a modified sense strand of SEQ ID NO: 478 conjugated to Formula IV at the 3′ end. In some embodiments, the RNAi agent has a modified antisense strand of SEQ ID NO: 286. In some embodiments, the RNAi agent has a modified sense strand of SEQ ID NO: 478 conjugated to Formula IV at the 3′ end and a modified antisense strand of SEQ ID NO: 286.

In some embodiments, the RNAi agent has a modified sense strand of SEQ ID NO: 479 conjugated to Formula IV at the 3′ end. In some embodiments, the RNAi agent has a modified antisense strand of SEQ ID NO: 287. In some embodiments, the RNAi agent has a modified sense strand of SEQ ID NO: 479 conjugated to Formula IV at the 3′ end and a modified antisense strand of SEQ ID NO: 287.

In some embodiments, the RNAi agent has a modified sense strand of SEQ ID NO: 480 conjugated to Formula IV at the 3′ end. In some embodiments, the RNAi agent has a modified antisense strand of SEQ ID NO: 288. In some embodiments, the RNAi agent has a modified sense strand of SEQ ID NO: 480 conjugated to Formula IV at the 3′ end and a modified antisense strand of SEQ ID NO: 288.

In some embodiments, the modified sense strand of the RNAi agent is conjugated to the targeting ligand and linking group of Formula V.

In some embodiments, the RNAi agent has a modified sense strand of SEQ ID NO: 385 conjugated to Formula V at the 3′ end. In some embodiments, the RNAi agent has a modified antisense strand of SEQ ID NO: 193. In some embodiments, the RNAi agent has a modified sense strand of SEQ ID NO: 385 conjugated to Formula V at the 3′ end and a modified antisense strand of SEQ ID NO: 193.

In some embodiments, the RNAi agent has a modified sense strand of SEQ ID NO: 386 conjugated to Formula V at the 3′ end. In some embodiments, the RNAi agent has a modified antisense strand of SEQ ID NO: 194. In some embodiments, the RNAi agent has a modified sense strand of SEQ ID NO: 386 conjugated to Formula V at the 3′ end and a modified antisense strand of SEQ ID NO: 194.

In some embodiments, the RNAi agent has a modified sense strand of SEQ ID NO: 387 conjugated to Formula V at the 3′ end. In some embodiments, the RNAi agent has a modified antisense strand of SEQ ID NO: 195. In some embodiments, the RNAi agent has a modified sense strand of SEQ ID NO: 387 conjugated to Formula V at the 3′ end and a modified antisense strand of SEQ ID NO: 195.

In some embodiments, the RNAi agent has a modified sense strand of SEQ ID NO: 388 conjugated to Formula V at the 3′ end. In some embodiments, the RNAi agent has a modified antisense strand of SEQ ID NO: 196. In some embodiments, the RNAi agent has a modified sense strand of SEQ ID NO: 388 conjugated to Formula V at the 3′ end and a modified antisense strand of SEQ ID NO: 196.

In some embodiments, the RNAi agent has a modified sense strand of SEQ ID NO: 389 conjugated to Formula V at the 3′ end. In some embodiments, the RNAi agent has a modified antisense strand of SEQ ID NO: 197. In some embodiments, the RNAi agent has a modified sense strand of SEQ ID NO: 389 conjugated to Formula V at the 3′ end and a modified antisense strand of SEQ ID NO: 197.

In some embodiments, the RNAi agent has a modified sense strand of SEQ ID NO: 390 conjugated to Formula V at the 3′ end. In some embodiments, the RNAi agent has a modified antisense strand of SEQ ID NO: 198. In some embodiments, the RNAi agent has a modified sense strand of SEQ ID NO: 390 conjugated to Formula V at the 3′ end and a modified antisense strand of SEQ ID NO: 198.

In some embodiments, the RNAi agent has a modified sense strand of SEQ ID NO: 391 conjugated to Formula V at the 3′ end. In some embodiments, the RNAi agent has a modified antisense strand of SEQ ID NO: 199. In some embodiments, the RNAi agent has a modified sense strand of SEQ ID NO: 391 conjugated to Formula V at the 3′ end and a modified antisense strand of SEQ ID NO: 199.

In some embodiments, the RNAi agent has a modified sense strand of SEQ ID NO: 392 conjugated to Formula V at the 3′ end. In some embodiments, the RNAi agent has a modified antisense strand of SEQ ID NO: 200. In some embodiments, the RNAi agent has a modified sense strand of SEQ ID NO: 392 conjugated to Formula V at the 3′ end and a modified antisense strand of SEQ ID NO: 200.

In some embodiments, the RNAi agent has a modified sense strand of SEQ ID NO: 393 conjugated to Formula V at the 3′ end. In some embodiments, the RNAi agent has a modified antisense strand of SEQ ID NO: 201. In some embodiments, the RNAi agent has a modified sense strand of SEQ ID NO: 393 conjugated to Formula V at the 3′ end and a modified antisense strand of SEQ ID NO: 201.

In some embodiments, the RNAi agent has a modified sense strand of SEQ ID NO: 394 conjugated to Formula V at the 3′ end. In some embodiments, the RNAi agent has a modified antisense strand of SEQ ID NO: 202. In some embodiments, the RNAi agent has a modified sense strand of SEQ ID NO: 394 conjugated to Formula V at the 3′ end and a modified antisense strand of SEQ ID NO: 202.

In some embodiments, the RNAi agent has a modified sense strand of SEQ ID NO: 395 conjugated to Formula V at the 3′ end. In some embodiments, the RNAi agent has a modified antisense strand of SEQ ID NO: 203. In some embodiments, the RNAi agent has a modified sense strand of SEQ ID NO: 395 conjugated to Formula V at the 3′ end and a modified antisense strand of SEQ ID NO: 203.

In some embodiments, the RNAi agent has a modified sense strand of SEQ ID NO: 396 conjugated to Formula V at the 3′ end. In some embodiments, the RNAi agent has a modified antisense strand of SEQ ID NO: 204. In some embodiments, the RNAi agent has a modified sense strand of SEQ ID NO: 306 conjugated to Formula V at the 3′ end and a modified antisense strand of SEQ ID NO: 204.

In some embodiments, the RNAi agent has a modified sense strand of SEQ ID NO: 397 conjugated to Formula V at the 3′ end. In some embodiments, the RNAi agent has a modified antisense strand of SEQ ID NO: 205. In some embodiments, the RNAi agent has a modified sense strand of SEQ ID NO: 397 conjugated to Formula V at the 3′ end and a modified antisense strand of SEQ ID NO: 205.

In some embodiments, the RNAi agent has a modified sense strand of SEQ ID NO: 398 conjugated to Formula V at the 3′ end. In some embodiments, the RNAi agent has a modified antisense strand of SEQ ID NO: 206. In some embodiments, the RNAi agent has a modified sense strand of SEQ ID NO: 398 conjugated to Formula V at the 3′ end and a modified antisense strand of SEQ ID NO: 206.

In some embodiments, the RNAi agent has a modified sense strand of SEQ ID NO: 399 conjugated to Formula V at the 3′ end. In some embodiments, the RNAi agent has a modified antisense strand of SEQ ID NO: 207. In some embodiments, the RNAi agent has a modified sense strand of SEQ ID NO: 399 conjugated to Formula V at the 3′ end and a modified antisense strand of SEQ ID NO: 207.

In some embodiments, the RNAi agent has a modified sense strand of SEQ ID NO: 400 conjugated to Formula V at the 3′ end. In some embodiments, the RNAi agent has a modified antisense strand of SEQ ID NO: 208. In some embodiments, the RNAi agent has a modified sense strand of SEQ ID NO: 400 conjugated to Formula V at the 3′ end and a modified antisense strand of SEQ ID NO: 208.

In some embodiments, the RNAi agent has a modified sense strand of SEQ ID NO: 401 conjugated to Formula V at the 3′ end. In some embodiments, the RNAi agent has a modified antisense strand of SEQ ID NO: 209. In some embodiments, the RNAi agent has a modified sense strand of SEQ ID NO: 401 conjugated to Formula V at the 3′ end and a modified antisense strand of SEQ ID NO: 209.

In some embodiments, the RNAi agent has a modified sense strand of SEQ ID NO: 402 conjugated to Formula V at the 3′ end. In some embodiments, the RNAi agent has a modified antisense strand of SEQ ID NO: 210. In some embodiments, the RNAi agent has a modified sense strand of SEQ ID NO: 402 conjugated to Formula V at the 3′ end and a modified antisense strand of SEQ ID NO: 210.

In some embodiments, the RNAi agent has a modified sense strand of SEQ ID NO: 403 conjugated to Formula V at the 3′ end. In some embodiments, the RNAi agent has a modified antisense strand of SEQ ID NO: 211. In some embodiments, the RNAi agent has a modified sense strand of SEQ ID NO: 403 conjugated to Formula V at the 3′ end and a modified antisense strand of SEQ ID NO: 211.

In some embodiments, the RNAi agent has a modified sense strand of SEQ ID NO: 404 conjugated to Formula V at the 3′ end. In some embodiments, the RNAi agent has a modified antisense strand of SEQ ID NO: 212. In some embodiments, the RNAi agent has a modified sense strand of SEQ ID NO: 404 conjugated to Formula V at the 3′ end and a modified antisense strand of SEQ ID NO: 212.

In some embodiments, the RNAi agent has a modified sense strand of SEQ ID NO: 405 conjugated to Formula V at the 3′ end. In some embodiments, the RNAi agent has a modified antisense strand of SEQ ID NO: 213. In some embodiments, the RNAi agent has a modified sense strand of SEQ ID NO: 405 conjugated to Formula V at the 3′ end and a modified antisense strand of SEQ ID NO: 213.

In some embodiments, the RNAi agent has a modified sense strand of SEQ ID NO: 406 conjugated to Formula V at the 3′ end. In some embodiments, the RNAi agent has a modified antisense strand of SEQ ID NO: 214. In some embodiments, the RNAi agent has a modified sense strand of SEQ ID NO: 406 conjugated to Formula V at the 3′ end and a modified antisense strand of SEQ ID NO: 214.

In some embodiments, the RNAi agent has a modified sense strand of SEQ ID NO: 407 conjugated to Formula V at the 3′ end. In some embodiments, the RNAi agent has a modified antisense strand of SEQ ID NO: 215. In some embodiments, the RNAi agent has a modified sense strand of SEQ ID NO: 407 conjugated to Formula V at the 3′ end and a modified antisense strand of SEQ ID NO: 215.

In some embodiments, the RNAi agent has a modified sense strand of SEQ ID NO: 408 conjugated to Formula V at the 3′ end. In some embodiments, the RNAi agent has a modified antisense strand of SEQ ID NO: 216. In some embodiments, the RNAi agent has a modified sense strand of SEQ ID NO: 408 conjugated to Formula V at the 3′ end and a modified antisense strand of SEQ ID NO: 216.

In some embodiments, the RNAi agent has a modified sense strand of SEQ ID NO: 409 conjugated to Formula V at the 3′ end. In some embodiments, the RNAi agent has a modified antisense strand of SEQ ID NO: 217. In some embodiments, the RNAi agent has a modified sense strand of SEQ ID NO: 409 conjugated to Formula V at the 3′ end and a modified antisense strand of SEQ ID NO: 217.

In some embodiments, the RNAi agent has a modified sense strand of SEQ ID NO: 410 conjugated to Formula V at the 3′ end. In some embodiments, the RNAi agent has a modified antisense strand of SEQ ID NO: 218. In some embodiments, the RNAi agent has a modified sense strand of SEQ ID NO: 410 conjugated to Formula V at the 3′ end and a modified antisense strand of SEQ ID NO: 218.

In some embodiments, the RNAi agent has a modified sense strand of SEQ ID NO: 411 conjugated to Formula V at the 3′ end. In some embodiments, the RNAi agent has a modified antisense strand of SEQ ID NO: 219. In some embodiments, the RNAi agent has a modified sense strand of SEQ ID NO: 411 conjugated to Formula V at the 3′ end and a modified antisense strand of SEQ ID NO: 219.

In some embodiments, the RNAi agent has a modified sense strand of SEQ ID NO: 412 conjugated to Formula V at the 3′ end. In some embodiments, the RNAi agent has a modified antisense strand of SEQ ID NO: 220. In some embodiments, the RNAi agent has a modified sense strand of SEQ ID NO: 412 conjugated to Formula V at the 3′ end and a modified antisense strand of SEQ ID NO: 220.

In some embodiments, the RNAi agent has a modified sense strand of SEQ ID NO: 413 conjugated to Formula V at the 3′ end. In some embodiments, the RNAi agent has a modified antisense strand of SEQ ID NO: 221. In some embodiments, the RNAi agent has a modified sense strand of SEQ ID NO: 413 conjugated to Formula V at the 3′ end and a modified antisense strand of SEQ ID NO: 221.

In some embodiments, the RNAi agent has a modified sense strand of SEQ ID NO: 414 conjugated to Formula V at the 3′ end. In some embodiments, the RNAi agent has a modified antisense strand of SEQ ID NO: 222. In some embodiments, the RNAi agent has a modified sense strand of SEQ ID NO: 414 conjugated to Formula V at the 3′ end and a modified antisense strand of SEQ ID NO: 222.

In some embodiments, the RNAi agent has a modified sense strand of SEQ ID NO: 415 conjugated to Formula V at the 3′ end. In some embodiments, the RNAi agent has a modified antisense strand of SEQ ID NO: 223. In some embodiments, the RNAi agent has a modified sense strand of SEQ ID NO: 415 conjugated to Formula V at the 3′ end and a modified antisense strand of SEQ ID NO: 223.

In some embodiments, the RNAi agent has a modified sense strand of SEQ ID NO: 416 conjugated to Formula V at the 3′ end. In some embodiments, the RNAi agent has a modified antisense strand of SEQ ID NO: 224. In some embodiments, the RNAi agent has a modified sense strand of SEQ ID NO: 416 conjugated to Formula V at the 3′ end and a modified antisense strand of SEQ ID NO: 224.

In some embodiments, the RNAi agent has a modified sense strand of SEQ ID NO: 417 conjugated to Formula V at the 3′ end. In some embodiments, the RNAi agent has a modified antisense strand of SEQ ID NO: 225. In some embodiments, the RNAi agent has a modified sense strand of SEQ ID NO: 417 conjugated to Formula V at the 3′ end and a modified antisense strand of SEQ ID NO: 225.

In some embodiments, the RNAi agent has a modified sense strand of SEQ ID NO: 418 conjugated to Formula V at the 3′ end. In some embodiments, the RNAi agent has a modified antisense strand of SEQ ID NO: 226. In some embodiments, the RNAi agent has a modified sense strand of SEQ ID NO: 418 conjugated to Formula V at the 3′ end and a modified antisense strand of SEQ ID NO: 226.

In some embodiments, the RNAi agent has a modified sense strand of SEQ ID NO: 419 conjugated to Formula V at the 3′ end. In some embodiments, the RNAi agent has a modified antisense strand of SEQ ID NO: 227. In some embodiments, the RNAi agent has a modified sense strand of SEQ ID NO: 419 conjugated to Formula V at the 3′ end and a modified antisense strand of SEQ ID NO: 227.

In some embodiments, the RNAi agent has a modified sense strand of SEQ ID NO: 420 conjugated to Formula V at the 3′ end. In some embodiments, the RNAi agent has a modified antisense strand of SEQ ID NO: 228. In some embodiments, the RNAi agent has a modified sense strand of SEQ ID NO: 420 conjugated to Formula V at the 3′ end and a modified antisense strand of SEQ ID NO: 228.

In some embodiments, the RNAi agent has a modified sense strand of SEQ ID NO: 421 conjugated to Formula V at the 3′ end. In some embodiments, the RNAi agent has a modified antisense strand of SEQ ID NO: 229. In some embodiments, the RNAi agent has a modified sense strand of SEQ ID NO: 421 conjugated to Formula V at the 3′ end and a modified antisense strand of SEQ ID NO: 229.

In some embodiments, the RNAi agent has a modified sense strand of SEQ ID NO: 422 conjugated to Formula V at the 3′ end. In some embodiments, the RNAi agent has a modified antisense strand of SEQ ID NO: 230. In some embodiments, the RNAi agent has a modified sense strand of SEQ ID NO: 422 conjugated to Formula V at the 3′ end and a modified antisense strand of SEQ ID NO: 230.

In some embodiments, the RNAi agent has a modified sense strand of SEQ ID NO: 423 conjugated to Formula V at the 3′ end. In some embodiments, the RNAi agent has a modified antisense strand of SEQ ID NO: 231. In some embodiments, the RNAi agent has a modified sense strand of SEQ ID NO: 423 conjugated to Formula V at the 3′ end and a modified antisense strand of SEQ ID NO: 231.

In some embodiments, the RNAi agent has a modified sense strand of SEQ ID NO: 424 conjugated to Formula V at the 3′ end. In some embodiments, the RNAi agent has a modified antisense strand of SEQ ID NO: 232. In some embodiments, the RNAi agent has a modified sense strand of SEQ ID NO: 424 conjugated to Formula V at the 3′ end and a modified antisense strand of SEQ ID NO: 232.

In some embodiments, the RNAi agent has a modified sense strand of SEQ ID NO: 425 conjugated to Formula V at the 3′ end. In some embodiments, the RNAi agent has a modified antisense strand of SEQ ID NO: 233. In some embodiments, the RNAi agent has a modified sense strand of SEQ ID NO: 425 conjugated to Formula V at the 3′ end and a modified antisense strand of SEQ ID NO: 233.

In some embodiments, the RNAi agent has a modified sense strand of SEQ ID NO: 426 conjugated to Formula V at the 3′ end. In some embodiments, the RNAi agent has a modified antisense strand of SEQ ID NO: 234. In some embodiments, the RNAi agent has a modified sense strand of SEQ ID NO: 426 conjugated to Formula V at the 3′ end and a modified antisense strand of SEQ ID NO: 234.

In some embodiments, the RNAi agent has a modified sense strand of SEQ ID NO: 427 conjugated to Formula V at the 3′ end. In some embodiments, the RNAi agent has a modified antisense strand of SEQ ID NO: 235. In some embodiments, the RNAi agent has a modified sense strand of SEQ ID NO: 427 conjugated to Formula V at the 3′ end and a modified antisense strand of SEQ ID NO: 235.

In some embodiments, the RNAi agent has a modified sense strand of SEQ ID NO: 428 conjugated to Formula V at the 3′ end. In some embodiments, the RNAi agent has a modified antisense strand of SEQ ID NO: 236. In some embodiments, the RNAi agent has a modified sense strand of SEQ ID NO: 428 conjugated to Formula V at the 3′ end and a modified antisense strand of SEQ ID NO: 236.

In some embodiments, the RNAi agent has a modified sense strand of SEQ ID NO: 429 conjugated to Formula V at the 3′ end. In some embodiments, the RNAi agent has a modified antisense strand of SEQ ID NO: 237. In some embodiments, the RNAi agent has a modified sense strand of SEQ ID NO: 429 conjugated to Formula V at the 3′ end and a modified antisense strand of SEQ ID NO: 237.

In some embodiments, the RNAi agent has a modified sense strand of SEQ ID NO: 430 conjugated to Formula V at the 3′ end. In some embodiments, the RNAi agent has a modified antisense strand of SEQ ID NO: 238. In some embodiments, the RNAi agent has a modified sense strand of SEQ ID NO: 430 conjugated to Formula V at the 3′ end and a modified antisense strand of SEQ ID NO: 238.

In some embodiments, the RNAi agent has a modified sense strand of SEQ ID NO: 431 conjugated to Formula V at the 3′ end. In some embodiments, the RNAi agent has a modified antisense strand of SEQ ID NO: 239. In some embodiments, the RNAi agent has a modified sense strand of SEQ ID NO: 431 conjugated to Formula V at the 3′ end and a modified antisense strand of SEQ ID NO: 239.

In some embodiments, the RNAi agent has a modified sense strand of SEQ ID NO: 432 conjugated to Formula V at the 3′ end. In some embodiments, the RNAi agent has a modified antisense strand of SEQ ID NO: 240. In some embodiments, the RNAi agent has a modified sense strand of SEQ ID NO: 432 conjugated to Formula V at the 3′ end and a modified antisense strand of SEQ ID NO: 240.

In some embodiments, the RNAi agent has a modified sense strand of SEQ ID NO: 433 conjugated to Formula V at the 3′ end. In some embodiments, the RNAi agent has a modified antisense strand of SEQ ID NO: 241. In some embodiments, the RNAi agent has a modified sense strand of SEQ ID NO: 433 conjugated to Formula V at the 3′ end and a modified antisense strand of SEQ ID NO: 241.

In some embodiments, the RNAi agent has a modified sense strand of SEQ ID NO: 434 conjugated to Formula V at the 3′ end. In some embodiments, the RNAi agent has a modified antisense strand of SEQ ID NO: 242. In some embodiments, the RNAi agent has a modified sense strand of SEQ ID NO: 434 conjugated to Formula V at the 3′ end and a modified antisense strand of SEQ ID NO: 242.

In some embodiments, the RNAi agent has a modified sense strand of SEQ ID NO: 435 conjugated to Formula V at the 3′ end. In some embodiments, the RNAi agent has a modified antisense strand of SEQ ID NO: 243. In some embodiments, the RNAi agent has a modified sense strand of SEQ ID NO: 435 conjugated to Formula V at the 3′ end and a modified antisense strand of SEQ ID NO: 243.

In some embodiments, the RNAi agent has a modified sense strand of SEQ ID NO: 436 conjugated to Formula V at the 3′ end. In some embodiments, the RNAi agent has a modified antisense strand of SEQ ID NO: 244. In some embodiments, the RNAi agent has a modified sense strand of SEQ ID NO: 436 conjugated to Formula V at the 3′ end and a modified antisense strand of SEQ ID NO: 244.

In some embodiments, the RNAi agent has a modified sense strand of SEQ ID NO: 437 conjugated to Formula V at the 3′ end. In some embodiments, the RNAi agent has a modified antisense strand of SEQ ID NO: 245. In some embodiments, the RNAi agent has a modified sense strand of SEQ ID NO: 437 conjugated to Formula V at the 3′ end and a modified antisense strand of SEQ ID NO: 245.

In some embodiments, the RNAi agent has a modified sense strand of SEQ ID NO: 438 conjugated to Formula V at the 3′ end. In some embodiments, the RNAi agent has a modified antisense strand of SEQ ID NO: 246. In some embodiments, the RNAi agent has a modified sense strand of SEQ ID NO: 438 conjugated to Formula V at the 3′ end and a modified antisense strand of SEQ ID NO: 246.

In some embodiments, the RNAi agent has a modified sense strand of SEQ ID NO: 439 conjugated to Formula V at the 3′ end. In some embodiments, the RNAi agent has a modified antisense strand of SEQ ID NO: 247. In some embodiments, the RNAi agent has a modified sense strand of SEQ ID NO: 439 conjugated to Formula V at the 3′ end and a modified antisense strand of SEQ ID NO: 247.

In some embodiments, the RNAi agent has a modified sense strand of SEQ ID NO: 440 conjugated to Formula V at the 3′ end. In some embodiments, the RNAi agent has a modified antisense strand of SEQ ID NO: 248. In some embodiments, the RNAi agent has a modified sense strand of SEQ ID NO: 440 conjugated to Formula V at the 3′ end and a modified antisense strand of SEQ ID NO: 248.

In some embodiments, the RNAi agent has a modified sense strand of SEQ ID NO: 441 conjugated to Formula V at the 3′ end. In some embodiments, the RNAi agent has a modified antisense strand of SEQ ID NO: 249. In some embodiments, the RNAi agent has a modified sense strand of SEQ ID NO: 441 conjugated to Formula V at the 3′ end and a modified antisense strand of SEQ ID NO: 249.

In some embodiments, the RNAi agent has a modified sense strand of SEQ ID NO: 442 conjugated to Formula V at the 3′ end. In some embodiments, the RNAi agent has a modified antisense strand of SEQ ID NO: 250. In some embodiments, the RNAi agent has a modified sense strand of SEQ ID NO: 442 conjugated to Formula V at the 3′ end and a modified antisense strand of SEQ ID NO: 250.

In some embodiments, the RNAi agent has a modified sense strand of SEQ ID NO: 443 conjugated to Formula V at the 3′ end. In some embodiments, the RNAi agent has a modified antisense strand of SEQ ID NO: 251. In some embodiments, the RNAi agent has a modified sense strand of SEQ ID NO: 443 conjugated to Formula V at the 3′ end and a modified antisense strand of SEQ ID NO: 251.

In some embodiments, the RNAi agent has a modified sense strand of SEQ ID NO: 444 conjugated to Formula V at the 3′ end. In some embodiments, the RNAi agent has a modified antisense strand of SEQ ID NO: 252. In some embodiments, the RNAi agent has a modified sense strand of SEQ ID NO: 444 conjugated to Formula V at the 3′ end and a modified antisense strand of SEQ ID NO: 252.

In some embodiments, the RNAi agent has a modified sense strand of SEQ ID NO: 445 conjugated to Formula V at the 3′ end. In some embodiments, the RNAi agent has a modified antisense strand of SEQ ID NO: 253. In some embodiments, the RNAi agent has a modified sense strand of SEQ ID NO: 445 conjugated to Formula V at the 3′ end and a modified antisense strand of SEQ ID NO: 253.

In some embodiments, the RNAi agent has a modified sense strand of SEQ ID NO: 446 conjugated to Formula V at the 3′ end. In some embodiments, the RNAi agent has a modified antisense strand of SEQ ID NO: 254. In some embodiments, the RNAi agent has a modified sense strand of SEQ ID NO: 446 conjugated to Formula V at the 3′ end and a modified antisense strand of SEQ ID NO: 254.

In some embodiments, the RNAi agent has a modified sense strand of SEQ ID NO: 447 conjugated to Formula V at the 3′ end. In some embodiments, the RNAi agent has a modified antisense strand of SEQ ID NO: 255. In some embodiments, the RNAi agent has a modified sense strand of SEQ ID NO: 447 conjugated to Formula V at the 3′ end and a modified antisense strand of SEQ ID NO: 255.

In some embodiments, the RNAi agent has a modified sense strand of SEQ ID NO: 448 conjugated to Formula V at the 3′ end. In some embodiments, the RNAi agent has a modified antisense strand of SEQ ID NO: 256. In some embodiments, the RNAi agent has a modified sense strand of SEQ ID NO: 448 conjugated to Formula V at the 3′ end and a modified antisense strand of SEQ ID NO: 256.

In some embodiments, the RNAi agent has a modified sense strand of SEQ ID NO: 449 conjugated to Formula V at the 3′ end. In some embodiments, the RNAi agent has a modified antisense strand of SEQ ID NO: 257. In some embodiments, the RNAi agent has a modified sense strand of SEQ ID NO: 449 conjugated to Formula V at the 3′ end and a modified antisense strand of SEQ ID NO: 257.

In some embodiments, the RNAi agent has a modified sense strand of SEQ ID NO: 450 conjugated to Formula V at the 3′ end. In some embodiments, the RNAi agent has a modified antisense strand of SEQ ID NO: 258. In some embodiments, the RNAi agent has a modified sense strand of SEQ ID NO: 450 conjugated to Formula V at the 3′ end and a modified antisense strand of SEQ ID NO: 258.

In some embodiments, the RNAi agent has a modified sense strand of SEQ ID NO: 451 conjugated to Formula V at the 3′ end. In some embodiments, the RNAi agent has a modified antisense strand of SEQ ID NO: 259. In some embodiments, the RNAi agent has a modified sense strand of SEQ ID NO: 451 conjugated to Formula V at the 3′ end and a modified antisense strand of SEQ ID NO: 259.

In some embodiments, the RNAi agent has a modified sense strand of SEQ ID NO: 452 conjugated to Formula V at the 3′ end. In some embodiments, the RNAi agent has a modified antisense strand of SEQ ID NO: 260. In some embodiments, the RNAi agent has a modified sense strand of SEQ ID NO: 452 conjugated to Formula V at the 3′ end and a modified antisense strand of SEQ ID NO: 260.

In some embodiments, the RNAi agent has a modified sense strand of SEQ ID NO: 453 conjugated to Formula V at the 3′ end. In some embodiments, the RNAi agent has a modified antisense strand of SEQ ID NO: 261. In some embodiments, the RNAi agent has a modified sense strand of SEQ ID NO: 453 conjugated to Formula V at the 3′ end and a modified antisense strand of SEQ ID NO: 261.

In some embodiments, the RNAi agent has a modified sense strand of SEQ ID NO: 454 conjugated to Formula V at the 3′ end. In some embodiments, the RNAi agent has a modified antisense strand of SEQ ID NO: 262. In some embodiments, the RNAi agent has a modified sense strand of SEQ ID NO: 454 conjugated to Formula V at the 3′ end and a modified antisense strand of SEQ ID NO: 262.

In some embodiments, the RNAi agent has a modified sense strand of SEQ ID NO: 455 conjugated to Formula V at the 3′ end. In some embodiments, the RNAi agent has a modified antisense strand of SEQ ID NO: 263. In some embodiments, the RNAi agent has a modified sense strand of SEQ ID NO: 455 conjugated to Formula V at the 3′ end and a modified antisense strand of SEQ ID NO: 263.

In some embodiments, the RNAi agent has a modified sense strand of SEQ ID NO: 456 conjugated to Formula V at the 3′ end. In some embodiments, the RNAi agent has a modified antisense strand of SEQ ID NO: 264. In some embodiments, the RNAi agent has a modified sense strand of SEQ ID NO: 456 conjugated to Formula V at the 3′ end and a modified antisense strand of SEQ ID NO: 264.

In some embodiments, the RNAi agent has a modified sense strand of SEQ ID NO: 457 conjugated to Formula V at the 3′ end. In some embodiments, the RNAi agent has a modified antisense strand of SEQ ID NO: 265. In some embodiments, the RNAi agent has a modified sense strand of SEQ ID NO: 457 conjugated to Formula V at the 3′ end and a modified antisense strand of SEQ ID NO: 265.

In some embodiments, the RNAi agent has a modified sense strand of SEQ ID NO: 458 conjugated to Formula V at the 3′ end. In some embodiments, the RNAi agent has a modified antisense strand of SEQ ID NO: 266. In some embodiments, the RNAi agent has a modified sense strand of SEQ ID NO: 458 conjugated to Formula V at the 3′ end and a modified antisense strand of SEQ ID NO: 266.

In some embodiments, the RNAi agent has a modified sense strand of SEQ ID NO: 459 conjugated to Formula V at the 3′ end. In some embodiments, the RNAi agent has a modified antisense strand of SEQ ID NO: 267. In some embodiments, the RNAi agent has a modified sense strand of SEQ ID NO: 459 conjugated to Formula V at the 3′ end and a modified antisense strand of SEQ ID NO: 267.

In some embodiments, the RNAi agent has a modified sense strand of SEQ ID NO: 460 conjugated to Formula V at the 3′ end. In some embodiments, the RNAi agent has a modified antisense strand of SEQ ID NO: 268. In some embodiments, the RNAi agent has a modified sense strand of SEQ ID NO: 460 conjugated to Formula V at the 3′ end and a modified antisense strand of SEQ ID NO: 268.

In some embodiments, the RNAi agent has a modified sense strand of SEQ ID NO: 461 conjugated to Formula V at the 3′ end. In some embodiments, the RNAi agent has a modified antisense strand of SEQ ID NO: 269. In some embodiments, the RNAi agent has a modified sense strand of SEQ ID NO: 461 conjugated to Formula V at the 3′ end and a modified antisense strand of SEQ ID NO: 269.

In some embodiments, the RNAi agent has a modified sense strand of SEQ ID NO: 462 conjugated to Formula V at the 3′ end. In some embodiments, the RNAi agent has a modified antisense strand of SEQ ID NO: 270. In some embodiments, the RNAi agent has a modified sense strand of SEQ ID NO: 462 conjugated to Formula V at the 3′ end and a modified antisense strand of SEQ ID NO: 270.

In some embodiments, the RNAi agent has a modified sense strand of SEQ ID NO: 463 conjugated to Formula V at the 3′ end. In some embodiments, the RNAi agent has a modified antisense strand of SEQ ID NO: 271. In some embodiments, the RNAi agent has a modified sense strand of SEQ ID NO: 463 conjugated to Formula V at the 3′ end and a modified antisense strand of SEQ ID NO: 271.

In some embodiments, the RNAi agent has a modified sense strand of SEQ ID NO: 464 conjugated to Formula V at the 3′ end. In some embodiments, the RNAi agent has a modified antisense strand of SEQ ID NO: 272. In some embodiments, the RNAi agent has a modified sense strand of SEQ ID NO: 464 conjugated to Formula V at the 3′ end and a modified antisense strand of SEQ ID NO: 272.

In some embodiments, the RNAi agent has a modified sense strand of SEQ ID NO: 465 conjugated to Formula V at the 3′ end. In some embodiments, the RNAi agent has a modified antisense strand of SEQ ID NO: 273. In some embodiments, the RNAi agent has a modified sense strand of SEQ ID NO: 465 conjugated to Formula V at the 3′ end and a modified antisense strand of SEQ ID NO: 273.

In some embodiments, the RNAi agent has a modified sense strand of SEQ ID NO: 466 conjugated to Formula V at the 3′ end. In some embodiments, the RNAi agent has a modified antisense strand of SEQ ID NO: 274. In some embodiments, the RNAi agent has a modified sense strand of SEQ ID NO: 466 conjugated to Formula V at the 3′ end and a modified antisense strand of SEQ ID NO: 274.

In some embodiments, the RNAi agent has a modified sense strand of SEQ ID NO: 467 conjugated to Formula V at the 3′ end. In some embodiments, the RNAi agent has a modified antisense strand of SEQ ID NO: 275. In some embodiments, the RNAi agent has a modified sense strand of SEQ ID NO: 467 conjugated to Formula V at the 3′ end and a modified antisense strand of SEQ ID NO: 275.

In some embodiments, the RNAi agent has a modified sense strand of SEQ ID NO: 468 conjugated to Formula V at the 3′ end. In some embodiments, the RNAi agent has a modified antisense strand of SEQ ID NO: 276. In some embodiments, the RNAi agent has a modified sense strand of SEQ ID NO: 468 conjugated to Formula V at the 3′ end and a modified antisense strand of SEQ ID NO: 276.

In some embodiments, the RNAi agent has a modified sense strand of SEQ ID NO: 469 conjugated to Formula V at the 3′ end. In some embodiments, the RNAi agent has a modified antisense strand of SEQ ID NO: 277. In some embodiments, the RNAi agent has a modified sense strand of SEQ ID NO: 469 conjugated to Formula V at the 3′ end and a modified antisense strand of SEQ ID NO: 277.

In some embodiments, the RNAi agent has a modified sense strand of SEQ ID NO: 470 conjugated to Formula V at the 3′ end. In some embodiments, the RNAi agent has a modified antisense strand of SEQ ID NO: 278. In some embodiments, the RNAi agent has a modified sense strand of SEQ ID NO: 470 conjugated to Formula V at the 3′ end and a modified antisense strand of SEQ ID NO: 278.

In some embodiments, the RNAi agent has a modified sense strand of SEQ ID NO: 471 conjugated to Formula V at the 3′ end. In some embodiments, the RNAi agent has a modified antisense strand of SEQ ID NO: 279. In some embodiments, the RNAi agent has a modified sense strand of SEQ ID NO: 471 conjugated to Formula V at the 3′ end and a modified antisense strand of SEQ ID NO: 279.

In some embodiments, the RNAi agent has a modified sense strand of SEQ ID NO: 472 conjugated to Formula V at the 3′ end. In some embodiments, the RNAi agent has a modified antisense strand of SEQ ID NO: 280. In some embodiments, the RNAi agent has a modified sense strand of SEQ ID NO: 472 conjugated to Formula V at the 3′ end and a modified antisense strand of SEQ ID NO: 280.

In some embodiments, the RNAi agent has a modified sense strand of SEQ ID NO: 473 conjugated to Formula V at the 3′ end. In some embodiments, the RNAi agent has a modified antisense strand of SEQ ID NO: 281. In some embodiments, the RNAi agent has a modified sense strand of SEQ ID NO: 473 conjugated to Formula V at the 3′ end and a modified antisense strand of SEQ ID NO: 281.

In some embodiments, the RNAi agent has a modified sense strand of SEQ ID NO: 474 conjugated to Formula V at the 3′ end. In some embodiments, the RNAi agent has a modified antisense strand of SEQ ID NO: 282. In some embodiments, the RNAi agent has a modified sense strand of SEQ ID NO: 474 conjugated to Formula V at the 3′ end and a modified antisense strand of SEQ ID NO: 282.

In some embodiments, the RNAi agent has a modified sense strand of SEQ ID NO: 475 conjugated to Formula V at the 3′ end. In some embodiments, the RNAi agent has a modified antisense strand of SEQ ID NO: 283. In some embodiments, the RNAi agent has a modified sense strand of SEQ ID NO: 475 conjugated to Formula V at the 3′ end and a modified antisense strand of SEQ ID NO: 283.

In some embodiments, the RNAi agent has a modified sense strand of SEQ ID NO: 476 conjugated to Formula V at the 3′ end. In some embodiments, the RNAi agent has a modified antisense strand of SEQ ID NO: 284. In some embodiments, the RNAi agent has a modified sense strand of SEQ ID NO: 476 conjugated to Formula V at the 3′ end and a modified antisense strand of SEQ ID NO: 284.

In some embodiments, the RNAi agent has a modified sense strand of SEQ ID NO: 477 conjugated to Formula V at the 3′ end. In some embodiments, the RNAi agent has a modified antisense strand of SEQ ID NO: 285. In some embodiments, the RNAi agent has a modified sense strand of SEQ ID NO: 477 conjugated to Formula V at the 3′ end and a modified antisense strand of SEQ ID NO: 285.

In some embodiments, the RNAi agent has a modified sense strand of SEQ ID NO: 478 conjugated to Formula V at the 3′ end. In some embodiments, the RNAi agent has a modified antisense strand of SEQ ID NO: 286. In some embodiments, the RNAi agent has a modified sense strand of SEQ ID NO: 478 conjugated to Formula V at the 3′ end and a modified antisense strand of SEQ ID NO: 286.

In some embodiments, the RNAi agent has a modified sense strand of SEQ ID NO: 479 conjugated to Formula V at the 3′ end. In some embodiments, the RNAi agent has a modified antisense strand of SEQ ID NO: 287. In some embodiments, the RNAi agent has a modified sense strand of SEQ ID NO: 479 conjugated to Formula V at the 3′ end and a modified antisense strand of SEQ ID NO: 287.

In some embodiments, the RNAi agent has a modified sense strand of SEQ ID NO: 480 conjugated to Formula V at the 3′ end. In some embodiments, the RNAi agent has a modified antisense strand of SEQ ID NO: 288. In some embodiments, the RNAi agent has a modified sense strand of SEQ ID NO: 480 conjugated to Formula V at the 3′ end and a modified antisense strand of SEQ ID NO: 288.

In some embodiments, the modified sense strand of the RNAi agent is conjugated to the targeting ligand and linking group of Formula VI.

In some embodiments, the RNAi agent has a modified sense strand of SEQ ID NO: 385 conjugated to Formula VI at the 3′ end. In some embodiments, the RNAi agent has a modified antisense strand of SEQ ID NO: 193. In some embodiments, the RNAi agent has a modified sense strand of SEQ ID NO: 385 conjugated to Formula VI at the 3′ end and a modified antisense strand of SEQ ID NO: 193.

In some embodiments, the RNAi agent has a modified sense strand of SEQ ID NO: 386 conjugated to Formula VI at the 3′ end. In some embodiments, the RNAi agent has a modified antisense strand of SEQ ID NO: 194. In some embodiments, the RNAi agent has a modified sense strand of SEQ ID NO: 386 conjugated to Formula VI at the 3′ end and a modified antisense strand of SEQ ID NO: 194.

In some embodiments, the RNAi agent has a modified sense strand of SEQ ID NO: 387 conjugated to Formula VI at the 3′ end. In some embodiments, the RNAi agent has a modified antisense strand of SEQ ID NO: 195. In some embodiments, the RNAi agent has a modified sense strand of SEQ ID NO: 387 conjugated to Formula VI at the 3′ end and a modified antisense strand of SEQ ID NO: 195.

In some embodiments, the RNAi agent has a modified sense strand of SEQ ID NO: 388 conjugated to Formula VI at the 3′ end. In some embodiments, the RNAi agent has a modified antisense strand of SEQ ID NO: 196. In some embodiments, the RNAi agent has a modified sense strand of SEQ ID NO: 388 conjugated to Formula VI at the 3′ end and a modified antisense strand of SEQ ID NO: 196.

In some embodiments, the RNAi agent has a modified sense strand of SEQ ID NO: 389 conjugated to Formula VI at the 3′ end. In some embodiments, the RNAi agent has a modified antisense strand of SEQ ID NO: 197. In some embodiments, the RNAi agent has a modified sense strand of SEQ ID NO: 389 conjugated to Formula VI at the 3′ end and a modified antisense strand of SEQ ID NO: 197.

In some embodiments, the RNAi agent has a modified sense strand of SEQ ID NO: 390 conjugated to Formula VI at the 3′ end. In some embodiments, the RNAi agent has a modified antisense strand of SEQ ID NO: 198. In some embodiments, the RNAi agent has a modified sense strand of SEQ ID NO: 390 conjugated to Formula VI at the 3′ end and a modified antisense strand of SEQ ID NO: 198.

In some embodiments, the RNAi agent has a modified sense strand of SEQ ID NO: 391 conjugated to Formula VI at the 3′ end. In some embodiments, the RNAi agent has a modified antisense strand of SEQ ID NO: 199. In some embodiments, the RNAi agent has a modified sense strand of SEQ ID NO: 391 conjugated to Formula VI at the 3′ end and a modified antisense strand of SEQ ID NO: 199.

In some embodiments, the RNAi agent has a modified sense strand of SEQ ID NO: 392 conjugated to Formula VI at the 3′ end. In some embodiments, the RNAi agent has a modified antisense strand of SEQ ID NO: 200. In some embodiments, the RNAi agent has a modified sense strand of SEQ ID NO: 392 conjugated to Formula VI at the 3′ end and a modified antisense strand of SEQ ID NO: 200.

In some embodiments, the RNAi agent has a modified sense strand of SEQ ID NO: 393 conjugated to Formula VI at the 3′ end. In some embodiments, the RNAi agent has a modified antisense strand of SEQ ID NO: 201. In some embodiments, the RNAi agent has a modified sense strand of SEQ ID NO: 393 conjugated to Formula VI at the 3′ end and a modified antisense strand of SEQ ID NO: 201.

In some embodiments, the RNAi agent has a modified sense strand of SEQ ID NO: 394 conjugated to Formula VI at the 3′ end. In some embodiments, the RNAi agent has a modified antisense strand of SEQ ID NO: 202. In some embodiments, the RNAi agent has a modified sense strand of SEQ ID NO: 394 conjugated to Formula VI at the 3′ end and a modified antisense strand of SEQ ID NO: 202.

In some embodiments, the RNAi agent has a modified sense strand of SEQ ID NO: 395 conjugated to Formula VI at the 3′ end. In some embodiments, the RNAi agent has a modified antisense strand of SEQ ID NO: 203. In some embodiments, the RNAi agent has a modified sense strand of SEQ ID NO: 395 conjugated to Formula VI at the 3′ end and a modified antisense strand of SEQ ID NO: 203.

In some embodiments, the RNAi agent has a modified sense strand of SEQ ID NO: 396 conjugated to Formula VI at the 3′ end. In some embodiments, the RNAi agent has a modified antisense strand of SEQ ID NO: 204. In some embodiments, the RNAi agent has a modified sense strand of SEQ ID NO: 306 conjugated to Formula VI at the 3′ end and a modified antisense strand of SEQ ID NO: 204.

In some embodiments, the RNAi agent has a modified sense strand of SEQ ID NO: 397 conjugated to Formula VI at the 3′ end. In some embodiments, the RNAi agent has a modified antisense strand of SEQ ID NO: 205. In some embodiments, the RNAi agent has a modified sense strand of SEQ ID NO: 397 conjugated to Formula VI at the 3′ end and a modified antisense strand of SEQ ID NO: 205.

In some embodiments, the RNAi agent has a modified sense strand of SEQ ID NO: 398 conjugated to Formula VI at the 3′ end. In some embodiments, the RNAi agent has a modified antisense strand of SEQ ID NO: 206. In some embodiments, the RNAi agent has a modified sense strand of SEQ ID NO: 398 conjugated to Formula VI at the 3′ end and a modified antisense strand of SEQ ID NO: 206.

In some embodiments, the RNAi agent has a modified sense strand of SEQ ID NO: 399 conjugated to Formula VI at the 3′ end. In some embodiments, the RNAi agent has a modified antisense strand of SEQ ID NO: 207. In some embodiments, the RNAi agent has a modified sense strand of SEQ ID NO: 399 conjugated to Formula VI at the 3′ end and a modified antisense strand of SEQ ID NO: 207.

In some embodiments, the RNAi agent has a modified sense strand of SEQ ID NO: 400 conjugated to Formula VI at the 3′ end. In some embodiments, the RNAi agent has a modified antisense strand of SEQ ID NO: 208. In some embodiments, the RNAi agent has a modified sense strand of SEQ ID NO: 400 conjugated to Formula VI at the 3′ end and a modified antisense strand of SEQ ID NO: 208.

In some embodiments, the RNAi agent has a modified sense strand of SEQ ID NO: 401 conjugated to Formula VI at the 3′ end. In some embodiments, the RNAi agent has a modified antisense strand of SEQ ID NO: 209. In some embodiments, the RNAi agent has a modified sense strand of SEQ ID NO: 401 conjugated to Formula VI at the 3′ end and a modified antisense strand of SEQ ID NO: 209.

In some embodiments, the RNAi agent has a modified sense strand of SEQ ID NO: 402 conjugated to Formula VI at the 3′ end. In some embodiments, the RNAi agent has a modified antisense strand of SEQ ID NO: 210. In some embodiments, the RNAi agent has a modified sense strand of SEQ ID NO: 402 conjugated to Formula VI at the 3′ end and a modified antisense strand of SEQ ID NO: 210.

In some embodiments, the RNAi agent has a modified sense strand of SEQ ID NO: 403 conjugated to Formula VI at the 3′ end. In some embodiments, the RNAi agent has a modified antisense strand of SEQ ID NO: 211. In some embodiments, the RNAi agent has a modified sense strand of SEQ ID NO: 403 conjugated to Formula VI at the 3′ end and a modified antisense strand of SEQ ID NO: 211.

In some embodiments, the RNAi agent has a modified sense strand of SEQ ID NO: 404 conjugated to Formula VI at the 3′ end. In some embodiments, the RNAi agent has a modified antisense strand of SEQ ID NO: 212. In some embodiments, the RNAi agent has a modified sense strand of SEQ ID NO: 404 conjugated to Formula VI at the 3′ end and a modified antisense strand of SEQ ID NO: 212.

In some embodiments, the RNAi agent has a modified sense strand of SEQ ID NO: 405 conjugated to Formula VI at the 3′ end. In some embodiments, the RNAi agent has a modified antisense strand of SEQ ID NO: 213. In some embodiments, the RNAi agent has a modified sense strand of SEQ ID NO: 405 conjugated to Formula VI at the 3′ end and a modified antisense strand of SEQ ID NO: 213.

In some embodiments, the RNAi agent has a modified sense strand of SEQ ID NO: 406 conjugated to Formula VI at the 3′ end. In some embodiments, the RNAi agent has a modified antisense strand of SEQ ID NO: 214. In some embodiments, the RNAi agent has a modified sense strand of SEQ ID NO: 406 conjugated to Formula VI at the 3′ end and a modified antisense strand of SEQ ID NO: 214.

In some embodiments, the RNAi agent has a modified sense strand of SEQ ID NO: 407 conjugated to Formula VI at the 3′ end. In some embodiments, the RNAi agent has a modified antisense strand of SEQ ID NO: 215. In some embodiments, the RNAi agent has a modified sense strand of SEQ ID NO: 407 conjugated to Formula VI at the 3′ end and a modified antisense strand of SEQ ID NO: 215.

In some embodiments, the RNAi agent has a modified sense strand of SEQ ID NO: 408 conjugated to Formula VI at the 3′ end. In some embodiments, the RNAi agent has a modified antisense strand of SEQ ID NO: 216. In some embodiments, the RNAi agent has a modified sense strand of SEQ ID NO: 408 conjugated to Formula VI at the 3′ end and a modified antisense strand of SEQ ID NO: 216.

In some embodiments, the RNAi agent has a modified sense strand of SEQ ID NO: 409 conjugated to Formula VI at the 3′ end. In some embodiments, the RNAi agent has a modified antisense strand of SEQ ID NO: 217. In some embodiments, the RNAi agent has a modified sense strand of SEQ ID NO: 409 conjugated to Formula VI at the 3′ end and a modified antisense strand of SEQ ID NO: 217.

In some embodiments, the RNAi agent has a modified sense strand of SEQ ID NO: 410 conjugated to Formula VI at the 3′ end. In some embodiments, the RNAi agent has a modified antisense strand of SEQ ID NO: 218. In some embodiments, the RNAi agent has a modified sense strand of SEQ ID NO: 410 conjugated to Formula VI at the 3′ end and a modified antisense strand of SEQ ID NO: 218.

In some embodiments, the RNAi agent has a modified sense strand of SEQ ID NO: 411 conjugated to Formula VI at the 3′ end. In some embodiments, the RNAi agent has a modified antisense strand of SEQ ID NO: 219. In some embodiments, the RNAi agent has a modified sense strand of SEQ ID NO: 411 conjugated to Formula VI at the 3′ end and a modified antisense strand of SEQ ID NO: 219.

In some embodiments, the RNAi agent has a modified sense strand of SEQ ID NO: 412 conjugated to Formula VI at the 3′ end. In some embodiments, the RNAi agent has a modified antisense strand of SEQ ID NO: 220. In some embodiments, the RNAi agent has a modified sense strand of SEQ ID NO: 412 conjugated to Formula VII at the 3′ end and a modified antisense strand of SEQ ID NO: 220.

In some embodiments, the RNAi agent has a modified sense strand of SEQ ID NO: 413 conjugated to Formula VI at the 3′ end. In some embodiments, the RNAi agent has a modified antisense strand of SEQ ID NO: 221. In some embodiments, the RNAi agent has a modified sense strand of SEQ ID NO: 413 conjugated to Formula VI at the 3′ end and a modified antisense strand of SEQ ID NO: 221.

In some embodiments, the RNAi agent has a modified sense strand of SEQ ID NO: 414 conjugated to Formula VI at the 3′ end. In some embodiments, the RNAi agent has a modified antisense strand of SEQ ID NO: 222. In some embodiments, the RNAi agent has a modified sense strand of SEQ ID NO: 414 conjugated to Formula VI at the 3′ end and a modified antisense strand of SEQ ID NO: 222.

In some embodiments, the RNAi agent has a modified sense strand of SEQ ID NO: 415 conjugated to Formula VI at the 3′ end. In some embodiments, the RNAi agent has a modified antisense strand of SEQ ID NO: 223. In some embodiments, the RNAi agent has a modified sense strand of SEQ ID NO: 415 conjugated to Formula VI at the 3′ end and a modified antisense strand of SEQ ID NO: 223.

In some embodiments, the RNAi agent has a modified sense strand of SEQ ID NO: 416 conjugated to Formula VI at the 3′ end. In some embodiments, the RNAi agent has a modified antisense strand of SEQ ID NO: 224. In some embodiments, the RNAi agent has a modified sense strand of SEQ ID NO: 416 conjugated to Formula VI at the 3′ end and a modified antisense strand of SEQ ID NO: 224.

In some embodiments, the RNAi agent has a modified sense strand of SEQ ID NO: 417 conjugated to Formula VI at the 3′ end. In some embodiments, the RNAi agent has a modified antisense strand of SEQ ID NO: 225. In some embodiments, the RNAi agent has a modified sense strand of SEQ ID NO: 417 conjugated to Formula VI at the 3′ end and a modified antisense strand of SEQ ID NO: 225.

In some embodiments, the RNAi agent has a modified sense strand of SEQ ID NO: 418 conjugated to Formula VI at the 3′ end. In some embodiments, the RNAi agent has a modified antisense strand of SEQ ID NO: 226. In some embodiments, the RNAi agent has a modified sense strand of SEQ ID NO: 418 conjugated to Formula VI at the 3′ end and a modified antisense strand of SEQ ID NO: 226.

In some embodiments, the RNAi agent has a modified sense strand of SEQ ID NO: 419 conjugated to Formula VI at the 3′ end. In some embodiments, the RNAi agent has a modified antisense strand of SEQ ID NO: 227. In some embodiments, the RNAi agent has a modified sense strand of SEQ ID NO: 419 conjugated to Formula VI at the 3′ end and a modified antisense strand of SEQ ID NO: 227.

In some embodiments, the RNAi agent has a modified sense strand of SEQ ID NO: 420 conjugated to Formula VI at the 3′ end. In some embodiments, the RNAi agent has a modified antisense strand of SEQ ID NO: 228. In some embodiments, the RNAi agent has a modified sense strand of SEQ ID NO: 420 conjugated to Formula VI at the 3′ end and a modified antisense strand of SEQ ID NO: 228.

In some embodiments, the RNAi agent has a modified sense strand of SEQ ID NO: 421 conjugated to Formula VI at the 3′ end. In some embodiments, the RNAi agent has a modified antisense strand of SEQ ID NO: 229. In some embodiments, the RNAi agent has a modified sense strand of SEQ ID NO: 421 conjugated to Formula VI at the 3′ end and a modified antisense strand of SEQ ID NO: 229.

In some embodiments, the RNAi agent has a modified sense strand of SEQ ID NO: 422 conjugated to Formula VI at the 3′ end. In some embodiments, the RNAi agent has a modified antisense strand of SEQ ID NO: 230. In some embodiments, the RNAi agent has a modified sense strand of SEQ ID NO: 422 conjugated to Formula VI at the 3′ end and a modified antisense strand of SEQ ID NO: 230.

In some embodiments, the RNAi agent has a modified sense strand of SEQ ID NO: 423 conjugated to Formula VI at the 3′ end. In some embodiments, the RNAi agent has a modified antisense strand of SEQ ID NO: 231. In some embodiments, the RNAi agent has a modified sense strand of SEQ ID NO: 423 conjugated to Formula VI at the 3′ end and a modified antisense strand of SEQ ID NO: 231.

In some embodiments, the RNAi agent has a modified sense strand of SEQ ID NO: 424 conjugated to Formula VI at the 3′ end. In some embodiments, the RNAi agent has a modified antisense strand of SEQ ID NO: 232. In some embodiments, the RNAi agent has a modified sense strand of SEQ ID NO: 424 conjugated to Formula VI at the 3′ end and a modified antisense strand of SEQ ID NO: 232.

In some embodiments, the RNAi agent has a modified sense strand of SEQ ID NO: 425 conjugated to Formula VI at the 3′ end. In some embodiments, the RNAi agent has a modified antisense strand of SEQ ID NO: 233. In some embodiments, the RNAi agent has a modified sense strand of SEQ ID NO: 425 conjugated to Formula VI at the 3′ end and a modified antisense strand of SEQ ID NO: 233.

In some embodiments, the RNAi agent has a modified sense strand of SEQ ID NO: 426 conjugated to Formula VI at the 3′ end. In some embodiments, the RNAi agent has a modified antisense strand of SEQ ID NO: 234. In some embodiments, the RNAi agent has a modified sense strand of SEQ ID NO: 426 conjugated to Formula VI at the 3′ end and a modified antisense strand of SEQ ID NO: 234.

In some embodiments, the RNAi agent has a modified sense strand of SEQ ID NO: 427 conjugated to Formula VI at the 3′ end. In some embodiments, the RNAi agent has a modified antisense strand of SEQ ID NO: 235. In some embodiments, the RNAi agent has a modified sense strand of SEQ ID NO: 427 conjugated to Formula VI at the 3′ end and a modified antisense strand of SEQ ID NO: 235.

In some embodiments, the RNAi agent has a modified sense strand of SEQ ID NO: 428 conjugated to Formula VI at the 3′ end. In some embodiments, the RNAi agent has a modified antisense strand of SEQ ID NO: 236. In some embodiments, the RNAi agent has a modified sense strand of SEQ ID NO: 428 conjugated to Formula VI at the 3′ end and a modified antisense strand of SEQ ID NO: 236.

In some embodiments, the RNAi agent has a modified sense strand of SEQ ID NO: 429 conjugated to Formula VI at the 3′ end. In some embodiments, the RNAi agent has a modified antisense strand of SEQ ID NO: 237. In some embodiments, the RNAi agent has a modified sense strand of SEQ ID NO: 429 conjugated to Formula VI at the 3′ end and a modified antisense strand of SEQ ID NO: 237.

In some embodiments, the RNAi agent has a modified sense strand of SEQ ID NO: 430 conjugated to Formula VI at the 3′ end. In some embodiments, the RNAi agent has a modified antisense strand of SEQ ID NO: 238. In some embodiments, the RNAi agent has a modified sense strand of SEQ ID NO: 430 conjugated to Formula VI at the 3′ end and a modified antisense strand of SEQ ID NO: 238.

In some embodiments, the RNAi agent has a modified sense strand of SEQ ID NO: 431 conjugated to Formula VI at the 3′ end. In some embodiments, the RNAi agent has a modified antisense strand of SEQ ID NO: 239. In some embodiments, the RNAi agent has a modified sense strand of SEQ ID NO: 431 conjugated to Formula VI at the 3′ end and a modified antisense strand of SEQ ID NO: 239.

In some embodiments, the RNAi agent has a modified sense strand of SEQ ID NO: 432 conjugated to Formula VI at the 3′ end. In some embodiments, the RNAi agent has a modified antisense strand of SEQ ID NO: 240. In some embodiments, the RNAi agent has a modified sense strand of SEQ ID NO: 432 conjugated to Formula VI at the 3′ end and a modified antisense strand of SEQ ID NO: 240.

In some embodiments, the RNAi agent has a modified sense strand of SEQ ID NO: 433 conjugated to Formula VI at the 3′ end. In some embodiments, the RNAi agent has a modified antisense strand of SEQ ID NO: 241. In some embodiments, the RNAi agent has a modified sense strand of SEQ ID NO: 433 conjugated to Formula VI at the 3′ end and a modified antisense strand of SEQ ID NO: 241.

In some embodiments, the RNAi agent has a modified sense strand of SEQ ID NO: 434 conjugated to Formula VI at the 3′ end. In some embodiments, the RNAi agent has a modified antisense strand of SEQ ID NO: 242. In some embodiments, the RNAi agent has a modified sense strand of SEQ ID NO: 434 conjugated to Formula VI at the 3′ end and a modified antisense strand of SEQ ID NO: 242.

In some embodiments, the RNAi agent has a modified sense strand of SEQ ID NO: 435 conjugated to Formula VI at the 3′ end. In some embodiments, the RNAi agent has a modified antisense strand of SEQ ID NO: 243. In some embodiments, the RNAi agent has a modified sense strand of SEQ ID NO: 435 conjugated to Formula VI at the 3′ end and a modified antisense strand of SEQ ID NO: 243.

In some embodiments, the RNAi agent has a modified sense strand of SEQ ID NO: 436 conjugated to Formula VI at the 3′ end. In some embodiments, the RNAi agent has a modified antisense strand of SEQ ID NO: 244. In some embodiments, the RNAi agent has a modified sense strand of SEQ ID NO: 436 conjugated to Formula VI at the 3′ end and a modified antisense strand of SEQ ID NO: 244.

In some embodiments, the RNAi agent has a modified sense strand of SEQ ID NO: 437 conjugated to Formula VI at the 3′ end. In some embodiments, the RNAi agent has a modified antisense strand of SEQ ID NO: 245. In some embodiments, the RNAi agent has a modified sense strand of SEQ ID NO: 437 conjugated to Formula VI at the 3′ end and a modified antisense strand of SEQ ID NO: 245.

In some embodiments, the RNAi agent has a modified sense strand of SEQ ID NO: 438 conjugated to Formula VI at the 3′ end. In some embodiments, the RNAi agent has a modified antisense strand of SEQ ID NO: 246. In some embodiments, the RNAi agent has a modified sense strand of SEQ ID NO: 438 conjugated to Formula VI at the 3′ end and a modified antisense strand of SEQ ID NO: 246.

In some embodiments, the RNAi agent has a modified sense strand of SEQ ID NO: 439 conjugated to Formula VI at the 3′ end. In some embodiments, the RNAi agent has a modified antisense strand of SEQ ID NO: 247. In some embodiments, the RNAi agent has a modified sense strand of SEQ ID NO: 439 conjugated to Formula VI at the 3′ end and a modified antisense strand of SEQ ID NO: 247.

In some embodiments, the RNAi agent has a modified sense strand of SEQ ID NO: 440 conjugated to Formula VI at the 3′ end. In some embodiments, the RNAi agent has a modified antisense strand of SEQ ID NO: 248. In some embodiments, the RNAi agent has a modified sense strand of SEQ ID NO: 440 conjugated to Formula VI at the 3′ end and a modified antisense strand of SEQ ID NO: 248.

In some embodiments, the RNAi agent has a modified sense strand of SEQ ID NO: 441 conjugated to Formula VI at the 3′ end. In some embodiments, the RNAi agent has a modified antisense strand of SEQ ID NO: 249. In some embodiments, the RNAi agent has a modified sense strand of SEQ ID NO: 441 conjugated to Formula VI at the 3′ end and a modified antisense strand of SEQ ID NO: 249.

In some embodiments, the RNAi agent has a modified sense strand of SEQ ID NO: 442 conjugated to Formula VI at the 3′ end. In some embodiments, the RNAi agent has a modified antisense strand of SEQ ID NO: 250. In some embodiments, the RNAi agent has a modified sense strand of SEQ ID NO: 442 conjugated to Formula VI at the 3′ end and a modified antisense strand of SEQ ID NO: 250.

In some embodiments, the RNAi agent has a modified sense strand of SEQ ID NO: 443 conjugated to Formula VI at the 3′ end. In some embodiments, the RNAi agent has a modified antisense strand of SEQ ID NO: 251. In some embodiments, the RNAi agent has a modified sense strand of SEQ ID NO: 443 conjugated to Formula VI at the 3′ end and a modified antisense strand of SEQ ID NO: 251.

In some embodiments, the RNAi agent has a modified sense strand of SEQ ID NO: 444 conjugated to Formula VI at the 3′ end. In some embodiments, the RNAi agent has a modified antisense strand of SEQ ID NO: 252. In some embodiments, the RNAi agent has a modified sense strand of SEQ ID NO: 444 conjugated to Formula VI at the 3′ end and a modified antisense strand of SEQ ID NO: 252.

In some embodiments, the RNAi agent has a modified sense strand of SEQ ID NO: 445 conjugated to Formula VI at the 3′ end. In some embodiments, the RNAi agent has a modified antisense strand of SEQ ID NO: 253. In some embodiments, the RNAi agent has a modified sense strand of SEQ ID NO: 445 conjugated to Formula VI at the 3′ end and a modified antisense strand of SEQ ID NO: 253.

In some embodiments, the RNAi agent has a modified sense strand of SEQ ID NO: 446 conjugated to Formula VI at the 3′ end. In some embodiments, the RNAi agent has a modified antisense strand of SEQ ID NO: 254. In some embodiments, the RNAi agent has a modified sense strand of SEQ ID NO: 446 conjugated to Formula VI at the 3′ end and a modified antisense strand of SEQ ID NO: 254.

In some embodiments, the RNAi agent has a modified sense strand of SEQ ID NO: 447 conjugated to Formula VI at the 3′ end. In some embodiments, the RNAi agent has a modified antisense strand of SEQ ID NO: 255. In some embodiments, the RNAi agent has a modified sense strand of SEQ ID NO: 447 conjugated to Formula VI at the 3′ end and a modified antisense strand of SEQ ID NO: 255.

In some embodiments, the RNAi agent has a modified sense strand of SEQ ID NO: 448 conjugated to Formula VI at the 3′ end. In some embodiments, the RNAi agent has a modified antisense strand of SEQ ID NO: 256. In some embodiments, the RNAi agent has a modified sense strand of SEQ ID NO: 448 conjugated to Formula VI at the 3′ end and a modified antisense strand of SEQ ID NO: 256.

In some embodiments, the RNAi agent has a modified sense strand of SEQ ID NO: 449 conjugated to Formula VI at the 3′ end. In some embodiments, the RNAi agent has a modified antisense strand of SEQ ID NO: 257. In some embodiments, the RNAi agent has a modified sense strand of SEQ ID NO: 449 conjugated to Formula VI at the 3′ end and a modified antisense strand of SEQ ID NO: 257.

In some embodiments, the RNAi agent has a modified sense strand of SEQ ID NO: 450 conjugated to Formula VI at the 3′ end. In some embodiments, the RNAi agent has a modified antisense strand of SEQ ID NO: 258. In some embodiments, the RNAi agent has a modified sense strand of SEQ ID NO: 450 conjugated to Formula VI at the 3′ end and a modified antisense strand of SEQ ID NO: 258.

In some embodiments, the RNAi agent has a modified sense strand of SEQ ID NO: 451 conjugated to Formula VI at the 3′ end. In some embodiments, the RNAi agent has a modified antisense strand of SEQ ID NO: 259. In some embodiments, the RNAi agent has a modified sense strand of SEQ ID NO: 451 conjugated to Formula VI at the 3′ end and a modified antisense strand of SEQ ID NO: 259.

In some embodiments, the RNAi agent has a modified sense strand of SEQ ID NO: 452 conjugated to Formula VI at the 3′ end. In some embodiments, the RNAi agent has a modified antisense strand of SEQ ID NO: 260. In some embodiments, the RNAi agent has a modified sense strand of SEQ ID NO: 452 conjugated to Formula VI at the 3′ end and a modified antisense strand of SEQ ID NO: 260.

In some embodiments, the RNAi agent has a modified sense strand of SEQ ID NO: 453 conjugated to Formula VI at the 3′ end. In some embodiments, the RNAi agent has a modified antisense strand of SEQ ID NO: 261. In some embodiments, the RNAi agent has a modified sense strand of SEQ ID NO: 453 conjugated to Formula VI at the 3′ end and a modified antisense strand of SEQ ID NO: 261.

In some embodiments, the RNAi agent has a modified sense strand of SEQ ID NO: 454 conjugated to Formula VI at the 3′ end. In some embodiments, the RNAi agent has a modified antisense strand of SEQ ID NO: 262. In some embodiments, the RNAi agent has a modified sense strand of SEQ ID NO: 454 conjugated to Formula VI at the 3′ end and a modified antisense strand of SEQ ID NO: 262.

In some embodiments, the RNAi agent has a modified sense strand of SEQ ID NO: 455 conjugated to Formula VI at the 3′ end. In some embodiments, the RNAi agent has a modified antisense strand of SEQ ID NO: 263. In some embodiments, the RNAi agent has a modified sense strand of SEQ ID NO: 455 conjugated to Formula VI at the 3′ end and a modified antisense strand of SEQ ID NO: 263.

In some embodiments, the RNAi agent has a modified sense strand of SEQ ID NO: 456 conjugated to Formula VI at the 3′ end. In some embodiments, the RNAi agent has a modified antisense strand of SEQ ID NO: 264. In some embodiments, the RNAi agent has a modified sense strand of SEQ ID NO: 456 conjugated to Formula VI at the 3′ end and a modified antisense strand of SEQ ID NO: 264.

In some embodiments, the RNAi agent has a modified sense strand of SEQ ID NO: 457 conjugated to Formula VI at the 3′ end. In some embodiments, the RNAi agent has a modified antisense strand of SEQ ID NO: 265. In some embodiments, the RNAi agent has a modified sense strand of SEQ ID NO: 457 conjugated to Formula VI at the 3′ end and a modified antisense strand of SEQ ID NO: 265.

In some embodiments, the RNAi agent has a modified sense strand of SEQ ID NO: 458 conjugated to Formula VI at the 3′ end. In some embodiments, the RNAi agent has a modified antisense strand of SEQ ID NO: 266. In some embodiments, the RNAi agent has a modified sense strand of SEQ ID NO: 458 conjugated to Formula VI at the 3′ end and a modified antisense strand of SEQ ID NO: 266.

In some embodiments, the RNAi agent has a modified sense strand of SEQ ID NO: 459 conjugated to Formula VI at the 3′ end. In some embodiments, the RNAi agent has a modified antisense strand of SEQ ID NO: 267. In some embodiments, the RNAi agent has a modified sense strand of SEQ ID NO: 459 conjugated to Formula VI at the 3′ end and a modified antisense strand of SEQ ID NO: 267.

In some embodiments, the RNAi agent has a modified sense strand of SEQ ID NO: 460 conjugated to Formula VI at the 3′ end. In some embodiments, the RNAi agent has a modified antisense strand of SEQ ID NO: 268. In some embodiments, the RNAi agent has a modified sense strand of SEQ ID NO: 460 conjugated to Formula VI at the 3′ end and a modified antisense strand of SEQ ID NO: 268.

In some embodiments, the RNAi agent has a modified sense strand of SEQ ID NO: 461 conjugated to Formula VI at the 3′ end. In some embodiments, the RNAi agent has a modified antisense strand of SEQ ID NO: 269. In some embodiments, the RNAi agent has a modified sense strand of SEQ ID NO: 461 conjugated to Formula VI at the 3′ end and a modified antisense strand of SEQ ID NO: 269.

In some embodiments, the RNAi agent has a modified sense strand of SEQ ID NO: 462 conjugated to Formula VI at the 3′ end. In some embodiments, the RNAi agent has a modified antisense strand of SEQ ID NO: 270. In some embodiments, the RNAi agent has a modified sense strand of SEQ ID NO: 462 conjugated to Formula VI at the 3′ end and a modified antisense strand of SEQ ID NO: 270.

In some embodiments, the RNAi agent has a modified sense strand of SEQ ID NO: 463 conjugated to Formula VI at the 3′ end. In some embodiments, the RNAi agent has a modified antisense strand of SEQ ID NO: 271. In some embodiments, the RNAi agent has a modified sense strand of SEQ ID NO: 463 conjugated to Formula VI at the 3′ end and a modified antisense strand of SEQ ID NO: 271.

In some embodiments, the RNAi agent has a modified sense strand of SEQ ID NO: 464 conjugated to Formula VI at the 3′ end. In some embodiments, the RNAi agent has a modified antisense strand of SEQ ID NO: 272. In some embodiments, the RNAi agent has a modified sense strand of SEQ ID NO: 464 conjugated to Formula VI at the 3′ end and a modified antisense strand of SEQ ID NO: 272.

In some embodiments, the RNAi agent has a modified sense strand of SEQ ID NO: 465 conjugated to Formula VI at the 3′ end. In some embodiments, the RNAi agent has a modified antisense strand of SEQ ID NO: 273. In some embodiments, the RNAi agent has a modified sense strand of SEQ ID NO: 465 conjugated to Formula VI at the 3′ end and a modified antisense strand of SEQ ID NO: 273.

In some embodiments, the RNAi agent has a modified sense strand of SEQ ID NO: 466 conjugated to Formula VI at the 3′ end. In some embodiments, the RNAi agent has a modified antisense strand of SEQ ID NO: 274. In some embodiments, the RNAi agent has a modified sense strand of SEQ ID NO: 466 conjugated to Formula VI at the 3′ end and a modified antisense strand of SEQ ID NO: 274.

In some embodiments, the RNAi agent has a modified sense strand of SEQ ID NO: 467 conjugated to Formula VI at the 3′ end. In some embodiments, the RNAi agent has a modified antisense strand of SEQ ID NO: 275. In some embodiments, the RNAi agent has a modified sense strand of SEQ ID NO: 467 conjugated to Formula VI at the 3′ end and a modified antisense strand of SEQ ID NO: 275.

In some embodiments, the RNAi agent has a modified sense strand of SEQ ID NO: 468 conjugated to Formula VI at the 3′ end. In some embodiments, the RNAi agent has a modified antisense strand of SEQ ID NO: 276. In some embodiments, the RNAi agent has a modified sense strand of SEQ ID NO: 468 conjugated to Formula VI at the 3′ end and a modified antisense strand of SEQ ID NO: 276.

In some embodiments, the RNAi agent has a modified sense strand of SEQ ID NO: 469 conjugated to Formula VI at the 3′ end. In some embodiments, the RNAi agent has a modified antisense strand of SEQ ID NO: 277. In some embodiments, the RNAi agent has a modified sense strand of SEQ ID NO: 469 conjugated to Formula VI at the 3′ end and a modified antisense strand of SEQ ID NO: 277.

In some embodiments, the RNAi agent has a modified sense strand of SEQ ID NO: 470 conjugated to Formula VI at the 3′ end. In some embodiments, the RNAi agent has a modified antisense strand of SEQ ID NO: 278. In some embodiments, the RNAi agent has a modified sense strand of SEQ ID NO: 470 conjugated to Formula VI at the 3′ end and a modified antisense strand of SEQ ID NO: 278.

In some embodiments, the RNAi agent has a modified sense strand of SEQ ID NO: 471 conjugated to Formula VI at the 3′ end. In some embodiments, the RNAi agent has a modified antisense strand of SEQ ID NO: 279. In some embodiments, the RNAi agent has a modified sense strand of SEQ ID NO: 471 conjugated to Formula VI at the 3′ end and a modified antisense strand of SEQ ID NO: 279.

In some embodiments, the RNAi agent has a modified sense strand of SEQ ID NO: 472 conjugated to Formula VI at the 3′ end. In some embodiments, the RNAi agent has a modified antisense strand of SEQ ID NO: 280. In some embodiments, the RNAi agent has a modified sense strand of SEQ ID NO: 472 conjugated to Formula VI at the 3′ end and a modified antisense strand of SEQ ID NO: 280.

In some embodiments, the RNAi agent has a modified sense strand of SEQ ID NO: 473 conjugated to Formula VI at the 3′ end. In some embodiments, the RNAi agent has a modified antisense strand of SEQ ID NO: 281. In some embodiments, the RNAi agent has a modified sense strand of SEQ ID NO: 473 conjugated to Formula VI at the 3′ end and a modified antisense strand of SEQ ID NO: 281.

In some embodiments, the RNAi agent has a modified sense strand of SEQ ID NO: 474 conjugated to Formula VI at the 3′ end. In some embodiments, the RNAi agent has a modified antisense strand of SEQ ID NO: 282. In some embodiments, the RNAi agent has a modified sense strand of SEQ ID NO: 474 conjugated to Formula VI at the 3′ end and a modified antisense strand of SEQ ID NO: 282.

In some embodiments, the RNAi agent has a modified sense strand of SEQ ID NO: 475 conjugated to Formula VI at the 3′ end. In some embodiments, the RNAi agent has a modified antisense strand of SEQ ID NO: 283. In some embodiments, the RNAi agent has a modified sense strand of SEQ ID NO: 475 conjugated to Formula VI at the 3′ end and a modified antisense strand of SEQ ID NO: 283.

In some embodiments, the RNAi agent has a modified sense strand of SEQ ID NO: 476 conjugated to Formula VI at the 3′ end. In some embodiments, the RNAi agent has a modified antisense strand of SEQ ID NO: 284. In some embodiments, the RNAi agent has a modified sense strand of SEQ ID NO: 476 conjugated to Formula VI at the 3′ end and a modified antisense strand of SEQ ID NO: 284.

In some embodiments, the RNAi agent has a modified sense strand of SEQ ID NO: 477 conjugated to Formula VI at the 3′ end. In some embodiments, the RNAi agent has a modified antisense strand of SEQ ID NO: 285. In some embodiments, the RNAi agent has a modified sense strand of SEQ ID NO: 477 conjugated to Formula VI at the 3′ end and a modified antisense strand of SEQ ID NO: 285.

In some embodiments, the RNAi agent has a modified sense strand of SEQ ID NO: 478 conjugated to Formula VI at the 3′ end. In some embodiments, the RNAi agent has a modified antisense strand of SEQ ID NO: 286. In some embodiments, the RNAi agent has a modified sense strand of SEQ ID NO: 478 conjugated to Formula VI at the 3′ end and a modified antisense strand of SEQ ID NO: 286.

In some embodiments, the RNAi agent has a modified sense strand of SEQ ID NO: 479 conjugated to Formula VI at the 3′ end. In some embodiments, the RNAi agent has a modified antisense strand of SEQ ID NO: 287. In some embodiments, the RNAi agent has a modified sense strand of SEQ ID NO: 479 conjugated to Formula VI at the 3′ end and a modified antisense strand of SEQ ID NO: 287.

In some embodiments, the RNAi agent has a modified sense strand of SEQ ID NO: 480 conjugated to Formula VI at the 3′ end. In some embodiments, the RNAi agent has a modified antisense strand of SEQ ID NO: 288. In some embodiments, the RNAi agent has a modified sense strand of SEQ ID NO: 480 conjugated to Formula VI at the 3′ end and a modified antisense strand of SEQ ID NO: 288.

### III. PHARMACEUTICAL COMPOSITIONS

Also provided are compositions including the RNAi agent, including pharmaceutical compositions and formulations. In some embodiments, provided herein is a pharmaceutical composition comprising any one of the RNAi agents provided herein, any one of the modified oligonucleotides provided herein, or any one of the oligomeric duplexes provided herein. The pharmaceutical compositions and formulations generally include one or more optional pharmaceutically acceptable carrier(s) or excipient(s). In some embodiments, provided herein is a pharmaceutical composition comprising any one of the RNAi agents provided herein, any one of the modified oligonucleotides provided herein, or any one of the oligomeric duplexes provided herein, and a pharmaceutically acceptable carrier. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being treated with the RNAI agent, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs.

In some embodiments, a delivery vehicle may be used to deliver an RNAi agent to a cell or tissue. A delivery vehicle is a compound that improves delivery of the RNAi agent to a cell or tissue. A delivery vehicle can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine.

In some embodiments, the RNAi agents, modified oligonucleotides, and oligomeric duplexes can be combined with lipids, nanoparticles, polymers, liposomes, micelles, DPCs or other delivery systems available in the art. The RNAi agents can also be chemically conjugated to targeting groups, lipids (including, but not limited to cholesterol and cholesteryl derivatives), nanoparticles, polymers, liposomes, micelles, DPCs (see, for example WO 2000/053722, WO 2008/0022309, WO 2011/104169, and WO 2012/083185, WO 2013/032829, WO 2013/158141, each of which is incorporated herein by reference), or other delivery systems available in the art. Additional lipid particle formulations are described in U.S. Pat. No. 10,683,501, which is incorporated herein by reference.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the host cells (e.g., T-cells or NK cells) to a particular tissue. M any methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and 5,019,369.

In some embodiments, the RNAi agents, modified oligonucleotides, and oligomeric duplexes may be fully encapsulated in a lipid formulation, e.g., a LNP, or other nucleic acid-lipid particle. As used herein, the term “LNP” refers to a stable nucleic acid-lipid particle. LNPs typically contain a cationic lipid, a non-cationic lipid, and a lipid that prevents aggregation of the particle (e.g., a PEG-lipid conjugate). LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5, 981, 501; 6,534,484; 6,586,410; 6,815,432; U.S. Publication No. 2010/0324120 and PCT Publication No. WO 96/4096.

In some embodiments, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the invention.

The cationic lipid can be, for example, N,N-dioleyl-N,N-dimethylammonium chloride (DODAC), N,N-distearyl-N,N-dimethylammonium bromide (DDAB), N—(I-(2,3-dioleoyloxy)propyl)-N, N,N-trimethylammonium chloride (DOTAP), N—(I-(2,3-dioleyloxy)propyl)-N, N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), 1,2-Dilinoleoyl-3-trimethylaminopropanechloride salt (DLin-TAP.CI), 1,2-Dilinoleyloxy-3-(N-methylpiperazino)propane(DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane(DLin-EG-DMA), 1,2-Dilinolenyloxy-N,N-dimethylaminopropane(DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine (ALN 100), (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (Tech G1), or a mixture thereof. The cationic lipid can comprise from about 20 mol % to about 50 mol % or about 40 mol % of the total lipid present in the particle.

In some embodiments, the compound 2,2-Dilinoleyl-4-dimethylaminoethyl-[1,3]-dioxolane can be used to prepare lipid-siRNA nanoparticles. Synthesis of 2,2-Dilinoleyl-4-dimethylaminoethyl-[1,3]-dioxolane is described in U.S. provisional patent application No. 61/107,998 filed on Oct. 23, 2008, which is herein incorporated by reference. In some embodiments, the lipid-siRNA particle includes 40% 2,2-Dilinoleyl-4-dimethylaminoethyl-[1,3]-dioxolane: 10% DSPC: 40% Cholesterol: 10% PEG-C-DOM G (mole percent) with a particle size of 63.0±20 nm and a 0.027 siRNA/Lipid Ratio.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be from about 5 mol % to about 90 mol %, about 10 mol %, or about 58 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C]₈). The conjugated lipid that prevents aggregation of particles can be from 0 mol % to about 20 mol % or about 2 mol % of the total lipid present in the particle.

In some embodiments, the nucleic acid-lipid particle further includes cholesterol at, e.g., about 10 mol % to about 60 mol % or about 48 mol % of the total lipid present in the particle.

In some embodiments, the lipidoid ND98.4HCl (MW 1487) (see U.S. patent application Ser. No. 12/056,230, filed Mar. 26, 2008, which is incorporated herein by reference), Cholesterol (Sigma-Aldrich), and PEG-CeramideC16 (Avanti Polar Lipids) can be used to prepare lipid-dsRNA nanoparticles (i.e., LNP01 particles). Stock solutions of each in ethanol can be prepared as follows: ND98, 133 mg/ml; Cholesterol, 25 mg/ml, PEG-Ceramide C16, 100 mg/ml. The ND98, Cholesterol, and PEG-Ceramide C16 stock solutions can then be combined in a, e.g., 42:48:10 molar ratio. The combined lipid solution can be mixed with aqueous dsRNA (e.g., in sodium acetate pH 5) such that the final ethanol concentration is about 35-45% and the final sodium acetate concentration is about 100-300 mM. Lipid-dsRNA nanoparticles typically form spontaneously upon mixing. Depending on the desired particle size distribution, the resultant nanoparticle mixture can be extruded through a polycarbonate membrane (e.g., 100 nm cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. Buffer can be exchanged with, for example, phosphate buffered saline (PBS) at about pH 7, e.g., about pH 6.9, about pH 7.0, about pH 7.1, about pH 7.2, about pH 7.3, or about pH 7.4.

In some embodiments, provided herein is a lipid nanoparticle (such as any of the lipid nanoparticles described herein) comprising any one of the RNAi agents described herein, any one of the modified oligonucleotides described herein, any one of the oligomeric duplexes described herein, or any one of the pharmaceutical compositions described herein.

The pharmaceutical composition in some aspects can employ time-released, delayed release, and sustained release delivery systems such that the delivery of the composition occurs prior to, and with sufficient time to cause, sensitization of the site to be treated. M any types of release delivery systems are available and known. Such systems can avoid repeated administrations of the composition, thereby increasing convenience to the subject and the physician.

The pharmaceutical composition in some embodiments contains the RNAi agent in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The pharmaceutical composition may be administered using standard administration techniques, formulations, and/or devices. Provided are formulations and devices, such as syringes and vials, for storage and administration of the compositions. Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, intracranial, intrathoracic, and intraperitoneal administration.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the RNAi in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

In some embodiments, pharmaceutical compositions for delivering an RNAi agent (e.g. a GCK RNAi) to a liver cell in vivo are described. Such pharmaceutical compositions can include, for example, an RNAi agent (e.g. a GCK RNAi) conjugated to a galactose derivative cluster. In some embodiments, the galactose derivative cluster is comprised of a galactose derivative trimer, which can be, for example, an N-acetyl-galactosamine trimer, or galactose derivative tetramer, which can be, for example, an N-acetyl-galactosamine tetramer.

The present disclosure also provides methods of making a pharmaceutical composition, such as any of the pharmaceutical compositions described herein. In some aspects, provided herein is a method of making a pharmaceutical composition, comprising combining any one of the RNAi agents described herein, any one of the modified oligonucleotides described herein, or any one of the oligomeric duplexes described herein, with a delivery vehicle. In some embodiments, the delivery vehicle is a delivery vehicle selected from the group consisting of a phosphate-buffered saline (PBS), lipid, a nanoparticle, a polymer, a liposome, a micelle, a dynamic polyconjugate (DPC), or an antibody-oligo conjugate.

### IV. USES AND METHODS

Also provided herein are methods of administering and uses of, such as therapeutic and prophylactic uses of, the RNAi agent (e.g., GCK RNAi) and/or compositions comprising the same. Such methods and uses include therapeutic methods and uses, for example, involving administration of the RNAi (e.g., GCK RNAi) or compositions containing the same, to a subject having a disease, condition, or disorder associated with GCK such as a disease, condition, or disorder associated with GCK expression, and/or in which cells or tissues express, e.g., specifically express, GCK.

In some embodiments, the RNAi agent and/or composition is administered in an effective amount to effect treatment of the disease or disorder. Provided herein are uses of the RNAi agent in such methods and treatments, and in the preparation of a medicament in order to carry out such therapeutic methods. In some embodiments, the methods are carried out by administering the RNAi agent or compositions comprising the same, to the subject having, having had, or suspected of having the disease or condition. In some embodiments, the methods thereby treat the disease or condition or disorder in the subject. Also provided herein are use of any of the compositions, such as pharmaceutical compositions provided herein, for the treatment of a disease or disorder associated with GCK, such as use in a treatment regimen. Also provided herein are use of any of the compositions, such as pharmaceutical compositions provided herein, for the treatment of Metabolic dysfunction-associated steatotic liver disease (MASLD) and/or metabolic dysfunction-associated steatohepatitis (MASH), such as use in a treatment regimen. Also provided herein are uses of any of the compositions, such as pharmaceutical compositions, for reducing, stopping progression of, or slowing progression of MASLD, MASH, steatosis, fibrosis, and/or cirrhosis.

The disease, condition, or disorder that is detected and diagnosed by the RNAi agent disclosed herein can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g., cancer), autoimmune or inflammatory disease, or an infectious disease, e.g., caused by a bacterial, viral or other pathogen.

In some embodiments, the disease, disorder, or condition is an infectious disease such as infection with a virus or other pathogen, e.g., HIV, HCV, HBV, CMV, HPV, and parasitic disease.

In some embodiments, the disease, disorder, or condition is a liver disease. In certain embodiments, the liver disease includes, but is not limited to, simple fatty liver (steatosis), metabolic dysfunction-associated steatohepatitis (MASH), fibrosis, and cirrhosis.

Among the disease, disorder, or condition to be treated is any disease or disorder associated with nonalcoholic fatty liver disease. Among the disease, disorder, or condition to be treated is any disease or disorder associated with GCK or any disease or disorder in which GCK is specifically expressed and/or in which GCK has been targeted for treatment. Glucokinase acts as a sensor of blood glucose levels in the pancreas, and a few other cells of the gut and brain, and as the rate-limiting step in glucose metabolism in the liver. It has been demonstrated that GCK mRNA expression is associated with markers of de novo lipogenesis and liver triglyceride content in humans. It is suggested that increased GCK activity may induce fatty liver and its metabolic and hepatic consequences in humans (Peter, et al., J Clin Endocrinol Metab. 2011 July; 96(7):E1126-30.). In some embodiments, the disease or disorder is non-alcoholic fatty liver. In some embodiments, the disease or disorder is metabolic dysfunction-associated steatohepatitis (MASH). In some embodiments, the disease or disorder is metabolic dysfunction-associated steatotic liver disease (MASLD).

In some embodiments, provided herein is a method of treating MASLD or MASH in a subject, comprising administering to the subject an effective amount of a liver-targeted modified oligonucleotide, a liver-targeted oligomeric duplex, or a liver-targeted RNAi agent that inhibits expression of GCK. In some embodiments, the liver-targeted modified oligonucleotide, the liver-targeted oligomeric duplex, and the liver-targeted RNAi agent is any one of the modified oligonucleotides, oligomeric duplexes, or RNAi agents described herein.

In some embodiments, the RNAi (e.g., GCK RNAi) or compositions containing the same are administered to a subject for reducing, stopping progression of, or slowing progression of steatosis, fibrosis, and/or cirrhosis.

In some embodiments, the RNAi (e.g., GCK RNAi) or compositions containing the same are administered to a subject for inhibiting expression of GCK. In some embodiments, inhibiting expression of GCK comprises reducing the expression of GCK compared to the expression of GCK prior to the administration of the RNAi. In some embodiments, inhibiting expression of GCK comprises eliminating the expression of GCK.

In some embodiments, provided herein is a method of controlling or reducing liver fat accumulation in a subject, comprising administering to the subject an effective amount of a liver-targeted RNAi agent that inhibits expression of GCK. In some embodiments, inhibiting expression of GCK comprises reducing the expression of GCK compared to the expression of GCK prior to the administration of the RNAi. In some embodiments, inhibiting expression of GCK comprises eliminating the expression of GCK. As used herein “reducing” liver fat accumulation refers to a decrease in liver fat compared to the amount of liver fat present prior to administration of the liver-targeting RNAi agent. In some embodiments, the liver fat accumulation is reduced any of about 1%, 5%, 10%, 15%, 20%, 25%, 30%, 35%, 40%, 45%, 50%, 55%, 60%, 65%, 70%, 75%, 80%, 85%, 90%, 95%, or more, compared to the amount of liver fat present prior to administration of the liver-targeting RNAi agent.

In some embodiments, the subject is an adult subject. In some embodiments, the subject is ≥18 years of age. In some embodiments, the subject to be treated is human. In some embodiments, the subject, to whom the RNAi agent or compositions are administered, is a primate, such as a human. In some embodiments, the subject, to whom the RNAi agent or compositions are administered, is a non-human primate. In some embodiments, the non-human primate is a monkey (e.g., cynomolgus monkey) or an ape. The subject can be male or female and can be any suitable age, including infant, juvenile, adolescent, adult, and geriatric subjects. In some embodiments, the subject is a non-primate mammal, such as a rodent (e.g., mouse, rat, etc.). In some examples, the patient or subject is a validated animal model for nonalcoholic fatty liver disease and/or for assessing toxic outcomes.

The RNAi agent can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, intracranial, intrathoracic, or subcutaneous administration. Dosing and administration may depend in part on whether the administration is brief or chronic. Various dosing schedules include but are not limited to single or multiple administrations over various time-points, bolus administration, and pulse infusion.

For the prevention or treatment of disease, the appropriate dosage of the RNAi agent may depend on the type of disease to be treated, the severity and course of the disease, whether the RNAi agent is administered for preventive or therapeutic purposes, the patient's clinical history and response to the RNAi agent, and the discretion of the attending physician. In some embodiments, the RNAi agent and/or composition are suitably administered to the patient at one time or over a series of treatments.

In some embodiments, the dose and/or frequency of administration is/are determined based on efficacy and/or response. In some embodiments, efficacy is determined by evaluating disease status. Exemplary methods for assessing disease status include: decrease of GCK gene expression, decrease of GCK protein, decrease of liver triglycerides, decrease in lipogenic gene expression (e.g. fatty acid synthase, acetyl-coenzyme A carboxylase-α, and acetyl-coenzyme A carboxylase-β), decrease of liver nodules, or decrease of inflammation in the liver.

In some embodiments, toxicity and/or side-effects of treatment can be monitored and used to adjust dose and/or frequency of administration of the RNAi agent and/or compositions. For example, adverse events and laboratory abnormalities can be monitored and used to adjust dose and/or frequency of administration. Any of such events may establish dose-limiting toxicities and warrant decrease in dose and/or a termination of treatment.

In some embodiments, the RNAi agent provided herein (e.g. GCK RNAi) is used to treat or manage a clinical presentation wherein a subject has or is suspected of having metabolic dysfunction-associated steatotic liver disease (MASLD). The subject is administered a therapeutically or effective amount of one or more of the RNAi agents (e.g. GCK RNAi) or RNAi agent-containing compositions described herein. In some embodiments, the method comprises administering a composition comprising an GCK RNAi agent described herein to a subject to be treated.

In some embodiments, the gene expression level and/or mRNA level of GCK gene in a subject to whom a described GCK RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the GCK RNAi agent or to a subject not receiving the GCK RNAi agent. The gene expression level and/or mRNA level in the subject may be reduced in a cell, group of cells, and/or tissue of the subject. In some embodiments, the expressed protein level of GCK protein in a subject to whom a described GCK RNAi agent has been administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the GCK RNAi agent or to a subject not receiving the GCK RNAi agent. The GCK protein level in the subject may be reduced in a cell, group of cells, tissue, blood, and/or other fluid of the subject.

In some embodiments, the amount or level of liver triglycerides in human liver biopsies of a subject to whom the described GCK RNAi agent has been administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the GCK RNAi agent or to a subject not receiving the GCK RNAi agent.

In some embodiments, the amount or level of lipogenic gene expression (e.g. fatty acid synthase, acetyl-coenzyme A carboxylase-α, and acetyl-coenzyme A carboxylase-β) of a subject to whom the described GCK RNAi agent has been administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the GCK RNAi agent or to a subject not receiving the GCK RNAi agent.

A reduction in the presence of GCK gene expression, GCK mRNA, or GCK protein amounts or levels may be assessed by methods known in the art. Reduction or decrease in GCK mRNA amount or level and/or expressed protein amount or level, and/or liver triglycerides and lipogenic gene expression amount or level are collectively referred to herein as treating or ameliorating metabolic dysfunction-associated steatotic liver disease (MASLD).

In some embodiments, the amount or size of liver nodules of the liver of the subject to whom the described GCK RNAi agent has been administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the GCK RNAi agent or to a subject not receiving the GCK RNAi agent. In some embodiments, imaging tests may be used to diagnose MASLD and/or MASH. These imaging tests may include, but are not limited to, ultrasound, computed tomography (CT) scan, magnetic resonance imaging (MRI), and elastography.

In some embodiments, the amount or level inflammation in the liver of a subject to whom the described GCK RNAi agent has been administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the GCK RNAi agent or to a subject not receiving the GCK RNAi agent. In some embodiments, the amount of fibrotic damage in the liver of a subject to whom the described GCK RNAi agent has been administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the GCK RNAi agent or to a subject not receiving the GCK RNAi agent. In some embodiments, reduction in liver inflammation or fibrotic damage may be determined by any method known in the art. In some embodiments, the subject may undergo a liver biopsy in order to diagnosis fibrosis.

In some embodiments, the RNAi agent or compositions containing the same are co-administered with one or more additional therapeutic agents or in connection with another therapeutic intervention, either simultaneously or sequentially in any order. In some contexts, the RNAi agent or compositions are co-administered with another therapy sufficiently close in time such that the RNAi agent enhances the effect of one or more additional therapeutic agents, or vice versa. In some embodiments, the RNAi agent or compositions are administered prior to the one or more additional therapeutic agents. In some embodiments, the RNAi agent or compositions are administered after to the one or more additional therapeutic agents.

### V. ARTICLES OF MANUFACTURE AND KITS

Also provided are articles of manufacture containing the RNAi agent and/or compositions thereof. The articles of manufacture may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition.

The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VI. EXEMPLARY EMBODIMENTS

The following embodiments are exemplary and are not intended to limit the scope of the invention or inventions described herein. Among the provided embodiments are:

Embodiment 1. An RNAi agent that inhibits expression of glucokinase (GCK) for use in the treatment of metabolic dysfunction-associated steatotic liver disease (MASLD) or metabolic dysfunction-associated steatotic liver (MASL) in a subject, wherein the RNAi agent comprises (i) an antisense strand and a sense strand that at least partially complements the antisense strand, and (ii) a targeting ligand conjugated to the antisense strand or the sense strand, wherein the targeting ligand targets a hepatocyte.

Embodiment 2. A n RNAi agent that inhibits expression of glucokinase (GCK) comprising an antisense strand and a sense strand that at least partially complements the antisense strand, and a targeting ligand conjugated to the antisense strand or the sense strand, wherein the targeting ligand targets a hepatocyte.

Embodiment 3. An RNAi agent that inhibits expression of glucokinase (GCK) comprising an antisense strand and a sense strand that at least partially complements the antisense strand, wherein:
(a) the antisense strand comprises at least 14 contiguous bases from any one of SE Q ID NO S: 1-96; and/or
(b) the sense strand comprises at least 14 contiguous bases from any one of SEQ ID NOS: 97-192.

Embodiment 4. The RNAi agent of any one of embodiments 1-3, wherein one or more bases of the antisense stand and/or the sense strand are modified.

Embodiment 5. The RNAi agent of embodiment 4, wherein the antisense strand has a modification pattern according to nsNfsnnnNfnnnnnnnNfnNfnnnnnsnsn.

Embodiment 6. The RNAi agent of embodiment 4 or 5, wherein the sense strand has a modification pattern according to nsnsnnnnNfnNfNfNfnnnnnnnnsnsn.

Embodiment 7. The RNAi agent of embodiment 4 or 5, wherein the sense strand has a modification pattern according to nsnsnnnnNfnNfNfNfnnnnnnnnnn.

Embodiment 8. The RNAi agent of any one of embodiments 1-7, wherein:
(a) the antisense strand comprises at least 14 contiguous bases from any one of SEQ ID NOS: 193-288; and/or
(b) the sense strand comprises at least 14 contiguous bases from any one of SEQ ID NOS: 289-384 and 385-480.

Embodiment 9. The RNAi agent of any one of embodiments 1-8, wherein the antisense strand comprises at least 14 contiguous bases that complement a sequence in an mRNA molecule that encodes GCK (SEQ ID NO: 481).

Embodiment 10. The RNAi agent of any one of embodiments 1-9, wherein the antisense strand and the sense strand form a duplex of at least 14 bases in length.

Embodiment 11. The RNAi agent of any one of embodiments 1-10, wherein the antisense strand and the sense strand form a duplex of between 15 and 30 bases in length.

Embodiment 12. The RNAi agent of any one of embodiments 1-11, wherein the antisense strand or a subsequence thereof differs by 0, 1, 2, 3, or 4 nucleotides from an equal length portion of any one of SEQ ID NOS: 1-96 and 193-288, wherein the subsequence is at least 14, 15, 16, 17, 18, 19, 20, 21, 22, or 23 bases in length.

Embodiment 13. The RNAi agent of embodiment 12, wherein the antisense strand comprises, in order, nucleotides 1-23 of any one of SEQ ID NOS: 1-96 and 193-288.

Embodiment 14. The RNAi agent of any one of embodiments 1-13, wherein the antisense strand comprises at least 14 contiguous bases from any one of SEQ ID NOS: 1-96 and 193-288.

Embodiment 15. The RNAi agent of any one of embodiments 1-14, wherein the antisense strand comprises, in order, nucleobases 1-14 of any one of SEQ ID NOS: 1-96 and 193-288.

Embodiment 16. The RNAi agent of any one of embodiments 1-15, wherein the antisense strand comprises at least 14 contiguous bases from any one of SEQ ID NOS: 8, 11, 14, 16, 35, 36, 38, 39, 41, 42, 52, 77, 84, 86, 87, 88, 89, 90, 93, 200, 203, 206, 208, 227, 228, 230, 231, 233, 234, 244, 269, 276, 278, 279, 280, 281, 282, and 285.

Embodiment 17. The RNAi agent of any one of embodiments 1-16, wherein the antisense strand comprises at least 14 contiguous bases from any one of SEQ ID NOS: 16, 35, 52, 84, 86, 89, 90, 208, 227, 244, 276, 278, 281, and 282.

Embodiment 18. The RNAi agent of any one of embodiments 1-17, wherein the sense strand or a subsequence thereof differs by 0, 1, 2, 3, or 4 nucleotides from an equal length portion of any one of SEQ ID NOS: 97-192, 289-384, and 385-480, wherein the subsequence is at least 14, 15, 16, 17, 18, 19, 20, or 21 bases in length.

Embodiment 19. The RNAi agent of embodiment 18, wherein the sense strand comprises, in order, nucleotides 1-21 of any one of SEQ ID NOS: 97-192, 289-384, and 385-480.

Embodiment 20. The RNAi agent of any one of embodiments 1-19, wherein the sense strand comprises at least 14 contiguous bases from any one of SEQ ID NOS: 97-192, 289-384, and 385-480.

Embodiment 21. The RNAi agent of any one of embodiments 1-20, wherein the sense strand comprises, in order, nucleobases 1-14 of any one of SEQ ID NOS: 97-192, 289-384, and 385-480.

Embodiment 22. The RNAi agent of embodiment 21, wherein the sense strand comprises at least 14 contiguous bases from any one of SEQ ID NOS:104, 107, 110, 112, 131, 132, 134, 135, 137, 138, 148, 173, 180, 182, 183, 184, 185, 186, 189, 296, 299, 302, 304, 323, 324, 326, 327, 329, 330, 340, 365, 372, 374, 375, 376, 377, 378, 381, 392, 395, 398, 400, 419, 420, 422, 423, 425 426, 436, 461, 468, 470, 471, 472, 473, 474, and 477.

Embodiment 23. The RNAi agent of embodiment 21 or 22, wherein the sense strand comprises at least 14 contiguous bases from any one of SEQ ID NOS: 112, 131, 148, 180, 182, 185, 186, 304, 323, 340, 372, 374, 377, 378, 400, 419, 436, 468, 470, 473, and 474.

Embodiment 24. The RNAi agent of any one of embodiments 1-23, wherein the antisense strand comprises at least 16 contiguous bases that complement a sequence in an mRNA molecule that encodes GCK (SEQ ID NO: 481).

Embodiment 25. The RNAi agent of any one of embodiments 1-24, wherein the antisense strand and the sense strand form a duplex of at least 16 bases in length.

Embodiment 26. The RNAi agent of any one of embodiments 1-25, wherein the antisense strand comprises at least 16 contiguous bases from any one of SEQ ID NOS: 1-96 and 193-288.

Embodiment 27. The RNAi agent of embodiment 26, wherein the antisense strand comprises at least 16 contiguous bases from any one of SEQ ID NOS: 8, 11, 14, 16, 35, 36, 38, 39, 41, 42, 52, 77, 84, 86, 87, 88, 89, 90, 93, 200, 203, 206, 208, 227, 228, 230, 231, 233, 234, 244, 269, 276, 278, 279, 280, 281, 282, and 285.

Embodiment 28. The RNAi agent of any one of embodiments 1-27, wherein the antisense strand comprises, in order, nucleobases 1-16, 1-17, 1-18, 1-19, 1-20, 1-21, 1-22, 1-23, 2-17, 2-18, 2-19, 2-20, 2-21, 2-22, 2-23, 3-18, 3-19, 3-20, 3-21, 3-22, 3-23, 4-19, 4-20, 4-21, 4-22, 4-23, 5-21, 5-22, 5-23, 6-20, 6-21, 6-22, 6-23, 7-22, 7-23, or 8-23 of any one of SEQ ID NOS: 1-96 and 193-288.

Embodiment 29. The RNAi agent of any one of embodiments 1-28, wherein the antisense strand comprises a nucleotide sequence according to any one of SEQ ID NOS: 1-96 and 193-288.

Embodiment 30. The RNAi agent of embodiment 29, wherein one or more nucleotides are modified.

Embodiment 31. The RNAi agent of embodiment 29, wherein the antisense strand comprises a nucleotide sequence according to any one of SEQ ID NOS: 8, 11, 14, 16, 35, 36, 38, 39, 41, 42, 52, 77, 84, 86, 87, 88, 89, 90, and 93.

Embodiment 32. The RNAi agent of embodiment 29 or 31, wherein the antisense strand comprises a nucleotide sequence according to any one of SEQ ID NOS: 16, 35, 52, 84, 86, 89, and 90.

Embodiment 33. The RNAi agent of embodiment 31 or 32, wherein one or more nucleotides are modified.

Embodiment 34. The RNAi agent of embodiment 30 or 33, wherein the antisense strand has a modification pattern according to nsNfsnnnNfnnnnnnnNfnNfnnnnnsnsn.

Embodiment 35. The RNAi agent of any one of embodiments 29-34, wherein the antisense strand comprises a modified nucleotide sequence according to any one of SEQ ID NOS: 193-288.

Embodiment 36. The RNAi agent of embodiment 35, wherein the antisense strand comprises a modified nucleotide sequence according to any one of SEQ ID NOS: 200, 203, 206, 208, 227, 228, 230, 231, 233, 234, 244, 269, 276, 278, 279, 280, 281, 282, and 285.

Embodiment 37. The RNAi agent of embodiment 35 or 36, wherein the antisense strand comprises a modified nucleotide sequence according to any one of SEQ ID NOS: 208, 227, 244, 276, 278, 281, and 282.

Embodiment 38. The RNAi agent of any one of embodiments 1-37, wherein the sense strand comprises at least 16 contiguous bases from any one of SEQ ID NOS: 97-192, 289-384, and 385-480.

Embodiment 39. The RNAi agent of embodiment 38, wherein the sense strand comprises at least 16 contiguous bases from any one of SEQ ID NOS: 104, 107, 110, 112, 131, 132, 134, 135, 137, 138, 148, 173, 180, 182, 183, 184, 185, 186, 189, 296, 299, 302, 304, 323, 324, 326, 327, 329, 330, 340, 365, 372, 374, 375, 376, 377, 378, 381, 392, 395, 398, 400, 419, 420, 422, 423, 425 426, 436, 461, 468, 470, 471, 472, 473, 474, and 477.

Embodiment 40. The RNAi agent of embodiment 38 or 39, wherein the sense strand comprises at least 16 contiguous bases from any one of SEQ ID NOS: 112, 131, 148, 180, 182, 185, 186, 304, 323, 340, 372, 374, 377, 378, 400, 419, 436, 468, 470, 473, and 474.

Embodiment 41. The RNAi agent of any one of embodiments 1-40, wherein the sense strand comprises, in order, nucleobases 1-16, 1-17, 1-18, 1-19, 1-20, 1-21, 1-22, 1-23, 2-17, 2-18, 2-19, 2-20, 2-21, 2-22, 2-23, 3-18, 3-19, 3-20, 3-21, 3-22, 3-23, 4-19, 4-20, 4-21, 4-22, 4-23, 5-21, 5-22, 5-23, 6-20, 6-21, 6-22, 6-23, 7-22, 7-23, or 8-23 of any one of SEQ ID NOS: 97-192, 289-384, and 385-480.

Embodiment 42. The RNAi agent of any one of embodiments 1-41, wherein the sense strand comprises a nucleobase sequence according to any one of SEQ ID NOS: 97-192, 289-384, and 385-480.

Embodiment 43. The RNAi agent of embodiment 42, wherein one or more nucleotides are modified.

Embodiment 44. The RNAi agent of embodiment 42, wherein the sense strand comprises a nucleobase sequence according to any one of SEQ ID NOS: 104, 107, 110, 112, 131, 132, 134, 135, 137, 138, 148, 173, 180, 182, 183, 184, 185, 186, and 189.

Embodiment 45. The RNAi agent of embodiment 42 or 44, wherein the sense strand comprises a nucleobase sequence according to any one of SEQ ID NOS: 112, 131, 148, 180, 182, 185, and 186.

Embodiment 46. The RNAi agent of embodiment 44 or 45, wherein one or more nucleotides are modified.

Embodiment 47. The RNAi agent of embodiment 43 or 46, wherein the sense strand has a modification pattern according to nsnsnnnnNfnNfNfNfnnnnnnnnsnsn.

Embodiment 48. The RNAi agent of embodiment 43 or 46, wherein the sense strand has a modification pattern according to nsnsnnnnNfnNfNfNfnnnnnnnnnn.

Embodiment 49. The RNAi agent of any one of embodiments 42-48, wherein the sense strand comprises a modified nucleotide sequence according to any one of SEQ ID NOS: 289-384 and 385-480.

Embodiment 50. The RNAi agent of embodiment 49, wherein the sense strand comprises a modified nucleotide sequence according to any one of SEQ ID NOS: 296, 299, 302, 304, 323, 324, 326, 327, 329, 330, 340, 365, 372, 374, 375, 376, 377, 378, 381, 392, 395, 398, 400, 419, 420, 422, 423, 425 426, 436, 461, 468, 470, 471, 472, 473, 474, and 477.

Embodiment 51. The RNAi agent of embodiment 49 or 50, wherein the sense strand comprises a modified nucleotide sequence according to any one of SEQ ID NOS: 304, 323, 340, 372, 374, 377, 378, 400, 419, 436, 468, 470, 473, and 474.

Embodiment 52. The RNAi agent of any one of embodiments 1-51, wherein:
(a) the sense strand comprises a nucleobase sequence according to SEQ ID NO: 97 and the antisense strand comprises a nucleobase sequence according to SEQ ID NO: 1;
(b) the sense strand comprises a nucleobase sequence according to SEQ ID NO: 98 and the antisense strand comprises a nucleobase sequence according to SEQ ID NO: 2;
(c) the sense strand comprises a nucleobase sequence according to SEQ ID NO: 99 and the antisense strand comprises a nucleobase sequence according to SEQ ID NO: 3;
(d) the sense strand comprises a nucleobase sequence according to SEQ ID NO: 100 and the antisense strand comprises a nucleobase sequence according to SEQ ID NO: 4;
(e) the sense strand comprises a nucleobase sequence according to SEQ ID NO: 101 and the antisense strand comprises a nucleobase sequence according to SEQ ID NO: 5;
(f) the sense strand comprises a nucleobase sequence according to SEQ ID NO: 102 and the antisense strand comprises a nucleobase sequence according to SEQ ID NO: 6;
(g) the sense strand comprises a nucleobase sequence according to SEQ ID NO: 103 and the antisense strand comprises a nucleobase sequence according to SEQ ID NO: 7;
(h) the sense strand comprises a nucleobase sequence according to SEQ ID NO: 104 and the antisense strand comprises a nucleobase sequence according to SEQ ID NO: 8;
(i) the sense strand comprises a nucleobase sequence according to SEQ ID NO: 105 and the antisense strand comprises a nucleobase sequence according to SEQ ID NO: 9;
(j) the sense strand comprises a nucleobase sequence according to SEQ ID NO: 106 and the antisense strand comprises a nucleobase sequence according to SEQ ID NO: 10;
(k) the sense strand comprises a nucleobase sequence according to SEQ ID NO: 107 and the antisense strand comprises a nucleobase sequence according to SEQ ID NO: 11;
(l) the sense strand comprises a nucleobase sequence according to SEQ ID NO: 108 and the antisense strand comprises a nucleobase sequence according to SEQ ID NO: 12;
(m) the sense strand comprises a nucleobase sequence according to SEQ ID NO: 109 and the antisense strand comprises a nucleobase sequence according to SEQ ID NO: 13;
(n) the sense strand comprises a nucleobase sequence according to SEQ ID NO: 110 and the antisense strand comprises a nucleobase sequence according to SEQ ID NO: 14;
(o) the sense strand comprises a nucleobase sequence according to SEQ ID NO: 111 and the antisense strand comprises a nucleobase sequence according to SEQ ID NO: 15;
(p) the sense strand comprises a nucleobase sequence according to SEQ ID NO: 112 and the antisense strand comprises a nucleobase sequence according to SEQ ID NO: 16;
(q) the sense strand comprises a nucleobase sequence according to SEQ ID NO: 113 and the antisense strand comprises a nucleobase sequence according to SEQ ID NO: 17;
(r) the sense strand comprises a nucleobase sequence according to SEQ ID NO: 114 and the antisense strand comprises a nucleobase sequence according to SEQ ID NO: 18;
(s) the sense strand comprises a nucleobase sequence according to SEQ ID NO: 115 and the antisense strand comprises a nucleobase sequence according to SEQ ID NO: 19;
(t) the sense strand comprises a nucleobase sequence according to SEQ ID NO: 116 and the antisense strand comprises a nucleobase sequence according to SEQ ID NO: 20;
(u) the sense strand comprises a nucleobase sequence according to SEQ ID NO: 117 and the antisense strand comprises a nucleobase sequence according to SEQ ID NO: 21;
(v) the sense strand comprises a nucleobase sequence according to SEQ ID NO: 118 and the antisense strand comprises a nucleobase sequence according to SEQ ID NO: 22;
(w) the sense strand comprises a nucleobase sequence according to SEQ ID NO: 119 and the antisense strand comprises a nucleobase sequence according to SEQ ID NO: 23;
(x) the sense strand comprises a nucleobase sequence according to SEQ ID NO: 120 and the antisense strand comprises a nucleobase sequence according to SEQ ID NO: 24;
(y) the sense strand comprises a nucleobase sequence according to SEQ ID NO: 121 and the antisense strand comprises a nucleobase sequence according to SEQ ID NO: 25;
(z) the sense strand comprises a nucleobase sequence according to SEQ ID NO: 122 and the antisense strand comprises a nucleobase sequence according to SEQ ID NO: 26;
(aa) the sense strand comprises a nucleobase sequence according to SEQ ID NO: 123 and the antisense strand comprises a nucleobase sequence according to SEQ ID NO: 27;
(bb) the sense strand comprises a nucleobase sequence according to SEQ ID NO: 124 and the antisense strand comprises a nucleobase sequence according to SEQ ID NO: 28;
(cc) the sense strand comprises a nucleobase sequence according to SEQ ID NO: 125 and the antisense strand comprises a nucleobase sequence according to SEQ ID NO: 29;
(dd) the sense strand comprises a nucleobase sequence according to SEQ ID NO: 126 and the antisense strand comprises a nucleobase sequence according to SEQ ID NO: 30;
(ee) the sense strand comprises a nucleobase sequence according to SEQ ID NO: 127 and the antisense strand comprises a nucleobase sequence according to SEQ ID NO: 31;
(ff) the sense strand comprises a nucleobase sequence according to SEQ ID NO: 128 and the antisense strand comprises a nucleobase sequence according to SEQ ID NO: 32;
(gg) the sense strand comprises a nucleobase sequence according to SEQ ID NO: 129 and the antisense strand comprises a nucleobase sequence according to SEQ ID NO: 33;
(hh) the sense strand comprises a nucleobase sequence according to SEQ ID NO: 130 and the antisense strand comprises a nucleobase sequence according to SEQ ID NO: 34;
(ii) the sense strand comprises a nucleobase sequence according to SEQ ID NO: 131 and the antisense strand comprises a nucleobase sequence according to SEQ ID NO: 35;
(jj) the sense strand comprises a nucleobase sequence according to SEQ ID NO: 132 and the antisense strand comprises a nucleobase sequence according to SEQ ID NO: 36;
(kk) the sense strand comprises a nucleobase sequence according to SEQ ID NO: 133 and the antisense strand comprises a nucleobase sequence according to SEQ ID NO: 37;
(ll) the sense strand comprises a nucleobase sequence according to SEQ ID NO: 134 and the antisense strand comprises a nucleobase sequence according to SEQ ID NO: 38;
(mm) the sense strand comprises a nucleobase sequence according to SEQ ID NO: 135 and the antisense strand comprises a nucleobase sequence according to SEQ ID NO: 39;
(nn) the sense strand comprises a nucleobase sequence according to SEQ ID NO: 136 and the antisense strand comprises a nucleobase sequence according to SEQ ID NO: 40;
(oo) the sense strand comprises a nucleobase sequence according to SEQ ID NO: 137 and the antisense strand comprises a nucleobase sequence according to SEQ ID NO: 41;
(pp) the sense strand comprises a nucleobase sequence according to SEQ ID NO: 138 and the antisense strand comprises a nucleobase sequence according to SEQ ID NO: 42;
(qq) the sense strand comprises a nucleobase sequence according to SEQ ID NO: 139 and the antisense strand comprises a nucleobase sequence according to SEQ ID NO: 43;
(rr) the sense strand comprises a nucleobase sequence according to SEQ ID NO: 140 and the antisense strand comprises a nucleobase sequence according to SEQ ID NO: 44;
(ss) the sense strand comprises a nucleobase sequence according to SEQ ID NO: 141 and the antisense strand comprises a nucleobase sequence according to SEQ ID NO: 45;
(tt) the sense strand comprises a nucleobase sequence according to SEQ ID NO: 142 and the antisense strand comprises a nucleobase sequence according to SEQ ID NO: 46;
(uu) the sense strand comprises a nucleobase sequence according to SEQ ID NO: 143 and the antisense strand comprises a nucleobase sequence according to SEQ ID NO: 47;
(vv) the sense strand comprises a nucleobase sequence according to SEQ ID NO: 144 and the antisense strand comprises a nucleobase sequence according to SEQ ID NO: 48;
(ww) the sense strand comprises a nucleobase sequence according to SEQ ID NO: 145 and the antisense strand comprises a nucleobase sequence according to SEQ ID NO: 49;
(xx) the sense strand comprises a nucleobase sequence according to SEQ ID NO: 146 and the antisense strand comprises a nucleobase sequence according to SEQ ID NO: 50;
(yy) the sense strand comprises a nucleobase sequence according to SEQ ID NO: 147 and the antisense strand comprises a nucleobase sequence according to SEQ ID NO: 51;
(zz) the sense strand comprises a nucleobase sequence according to SEQ ID NO: 148 and the antisense strand comprises a nucleobase sequence according to SEQ ID NO: 52;
(aaa) the sense strand comprises a nucleobase sequence according to SEQ ID NO: 149 and the antisense strand comprises a nucleobase sequence according to SEQ ID NO: 53;
(bbb) the sense strand comprises a nucleobase sequence according to SEQ ID NO: 150 and the antisense strand comprises a nucleobase sequence according to SEQ ID NO: 54;
(ccc) the sense strand comprises a nucleobase sequence according to SEQ ID NO: 151 and the antisense strand comprises a nucleobase sequence according to SEQ ID NO: 55;
(ddd) the sense strand comprises a nucleobase sequence according to SEQ ID NO: 152 and the antisense strand comprises a nucleobase sequence according to SEQ ID NO: 56;
(eee) the sense strand comprises a nucleobase sequence according to SEQ ID NO: 153 and the antisense strand comprises a nucleobase sequence according to SEQ ID NO: 57;
(fff) the sense strand comprises a nucleobase sequence according to SEQ ID NO: 154 and the antisense strand comprises a nucleobase sequence according to SEQ ID NO: 58;
(ggg) the sense strand comprises a nucleobase sequence according to SEQ ID NO: 155 and the antisense strand comprises a nucleobase sequence according to SEQ ID NO: 59;
(hhh) the sense strand comprises a nucleobase sequence according to SEQ ID NO: 156 and the antisense strand comprises a nucleobase sequence according to SEQ ID NO: 60;
(iii) the sense strand comprises a nucleobase sequence according to SEQ ID NO: 157 and the antisense strand comprises a nucleobase sequence according to SEQ ID NO: 61;
(jjj) the sense strand comprises a nucleobase sequence according to SEQ ID NO: 158 and the antisense strand comprises a nucleobase sequence according to SEQ ID NO: 62;
(kkk) the sense strand comprises a nucleobase sequence according to SEQ ID NO: 159 and the antisense strand comprises a nucleobase sequence according to SEQ ID NO: 63;
(lll) the sense strand comprises a nucleobase sequence according to SEQ ID NO: 160 and the antisense strand comprises a nucleobase sequence according to SEQ ID NO: 64;
(mmm) the sense strand comprises a nucleobase sequence according to SEQ ID NO: 161 and the antisense strand comprises a nucleobase sequence according to SEQ ID NO: 65;
(nnn) the sense strand comprises a nucleobase sequence according to SEQ ID NO: 162 and the antisense strand comprises a nucleobase sequence according to SEQ ID NO: 66;
(ooo) the sense strand comprises a nucleobase sequence according to SEQ ID NO: 163 and the antisense strand comprises a nucleobase sequence according to SEQ ID NO: 67;
(ppp) the sense strand comprises a nucleobase sequence according to SEQ ID NO: 164 and the antisense strand comprises a nucleobase sequence according to SEQ ID NO: 68;
(qqq) the sense strand comprises a nucleobase sequence according to SEQ ID NO: 165 and the antisense strand comprises a nucleobase sequence according to SEQ ID NO: 69;
(rrr) the sense strand comprises a nucleobase sequence according to SEQ ID NO: 166 and the antisense strand comprises a nucleobase sequence according to SEQ ID NO: 70;
(sss) the sense strand comprises a nucleobase sequence according to SEQ ID NO: 167 and the antisense strand comprises a nucleobase sequence according to SEQ ID NO: 71;
(ttt) the sense strand comprises a nucleobase sequence according to SEQ ID NO: 168 and the antisense strand comprises a nucleobase sequence according to SEQ ID NO: 72;
(uuu) the sense strand comprises a nucleobase sequence according to SEQ ID NO: 169 and the antisense strand comprises a nucleobase sequence according to SEQ ID NO: 73;
(vvv) the sense strand comprises a nucleobase sequence according to SEQ ID NO: 170 and the antisense strand comprises a nucleobase sequence according to SEQ ID NO: 74;
(www) the sense strand comprises a nucleobase sequence according to SEQ ID NO: 171 and the antisense strand comprises a nucleobase sequence according to SEQ ID NO: 75;
(xxx) the sense strand comprises a nucleobase sequence according to SEQ ID NO: 172 and the antisense strand comprises a nucleobase sequence according to SEQ ID NO: 76;
(yyy) the sense strand comprises a nucleobase sequence according to SEQ ID NO: 173 and the antisense strand comprises a nucleobase sequence according to SEQ ID NO: 77;
(zzz) the sense strand comprises a nucleobase sequence according to SEQ ID NO: 174 and the antisense strand comprises a nucleobase sequence according to SEQ ID NO: 78;
(aaaa) the sense strand comprises a nucleobase sequence according to SEQ ID NO: 175 and the antisense strand comprises a nucleobase sequence according to SEQ ID NO: 79;
(bbbb) the sense strand comprises a nucleobase sequence according to SEQ ID NO: 176 and the antisense strand comprises a nucleobase sequence according to SEQ ID NO: 80;
(cccc) the sense strand comprises a nucleobase sequence according to SEQ ID NO: 177 and the antisense strand comprises a nucleobase sequence according to SEQ ID NO: 81;
(dddd) the sense strand comprises a nucleobase sequence according to SEQ ID NO: 178 and the antisense strand comprises a nucleobase sequence according to SEQ ID NO: 82;
(eeee) the sense strand comprises a nucleobase sequence according to SEQ ID NO: 179 and the antisense strand comprises a nucleobase sequence according to SEQ ID NO: 83;
(ffff) the sense strand comprises a nucleobase sequence according to SEQ ID NO: 180 and the antisense strand comprises a nucleobase sequence according to SEQ ID NO: 84;
(gggg) the sense strand comprises a nucleobase sequence according to SEQ ID NO: 181 and the antisense strand comprises a nucleobase sequence according to SEQ ID NO: 85;
(hhhh) the sense strand comprises a nucleobase sequence according to SEQ ID NO: 182 and the antisense strand comprises a nucleobase sequence according to SEQ ID NO: 86;
(iiii) the sense strand comprises a nucleobase sequence according to SEQ ID NO: 183 and the antisense strand comprises a nucleobase sequence according to SEQ ID NO: 87;
(jjjj) the sense strand comprises a nucleobase sequence according to SEQ ID NO: 184 and the antisense strand comprises a nucleobase sequence according to SEQ ID NO: 88;
(kkkk) the sense strand comprises a nucleobase sequence according to SEQ ID NO: 185 and the antisense strand comprises a nucleobase sequence according to SEQ ID NO: 89;
(llll) the sense strand comprises a nucleobase sequence according to SEQ ID NO: 186 and the antisense strand comprises a nucleobase sequence according to SEQ ID NO: 90;
(mmmm) the sense strand comprises a nucleobase sequence according to SEQ ID NO: 187 and the antisense strand comprises a nucleobase sequence according to SEQ ID NO: 91;
(nnnn) the sense strand comprises a nucleobase sequence according to SEQ ID NO: 188 and the antisense strand comprises a nucleobase sequence according to SEQ ID NO: 92;
(oooo) the sense strand comprises a nucleobase sequence according to SEQ ID NO: 189 and the antisense strand comprises a nucleobase sequence according to SEQ ID NO: 93;
(pppp) the sense strand comprises a nucleobase sequence according to SEQ ID NO: 190 and the antisense strand comprises a nucleobase sequence according to SEQ ID NO: 94;
(qqqq) the sense strand comprises a nucleobase sequence according to SEQ ID NO: 191 and the antisense strand comprises a nucleobase sequence according to SEQ ID NO: 95; or
(rrrr) the sense strand comprises a nucleobase sequence according to SEQ ID NO: 192 and the antisense strand comprises a nucleobase sequence according to SEQ ID NO: 96.

Embodiment 53. The RNAi agent of embodiment 52, wherein:
(a) the sense strand comprises a nucleobase sequence according to SEQ ID NO: 112 and the antisense strand comprises a nucleobase sequence according to SEQ ID NO: 16;
(b) the sense strand comprises a nucleobase sequence according to SEQ ID NO: 131 and the antisense strand comprises a nucleobase sequence according to SEQ ID NO: 35;
(c) the sense strand comprises a nucleobase sequence according to SEQ ID NO: 148 and the antisense strand comprises a nucleobase sequence according to SEQ ID NO: 52;
(d) the sense strand comprises a nucleobase sequence according to SEQ ID NO: 180 and the antisense strand comprises a nucleobase sequence according to SEQ ID NO: 84;
(e) the sense strand comprises a nucleobase sequence according to SEQ ID NO: 182 and the antisense strand comprises a nucleobase sequence according to SEQ ID NO: 86;
(f) the sense strand comprises a nucleobase sequence according to SEQ ID NO: 185 and the antisense strand comprises a nucleobase sequence according to SEQ ID NO: 89; or
(g) the sense strand comprises a nucleobase sequence according to SEQ ID NO: 186 and the antisense strand comprises a nucleobase sequence according to SEQ ID NO: 90.

Embodiment 54. The RNAi agent of embodiment 52 or 53, wherein:
(a) the sense strand comprises a modified nucleotide sequence according to SEQ ID NO: 289 and the antisense strand comprises a modified nucleotide sequence according to SEQ ID NO: 193;
(b) the sense strand comprises a modified nucleotide sequence according to SEQ ID NO: 290 and the antisense strand comprises a modified nucleotide sequence according to SEQ ID NO: 194;
(c) the sense strand comprises a modified nucleotide sequence according to SEQ ID NO: 291 and the antisense strand comprises a modified nucleotide sequence according to SEQ ID NO: 195;
(d) the sense strand comprises a modified nucleotide sequence according to SEQ ID NO: 292 and the antisense strand comprises a modified nucleotide sequence according to SEQ ID NO: 196;
(e) the sense strand comprises a modified nucleotide sequence according to SEQ ID NO: 293 and the antisense strand comprises a modified nucleotide sequence according to SEQ ID NO: 197;
(f) the sense strand comprises a modified nucleotide sequence according to SEQ ID NO: 294 and the antisense strand comprises a modified nucleotide sequence according to SEQ ID NO: 198;
(g) the sense strand comprises a modified nucleotide sequence according to SEQ ID NO: 295 and the antisense strand comprises a modified nucleotide sequence according to SEQ ID NO: 199;
(h) the sense strand comprises a modified nucleotide sequence according to SEQ ID NO: 296 and the antisense strand comprises a modified nucleotide sequence according to SEQ ID NO: 200;
(i) the sense strand comprises a modified nucleotide sequence according to SEQ ID NO: 297 and the antisense strand comprises a modified nucleotide sequence according to SEQ ID NO: 201;
(j) the sense strand comprises a modified nucleotide sequence according to SEQ ID NO: 298 and the antisense strand comprises a modified nucleotide sequence according to SEQ ID NO: 202;
(k) the sense strand comprises a modified nucleotide sequence according to SEQ ID NO: 299 and the antisense strand comprises a modified nucleotide sequence according to SEQ ID NO: 203;
(l) the sense strand comprises a modified nucleotide sequence according to SEQ ID NO: 300 and the antisense strand comprises a modified nucleotide sequence according to SEQ ID NO: 204;
(m) the sense strand comprises a modified nucleotide sequence according to SEQ ID NO: 301 and the antisense strand comprises a modified nucleotide sequence according to SEQ ID NO: 205;
(n) the sense strand comprises a modified nucleotide sequence according to SEQ ID NO: 302 and the antisense strand comprises a modified nucleotide sequence according to SEQ ID NO: 206;
(o) the sense strand comprises a modified nucleotide sequence according to SEQ ID NO: 303 and the antisense strand comprises a modified nucleotide sequence according to SEQ ID NO: 207;
(p) the sense strand comprises a modified nucleotide sequence according to SEQ ID NO: 304 and the antisense strand comprises a modified nucleotide sequence according to SEQ ID NO: 208;
(q) the sense strand comprises a modified nucleotide sequence according to SEQ ID NO: 305 and the antisense strand comprises a modified nucleotide sequence according to SEQ ID NO: 209;
(r) the sense strand comprises a modified nucleotide sequence according to SEQ ID NO: 306 and the antisense strand comprises a modified nucleotide sequence according to SEQ ID NO: 210;
(s) the sense strand comprises a modified nucleotide sequence according to SEQ ID NO: 307 and the antisense strand comprises a modified nucleotide sequence according to SEQ ID NO: 211;
(t) the sense strand comprises a modified nucleotide sequence according to SEQ ID NO: 308 and the antisense strand comprises a modified nucleotide sequence according to SEQ ID NO: 212;
(u) the sense strand comprises a modified nucleotide sequence according to SEQ ID NO: 309 and the antisense strand comprises a modified nucleotide sequence according to SEQ ID NO: 213;
(v) the sense strand comprises a modified nucleotide sequence according to SEQ ID NO: 310 and the antisense strand comprises a modified nucleotide sequence according to SEQ ID NO: 214;
(w) the sense strand comprises a modified nucleotide sequence according to SEQ ID NO: 311 and the antisense strand comprises a modified nucleotide sequence according to SEQ ID NO: 215;
(x) the sense strand comprises a modified nucleotide sequence according to SEQ ID NO: 312 and the antisense strand comprises a modified nucleotide sequence according to SEQ ID NO: 216;
(y) the sense strand comprises a modified nucleotide sequence according to SEQ ID NO: 313 and the antisense strand comprises a modified nucleotide sequence according to SEQ ID NO: 217;
(z) the sense strand comprises a modified nucleotide sequence according to SEQ ID NO: 314 and the antisense strand comprises a modified nucleotide sequence according to SEQ ID NO: 218;
(aa) the sense strand comprises a modified nucleotide sequence according to SEQ ID NO: 315 and the antisense strand comprises a modified nucleotide sequence according to SEQ ID NO: 219;
(bb) the sense strand comprises a modified nucleotide sequence according to SEQ ID NO: 316 and the antisense strand comprises a modified nucleotide sequence according to SEQ ID NO: 220;
(cc) the sense strand comprises a modified nucleotide sequence according to SEQ ID NO: 317 and the antisense strand comprises a modified nucleotide sequence according to SEQ ID NO: 221;
(dd) the sense strand comprises a modified nucleotide sequence according to SEQ ID NO: 318 and the antisense strand comprises a modified nucleotide sequence according to SEQ ID NO: 222;
(ee) the sense strand comprises a modified nucleotide sequence according to SEQ ID NO: 319 and the antisense strand comprises a modified nucleotide sequence according to SEQ ID NO: 223;
(ff) the sense strand comprises a modified nucleotide sequence according to SEQ ID NO: 320 and the antisense strand comprises a modified nucleotide sequence according to SEQ ID NO: 224;
(gg) the sense strand comprises a modified nucleotide sequence according to SEQ ID NO: 321 and the antisense strand comprises a modified nucleotide sequence according to SEQ ID NO: 225;
(hh) the sense strand comprises a modified nucleotide sequence according to SEQ ID NO: 322 and the antisense strand comprises a modified nucleotide sequence according to SEQ ID NO: 226;
(ii) the sense strand comprises a modified nucleotide sequence according to SEQ ID NO: 323 and the antisense strand comprises a modified nucleotide sequence according to SEQ ID NO: 227;
(jj) the sense strand comprises a modified nucleotide sequence according to SEQ ID NO: 324 and the antisense strand comprises a modified nucleotide sequence according to SEQ ID NO: 228;
(kk) the sense strand comprises a modified nucleotide sequence according to SEQ ID NO: 325 and the antisense strand comprises a modified nucleotide sequence according to SEQ ID NO: 229;
(ll) the sense strand comprises a modified nucleotide sequence according to SEQ ID NO: 326 and the antisense strand comprises a modified nucleotide sequence according to SEQ ID NO: 230;
(mm) the sense strand comprises a modified nucleotide sequence according to SEQ ID NO: 327 and the antisense strand comprises a modified nucleotide sequence according to SEQ ID NO: 231;
(nn) the sense strand comprises a modified nucleotide sequence according to SEQ ID NO: 328 and the antisense strand comprises a modified nucleotide sequence according to SEQ ID NO: 232;
(oo) the sense strand comprises a modified nucleotide sequence according to SEQ ID NO: 329 and the antisense strand comprises a modified nucleotide sequence according to SEQ ID NO: 233;
(pp) the sense strand comprises a modified nucleotide sequence according to SEQ ID NO: 330 and the antisense strand comprises a modified nucleotide sequence according to SEQ ID NO: 234;
(qq) the sense strand comprises a modified nucleotide sequence according to SEQ ID NO: 331 and the antisense strand comprises a modified nucleotide sequence according to SEQ ID NO: 235;
(rr) the sense strand comprises a modified nucleotide sequence according to SEQ ID NO: 332 and the antisense strand comprises a modified nucleotide sequence according to SEQ ID NO: 236;
(ss) the sense strand comprises a modified nucleotide sequence according to SEQ ID NO: 333 and the antisense strand comprises a modified nucleotide sequence according to SEQ ID NO: 237;
(tt) the sense strand comprises a modified nucleotide sequence according to SEQ ID NO: 334 and the antisense strand comprises a modified nucleotide sequence according to SEQ ID NO: 238;
(uu) the sense strand comprises a modified nucleotide sequence according to SEQ ID NO: 335 and the antisense strand comprises a modified nucleotide sequence according to SEQ ID NO: 239;
(vv) the sense strand comprises a modified nucleotide sequence according to SEQ ID NO: 336 and the antisense strand comprises a modified nucleotide sequence according to SEQ ID NO: 240;
(ww) the sense strand comprises a modified nucleotide sequence according to SEQ ID NO: 337 and the antisense strand comprises a modified nucleotide sequence according to SEQ ID NO: 241;
(xx) the sense strand comprises a modified nucleotide sequence according to SEQ ID NO: 338 and the antisense strand comprises a modified nucleotide sequence according to SEQ ID NO: 242;
(yy) the sense strand comprises a modified nucleotide sequence according to SEQ ID NO: 339 and the antisense strand comprises a modified nucleotide sequence according to SEQ ID NO: 243;
(zz) the sense strand comprises a modified nucleotide sequence according to SEQ ID NO: 340 and the antisense strand comprises a modified nucleotide sequence according to SEQ ID NO: 244;
(aaa) the sense strand comprises a modified nucleotide sequence according to SEQ ID NO: 341 and the antisense strand comprises a modified nucleotide sequence according to SEQ ID NO: 245;
(bbb) the sense strand comprises a modified nucleotide sequence according to SEQ ID NO: 342 and the antisense strand comprises a modified nucleotide sequence according to SEQ ID NO: 246;
(ccc) the sense strand comprises a modified nucleotide sequence according to SEQ ID NO: 343 and the antisense strand comprises a modified nucleotide sequence according to SEQ ID NO: 247;
(ddd) the sense strand comprises a modified nucleotide sequence according to SEQ ID NO: 344 and the antisense strand comprises a modified nucleotide sequence according to SEQ ID NO: 248;
(eee) the sense strand comprises a modified nucleotide sequence according to SEQ ID NO: 345 and the antisense strand comprises a modified nucleotide sequence according to SEQ ID NO: 249;
(fff) the sense strand comprises a modified nucleotide sequence according to SEQ ID NO: 346 and the antisense strand comprises a modified nucleotide sequence according to SEQ ID NO: 250;
(ggg) the sense strand comprises a modified nucleotide sequence according to SEQ ID NO: 347 and the antisense strand comprises a modified nucleotide sequence according to SEQ ID NO: 251;
(hhh) the sense strand comprises a modified nucleotide sequence according to SEQ ID NO: 348 and the antisense strand comprises a modified nucleotide sequence according to SEQ ID NO: 252;
(iii) the sense strand comprises a modified nucleotide sequence according to SEQ ID NO: 349 and the antisense strand comprises a modified nucleotide sequence according to SEQ ID NO: 253;
(jjj) the sense strand comprises a modified nucleotide sequence according to SEQ ID NO: 350 and the antisense strand comprises a modified nucleotide sequence according to SEQ ID NO: 254;
(kkk) the sense strand comprises a modified nucleotide sequence according to SEQ ID NO: 351 and the antisense strand comprises a modified nucleotide sequence according to SEQ ID NO: 255;
(lll) the sense strand comprises a modified nucleotide sequence according to SEQ ID NO: 352 and the antisense strand comprises a modified nucleotide sequence according to SEQ ID NO: 256;
(mmm) the sense strand comprises a modified nucleotide sequence according to SEQ ID NO: 353 and the antisense strand comprises a modified nucleotide sequence according to SEQ ID NO: 257;
(nnn) the sense strand comprises a modified nucleotide sequence according to SEQ ID NO: 354 and the antisense strand comprises a modified nucleotide sequence according to SEQ ID NO: 258;
(ooo) the sense strand comprises a modified nucleotide sequence according to SEQ ID NO: 355 and the antisense strand comprises a modified nucleotide sequence according to SEQ ID NO: 259;
(ppp) the sense strand comprises a modified nucleotide sequence according to SEQ ID NO: 356 and the antisense strand comprises a modified nucleotide sequence according to SEQ ID NO: 260;
(qqq) the sense strand comprises a modified nucleotide sequence according to SEQ ID NO: 357 and the antisense strand comprises a modified nucleotide sequence according to SEQ ID NO: 261;
(rrr) the sense strand comprises a modified nucleotide sequence according to SEQ ID NO: 358 and the antisense strand comprises a modified nucleotide sequence according to SEQ ID NO: 262;
(sss) the sense strand comprises a modified nucleotide sequence according to SEQ ID NO: 359 and the antisense strand comprises a modified nucleotide sequence according to SEQ ID NO: 263;
(ttt) the sense strand comprises a modified nucleotide sequence according to SEQ ID NO: 360 and the antisense strand comprises a modified nucleotide sequence according to SEQ ID NO: 264;
(uuu) the sense strand comprises a modified nucleotide sequence according to SEQ ID NO: 361 and the antisense strand comprises a modified nucleotide sequence according to SEQ ID NO: 265;
(vvv) the sense strand comprises a modified nucleotide sequence according to SEQ ID NO: 362 and the antisense strand comprises a modified nucleotide sequence according to SEQ ID NO: 266;
(www) the sense strand comprises a modified nucleotide sequence according to SEQ ID NO: 363 and the antisense strand comprises a modified nucleotide sequence according to SEQ ID NO: 267;
(xxx) the sense strand comprises a modified nucleotide sequence according to SEQ ID NO: 364 and the antisense strand comprises a modified nucleotide sequence according to SEQ ID NO: 268;
(yyy) the sense strand comprises a modified nucleotide sequence according to SEQ ID NO: 365 and the antisense strand comprises a modified nucleotide sequence according to SEQ ID NO: 269;
(zzz) the sense strand comprises a modified nucleotide sequence according to SEQ ID NO: 366 and the antisense strand comprises a modified nucleotide sequence according to SEQ ID NO: 270;
(aaaa) the sense strand comprises a modified nucleotide sequence according to SEQ ID NO: 367 and the antisense strand comprises a modified nucleotide sequence according to SEQ ID NO: 271;
(bbbb) the sense strand comprises a modified nucleotide sequence according to SEQ ID NO: 368 and the antisense strand comprises a modified nucleotide sequence according to SEQ ID NO: 272;
(cccc) the sense strand comprises a modified nucleotide sequence according to SEQ ID NO: 369 and the antisense strand comprises a modified nucleotide sequence according to SEQ ID NO: 273;
(dddd) the sense strand comprises a modified nucleotide sequence according to SEQ ID NO: 370 and the antisense strand comprises a modified nucleotide sequence according to SEQ ID NO: 274;
(eeee) the sense strand comprises a modified nucleotide sequence according to SEQ ID NO: 371 and the antisense strand comprises a modified nucleotide sequence according to SEQ ID NO: 275;
(ffff) the sense strand comprises a modified nucleotide sequence according to SEQ ID NO: 372 and the antisense strand comprises a modified nucleotide sequence according to SEQ ID NO: 276;
(gggg) the sense strand comprises a modified nucleotide sequence according to SEQ ID NO: 373 and the antisense strand comprises a modified nucleotide sequence according to SEQ ID NO: 277;
(hhhh) the sense strand comprises a modified nucleotide sequence according to SEQ ID NO: 374 and the antisense strand comprises a modified nucleotide sequence according to SEQ ID NO: 278;
(iiii) the sense strand comprises a modified nucleotide sequence according to SEQ ID NO: 375 and the antisense strand comprises a modified nucleotide sequence according to SEQ ID NO: 279;
(jjjj) the sense strand comprises a modified nucleotide sequence according to SEQ ID NO: 376 and the antisense strand comprises a modified nucleotide sequence according to SEQ ID NO: 280;
(kkkk) the sense strand comprises a modified nucleotide sequence according to SEQ ID NO: 377 and the antisense strand comprises a modified nucleotide sequence according to SEQ ID NO: 281;
(llll) the sense strand comprises a modified nucleotide sequence according to SEQ ID NO: 378 and the antisense strand comprises a modified nucleotide sequence according to SEQ ID NO: 282;
(mmmm) the sense strand comprises a modified nucleotide sequence according to SEQ ID NO: 379 and the antisense strand comprises a modified nucleotide sequence according to SEQ ID NO: 283;
(nnnn) the sense strand comprises a modified nucleotide sequence according to SEQ ID NO: 380 and the antisense strand comprises a modified nucleotide sequence according to SEQ ID NO: 284;
(oooo) the sense strand comprises a modified nucleotide sequence according to SEQ ID NO: 381 and the antisense strand comprises a modified nucleotide sequence according to SEQ ID NO: 285;
(pppp) the sense strand comprises a modified nucleotide sequence according to SEQ ID NO: 382 and the antisense strand comprises a modified nucleotide sequence according to SEQ ID NO: 286;
(qqqq) the sense strand comprises a modified nucleotide sequence according to SEQ ID NO: 383 and the antisense strand comprises a modified nucleotide sequence according to SEQ ID NO: 287;
(rrrr) the sense strand comprises a modified nucleotide sequence according to SEQ ID NO: 384 and the antisense strand comprises a modified nucleotide sequence according to SEQ ID NO: 288;
(ssss) the sense strand comprises a modified nucleotide sequence according to SEQ ID NO: 385 and the antisense strand comprises a modified nucleotide sequence according to SEQ ID NO: 193;
(tttt) the sense strand comprises a modified nucleotide sequence according to SEQ ID NO: 386 and the antisense strand comprises a modified nucleotide sequence according to SEQ ID NO: 194;
(uuuu) the sense strand comprises a modified nucleotide sequence according to SEQ ID NO: 387 and the antisense strand comprises a modified nucleotide sequence according to SEQ ID NO: 195;
(vvvv) the sense strand comprises a modified nucleotide sequence according to SEQ ID NO: 388 and the antisense strand comprises a modified nucleotide sequence according to SEQ ID NO: 196;
(wwww) the sense strand comprises a modified nucleotide sequence according to SEQ ID NO: 389 and the antisense strand comprises a modified nucleotide sequence according to SEQ ID NO: 197;
(xxxx) the sense strand comprises a modified nucleotide sequence according to SEQ ID NO: 390 and the antisense strand comprises a modified nucleotide sequence according to SEQ ID NO: 198;
(yyyy) the sense strand comprises a modified nucleotide sequence according to SEQ ID NO: 391 and the antisense strand comprises a modified nucleotide sequence according to SEQ ID NO: 199;
(zzzz) the sense strand comprises a modified nucleotide sequence according to SEQ ID NO: 392 and the antisense strand comprises a modified nucleotide sequence according to SEQ ID NO: 200;
(aaaaa) the sense strand comprises a modified nucleotide sequence according to SEQ ID NO: 393 and the antisense strand comprises a modified nucleotide sequence according to SEQ ID NO: 201;
(bbbbb) the sense strand comprises a modified nucleotide sequence according to SEQ ID NO: 394 and the antisense strand comprises a modified nucleotide sequence according to SEQ ID NO: 202;
(ccccc) the sense strand comprises a modified nucleotide sequence according to SEQ ID NO: 395 and the antisense strand comprises a modified nucleotide sequence according to SEQ ID NO: 203;
(ddddd) the sense strand comprises a modified nucleotide sequence according to SEQ ID NO: 396 and the antisense strand comprises a modified nucleotide sequence according to SEQ ID NO: 204;
(eeeee) the sense strand comprises a modified nucleotide sequence according to SEQ ID NO: 397 and the antisense strand comprises a modified nucleotide sequence according to SEQ ID NO: 205;
(fffff) the sense strand comprises a modified nucleotide sequence according to SEQ ID NO: 398 and the antisense strand comprises a modified nucleotide sequence according to SEQ ID NO: 206;
(ggggg) the sense strand comprises a modified nucleotide sequence according to SEQ ID NO: 399 and the antisense strand comprises a modified nucleotide sequence according to SEQ ID NO: 207;
(hhhhh) the sense strand comprises a modified nucleotide sequence according to SEQ ID NO: 400 and the antisense strand comprises a modified nucleotide sequence according to SEQ ID NO: 208;
(iiiii) the sense strand comprises a modified nucleotide sequence according to SEQ ID NO: 401 and the antisense strand comprises a modified nucleotide sequence according to SEQ ID NO: 209;
(jjjjj) the sense strand comprises a modified nucleotide sequence according to SEQ ID NO: 402 and the antisense strand comprises a modified nucleotide sequence according to SEQ ID NO: 210;
(kkkkk) the sense strand comprises a modified nucleotide sequence according to SEQ ID NO: 403 and the antisense strand comprises a modified nucleotide sequence according to SEQ ID NO: 211;
(lllll) the sense strand comprises a modified nucleotide sequence according to SEQ ID NO: 404 and the antisense strand comprises a modified nucleotide sequence according to SEQ ID NO: 212;
(mmmmm) the sense strand comprises a modified nucleotide sequence according to SEQ ID NO: 405 and the antisense strand comprises a modified nucleotide sequence according to SEQ ID NO: 213;
(nnnnn) the sense strand comprises a modified nucleotide sequence according to SEQ ID NO: 406 and the antisense strand comprises a modified nucleotide sequence according to SEQ ID NO: 214;
(ooooo) the sense strand comprises a modified nucleotide sequence according to SEQ ID NO: 407 and the antisense strand comprises a modified nucleotide sequence according to SEQ ID NO: 215;
(ppppp) the sense strand comprises a modified nucleotide sequence according to SEQ ID NO: 408 and the antisense strand comprises a modified nucleotide sequence according to SEQ ID NO: 216;
(qqqqq) the sense strand comprises a modified nucleotide sequence according to SEQ ID NO: 409 and the antisense strand comprises a modified nucleotide sequence according to SEQ ID NO: 217;
(rrrrr) the sense strand comprises a modified nucleotide sequence according to SEQ ID NO: 410 and the antisense strand comprises a modified nucleotide sequence according to SEQ ID NO: 218;
(sssss) the sense strand comprises a modified nucleotide sequence according to SEQ ID NO: 411 and the antisense strand comprises a modified nucleotide sequence according to SEQ ID NO: 219;
(ttttt) the sense strand comprises a modified nucleotide sequence according to SEQ ID NO: 412 and the antisense strand comprises a modified nucleotide sequence according to SEQ ID NO: 220;
(uuuuu) the sense strand comprises a modified nucleotide sequence according to SEQ ID NO: 413 and the antisense strand comprises a modified nucleotide sequence according to SEQ ID NO: 221;
(vvvvv) the sense strand comprises a modified nucleotide sequence according to SEQ ID NO: 414 and the antisense strand comprises a modified nucleotide sequence according to SEQ ID NO: 222;
(wwwww) the sense strand comprises a modified nucleotide sequence according to SEQ ID NO: 415 and the antisense strand comprises a modified nucleotide sequence according to SEQ ID NO: 223;
(xxxxx) the sense strand comprises a modified nucleotide sequence according to SEQ ID NO: 416 and the antisense strand comprises a modified nucleotide sequence according to SEQ ID NO: 224;
(yyyyy) the sense strand comprises a modified nucleotide sequence according to SEQ ID NO: 417 and the antisense strand comprises a modified nucleotide sequence according to SEQ ID NO: 225;
(zzzzz) the sense strand comprises a modified nucleotide sequence according to SEQ ID NO: 418 and the antisense strand comprises a modified nucleotide sequence according to SEQ ID NO: 226;
(aaaaaa) the sense strand comprises a modified nucleotide sequence according to SEQ ID NO: 419 and the antisense strand comprises a modified nucleotide sequence according to SEQ ID NO: 227;
(bbbbbb) the sense strand comprises a modified nucleotide sequence according to SEQ ID NO: 420 and the antisense strand comprises a modified nucleotide sequence according to SEQ ID NO: 228;
(cccccc) the sense strand comprises a modified nucleotide sequence according to SEQ ID NO: 421 and the antisense strand comprises a modified nucleotide sequence according to SEQ ID NO: 229;
(dddddd) the sense strand comprises a modified nucleotide sequence according to SEQ ID NO: 422 and the antisense strand comprises a modified nucleotide sequence according to SEQ ID NO: 230;
(eeeeee) the sense strand comprises a modified nucleotide sequence according to SEQ ID NO: 423 and the antisense strand comprises a modified nucleotide sequence according to SEQ ID NO: 231;
(ffffff) the sense strand comprises a modified nucleotide sequence according to SEQ ID NO: 424 and the antisense strand comprises a modified nucleotide sequence according to SEQ ID NO: 232;
(gggggg) the sense strand comprises a modified nucleotide sequence according to SEQ ID NO: 425 and the antisense strand comprises a modified nucleotide sequence according to SEQ ID NO: 233;
(hhhhhh) the sense strand comprises a modified nucleotide sequence according to SEQ ID NO: 426 and the antisense strand comprises a modified nucleotide sequence according to SEQ ID NO: 234;
(iiiiii) the sense strand comprises a modified nucleotide sequence according to SEQ ID NO: 427 and the antisense strand comprises a modified nucleotide sequence according to SEQ ID NO: 235;
(jjjjjj) the sense strand comprises a modified nucleotide sequence according to SEQ ID NO: 428 and the antisense strand comprises a modified nucleotide sequence according to SEQ ID NO: 236;
(kkkkkk) the sense strand comprises a modified nucleotide sequence according to SEQ ID NO: 429 and the antisense strand comprises a modified nucleotide sequence according to SEQ ID NO: 237;
(llllll) the sense strand comprises a modified nucleotide sequence according to SEQ ID NO: 430 and the antisense strand comprises a modified nucleotide sequence according to SEQ ID NO: 238;
(mmmmmm) the sense strand comprises a modified nucleotide sequence according to SEQ ID NO: 431 and the antisense strand comprises a modified nucleotide sequence according to SEQ ID NO: 239;
(nnnnnn) the sense strand comprises a modified nucleotide sequence according to SEQ ID NO: 432 and the antisense strand comprises a modified nucleotide sequence according to SEQ ID NO: 240;
(oooooo) the sense strand comprises a modified nucleotide sequence according to SEQ ID NO: 433 and the antisense strand comprises a modified nucleotide sequence according to SEQ ID NO: 241;
(pppppp) the sense strand comprises a modified nucleotide sequence according to SEQ ID NO: 434 and the antisense strand comprises a modified nucleotide sequence according to SEQ ID NO: 242;
(qqqqqq) the sense strand comprises a modified nucleotide sequence according to SEQ ID NO: 435 and the antisense strand comprises a modified nucleotide sequence according to SEQ ID NO: 243;
(rrrrrr) the sense strand comprises a modified nucleotide sequence according to SEQ ID NO: 436 and the antisense strand comprises a modified nucleotide sequence according to SEQ ID NO: 244;
(ssssss) the sense strand comprises a modified nucleotide sequence according to SEQ ID NO: 437 and the antisense strand comprises a modified nucleotide sequence according to SEQ ID NO: 245;
(tttttt) the sense strand comprises a modified nucleotide sequence according to SEQ ID NO: 438 and the antisense strand comprises a modified nucleotide sequence according to SEQ ID NO: 246;
(uuuuuu) the sense strand comprises a modified nucleotide sequence according to SEQ ID NO: 439 and the antisense strand comprises a modified nucleotide sequence according to SEQ ID NO: 247;
(vvvvvv) the sense strand comprises a modified nucleotide sequence according to SEQ ID NO: 440 and the antisense strand comprises a modified nucleotide sequence according to SEQ ID NO: 248;
(wwwwww) the sense strand comprises a modified nucleotide sequence according to SEQ ID NO: 441 and the antisense strand comprises a modified nucleotide sequence according to SEQ ID NO: 249;
(xxxxxx) the sense strand comprises a modified nucleotide sequence according to SEQ ID NO: 442 and the antisense strand comprises a modified nucleotide sequence according to SEQ ID NO: 250;
(yyyyyy) the sense strand comprises a modified nucleotide sequence according to SEQ ID NO: 443 and the antisense strand comprises a modified nucleotide sequence according to SEQ ID NO: 251;
(zzzzzz) the sense strand comprises a modified nucleotide sequence according to SEQ ID NO: 444 and the antisense strand comprises a modified nucleotide sequence according to SEQ ID NO: 252;
(aaaaaaa) the sense strand comprises a modified nucleotide sequence according to SEQ ID NO: 445 and the antisense strand comprises a modified nucleotide sequence according to SEQ ID NO: 253;
(bbbbbbb) the sense strand comprises a modified nucleotide sequence according to SEQ ID NO: 446 and the antisense strand comprises a modified nucleotide sequence according to SEQ ID NO: 254;
(ccccccc) the sense strand comprises a modified nucleotide sequence according to SEQ ID NO: 447 and the antisense strand comprises a modified nucleotide sequence according to SEQ ID NO: 255;
(ddddddd) the sense strand comprises a modified nucleotide sequence according to SEQ ID NO: 448 and the antisense strand comprises a modified nucleotide sequence according to SEQ ID NO: 256;
(eeeeeee) the sense strand comprises a modified nucleotide sequence according to SEQ ID NO: 449 and the antisense strand comprises a modified nucleotide sequence according to SEQ ID NO: 257;
(fffffff) the sense strand comprises a modified nucleotide sequence according to SEQ ID NO: 450 and the antisense strand comprises a modified nucleotide sequence according to SEQ ID NO: 258;
(ggggggg) the sense strand comprises a modified nucleotide sequence according to SEQ ID NO: 451 and the antisense strand comprises a modified nucleotide sequence according to SEQ ID NO: 259;
(hhhhhhh) the sense strand comprises a modified nucleotide sequence according to SEQ ID NO: 452 and the antisense strand comprises a modified nucleotide sequence according to SEQ ID NO: 260;
(iiiiii) the sense strand comprises a modified nucleotide sequence according to SEQ ID NO: 453 and the antisense strand comprises a modified nucleotide sequence according to SEQ ID NO: 261;
(jjjjjjj) the sense strand comprises a modified nucleotide sequence according to SEQ ID NO: 454 and the antisense strand comprises a modified nucleotide sequence according to SEQ ID NO: 262;
(kkkkkkk) the sense strand comprises a modified nucleotide sequence according to SEQ ID NO: 455 and the antisense strand comprises a modified nucleotide sequence according to SEQ ID NO: 263;
(lllllll) the sense strand comprises a modified nucleotide sequence according to SEQ ID NO: 456 and the antisense strand comprises a modified nucleotide sequence according to SEQ ID NO: 264;
(mmmmmmm) the sense strand comprises a modified nucleotide sequence according to SEQ ID NO: 457 and the antisense strand comprises a modified nucleotide sequence according to SEQ ID NO: 265;
(nnnnnnn) the sense strand comprises a modified nucleotide sequence according to SEQ ID NO: 458 and the antisense strand comprises a modified nucleotide sequence according to SEQ ID NO: 266;
(ooooooo) the sense strand comprises a modified nucleotide sequence according to SEQ ID NO: 459 and the antisense strand comprises a modified nucleotide sequence according to SEQ ID NO: 267;
(ppppppp) the sense strand comprises a modified nucleotide sequence according to SEQ ID NO: 460 and the antisense strand comprises a modified nucleotide sequence according to SEQ ID NO: 268;
(qqqqqqq) the sense strand comprises a modified nucleotide sequence according to SEQ ID NO: 461 and the antisense strand comprises a modified nucleotide sequence according to SEQ ID NO: 269;
(rrrrrrrr) the sense strand comprises a modified nucleotide sequence according to SEQ ID NO: 462 and the antisense strand comprises a modified nucleotide sequence according to SEQ ID NO: 270;
(sssssss) the sense strand comprises a modified nucleotide sequence according to SEQ ID NO: 463 and the antisense strand comprises a modified nucleotide sequence according to SEQ ID NO: 271;
(ttttttt) the sense strand comprises a modified nucleotide sequence according to SEQ ID NO: 464 and the antisense strand comprises a modified nucleotide sequence according to SEQ ID NO: 272;
(uuuuuuu) the sense strand comprises a modified nucleotide sequence according to SEQ ID NO: 465 and the antisense strand comprises a modified nucleotide sequence according to SEQ ID NO: 273;
(vvvvvvv) the sense strand comprises a modified nucleotide sequence according to SEQ ID NO: 466 and the antisense strand comprises a modified nucleotide sequence according to SEQ ID NO: 274;
(wwwwwww) the sense strand comprises a modified nucleotide sequence according to SEQ ID NO: 467 and the antisense strand comprises a modified nucleotide sequence according to SEQ ID NO: 275;
(xxxxxxx) the sense strand comprises a modified nucleotide sequence according to SEQ ID NO: 468 and the antisense strand comprises a modified nucleotide sequence according to SEQ ID NO: 276;
(yyyyyyy) the sense strand comprises a modified nucleotide sequence according to SEQ ID NO: 469 and the antisense strand comprises a modified nucleotide sequence according to SEQ ID NO: 277;
(zzzzzzz) the sense strand comprises a modified nucleotide sequence according to SEQ ID NO: 470 and the antisense strand comprises a modified nucleotide sequence according to SEQ ID NO: 278;
(aaaaaaaa) the sense strand comprises a modified nucleotide sequence according to SEQ ID NO: 471 and the antisense strand comprises a modified nucleotide sequence according to SEQ ID NO: 279;
(bbbbbbbb) the sense strand comprises a modified nucleotide sequence according to SEQ ID NO: 472 and the antisense strand comprises a modified nucleotide sequence according to SEQ ID NO: 280;
(cccccccc) the sense strand comprises a modified nucleotide sequence according to SEQ ID NO: 473 and the antisense strand comprises a modified nucleotide sequence according to SEQ ID NO: 281;
(dddddddd) the sense strand comprises a modified nucleotide sequence according to SEQ ID NO: 474 and the antisense strand comprises a modified nucleotide sequence according to SEQ ID NO: 282;
(eeeeeeee) the sense strand comprises a modified nucleotide sequence according to SEQ ID NO: 475 and the antisense strand comprises a modified nucleotide sequence according to SEQ ID NO: 283;
(ffffffff) the sense strand comprises a modified nucleotide sequence according to SEQ ID NO: 476 and the antisense strand comprises a modified nucleotide sequence according to SEQ ID NO: 284;
(gggggggg) the sense strand comprises a modified nucleotide sequence according to SEQ ID NO: 477 and the antisense strand comprises a modified nucleotide sequence according to SEQ ID NO: 285;
(hhhhhhhh) the sense strand comprises a modified nucleotide sequence according to SEQ ID NO: 478 and the antisense strand comprises a modified nucleotide sequence according to SEQ ID NO: 286;
(iiiiiii) the sense strand comprises a modified nucleotide sequence according to SEQ ID NO: 479 and the antisense strand comprises a modified nucleotide sequence according to SEQ ID NO: 287; or
(jjjjjjjj) the sense strand comprises a modified nucleotide sequence according to SEQ ID NO: 480 and the antisense strand comprises a modified nucleotide sequence according to SEQ ID NO: 288.

Embodiment 55. The RNAi agent of embodiment 54, wherein:
(a) the sense strand comprises a modified nucleotide sequence according to SEQ ID NO: 400 and the antisense strand comprises a modified nucleotide sequence according to SEQ ID NO: 208;
(b) the sense strand comprises a modified nucleotide sequence according to SEQ ID NO: 419 and the antisense strand comprises a modified nucleotide sequence according to SEQ ID NO: 227;
(c) the sense strand comprises a modified nucleotide sequence according to SEQ ID NO: 436 and the antisense strand comprises a modified nucleotide sequence according to SEQ ID NO: 244;
(d) the sense strand comprises a modified nucleotide sequence according to SEQ ID NO: 468 and the antisense strand comprises a modified nucleotide sequence according to SEQ ID NO: 276;
(e) the sense strand comprises a modified nucleotide sequence according to SEQ ID NO: 470 and the antisense strand comprises a modified nucleotide sequence according to SEQ ID NO: 278;
(f) the sense strand comprises a modified nucleotide sequence according to SEQ ID NO: 473 and the antisense strand comprises a modified nucleotide sequence according to SEQ ID NO: 281; or
(g) the sense strand comprises a modified nucleotide sequence according to SEQ ID NO: 474 and the antisense strand comprises a modified nucleotide sequence according to SEQ ID NO: 282.

Embodiment 56. The RNAi agent of embodiment 55, wherein the RNAi agent comprises a targeting ligand comprising a structure according to Formula I:

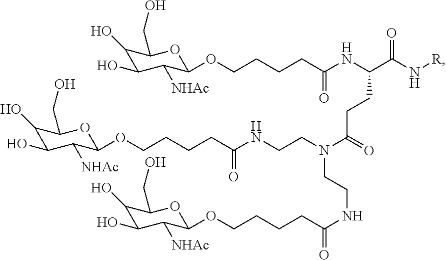

wherein R is a linker conjugated to the wherein the targeting ligand is conjugated to the 3′ end of the sense strand, wherein R comprises a structure according to Formula III:

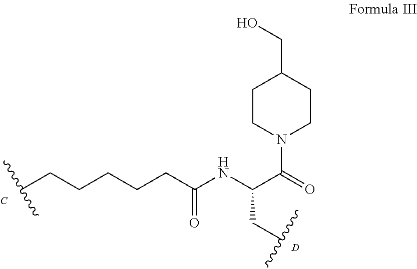

and wherein connection point C of Formula III is conjugated to Formula I, and connection point D of Formula III is conjugated to the 3′ end of the sense strand;
or wherein the RNAi agent comprises a targeting ligand comprising a structure according to Formula V:

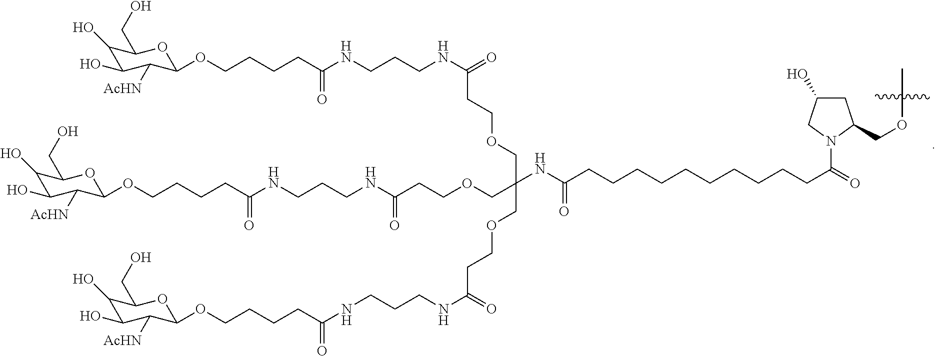 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

Embodiment 57. The RNAi agent of any one of embodiments 1-53, wherein the sense strand comprises a 3′ overhang of 1-5 nucleotides in length.

Embodiment 58. The RNAi agent of any one of embodiments 1-53 and 57, wherein the sense strand comprises a 5′ overhang of 1-5 nucleotides in length.

Embodiment 59. The RNAi agent of any one of embodiments 1-53, wherein the antisense strand comprises a 3′ overhang of 1-5 nucleotides in length.

Embodiment 60. The RNAi agent of any one of embodiments 1-53 and 59, wherein the antisense strand comprises a 5′ overhang of 1-5 nucleotides in length.

Embodiment 61. The RNAi agent of any one of embodiments 1-60, wherein the sense strand or the antisense strand comprises at least one modified nucleotide or at least one modified internucleoside linkage.

Embodiment 62. The RNAi agent of embodiment 61, wherein the sense strand and or the antisense strand comprises a modified internucleoside linkage at a first, second, penultimate, or final internucleoside linkage position.

Embodiment 63. The RNAi agent of embodiment 61 or 62, wherein the at least one modified nucleotide or at least one modified internucleoside linkage comprises a 2′-O-methyl modified nucleotide, a nucleotide comprising a 5′-phosphorothioate group, a 2′-deoxy-2′-fluoro modified nucleotide, a 2′-deoxy-modified nucleotide, a locked nucleotide, an abasic nucleotide, deoxythymidine, an inverted deoxythymidine, a 2′-amino-modified nucleotide, a 2′-alkyl-modified nucleotide, a morpholino nucleotide, a phosphoramidate, or a non-natural base comprising nucleotide.

Embodiment 64. The RNAi agent of any one of embodiments 1-55 and 57-63, wherein the targeting ligand is conjugated to the sense strand.

Embodiment 65. The RNAi agent of embodiment 64, wherein the targeting ligand is conjugated to the 3′ end of the sense strand.

Embodiment 66. The RNAi agent of embodiment 64, wherein the targeting ligand is conjugated to the 5′ end of the sense strand.

Embodiment 67. The RNAi agent of any one of embodiments 1-55 and 57-63, wherein the targeting ligand is conjugated to the antisense strand.

Embodiment 68. The RNAi agent of embodiment 67, wherein the targeting ligand is conjugated to the 3′ end of the antisense strand.

Embodiment 69. The RNAi agent of embodiment 67, wherein the targeting ligand is conjugated to the 5′ end of the antisense strand.

Embodiment 70. The RNAi agent of any one of embodiments 1-55 and 57-69, wherein the targeting ligand comprises a galactose trimer.

Embodiment 71. The RNAi agent of any one of embodiments 1-55 and 57-70, wherein the targeting ligand comprises N-acetylgalactosamine (GalNAc).

Embodiment 72. The RNAi agent of any one of embodiments 1-55 and 57-71, wherein the targeting ligand comprises a structure according to Formula I:

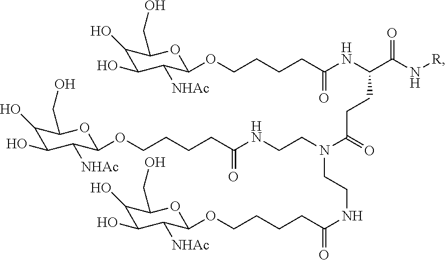

wherein R is:
(i) the sense strand or the antisense strand; or
(ii) a linker conjugated to the sense strand or the antisense strand.

Embodiment 73. The RNAi agent of embodiment 72, wherein R is a linker conjugated to the sense strand or the antisense strand having a structure according to Formula III:

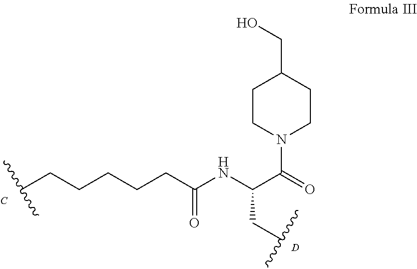

wherein:
(i) connection point C of Formula III is conjugated to Formula I, and connection point D of Formula III is conjugated to the sense strand or the antisense strand; or
(ii) connection point C of Formula III is conjugated to Formula I, and connection point D of Formula III is conjugated to a phosphate group which is conjugated to the sense strand or the antisense strand.

Embodiment 74. The RNAi agent of any one of embodiments 1-55 and 56-71, wherein the RNAi agent comprises a targeting ligand and linker according to Formula V:

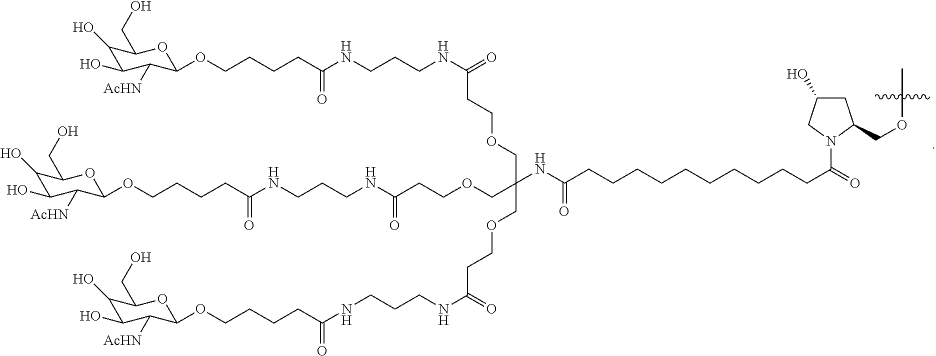 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

Embodiment 75. The RNAi agent of any one of claims 72-74, wherein Formula III or the phosphate group is conjugated to the 3′ end or the 5′ end of the sense strand or the antisense strand.

Embodiment 76. The RNAi agent of any one of claims 72-74, wherein Formula III or the phosphate group is conjugated to an internal nucleotide or nucleoside base of the sense strand or the antisense strand.

Embodiment 77. The RNAi agent of any one of embodiments 1-76, wherein the RNAi agent is an siRNA molecule.

Embodiment 78. The RNAi agent of any one of embodiments 1-76, wherein the RNAi agent is an shRNA molecule.

Embodiment 79. A modified oligonucleotide consisting of 12 to 30 or 12 to 50 linked nucleosides, wherein the modified oligonucleotide has a nucleobase sequence comprising at least 12, at least 13, at least 14, at least 15, at least 16, at least 17, at least 18, at least 19, at least 20, at least 21, at least 22, at least 23, at least 24, or at least 25 contiguous nucleobases complementary to an equal length portion of SEQ ID NO: 481, and wherein the modified oligonucleotide comprises at least one synthetically modified internucleoside linkage or at least one synthetically modified sugar-phosphate backbone.

Embodiment 80. The modified oligonucleotide of embodiment 79, wherein the modified oligonucleotide comprises a synthetically modified internucleoside linkage at a first, second, penultimate, or final internucleoside linkage position.

Embodiment 81. The modified oligonucleotide of embodiment 79 or 80, wherein the modified oligonucleotide comprises a 2′-O-methyl modified nucleotide, a nucleotide comprising a 5′-phosphorothioate group, a 2′-deoxy-2′-fluoro modified nucleotide, a 2′-deoxy-modified nucleotide, a locked nucleotide, an abasic nucleotide, deoxythymidine, an inverted deoxythymidine, a 2′-amino-modified nucleotide, a 2′-alkyl-modified nucleotide, a morpholino nucleotide, a phosphoramidate, or a non-natural base comprising nucleotide.

Embodiment 82. The modified oligonucleotide of any one of embodiments 79-81, wherein the modified oligonucleotide or a subsequence thereof differs by 0, 1, 2, 3, or 4 nucleotides from an equal length portion of any one of SEQ ID NOS: 193-288, wherein the subsequence is at least 14, 15, 16, 17, 18, 19, 20, 21, 22, or 23 bases in length.

Embodiment 83. The modified oligonucleotide of any one of embodiments 79-82, wherein the modified oligonucleotide comprises, in order, nucleotides 1-23 of any one of SEQ ID NOS: 193-288.

Embodiment 84. The modified oligonucleotide of any one of embodiments 79-83, wherein the modified oligonucleotide comprises a modified nucleotide sequence according to any one of SEQ ID NOS: 193-288.

Embodiment 85. The modified oligonucleotide of any one of embodiments 79-84, wherein the modified oligonucleotide is conjugated to a targeting ligand that targets a hepatocyte.

Embodiment 86. The modified oligonucleotide of embodiment 85, wherein the modified oligonucleotide is conjugated to the targeting ligand conjugated to its 3′ end.

Embodiment 87. The modified oligonucleotide of embodiment 85, wherein the modified oligonucleotide is conjugated to the targeting ligand conjugated to its 5′ end.

Embodiment 88. The modified oligonucleotide of any one of embodiments 85-87, wherein the targeting ligand comprises a galactose trimer.

Embodiment 89. The modified oligonucleotide of any one of embodiments 85-88, wherein the targeting ligand comprises N-acetylgalactosamine (GalNAc).

Embodiment 90. The modified oligonucleotide of any one of embodiments 85-89, wherein the targeting ligand comprises a structure according to Formula I:

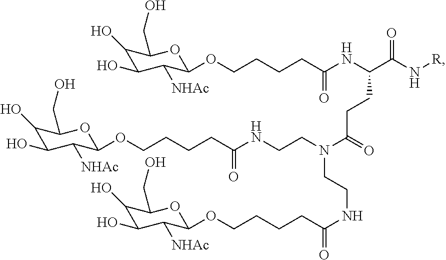

wherein R is:
(i) the modified oligonucleotide; or
(ii) a linker conjugated to the modified oligonucleotide.

Embodiment 91. The modified oligonucleotide of embodiment 90, wherein R is a linker conjugated to the modified oligonucleotide having a structure according to Formula III:

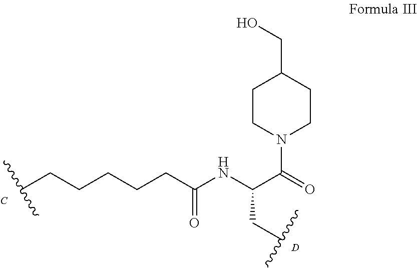

wherein:
(i) connection point C of Formula III is conjugated to Formula I, and connection point D of Formula III is conjugated to the modified oligonucleotide; or
(ii) connection point C of Formula III is conjugated to Formula I, and connection point D of Formula III is conjugated to a phosphate group which is conjugated to the modified oligonucleotide.

Embodiment 92. The modified oligonucleotide of embodiment 91, wherein Formula III or the phosphate group is conjugated to the 3′ end or the 5′ end of the modified oligonucleotide.

Embodiment 93. The modified oligonucleotide of embodiment 91, wherein Formula III or the phosphate group is conjugated to an internal nucleotide or nucleoside base of the modified oligonucleotide.

Embodiment 94. The modified oligonucleotide of any one of embodiments 85-89, wherein the modified oligonucleotide comprises a targeting ligand and linker according to Formula V:

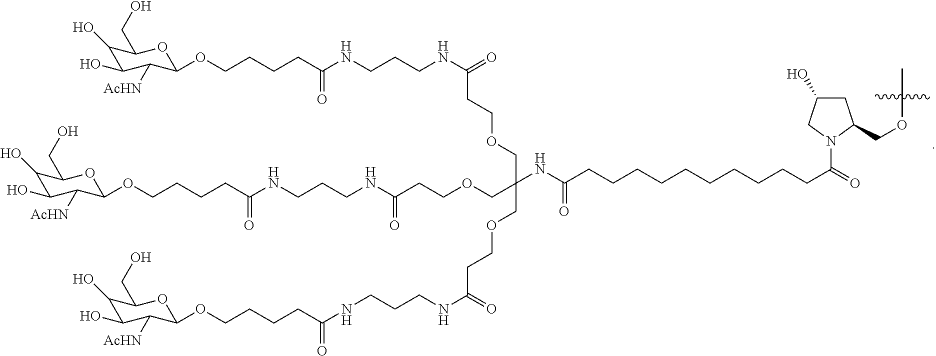 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

Embodiment 95. The modified oligonucleotide of any one of embodiments 79-94, wherein the modified oligonucleotide is an siRNA molecule.

Embodiment 96. The modified oligonucleotide of any one of embodiments 79-94, wherein the modified oligonucleotide is an shRNA molecule.

Embodiment 97. An oligomeric duplex comprising:
(a) a first modified oligonucleotide consisting of 12 to 30 or 12 to 50 linked nucleosides, wherein a nucleobase sequence of the first modified oligonucleotide comprises at least 12, at least 13, at least 14, at least 15, at least 16, at least 17, at least 18, at least 19, at least 20, at least 21, at least 22, at least 23, at least 24, or at least 25 contiguous nucleobases that are at least 80%, 81%, 82%, 83%, 84%, 85%, 86%, 87%, 88%, 89%, 90%, 91%, 92%, 93%, 94%, 95%, 96%, 97%, 98%, 99%, or 100% complementary to an equal length portion of SEQ ID NO: 481; and
(b) a second modified oligonucleotide consisting of 12 to 30 or 12 to 50 linked nucleosides, wherein a nucleobase sequence of the second modified oligonucleotide comprises a complementary region of at least 8 nucleobases that is at least 90% complementary to the nucleobase sequence of an equal length portion of the first modified oligonucleotide.

Embodiment 98. The oligomeric duplex of embodiment 97, wherein the first modified oligonucleotide and/or second modified oligonucleotide comprises at least one synthetically modified internucleoside linkage or at least one synthetically modified sugar-phosphate backbone.

Embodiment 99. The oligomeric duplex of embodiment 97 or 98, wherein the first modified oligonucleotide and/or the second comprises a 2′-O-methyl modified nucleotide, a nucleotide comprising a 5′-phosphorothioate group, a 2′-deoxy-2′-fluoro modified nucleotide, a 2′-deoxy-modified nucleotide, a locked nucleotide, an abasic nucleotide, deoxythymidine, an inverted deoxythymidine, a 2′-amino-modified nucleotide, a 2′-alkyl-modified nucleotide, a morpholino nucleotide, a phosphoramidate, or a non-natural base comprising nucleotide.

Embodiment 100. The oligomeric duplex of any one of embodiments 97-99, wherein the first modified oligonucleotide or a subsequence thereof differs by 0, 1, 2, 3, or 4 nucleotides from an equal length portion of any one of SEQ ID NOS: 193-288, wherein the subsequence is at least 14, 15, 16, 17, 18, 19, 20, 21, 22, or 23 bases in length.

Embodiment 101. The oligomeric duplex of any one of embodiments 97-100, wherein the first modified oligonucleotide comprises, in order, nucleotides 1-23 of any one of SEQ ID NOS: 193-288.

Embodiment 102. The oligomeric duplex of any one of embodiments 97-100, wherein the first modified oligonucleotide comprises a modified nucleotide sequence according to any one of SEQ ID NOS: 193-288.

Embodiment 103. The oligomeric duplex of embodiment 102, wherein the first modified oligonucleotide comprises a modified nucleotide sequence according to any one of SEQ ID NOS: 200, 203, 206, 208, 227, 228, 230, 231, 233, 234, 244, 269, 276, 278, 279, 280, 281, 282, and 285.

Embodiment 104. The oligomeric duplex of any one of embodiments 97-103, wherein the second modified oligonucleotide or subsequence thereof differs by 0, 1, 2, 3, or 4 nucleotides from an equal length portion of any one of SEQ ID NOS: 289-384 and 385-480, wherein the subsequence is at least 14, 15, 16, 17, 18, 19, 20, or 21 bases in length.

Embodiment 105. The oligomeric duplex of any one of embodiments 97-104, wherein the second modified oligonucleotide comprises, in order, nucleotides 1-21 of any one of SEQ ID NOS: 289-384 and 385-480.

Embodiment 106. The oligomeric duplex of any one of embodiments 97-105, wherein the second modified oligonucleotide comprises a modified nucleotide sequence according to any one of SEQ ID NOS: 289-384 and 385-480.

Embodiment 107. The oligomeric duplex of embodiment 106, wherein the second modified oligonucleotide comprises a modified nucleotide sequence according to any one of SEQ ID NOS: 104, 107, 110, 112, 131, 132, 134, 135, 137, 138, 148, 173, 180, 182, 183, 184, 185, 186, 189, 296, 299, 302, 304, 323, 324, 326, 327, 329, 330, 340, 365, 372, 374, 375, 376, 377, 378, 381, 392, 395, 398, 400, 419, 420, 422, 423, 425 426, 436, 461, 468, 470, 471, 472, 473, 474, and 477.

Embodiment 108. The oligomeric duplex of any one of embodiments 97-107, wherein:
(a) the second modified oligonucleotide comprises a modified nucleotide sequence according to SEQ ID NO: 289 and the first modified oligonucleotide comprises a modified nucleotide sequence according to SEQ ID NO: 193;
(b) the second modified oligonucleotide comprises a modified nucleotide sequence according to SEQ ID NO: 290 and the first modified oligonucleotide comprises a modified nucleotide sequence according to SEQ ID NO: 194;
(c) the second modified oligonucleotide comprises a modified nucleotide sequence according to SEQ ID NO: 291 and the first modified oligonucleotide comprises a modified nucleotide sequence according to SEQ ID NO: 195;
(d) the second modified oligonucleotide comprises a modified nucleotide sequence according to SEQ ID NO: 292 and the first modified oligonucleotide comprises a modified nucleotide sequence according to SEQ ID NO: 196;
(e) the second modified oligonucleotide comprises a modified nucleotide sequence according to SEQ ID NO: 293 and the first modified oligonucleotide comprises a modified nucleotide sequence according to SEQ ID NO: 197;
(f) the second modified oligonucleotide comprises a modified nucleotide sequence according to SEQ ID NO: 294 and the first modified oligonucleotide comprises a modified nucleotide sequence according to SEQ ID NO: 198;
(g) the second modified oligonucleotide comprises a modified nucleotide sequence according to SEQ ID NO: 295 and the first modified oligonucleotide comprises a modified nucleotide sequence according to SEQ ID NO: 199;
(h) the second modified oligonucleotide comprises a modified nucleotide sequence according to SEQ ID NO: 296 and the first modified oligonucleotide comprises a modified nucleotide sequence according to SEQ ID NO: 200;
(i) the second modified oligonucleotide comprises a modified nucleotide sequence according to SEQ ID NO: 297 and the first modified oligonucleotide comprises a modified nucleotide sequence according to SEQ ID NO: 201;
(j) the second modified oligonucleotide comprises a modified nucleotide sequence according to SEQ ID NO: 298 and the first modified oligonucleotide comprises a modified nucleotide sequence according to SEQ ID NO: 202;
(k) the second modified oligonucleotide comprises a modified nucleotide sequence according to SEQ ID NO: 299 and the first modified oligonucleotide comprises a modified nucleotide sequence according to SEQ ID NO: 203;
(l) the second modified oligonucleotide comprises a modified nucleotide sequence according to SEQ ID NO: 300 and the first modified oligonucleotide comprises a modified nucleotide sequence according to SEQ ID NO: 204;
(m) the second modified oligonucleotide comprises a modified nucleotide sequence according to SEQ ID NO: 301 and the first modified oligonucleotide comprises a modified nucleotide sequence according to SEQ ID NO: 205;
(n) the second modified oligonucleotide comprises a modified nucleotide sequence according to SEQ ID NO: 302 and the first modified oligonucleotide comprises a modified nucleotide sequence according to SEQ ID NO: 206;
(o) the second modified oligonucleotide comprises a modified nucleotide sequence according to SEQ ID NO: 303 and the first modified oligonucleotide comprises a modified nucleotide sequence according to SEQ ID NO: 207;
(p) the second modified oligonucleotide comprises a modified nucleotide sequence according to SEQ ID NO: 304 and the first modified oligonucleotide comprises a modified nucleotide sequence according to SEQ ID NO: 208;
(q) the second modified oligonucleotide comprises a modified nucleotide sequence according to SEQ ID NO: 305 and the first modified oligonucleotide comprises a modified nucleotide sequence according to SEQ ID NO: 209;
(r) the second modified oligonucleotide comprises a modified nucleotide sequence according to SEQ ID NO: 306 and the first modified oligonucleotide comprises a modified nucleotide sequence according to SEQ ID NO: 210;
(s) the second modified oligonucleotide comprises a modified nucleotide sequence according to SEQ ID NO: 307 and the first modified oligonucleotide comprises a modified nucleotide sequence according to SEQ ID NO: 211;
(t) the second modified oligonucleotide comprises a modified nucleotide sequence according to SEQ ID NO: 308 and the first modified oligonucleotide comprises a modified nucleotide sequence according to SEQ ID NO: 212;
(u) the second modified oligonucleotide comprises a modified nucleotide sequence according to SEQ ID NO: 309 and the first modified oligonucleotide comprises a modified nucleotide sequence according to SEQ ID NO: 213;
(v) the second modified oligonucleotide comprises a modified nucleotide sequence according to SEQ ID NO: 310 and the first modified oligonucleotide comprises a modified nucleotide sequence according to SEQ ID NO: 214;
(w) the second modified oligonucleotide comprises a modified nucleotide sequence according to SEQ ID NO: 311 and the first modified oligonucleotide comprises a modified nucleotide sequence according to SEQ ID NO: 215;
(x) the second modified oligonucleotide comprises a modified nucleotide sequence according to SEQ ID NO: 312 and the first modified oligonucleotide comprises a modified nucleotide sequence according to SEQ ID NO: 216;
(y) the second modified oligonucleotide comprises a modified nucleotide sequence according to SEQ ID NO: 313 and the first modified oligonucleotide comprises a modified nucleotide sequence according to SEQ ID NO: 217;
(z) the second modified oligonucleotide comprises a modified nucleotide sequence according to SEQ ID NO: 314 and the first modified oligonucleotide comprises a modified nucleotide sequence according to SEQ ID NO: 218;
(aa) the second modified oligonucleotide comprises a modified nucleotide sequence according to SEQ ID NO: 315 and the first modified oligonucleotide comprises a modified nucleotide sequence according to SEQ ID NO: 219;
(bb) the second modified oligonucleotide comprises a modified nucleotide sequence according to SEQ ID NO: 316 and the first modified oligonucleotide comprises a modified nucleotide sequence according to SEQ ID NO: 220;
(cc) the second modified oligonucleotide comprises a modified nucleotide sequence according to SEQ ID NO: 317 and the first modified oligonucleotide comprises a modified nucleotide sequence according to SEQ ID NO: 221;
(dd) the second modified oligonucleotide comprises a modified nucleotide sequence according to SEQ ID NO: 318 and the first modified oligonucleotide comprises a modified nucleotide sequence according to SEQ ID NO: 222;
(ee) the second modified oligonucleotide comprises a modified nucleotide sequence according to SEQ ID NO: 319 and the first modified oligonucleotide comprises a modified nucleotide sequence according to SEQ ID NO: 223;
(ff) the second modified oligonucleotide comprises a modified nucleotide sequence according to SEQ ID NO: 320 and the first modified oligonucleotide comprises a modified nucleotide sequence according to SEQ ID NO: 224;
(gg) the second modified oligonucleotide comprises a modified nucleotide sequence according to SEQ ID NO: 321 and the first modified oligonucleotide comprises a modified nucleotide sequence according to SEQ ID NO: 225;
(hh) the second modified oligonucleotide comprises a modified nucleotide sequence according to SEQ ID NO: 322 and the first modified oligonucleotide comprises a modified nucleotide sequence according to SEQ ID NO: 226;
(ii) the second modified oligonucleotide comprises a modified nucleotide sequence according to SEQ ID NO: 323 and the first modified oligonucleotide comprises a modified nucleotide sequence according to SEQ ID NO: 227;
(jj) the second modified oligonucleotide comprises a modified nucleotide sequence according to SEQ ID NO: 324 and the first modified oligonucleotide comprises a modified nucleotide sequence according to SEQ ID NO: 228;
(kk) the second modified oligonucleotide comprises a modified nucleotide sequence according to SEQ ID NO: 325 and the first modified oligonucleotide comprises a modified nucleotide sequence according to SEQ ID NO: 229;
(ll) the second modified oligonucleotide comprises a modified nucleotide sequence according to SEQ ID NO: 326 and the first modified oligonucleotide comprises a modified nucleotide sequence according to SEQ ID NO: 230;
(mm) the second modified oligonucleotide comprises a modified nucleotide sequence according to SEQ ID NO: 327 and the first modified oligonucleotide comprises a modified nucleotide sequence according to SEQ ID NO: 231;
(nn) the second modified oligonucleotide comprises a modified nucleotide sequence according to SEQ ID NO: 328 and the first modified oligonucleotide comprises a modified nucleotide sequence according to SEQ ID NO: 232;
(oo) the second modified oligonucleotide comprises a modified nucleotide sequence according to SEQ ID NO: 329 and the first modified oligonucleotide comprises a modified nucleotide sequence according to SEQ ID NO: 233;
(pp) the second modified oligonucleotide comprises a modified nucleotide sequence according to SEQ ID NO: 330 and the first modified oligonucleotide comprises a modified nucleotide sequence according to SEQ ID NO: 234;
(qq) the second modified oligonucleotide comprises a modified nucleotide sequence according to SEQ ID NO: 331 and the first modified oligonucleotide comprises a modified nucleotide sequence according to SEQ ID NO: 235;
(rr) the second modified oligonucleotide comprises a modified nucleotide sequence according to SEQ ID NO: 332 and the first modified oligonucleotide comprises a modified nucleotide sequence according to SEQ ID NO: 236;
(ss) the second modified oligonucleotide comprises a modified nucleotide sequence according to SEQ ID NO: 333 and the first modified oligonucleotide comprises a modified nucleotide sequence according to SEQ ID NO: 237;
(tt) the second modified oligonucleotide comprises a modified nucleotide sequence according to SEQ ID NO: 334 and the first modified oligonucleotide comprises a modified nucleotide sequence according to SEQ ID NO: 238;
(uu) the second modified oligonucleotide comprises a modified nucleotide sequence according to SEQ ID NO: 335 and the first modified oligonucleotide comprises a modified nucleotide sequence according to SEQ ID NO: 239;
(vv) the second modified oligonucleotide comprises a modified nucleotide sequence according to SEQ ID NO: 336 and the first modified oligonucleotide comprises a modified nucleotide sequence according to SEQ ID NO: 240;
(ww) the second modified oligonucleotide comprises a modified nucleotide sequence according to SEQ ID NO: 337 and the first modified oligonucleotide comprises a modified nucleotide sequence according to SEQ ID NO: 241;
(xx) the second modified oligonucleotide comprises a modified nucleotide sequence according to SEQ ID NO: 338 and the first modified oligonucleotide comprises a modified nucleotide sequence according to SEQ ID NO: 242;
(yy) the second modified oligonucleotide comprises a modified nucleotide sequence according to SEQ ID NO: 339 and the first modified oligonucleotide comprises a modified nucleotide sequence according to SEQ ID NO: 243;
(zz) the second modified oligonucleotide comprises a modified nucleotide sequence according to SEQ ID NO: 340 and the first modified oligonucleotide comprises a modified nucleotide sequence according to SEQ ID NO: 244;
(aaa) the second modified oligonucleotide comprises a modified nucleotide sequence according to SEQ ID NO: 341 and the first modified oligonucleotide comprises a modified nucleotide sequence according to SEQ ID NO: 245;
(bbb) the second modified oligonucleotide comprises a modified nucleotide sequence according to SEQ ID NO: 342 and the first modified oligonucleotide comprises a modified nucleotide sequence according to SEQ ID NO: 246;
(ccc) the second modified oligonucleotide comprises a modified nucleotide sequence according to SEQ ID NO: 343 and the first modified oligonucleotide comprises a modified nucleotide sequence according to SEQ ID NO: 247;
(ddd) the second modified oligonucleotide comprises a modified nucleotide sequence according to SEQ ID NO: 344 and the first modified oligonucleotide comprises a modified nucleotide sequence according to SEQ ID NO: 248;
(eee) the second modified oligonucleotide comprises a modified nucleotide sequence according to SEQ ID NO: 345 and the first modified oligonucleotide comprises a modified nucleotide sequence according to SEQ ID NO: 249;
(fff) the second modified oligonucleotide comprises a modified nucleotide sequence according to SEQ ID NO: 346 and the first modified oligonucleotide comprises a modified nucleotide sequence according to SEQ ID NO: 250;
(ggg) the second modified oligonucleotide comprises a modified nucleotide sequence according to SEQ ID NO: 347 and the first modified oligonucleotide comprises a modified nucleotide sequence according to SEQ ID NO: 251;
(hhh) the second modified oligonucleotide comprises a modified nucleotide sequence according to SEQ ID NO: 348 and the first modified oligonucleotide comprises a modified nucleotide sequence according to SEQ ID NO: 252;
(iii) the second modified oligonucleotide comprises a modified nucleotide sequence according to SEQ ID NO: 349 and the first modified oligonucleotide comprises a modified nucleotide sequence according to SEQ ID NO: 253;
(jjj) the second modified oligonucleotide comprises a modified nucleotide sequence according to SEQ ID NO: 350 and the first modified oligonucleotide comprises a modified nucleotide sequence according to SEQ ID NO: 254;
(kkk) the second modified oligonucleotide comprises a modified nucleotide sequence according to SEQ ID NO: 351 and the first modified oligonucleotide comprises a modified nucleotide sequence according to SEQ ID NO: 255;
(lll) the second modified oligonucleotide comprises a modified nucleotide sequence according to SEQ ID NO: 352 and the first modified oligonucleotide comprises a modified nucleotide sequence according to SEQ ID NO: 256;
(mmm) the second modified oligonucleotide comprises a modified nucleotide sequence according to SEQ ID NO: 353 and the first modified oligonucleotide comprises a modified nucleotide sequence according to SEQ ID NO: 257;
(nnn) the second modified oligonucleotide comprises a modified nucleotide sequence according to SEQ ID NO: 354 and the first modified oligonucleotide comprises a modified nucleotide sequence according to SEQ ID NO: 258;
(ooo) the second modified oligonucleotide comprises a modified nucleotide sequence according to SEQ ID NO: 355 and the first modified oligonucleotide comprises a modified nucleotide sequence according to SEQ ID NO: 259;
(ppp) the second modified oligonucleotide comprises a modified nucleotide sequence according to SEQ ID NO: 356 and the first modified oligonucleotide comprises a modified nucleotide sequence according to SEQ ID NO: 260;
(qqq) the second modified oligonucleotide comprises a modified nucleotide sequence according to SEQ ID NO: 357 and the first modified oligonucleotide comprises a modified nucleotide sequence according to SEQ ID NO: 261;
(rrr) the second modified oligonucleotide comprises a modified nucleotide sequence according to SEQ ID NO: 358 and the first modified oligonucleotide comprises a modified nucleotide sequence according to SEQ ID NO: 262;
(sss) the second modified oligonucleotide comprises a modified nucleotide sequence according to SEQ ID NO: 359 and the first modified oligonucleotide comprises a modified nucleotide sequence according to SEQ ID NO: 263;
(ttt) the second modified oligonucleotide comprises a modified nucleotide sequence according to SEQ ID NO: 360 and the first modified oligonucleotide comprises a modified nucleotide sequence according to SEQ ID NO: 264;
(uuu) the second modified oligonucleotide comprises a modified nucleotide sequence according to SEQ ID NO: 361 and the first modified oligonucleotide comprises a modified nucleotide sequence according to SEQ ID NO: 265;
(vvv) the second modified oligonucleotide comprises a modified nucleotide sequence according to SEQ ID NO: 362 and the first modified oligonucleotide comprises a modified nucleotide sequence according to SEQ ID NO: 266;
(www) the second modified oligonucleotide comprises a modified nucleotide sequence according to SEQ ID NO: 363 and the first modified oligonucleotide comprises a modified nucleotide sequence according to SEQ ID NO: 267;
(xxx) the second modified oligonucleotide comprises a modified nucleotide sequence according to SEQ ID NO: 364 and the first modified oligonucleotide comprises a modified nucleotide sequence according to SEQ ID NO: 268;
(yyy) the second modified oligonucleotide comprises a modified nucleotide sequence according to SEQ ID NO: 365 and the first modified oligonucleotide comprises a modified nucleotide sequence according to SEQ ID NO: 269;
(zzz) the second modified oligonucleotide comprises a modified nucleotide sequence according to SEQ ID NO: 366 and the first modified oligonucleotide comprises a modified nucleotide sequence according to SEQ ID NO: 270;
(aaaa) the second modified oligonucleotide comprises a modified nucleotide sequence according to SEQ ID NO: 367 and the first modified oligonucleotide comprises a modified nucleotide sequence according to SEQ ID NO: 271;
(bbbb) the second modified oligonucleotide comprises a modified nucleotide sequence according to SEQ ID NO: 368 and the first modified oligonucleotide comprises a modified nucleotide sequence according to SEQ ID NO: 272;
(cccc) the second modified oligonucleotide comprises a modified nucleotide sequence according to SEQ ID NO: 369 and the first modified oligonucleotide comprises a modified nucleotide sequence according to SEQ ID NO: 273;
(dddd) the second modified oligonucleotide comprises a modified nucleotide sequence according to SEQ ID NO: 370 and the first modified oligonucleotide comprises a modified nucleotide sequence according to SEQ ID NO: 274;
(eeee) the second modified oligonucleotide comprises a modified nucleotide sequence according to SEQ ID NO: 371 and the first modified oligonucleotide comprises a modified nucleotide sequence according to SEQ ID NO: 275;
(ffff) the second modified oligonucleotide comprises a modified nucleotide sequence according to SEQ ID NO: 372 and the first modified oligonucleotide comprises a modified nucleotide sequence according to SEQ ID NO: 276;
(gggg) the second modified oligonucleotide comprises a modified nucleotide sequence according to SEQ ID NO: 373 and the first modified oligonucleotide comprises a modified nucleotide sequence according to SEQ ID NO: 277;
(hhhh) the second modified oligonucleotide comprises a modified nucleotide sequence according to SEQ ID NO: 374 and the first modified oligonucleotide comprises a modified nucleotide sequence according to SEQ ID NO: 278;
(iiii) the second modified oligonucleotide comprises a modified nucleotide sequence according to SEQ ID NO: 375 and the first modified oligonucleotide comprises a modified nucleotide sequence according to SEQ ID NO: 279;
(jjjj) the second modified oligonucleotide comprises a modified nucleotide sequence according to SEQ ID NO: 376 and the first modified oligonucleotide comprises a modified nucleotide sequence according to SEQ ID NO: 280;
(kkkk) the second modified oligonucleotide comprises a modified nucleotide sequence according to SEQ ID NO: 377 and the first modified oligonucleotide comprises a modified nucleotide sequence according to SEQ ID NO: 281;
(llll) the second modified oligonucleotide comprises a modified nucleotide sequence according to SEQ ID NO: 378 and the first modified oligonucleotide comprises a modified nucleotide sequence according to SEQ ID NO: 282;
(mmmm) the second modified oligonucleotide comprises a modified nucleotide sequence according to SEQ ID NO: 379 and the first modified oligonucleotide comprises a modified nucleotide sequence according to SEQ ID NO: 283;
(nnnn) the second modified oligonucleotide comprises a modified nucleotide sequence according to SEQ ID NO: 380 and the first modified oligonucleotide comprises a modified nucleotide sequence according to SEQ ID NO: 284;
(oooo) the second modified oligonucleotide comprises a modified nucleotide sequence according to SEQ ID NO: 381 and the first modified oligonucleotide comprises a modified nucleotide sequence according to SEQ ID NO: 285;
(pppp) the second modified oligonucleotide comprises a modified nucleotide sequence according to SEQ ID NO: 382 and the first modified oligonucleotide comprises a modified nucleotide sequence according to SEQ ID NO: 286;
(qqqq) the second modified oligonucleotide comprises a modified nucleotide sequence according to SEQ ID NO: 383 and the first modified oligonucleotide comprises a modified nucleotide sequence according to SEQ ID NO: 287;
(rrrr) the second modified oligonucleotide comprises a modified nucleotide sequence according to SEQ ID NO: 384 and the first modified oligonucleotide comprises a modified nucleotide sequence according to SEQ ID NO: 288
(ssss) the sense strand comprises a modified nucleotide sequence according to SEQ ID NO: 385 and the antisense strand comprises a modified nucleotide sequence according to SEQ ID NO: 193;
(tttt) the sense strand comprises a modified nucleotide sequence according to SEQ ID NO: 386 and the antisense strand comprises a modified nucleotide sequence according to SEQ ID NO: 194;
(uuuu) the sense strand comprises a modified nucleotide sequence according to SEQ ID NO: 387 and the antisense strand comprises a modified nucleotide sequence according to SEQ ID NO: 195;
(vvvv) the sense strand comprises a modified nucleotide sequence according to SEQ ID NO: 388 and the antisense strand comprises a modified nucleotide sequence according to SEQ ID NO: 196;
(wwww) the sense strand comprises a modified nucleotide sequence according to SEQ ID NO: 389 and the antisense strand comprises a modified nucleotide sequence according to SEQ ID NO: 197;
(xxxx) the sense strand comprises a modified nucleotide sequence according to SEQ ID NO: 390 and the antisense strand comprises a modified nucleotide sequence according to SEQ ID NO: 198;
(yyyy) the sense strand comprises a modified nucleotide sequence according to SEQ ID NO: 391 and the antisense strand comprises a modified nucleotide sequence according to SEQ ID NO: 199;
(zzzz) the sense strand comprises a modified nucleotide sequence according to SEQ ID NO: 392 and the antisense strand comprises a modified nucleotide sequence according to SEQ ID NO: 200;
(aaaaa) the sense strand comprises a modified nucleotide sequence according to SEQ ID NO: 393 and the antisense strand comprises a modified nucleotide sequence according to SEQ ID NO: 201;
(bbbbb) the sense strand comprises a modified nucleotide sequence according to SEQ ID NO: 394 and the antisense strand comprises a modified nucleotide sequence according to SEQ ID NO: 202;
(ccccc) the sense strand comprises a modified nucleotide sequence according to SEQ ID NO: 395 and the antisense strand comprises a modified nucleotide sequence according to SEQ ID NO: 203;
(ddddd) the sense strand comprises a modified nucleotide sequence according to SEQ ID NO: 396 and the antisense strand comprises a modified nucleotide sequence according to SEQ ID NO: 204;
(eeeee) the sense strand comprises a modified nucleotide sequence according to SEQ ID NO: 397 and the antisense strand comprises a modified nucleotide sequence according to SEQ ID NO: 205;
(fffff) the sense strand comprises a modified nucleotide sequence according to SEQ ID NO: 398 and the antisense strand comprises a modified nucleotide sequence according to SEQ ID NO: 206;
(ggggg) the sense strand comprises a modified nucleotide sequence according to SEQ ID NO: 399 and the antisense strand comprises a modified nucleotide sequence according to SEQ ID NO: 207;
(hhhhh) the sense strand comprises a modified nucleotide sequence according to SEQ ID NO: 400 and the antisense strand comprises a modified nucleotide sequence according to SEQ ID NO: 208;
(iiiii) the sense strand comprises a modified nucleotide sequence according to SEQ ID NO: 401 and the antisense strand comprises a modified nucleotide sequence according to SEQ ID NO: 209;
(jjjjj) the sense strand comprises a modified nucleotide sequence according to SEQ ID NO: 402 and the antisense strand comprises a modified nucleotide sequence according to SEQ ID NO: 210;
(kkkkk) the sense strand comprises a modified nucleotide sequence according to SEQ ID NO: 403 and the antisense strand comprises a modified nucleotide sequence according to SEQ ID NO: 211;
(lllll) the sense strand comprises a modified nucleotide sequence according to SEQ ID NO: 404 and the antisense strand comprises a modified nucleotide sequence according to SEQ ID NO: 212;
(mmmmm) the sense strand comprises a modified nucleotide sequence according to SEQ ID NO: 405 and the antisense strand comprises a modified nucleotide sequence according to SEQ ID NO: 213;
(nnnnn) the sense strand comprises a modified nucleotide sequence according to SEQ ID NO: 406 and the antisense strand comprises a modified nucleotide sequence according to SEQ ID NO: 214;
(ooooo) the sense strand comprises a modified nucleotide sequence according to SEQ ID NO: 407 and the antisense strand comprises a modified nucleotide sequence according to SEQ ID NO: 215;
(ppppp) the sense strand comprises a modified nucleotide sequence according to SEQ ID NO: 408 and the antisense strand comprises a modified nucleotide sequence according to SEQ ID NO: 216;
(qqqqq) the sense strand comprises a modified nucleotide sequence according to SEQ ID NO: 409 and the antisense strand comprises a modified nucleotide sequence according to SEQ ID NO: 217;
(rrrrr) the sense strand comprises a modified nucleotide sequence according to SEQ ID NO: 410 and the antisense strand comprises a modified nucleotide sequence according to SEQ ID NO: 218;
(sssss) the sense strand comprises a modified nucleotide sequence according to SEQ ID NO: 411 and the antisense strand comprises a modified nucleotide sequence according to SEQ ID NO: 219;
(ttttt) the sense strand comprises a modified nucleotide sequence according to SEQ ID NO: 412 and the antisense strand comprises a modified nucleotide sequence according to SEQ ID NO: 220;
(uuuuu) the sense strand comprises a modified nucleotide sequence according to SEQ ID NO: 413 and the antisense strand comprises a modified nucleotide sequence according to SEQ ID NO: 221;
(vvvvv) the sense strand comprises a modified nucleotide sequence according to SEQ ID NO: 414 and the antisense strand comprises a modified nucleotide sequence according to SEQ ID NO: 222;
(wwwww) the sense strand comprises a modified nucleotide sequence according to SEQ ID NO: 415 and the antisense strand comprises a modified nucleotide sequence according to SEQ ID NO: 223;
(xxxxx) the sense strand comprises a modified nucleotide sequence according to SEQ ID NO: 416 and the antisense strand comprises a modified nucleotide sequence according to SEQ ID NO: 224;
(yyyyy) the sense strand comprises a modified nucleotide sequence according to SEQ ID NO: 417 and the antisense strand comprises a modified nucleotide sequence according to SEQ ID NO: 225;
(zzzzz) the sense strand comprises a modified nucleotide sequence according to SEQ ID NO: 418 and the antisense strand comprises a modified nucleotide sequence according to SEQ ID NO: 226;
(aaaaaa) the sense strand comprises a modified nucleotide sequence according to SEQ ID NO: 419 and the antisense strand comprises a modified nucleotide sequence according to SEQ ID NO: 227;
(bbbbbb) the sense strand comprises a modified nucleotide sequence according to SEQ ID NO: 420 and the antisense strand comprises a modified nucleotide sequence according to SEQ ID NO: 228;
(cccccc) the sense strand comprises a modified nucleotide sequence according to SEQ ID NO: 421 and the antisense strand comprises a modified nucleotide sequence according to SEQ ID NO: 229;
(dddddd) the sense strand comprises a modified nucleotide sequence according to SEQ ID NO: 422 and the antisense strand comprises a modified nucleotide sequence according to SEQ ID NO: 230;
(eeeeee) the sense strand comprises a modified nucleotide sequence according to SEQ ID NO: 423 and the antisense strand comprises a modified nucleotide sequence according to SEQ ID NO: 231;
(ffffff) the sense strand comprises a modified nucleotide sequence according to SEQ ID NO: 424 and the antisense strand comprises a modified nucleotide sequence according to SEQ ID NO: 232;
(gggggg) the sense strand comprises a modified nucleotide sequence according to SEQ ID NO: 425 and the antisense strand comprises a modified nucleotide sequence according to SEQ ID NO: 233;
(hhhhhh) the sense strand comprises a modified nucleotide sequence according to SEQ ID NO: 426 and the antisense strand comprises a modified nucleotide sequence according to SEQ ID NO: 234;
(iiiiii) the sense strand comprises a modified nucleotide sequence according to SEQ ID NO: 427 and the antisense strand comprises a modified nucleotide sequence according to SEQ ID NO: 235;
(jjjjjj) the sense strand comprises a modified nucleotide sequence according to SEQ ID NO: 428 and the antisense strand comprises a modified nucleotide sequence according to SEQ ID NO: 236;
(kkkkkk) the sense strand comprises a modified nucleotide sequence according to SEQ ID NO: 429 and the antisense strand comprises a modified nucleotide sequence according to SEQ ID NO: 237;
(llllll) the sense strand comprises a modified nucleotide sequence according to SEQ ID NO: 430 and the antisense strand comprises a modified nucleotide sequence according to SEQ ID NO: 238;
(mmmmmm) the sense strand comprises a modified nucleotide sequence according to SEQ ID NO: 431 and the antisense strand comprises a modified nucleotide sequence according to SEQ ID NO: 239;
(nnnnnn) the sense strand comprises a modified nucleotide sequence according to SEQ ID NO: 432 and the antisense strand comprises a modified nucleotide sequence according to SEQ ID NO: 240;
(oooooo) the sense strand comprises a modified nucleotide sequence according to SEQ ID NO: 433 and the antisense strand comprises a modified nucleotide sequence according to SEQ ID NO: 241;
(pppppp) the sense strand comprises a modified nucleotide sequence according to SEQ ID NO: 434 and the antisense strand comprises a modified nucleotide sequence according to SEQ ID NO: 242;
(qqqqqq) the sense strand comprises a modified nucleotide sequence according to SEQ ID NO: 435 and the antisense strand comprises a modified nucleotide sequence according to SEQ ID NO: 243;
(rrrrrr) the sense strand comprises a modified nucleotide sequence according to SEQ ID NO: 436 and the antisense strand comprises a modified nucleotide sequence according to SEQ ID NO: 244;
(ssssss) the sense strand comprises a modified nucleotide sequence according to SEQ ID NO: 437 and the antisense strand comprises a modified nucleotide sequence according to SEQ ID NO: 245;
(tttttt) the sense strand comprises a modified nucleotide sequence according to SEQ ID NO: 438 and the antisense strand comprises a modified nucleotide sequence according to SEQ ID NO: 246;
(uuuuuu) the sense strand comprises a modified nucleotide sequence according to SEQ ID NO: 439 and the antisense strand comprises a modified nucleotide sequence according to SEQ ID NO: 247;
(vvvvvv) the sense strand comprises a modified nucleotide sequence according to SEQ ID NO: 440 and the antisense strand comprises a modified nucleotide sequence according to SEQ ID NO: 248;
(wwwwww) the sense strand comprises a modified nucleotide sequence according to SEQ ID NO: 441 and the antisense strand comprises a modified nucleotide sequence according to SEQ ID NO: 249;
(xxxxxx) the sense strand comprises a modified nucleotide sequence according to SEQ ID NO: 442 and the antisense strand comprises a modified nucleotide sequence according to SEQ ID NO: 250;
(yyyyyy) the sense strand comprises a modified nucleotide sequence according to SEQ ID NO: 443 and the antisense strand comprises a modified nucleotide sequence according to SEQ ID NO: 251;
(zzzzzz) the sense strand comprises a modified nucleotide sequence according to SEQ ID NO: 444 and the antisense strand comprises a modified nucleotide sequence according to SEQ ID NO: 252;
(aaaaaaa) the sense strand comprises a modified nucleotide sequence according to SEQ ID NO: 445 and the antisense strand comprises a modified nucleotide sequence according to SEQ ID NO: 253;
(bbbbbbb) the sense strand comprises a modified nucleotide sequence according to SEQ ID NO: 446 and the antisense strand comprises a modified nucleotide sequence according to SEQ ID NO: 254;
(ccccccc) the sense strand comprises a modified nucleotide sequence according to SEQ ID NO: 447 and the antisense strand comprises a modified nucleotide sequence according to SEQ ID NO: 255;
(ddddddd) the sense strand comprises a modified nucleotide sequence according to SEQ ID NO: 448 and the antisense strand comprises a modified nucleotide sequence according to SEQ ID NO: 256;
(eeeeeee) the sense strand comprises a modified nucleotide sequence according to SEQ ID NO: 449 and the antisense strand comprises a modified nucleotide sequence according to SEQ ID NO: 257;
(fffffff) the sense strand comprises a modified nucleotide sequence according to SEQ ID NO: 450 and the antisense strand comprises a modified nucleotide sequence according to SEQ ID NO: 258;
(ggggggg) the sense strand comprises a modified nucleotide sequence according to SEQ ID NO: 451 and the antisense strand comprises a modified nucleotide sequence according to SEQ ID NO: 259;
(hhhhhhh) the sense strand comprises a modified nucleotide sequence according to SEQ ID NO: 452 and the antisense strand comprises a modified nucleotide sequence according to SEQ ID NO: 260;
(iiiiii) the sense strand comprises a modified nucleotide sequence according to SEQ ID NO: 453 and the antisense strand comprises a modified nucleotide sequence according to SEQ ID NO: 261;
(jjjjjjj) the sense strand comprises a modified nucleotide sequence according to SEQ ID NO: 454 and the antisense strand comprises a modified nucleotide sequence according to SEQ ID NO: 262;
(kkkkkkk) the sense strand comprises a modified nucleotide sequence according to SEQ ID NO: 455 and the antisense strand comprises a modified nucleotide sequence according to SEQ ID NO: 263;
(lllllll) the sense strand comprises a modified nucleotide sequence according to SEQ ID NO: 456 and the antisense strand comprises a modified nucleotide sequence according to SEQ ID NO: 264;
(mmmmmmm) the sense strand comprises a modified nucleotide sequence according to SEQ ID NO: 457 and the antisense strand comprises a modified nucleotide sequence according to SEQ ID NO: 265;
(nnnnnnn) the sense strand comprises a modified nucleotide sequence according to SEQ ID NO: 458 and the antisense strand comprises a modified nucleotide sequence according to SEQ ID NO: 266;
(ooooooo) the sense strand comprises a modified nucleotide sequence according to SEQ ID NO: 459 and the antisense strand comprises a modified nucleotide sequence according to SEQ ID NO: 267;
(ppppppp) the sense strand comprises a modified nucleotide sequence according to SEQ ID NO: 460 and the antisense strand comprises a modified nucleotide sequence according to SEQ ID NO: 268;
(qqqqqqq) the sense strand comprises a modified nucleotide sequence according to SEQ ID NO: 461 and the antisense strand comprises a modified nucleotide sequence according to SEQ ID NO: 269;
(rrrrrrrr) the sense strand comprises a modified nucleotide sequence according to SEQ ID NO: 462 and the antisense strand comprises a modified nucleotide sequence according to SEQ ID NO: 270;
(sssssss) the sense strand comprises a modified nucleotide sequence according to SEQ ID NO: 463 and the antisense strand comprises a modified nucleotide sequence according to SEQ ID NO: 271;
(ttttttt) the sense strand comprises a modified nucleotide sequence according to SEQ ID NO: 464 and the antisense strand comprises a modified nucleotide sequence according to SEQ ID NO: 272;
(uuuuuuu) the sense strand comprises a modified nucleotide sequence according to SEQ ID NO: 465 and the antisense strand comprises a modified nucleotide sequence according to SEQ ID NO: 273;
(vvvvvvv) the sense strand comprises a modified nucleotide sequence according to SEQ ID NO: 466 and the antisense strand comprises a modified nucleotide sequence according to SEQ ID NO: 274;
(wwwwwww) the sense strand comprises a modified nucleotide sequence according to SEQ ID NO: 467 and the antisense strand comprises a modified nucleotide sequence according to SEQ ID NO: 275;
(xxxxxxx) the sense strand comprises a modified nucleotide sequence according to SEQ ID NO: 468 and the antisense strand comprises a modified nucleotide sequence according to SEQ ID NO: 276;
(yyyyyyy) the sense strand comprises a modified nucleotide sequence according to SEQ ID NO: 469 and the antisense strand comprises a modified nucleotide sequence according to SEQ ID NO: 277;
(zzzzzzz) the sense strand comprises a modified nucleotide sequence according to SEQ ID NO: 470 and the antisense strand comprises a modified nucleotide sequence according to SEQ ID NO: 278;
(aaaaaaaa) the sense strand comprises a modified nucleotide sequence according to SEQ ID NO: 471 and the antisense strand comprises a modified nucleotide sequence according to SEQ ID NO: 279;
(bbbbbbbb) the sense strand comprises a modified nucleotide sequence according to SEQ ID NO: 472 and the antisense strand comprises a modified nucleotide sequence according to SEQ ID NO: 280;
(cccccccc) the sense strand comprises a modified nucleotide sequence according to SEQ ID NO: 473 and the antisense strand comprises a modified nucleotide sequence according to SEQ ID NO: 281;
(dddddddd) the sense strand comprises a modified nucleotide sequence according to SEQ ID NO: 474 and the antisense strand comprises a modified nucleotide sequence according to SEQ ID NO: 282;
(eeeeeeee) the sense strand comprises a modified nucleotide sequence according to SEQ ID NO: 475 and the antisense strand comprises a modified nucleotide sequence according to SEQ ID NO: 283;
(ffffffff) the sense strand comprises a modified nucleotide sequence according to SEQ ID NO: 476 and the antisense strand comprises a modified nucleotide sequence according to SEQ ID NO: 284;
(gggggggg) the sense strand comprises a modified nucleotide sequence according to SEQ ID NO: 477 and the antisense strand comprises a modified nucleotide sequence according to SEQ ID NO: 285;
(hhhhhhhh) the sense strand comprises a modified nucleotide sequence according to SEQ ID NO: 478 and the antisense strand comprises a modified nucleotide sequence according to SEQ ID NO: 286;
(iiiiiii) the sense strand comprises a modified nucleotide sequence according to SEQ ID NO: 479 and the antisense strand comprises a modified nucleotide sequence according to SEQ ID NO: 287; or
(jjjjjjjj) the sense strand comprises a modified nucleotide sequence according to SEQ ID NO: 480 and the antisense strand comprises a modified nucleotide sequence according to SEQ ID NO: 288.

Embodiment 109. The oligomeric duplex of any one of embodiments 97-108, wherein the first modified oligonucleotide and/or second modified oligonucleotide comprises a targeting ligand.

Embodiment 110. The oligomeric duplex of embodiment 109, wherein the first modified oligonucleotide comprises a targeting ligand conjugated to its 3′ end.

Embodiment 111. The oligomeric duplex of embodiment 109, wherein the first modified oligonucleotide comprises a targeting ligand conjugated to its 5′ end.

Embodiment 112. The oligomeric duplex of embodiment 109, wherein the second modified oligonucleotide comprises a targeting ligand conjugated to its 3′ end.

Embodiment 113. The oligomeric duplex of embodiment 109, wherein the second modified oligonucleotide comprises a targeting ligand conjugated to its 5′ end.

Embodiment 114. The oligomeric duplex of any one of embodiments 109-113, wherein the targeting ligand targets a hepatocyte, and optionally wherein the targeting ligand comprises a galactose trimer.

Embodiment 115. The oligomeric duplex of any one of embodiments 109-114, wherein the targeting ligand comprises N-acetylgalactosamine (GalNAc).

Embodiment 116. The oligomeric duplex of any one of embodiments 109-115, wherein the targeting ligand comprises a structure according to Formula I:

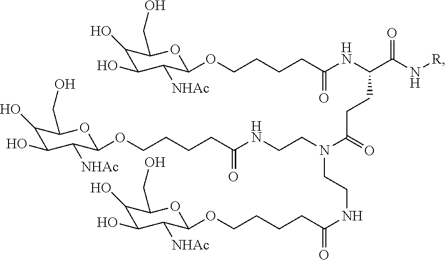

wherein R is:
(i) the first modified oligonucleotide or the second modified oligonucleotide; or
(ii) a linker conjugated to the first modified oligonucleotide or the second modified oligonucleotide.

Embodiment 117. The oligomeric duplex of embodiment 116, wherein R is a linker conjugated to the first modified oligonucleotide or the second modified oligonucleotide having a structure according to Formula III:

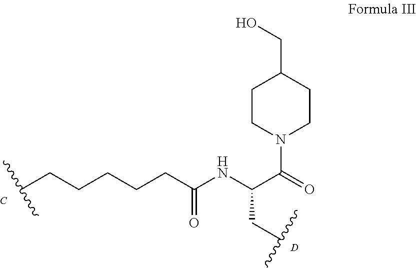

wherein:
(i) connection point C of Formula III is conjugated to Formula I, and connection point D of Formula III is conjugated to the first modified oligonucleotide or the second modified oligonucleotide; or
(ii) connection point C of Formula III is conjugated to Formula I, and connection point D of Formula III is conjugated to a phosphate group which is conjugated to the first modified oligonucleotide or the second modified oligonucleotide.

Embodiment 118. The oligomeric duplex of embodiment 117, wherein Formula III or the phosphate group is conjugated to the 3′ end or the 5′ end of the first modified oligonucleotide or the second modified oligonucleotide.

Embodiment 119. The oligomeric duplex of embodiment 117, wherein Formula III or the phosphate group is conjugated to an internal nucleotide or nucleoside base of the first modified oligonucleotide or the second modified oligonucleotide.

Embodiment 120. The oligomeric duplex of any one of embodiments 109-115, wherein the first modified oligonucleotide or the second modified oligonucleotide comprises a targeting ligand and linker according to Formula V:

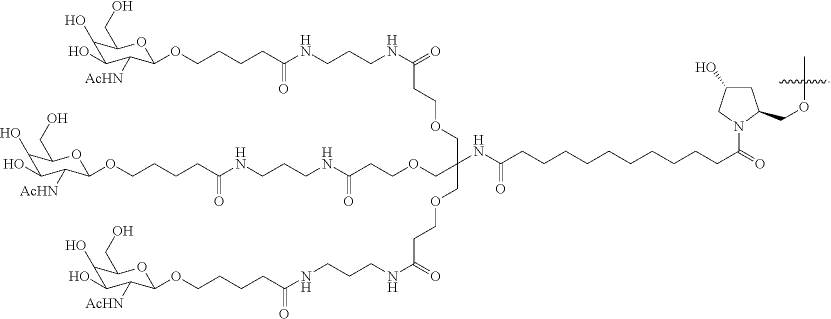

Embodiment 121. The oligomeric duplex of any one of embodiments 97-120, wherein the oligomeric duplex is an siRNA molecule.

Embodiment 122. The oligomeric duplex of any one of embodiments 97-120, wherein the oligomeric duplex is an shRNA molecule.

Embodiment 123. A pharmaceutical composition comprising the RNAi agent of any one of embodiments 1-78, the modified oligonucleotide of any one of embodiments 79-96, or the oligomeric duplex of any one of embodiments 97-122, and a pharmaceutically acceptable carrier.

Embodiment 124. A lipid nanoparticle comprising the RNAi agent of any one of embodiments 1-78, the modified oligonucleotide of any one of embodiments 79-96, the oligomeric duplex of any one of embodiments 97-122, or the pharmaceutical composition of embodiment 123.

Embodiment 125. A method of making a pharmaceutical composition, comprising combining the RNAi agent of any one of embodiments 1-78, the modified oligonucleotide of any one of embodiments 79-96, or the oligomeric duplex of any one of embodiments 97-122, with a delivery vehicle selected from the group consisting of a phosphate-buffered saline (PBS), lipid, a nanoparticle, a polymer, a liposome, a micelle, a dynamic polyconjugate (DPC), and antibody-oligo conjugate.

Embodiment 126. A method of treating metabolic dysfunction-associated steatotic liver disease (MASLD) or metabolic dysfunction-associated steatotic liver (MASL) in a subject, comprising:
administering to the subject an effective amount of a liver-targeted modified oligonucleotide, a liver-targeted oligomeric duplex, or a liver-targeted RNAi agent that inhibits expression of glucokinase (GCK).

Embodiment 127. Use of a liver-targeted modified oligonucleotide, a liver-targeted oligomeric duplex, or a liver-targeted RNAi agent that inhibits expression of glucokinase (GCK) for the manufacture of a medicament for the treatment of metabolic dysfunction-associated steatotic liver disease (MASLD) or metabolic dysfunction-associated steatotic liver (MASL) in a subject.

Embodiment 128. A method of inhibiting expression of glucokinase (GCK) in a subject, comprising:
administering to the subject an effective amount of a liver-targeted modified oligonucleotide, a liver-targeted oligomeric duplex, or a liver-targeted RNAi agent that inhibits expression of GCK.

Embodiment 129. Use of a liver-targeted modified oligonucleotide, a liver-targeted oligomeric duplex, or a liver-targeted RNAi agent in the manufacture of a medicament for inhibiting expression of glucokinase (GCK) in a hepatocyte of a subject.

Embodiment 130. A method of controlling or reducing liver fat accumulation in a subject, comprising:
administering to the subject an effective amount of a liver-targeted modified oligonucleotide, a liver-targeted oligomeric duplex, or a liver-targeted RNAi agent that inhibits expression of glucokinase (GCK).

Embodiment 131. Use of a liver-targeted modified oligonucleotide, a liver-targeted oligomeric duplex, or a liver-targeted RNAi agent that inhibits expression of glucokinase (GCK) in the manufacture of a medicament for controlling or reducing liver fat accumulation in a subject.

Embodiment 132. The method or use of embodiment 130 or 131, wherein the method treats metabolic dysfunction-associated steatotic liver disease (MASLD) or metabolic dysfunction-associated steatohepatitis (MASH) in the subject.

Embodiment 133. The method or use of embodiment 130 or 131, wherein the method treats nonalcoholic fatty liver disease (NAFLD) or nonalcoholic steatohepatitis (NASH) in the subject.

Embodiment 134. The method or use of any one of embodiments 126-133, wherein the liver-targeted modified oligonucleotide is the modified oligonucleotide of any one of embodiments 79-96.

Embodiment 135. The method or use of any one of embodiments 126-133, wherein the liver-targeted oligomeric duplex is the oligomeric duplex of any one of embodiments 97-122.

Embodiment 136. The method or use of any one of embodiments 126-133, wherein the RNAi agent comprises an antisense strand and a sense strand that at least partially complements the antisense strand, wherein:
(a) the antisense strand comprises at least 14 contiguous bases from any one of SEQ ID NO S: 1-96; and/or
(b) the sense strand comprises at least 14 contiguous bases from any one of SEQ ID NOS: 97-192.

Embodiment 137. The method or use of embodiment 136, wherein one or more bases of the antisense stand and/or the sense strand are modified.

Embodiment 138. The method or use of embodiment 137, wherein:
(a) the antisense strand comprises at least 14 contiguous bases from any one of SEQ ID NOS: 193-288; and/or
(b) the sense strand comprises at least 14 contiguous bases from any one of SEQ ID NOS: 289-384 and 385-480.

Embodiment 139. The method or use of any one of embodiments 126-133, wherein the RNAi agent comprises an antisense strand and a sense strand that at least partially complements the antisense strand, and a targeting ligand conjugated to the antisense strand or the sense strand, wherein the targeting ligand targets a hepatocyte.

Embodiment 140. The method or use of any one of embodiments 136-139, wherein the antisense strand comprises at least 14 contiguous bases that complement a sequence in an mRNA molecule that encodes GCK (SEQ ID NO: 481).

Embodiment 141. The method or use of any one of embodiments 136-140, wherein the antisense strand and the sense strand form a duplex of at least 14 bases in length.

Embodiment 142. The method or use of any one of embodiments 136-141, wherein the antisense strand and the sense strand form a duplex of between 15 and 30 bases in length.

Embodiment 143. The method or use of any one of embodiments 136-142, wherein the antisense strand or a subsequence thereof differs by 0, 1, 2, 3, or 4 nucleotides from an equal length portion of any one of SEQ ID NOS: 1-96 and 193-288, wherein the subsequence is at least 14, 15, 16, 17, 18, 19, 20, 21, 22, or 23 bases in length.

Embodiment 144. The method or use of embodiment 143, wherein the antisense strand comprises, in order, nucleotides 1-23 of any one of SEQ ID NOS: 1-96 and 193-288.

Embodiment 145. The method or use of any one of embodiments 139-144, wherein the antisense strand comprises at least 14 contiguous bases from any one of SEQ ID NOS: 1-96 and 193-288.

Embodiment 146. The method or use of any one of embodiments 136-145, wherein the antisense strand comprises, in order, nucleobases 1-14 of any one of SEQ ID NOS: 1-96 and 193-288.

Embodiment 147. The method or use of any one of embodiments 136-146, wherein the antisense strand comprises at least 14 contiguous bases from any one of SEQ ID NOS: 8, 11, 14, 16, 35, 36, 38, 39, 41, 42, 52, 77, 84, 86, 87, 88, 89, 90, 93, 200, 203, 206, 208, 227, 228, 230, 231, 233, 234, 244, 269, 276, 278, 279, 280, 281, 282, and 285.

Embodiment 148. The method or use of any one of embodiments 136-147, wherein the sense strand or a subsequence thereof differs by 0, 1, 2, 3, or 4 nucleotides from an equal length portion of any one of SEQ ID NOS: 97-192, 289-384, and 385-480, wherein the subsequence is at least 14, 15, 16, 17, 18, 19, 20, or 21 bases in length.

Embodiment 149. The method or use of embodiment 148, wherein the sense strand comprises, in order, nucleotides 1-21 of any one of SEQ ID NOS: 97-192, 289-384, and 385-480.

Embodiment 150. The method or use of any one of embodiments 139-149, wherein the sense strand comprises at least 14 contiguous bases from any one of SEQ ID NOS: 97-192, 289-384, and 385-480.

Embodiment 151. The method or use of any one of embodiments 136-150, wherein the sense strand comprises, in order, nucleobases 1-14 of any one of SEQ ID NOS: 97-192, 289-384, and 385-480.

Embodiment 152. The method or use of any one of embodiments 136-151, wherein the sense strand comprises at least 14 contiguous bases from any one of SEQ ID NOS: 104, 107, 110, 112, 131, 132, 134, 135, 137, 138, 148, 173, 180, 182, 183, 184, 185, 186, 189, 296, 299, 302, 304, 323, 324, 326, 327, 329, 330, 340, 365, 372, 374, 375, 376, 377, 378, 381, 392, 395, 398, 400, 419, 420, 422, 423, 425 426, 436, 461, 468, 470, 471, 472, 473, 474, and 477.

Embodiment 153. The method or use of any one of embodiments 136-152, wherein the antisense strand comprises at least 16 contiguous bases that complement a sequence in an mRNA molecule that encodes GCK (SEQ ID NO: 481).

Embodiment 154. The method or use of any one of embodiments 136-153, wherein the antisense strand and the sense strand form a duplex of at least 16 bases in length.

Embodiment 155. The method or use of any one of embodiments 136-154, wherein the antisense strand comprises at least 16 contiguous bases from any one of SEQ ID NOS: 1-96 and 193-288.

Embodiment 156. The method or use of any one of embodiments 136-155, wherein the antisense strand comprises at least 16 contiguous bases from any one of SEQ ID NOS: 8, 11, 14, 16, 35, 36, 38, 39, 41, 42, 52, 77, 84, 86, 87, 88, 89, 90, 93, 200, 203, 206, 208, 227, 228, 230, 231, 233, 234, 244, 269, 276, 278, 279, 280, 281, 282, and 285.

Embodiment 157. The method or use of any one of embodiments 136-156, wherein the antisense strand comprises, in order, nucleobases 1-16, 1-17, 1-18, 1-19, 1-20, 1-21, 1-22, 1-23, 2-17, 2-18, 2-19, 2-20, 2-21, 2-22, 2-23, 3-18, 3-19, 3-20, 3-21, 3-22, 3-23, 4-19, 4-20, 4-21, 4-22, 4-23, 5-21, 5-22, 5-23, 6-20, 6-21, 6-22, 6-23, 7-22, 7-23, or 8-23 of any one of SEQ ID NOS: 1-96 and 193-288.

Embodiment 158. The method or use of any one of embodiments 136-157, wherein the antisense strand comprises a nucleotide sequence according to any one of SEQ ID NOS: 1-96.

Embodiment 159. The method or use of embodiment 158, wherein one or more nucleotides of the antisense strand are modified.

Embodiment 160. The method or use of embodiment 158, wherein the antisense strand comprises a nucleotide sequence according to any one of SEQ ID NOS: 8, 11, 14, 16, 35, 36, 38, 39, 41, 42, 52, 77, 84, 86, 87, 88, 89, 90, 93.

Embodiment 161. The method or use of embodiment 160, wherein one or more nucleotides of the antisense strand are modified.

Embodiment 162. The method or use of embodiment 159 or 161, wherein the antisense strand has a modification pattern according to nsNfsnnnNfnnnnnnnNfnNfnnnnnsnsn.

Embodiment 163. The method or use of any one of embodiments 158-161, wherein the antisense strand comprises a modified nucleotide sequence according to any one of SEQ ID NOS: 193-288.

Embodiment 164. The method or use of embodiment 163, wherein the antisense strand comprises a modified nucleotide sequence according to any one of SEQ ID NOS: 200, 203, 206, 208, 227, 228, 230, 231, 233, 234, 244, 269, 276, 278, 279, 280, 281, 282, and 285.

Embodiment 165. The method or use of any one of embodiments 136-164, wherein the sense strand comprises at least 16 contiguous bases from any one of SEQ ID NOS: 97-192, 289-384, and 385-480.

Embodiment 166. The method or use of embodiment 165, wherein the sense strand comprises at least 16 contiguous bases from any one of SEQ ID NOS: 104, 107, 110, 112, 131, 132, 134, 135, 137, 138, 148, 173, 180, 182, 183, 184, 185, 186, 189, 296, 299, 302, 304, 323, 324, 326, 327, 329, 330, 340, 365, 372, 374, 375, 376, 377, 378, 381, 392, 395, 398, 400, 419, 420, 422, 423, 425 426, 436, 461, 468, 470, 471, 472, 473, 474, and 477.

Embodiment 167. The method or use of any one of embodiments 136-166, wherein the sense strand comprises, in order, nucleobases 1-16, 1-17, 1-18, 1-19, 1-20, 1-21, 1-22, 1-23, 2-17, 2-18, 2-19, 2-20, 2-21, 2-22, 2-23, 3-18, 3-19, 3-20, 3-21, 3-22, 3-23, 4-19, 4-20, 4-21, 4-22, 4-23, 5-21, 5-22, 5-23, 6-20, 6-21, 6-22, 6-23, 7-22, 7-23, or 8-23 of any one of SEQ ID NOS: 97-192, 289-384, and 385-480.

Embodiment 168. The method or use of any one of embodiments 136-167, wherein the sense strand comprises a nucleobase sequence according to any one of SEQ ID NOS: 97-192.

Embodiment 169. The method or use of embodiment 168, wherein one or more nucleotides of the sense strand are modified.

Embodiment 170. The method or use of embodiment 168, wherein the sense strand comprises a nucleobase sequence according to any one of SEQ ID NOS: 104, 107, 110, 112, 131, 132, 134, 135, 137, 138, 148, 173, 180, 182, 183, 184, 185, 186, and 189.

Embodiment 171. The method or use of embodiment 170, wherein one or more nucleotides of the sense strand are modified.

Embodiment 172. The method or use of embodiment 169 or 171, wherein the sense strand has a modification pattern according to nsnsnnnnNfnNfNfNfnnnnnnnnsnsn.

Embodiment 173. The method or use of embodiment 169 or 171, wherein the sense strand has a modification pattern according to nsnsnnnnNfnNfNfNfnnnnnnnnnn.

Embodiment 174. The method or use of any one of embodiments 168-173, wherein the sense strand comprises a modified nucleotide sequence according to any one of SEQ ID NOS: 289-384 and 385-480.

Embodiment 175. The method or use of embodiment 174, wherein the sense strand comprises a modified nucleotide sequence according to any one of SEQ ID NOS: 296, 299, 302, 304, 323, 324, 326, 327, 329, 330, 340, 365, 372, 374, 375, 376, 377, 378, 381, 392, 395, 398, 400, 419, 420, 422, 423, 425 426, 436, 461, 468, 470, 471, 472, 473, 474, and 477.

Embodiment 176. The method or use of any one of embodiments 136-175, wherein:
(a) the sense strand comprises a nucleobase sequence according to SEQ ID NO: 97 and the antisense strand comprises a nucleobase sequence according to SEQ ID NO: 1;
(b) the sense strand comprises a nucleobase sequence according to SEQ ID NO: 98 and the antisense strand comprises a nucleobase sequence according to SEQ ID NO: 2;
(c) the sense strand comprises a nucleobase sequence according to SEQ ID NO: 99 and the antisense strand comprises a nucleobase sequence according to SEQ ID NO: 3;
(d) the sense strand comprises a nucleobase sequence according to SEQ ID NO: 100 and the antisense strand comprises a nucleobase sequence according to SEQ ID NO: 4;
(e) the sense strand comprises a nucleobase sequence according to SEQ ID NO: 101 and the antisense strand comprises a nucleobase sequence according to SEQ ID NO: 5;
(f) the sense strand comprises a nucleobase sequence according to SEQ ID NO: 102 and the antisense strand comprises a nucleobase sequence according to SEQ ID NO: 6;
(g) the sense strand comprises a nucleobase sequence according to SEQ ID NO: 103 and the antisense strand comprises a nucleobase sequence according to SEQ ID NO: 7;
(h) the sense strand comprises a nucleobase sequence according to SEQ ID NO: 104 and the antisense strand comprises a nucleobase sequence according to SEQ ID NO: 8;
(i) the sense strand comprises a nucleobase sequence according to SEQ ID NO: 105 and the antisense strand comprises a nucleobase sequence according to SEQ ID NO: 9;
(j) the sense strand comprises a nucleobase sequence according to SEQ ID NO: 106 and the antisense strand comprises a nucleobase sequence according to SEQ ID NO: 10;
(k) the sense strand comprises a nucleobase sequence according to SEQ ID NO: 107 and the antisense strand comprises a nucleobase sequence according to SEQ ID NO: 11;
(l) the sense strand comprises a nucleobase sequence according to SEQ ID NO: 108 and the antisense strand comprises a nucleobase sequence according to SEQ ID NO: 12;
(m) the sense strand comprises a nucleobase sequence according to SEQ ID NO: 109 and the antisense strand comprises a nucleobase sequence according to SEQ ID NO: 13;
(n) the sense strand comprises a nucleobase sequence according to SEQ ID NO: 110 and the antisense strand comprises a nucleobase sequence according to SEQ ID NO: 14;
(o) the sense strand comprises a nucleobase sequence according to SEQ ID NO: 111 and the antisense strand comprises a nucleobase sequence according to SEQ ID NO: 15;
(p) the sense strand comprises a nucleobase sequence according to SEQ ID NO: 112 and the antisense strand comprises a nucleobase sequence according to SEQ ID NO: 16;
(q) the sense strand comprises a nucleobase sequence according to SEQ ID NO: 113 and the antisense strand comprises a nucleobase sequence according to SEQ ID NO: 17;
(r) the sense strand comprises a nucleobase sequence according to SEQ ID NO: 114 and the antisense strand comprises a nucleobase sequence according to SEQ ID NO: 18;
(s) the sense strand comprises a nucleobase sequence according to SEQ ID NO: 115 and the antisense strand comprises a nucleobase sequence according to SEQ ID NO: 19;
(t) the sense strand comprises a nucleobase sequence according to SEQ ID NO: 116 and the antisense strand comprises a nucleobase sequence according to SEQ ID NO: 20;
(u) the sense strand comprises a nucleobase sequence according to SEQ ID NO: 117 and the antisense strand comprises a nucleobase sequence according to SEQ ID NO: 21;
(v) the sense strand comprises a nucleobase sequence according to SEQ ID NO: 118 and the antisense strand comprises a nucleobase sequence according to SEQ ID NO: 22;
(w) the sense strand comprises a nucleobase sequence according to SEQ ID NO: 119 and the antisense strand comprises a nucleobase sequence according to SEQ ID NO: 23;
(x) the sense strand comprises a nucleobase sequence according to SEQ ID NO: 120 and the antisense strand comprises a nucleobase sequence according to SEQ ID NO: 24;
(y) the sense strand comprises a nucleobase sequence according to SEQ ID NO: 121 and the antisense strand comprises a nucleobase sequence according to SEQ ID NO: 25;
(z) the sense strand comprises a nucleobase sequence according to SEQ ID NO: 122 and the antisense strand comprises a nucleobase sequence according to SEQ ID NO: 26;
(aa) the sense strand comprises a nucleobase sequence according to SEQ ID NO: 123 and the antisense strand comprises a nucleobase sequence according to SEQ ID NO: 27;
(bb) the sense strand comprises a nucleobase sequence according to SEQ ID NO: 124 and the antisense strand comprises a nucleobase sequence according to SEQ ID NO: 28;
(cc) the sense strand comprises a nucleobase sequence according to SEQ ID NO: 125 and the antisense strand comprises a nucleobase sequence according to SEQ ID NO: 29;
(dd) the sense strand comprises a nucleobase sequence according to SEQ ID NO: 126 and the antisense strand comprises a nucleobase sequence according to SEQ ID NO: 30;
(ee) the sense strand comprises a nucleobase sequence according to SEQ ID NO: 127 and the antisense strand comprises a nucleobase sequence according to SEQ ID NO: 31;
(ff) the sense strand comprises a nucleobase sequence according to SEQ ID NO: 128 and the antisense strand comprises a nucleobase sequence according to SEQ ID NO: 32;
(gg) the sense strand comprises a nucleobase sequence according to SEQ ID NO: 129 and the antisense strand comprises a nucleobase sequence according to SEQ ID NO: 33;
(hh) the sense strand comprises a nucleobase sequence according to SEQ ID NO: 130 and the antisense strand comprises a nucleobase sequence according to SEQ ID NO: 34;
(ii) the sense strand comprises a nucleobase sequence according to SEQ ID NO: 131 and the antisense strand comprises a nucleobase sequence according to SEQ ID NO: 35;
(jj) the sense strand comprises a nucleobase sequence according to SEQ ID NO: 132 and the antisense strand comprises a nucleobase sequence according to SEQ ID NO: 36;
(kk) the sense strand comprises a nucleobase sequence according to SEQ ID NO: 133 and the antisense strand comprises a nucleobase sequence according to SEQ ID NO: 37;
(ll) the sense strand comprises a nucleobase sequence according to SEQ ID NO: 134 and the antisense strand comprises a nucleobase sequence according to SEQ ID NO: 38;
(mm) the sense strand comprises a nucleobase sequence according to SEQ ID NO: 135 and the antisense strand comprises a nucleobase sequence according to SEQ ID NO: 39;
(nn) the sense strand comprises a nucleobase sequence according to SEQ ID NO: 136 and the antisense strand comprises a nucleobase sequence according to SEQ ID NO: 40;
(oo) the sense strand comprises a nucleobase sequence according to SEQ ID NO: 137 and the antisense strand comprises a nucleobase sequence according to SEQ ID NO: 41;
(pp) the sense strand comprises a nucleobase sequence according to SEQ ID NO: 138 and the antisense strand comprises a nucleobase sequence according to SEQ ID NO: 42;
(qq) the sense strand comprises a nucleobase sequence according to SEQ ID NO: 139 and the antisense strand comprises a nucleobase sequence according to SEQ ID NO: 43;
(rr) the sense strand comprises a nucleobase sequence according to SEQ ID NO: 140 and the antisense strand comprises a nucleobase sequence according to SEQ ID NO: 44;
(ss) the sense strand comprises a nucleobase sequence according to SEQ ID NO: 141 and the antisense strand comprises a nucleobase sequence according to SEQ ID NO: 45;
(tt) the sense strand comprises a nucleobase sequence according to SEQ ID NO: 142 and the antisense strand comprises a nucleobase sequence according to SEQ ID NO: 46;
(uu) the sense strand comprises a nucleobase sequence according to SEQ ID NO: 143 and the antisense strand comprises a nucleobase sequence according to SEQ ID NO: 47;
(vv) the sense strand comprises a nucleobase sequence according to SEQ ID NO: 144 and the antisense strand comprises a nucleobase sequence according to SEQ ID NO: 48;
(ww) the sense strand comprises a nucleobase sequence according to SEQ ID NO: 145 and the antisense strand comprises a nucleobase sequence according to SEQ ID NO: 49;
(xx) the sense strand comprises a nucleobase sequence according to SEQ ID NO: 146 and the antisense strand comprises a nucleobase sequence according to SEQ ID NO: 50;
(yy) the sense strand comprises a nucleobase sequence according to SEQ ID NO: 147 and the antisense strand comprises a nucleobase sequence according to SEQ ID NO: 51;
(zz) the sense strand comprises a nucleobase sequence according to SEQ ID NO: 148 and the antisense strand comprises a nucleobase sequence according to SEQ ID NO: 52;
(aaa) the sense strand comprises a nucleobase sequence according to SEQ ID NO: 149 and the antisense strand comprises a nucleobase sequence according to SEQ ID NO: 53;
(bbb) the sense strand comprises a nucleobase sequence according to SEQ ID NO: 150 and the antisense strand comprises a nucleobase sequence according to SEQ ID NO: 54;
(ccc) the sense strand comprises a nucleobase sequence according to SEQ ID NO: 151 and the antisense strand comprises a nucleobase sequence according to SEQ ID NO: 55;
(ddd) the sense strand comprises a nucleobase sequence according to SEQ ID NO: 152 and the antisense strand comprises a nucleobase sequence according to SEQ ID NO: 56;
(eee) the sense strand comprises a nucleobase sequence according to SEQ ID NO: 153 and the antisense strand comprises a nucleobase sequence according to SEQ ID NO: 57;
(fff) the sense strand comprises a nucleobase sequence according to SEQ ID NO: 154 and the antisense strand comprises a nucleobase sequence according to SEQ ID NO: 58;
(ggg) the sense strand comprises a nucleobase sequence according to SEQ ID NO: 155 and the antisense strand comprises a nucleobase sequence according to SEQ ID NO: 59;
(hhh) the sense strand comprises a nucleobase sequence according to SEQ ID NO: 156 and the antisense strand comprises a nucleobase sequence according to SEQ ID NO: 60;
(iii) the sense strand comprises a nucleobase sequence according to SEQ ID NO: 157 and the antisense strand comprises a nucleobase sequence according to SEQ ID NO: 61;
(jjj) the sense strand comprises a nucleobase sequence according to SEQ ID NO: 158 and the antisense strand comprises a nucleobase sequence according to SEQ ID NO: 62;
(kkk) the sense strand comprises a nucleobase sequence according to SEQ ID NO: 159 and the antisense strand comprises a nucleobase sequence according to SEQ ID NO: 63;
(lll) the sense strand comprises a nucleobase sequence according to SEQ ID NO: 160 and the antisense strand comprises a nucleobase sequence according to SEQ ID NO: 64;
(mmm) the sense strand comprises a nucleobase sequence according to SEQ ID NO: 161 and the antisense strand comprises a nucleobase sequence according to SEQ ID NO: 65;
(nnn) the sense strand comprises a nucleobase sequence according to SEQ ID NO: 162 and the antisense strand comprises a nucleobase sequence according to SEQ ID NO: 66;
(ooo) the sense strand comprises a nucleobase sequence according to SEQ ID NO: 163 and the antisense strand comprises a nucleobase sequence according to SEQ ID NO: 67;
(ppp) the sense strand comprises a nucleobase sequence according to SEQ ID NO: 164 and the antisense strand comprises a nucleobase sequence according to SEQ ID NO: 68;
(qqq) the sense strand comprises a nucleobase sequence according to SEQ ID NO: 165 and the antisense strand comprises a nucleobase sequence according to SEQ ID NO: 69;
(rrr) the sense strand comprises a nucleobase sequence according to SEQ ID NO: 166 and the antisense strand comprises a nucleobase sequence according to SEQ ID NO: 70;
(sss) the sense strand comprises a nucleobase sequence according to SEQ ID NO: 167 and the antisense strand comprises a nucleobase sequence according to SEQ ID NO: 71;
(ttt) the sense strand comprises a nucleobase sequence according to SEQ ID NO: 168 and the antisense strand comprises a nucleobase sequence according to SEQ ID NO: 72;
(uuu) the sense strand comprises a nucleobase sequence according to SEQ ID NO: 169 and the antisense strand comprises a nucleobase sequence according to SEQ ID NO: 73;
(vvv) the sense strand comprises a nucleobase sequence according to SEQ ID NO: 170 and the antisense strand comprises a nucleobase sequence according to SEQ ID NO: 74;
(www) the sense strand comprises a nucleobase sequence according to SEQ ID NO: 171 and the antisense strand comprises a nucleobase sequence according to SEQ ID NO: 75;
(xxx) the sense strand comprises a nucleobase sequence according to SEQ ID NO: 172 and the antisense strand comprises a nucleobase sequence according to SEQ ID NO: 76;
(yyy) the sense strand comprises a nucleobase sequence according to SEQ ID NO: 173 and the antisense strand comprises a nucleobase sequence according to SEQ ID NO: 77;
(zzz) the sense strand comprises a nucleobase sequence according to SEQ ID NO: 174 and the antisense strand comprises a nucleobase sequence according to SEQ ID NO: 78;
(aaaa) the sense strand comprises a nucleobase sequence according to SEQ ID NO: 175 and the antisense strand comprises a nucleobase sequence according to SEQ ID NO: 79;
(bbbb) the sense strand comprises a nucleobase sequence according to SEQ ID NO: 176 and the antisense strand comprises a nucleobase sequence according to SEQ ID NO: 80;
(cccc) the sense strand comprises a nucleobase sequence according to SEQ ID NO: 177 and the antisense strand comprises a nucleobase sequence according to SEQ ID NO: 81;
(dddd) the sense strand comprises a nucleobase sequence according to SEQ ID NO: 178 and the antisense strand comprises a nucleobase sequence according to SEQ ID NO: 82;
(eeee) the sense strand comprises a nucleobase sequence according to SEQ ID NO: 179 and the antisense strand comprises a nucleobase sequence according to SEQ ID NO: 83;
(ffff) the sense strand comprises a nucleobase sequence according to SEQ ID NO: 180 and the antisense strand comprises a nucleobase sequence according to SEQ ID NO: 84;
(gggg) the sense strand comprises a nucleobase sequence according to SEQ ID NO: 181 and the antisense strand comprises a nucleobase sequence according to SEQ ID NO: 85;
(hhhh) the sense strand comprises a nucleobase sequence according to SEQ ID NO: 182 and the antisense strand comprises a nucleobase sequence according to SEQ ID NO: 86;
(iiii) the sense strand comprises a nucleobase sequence according to SEQ ID NO: 183 and the antisense strand comprises a nucleobase sequence according to SEQ ID NO: 87;
(jjjj) the sense strand comprises a nucleobase sequence according to SEQ ID NO: 184 and the antisense strand comprises a nucleobase sequence according to SEQ ID NO: 88;
(kkkk) the sense strand comprises a nucleobase sequence according to SEQ ID NO: 185 and the antisense strand comprises a nucleobase sequence according to SEQ ID NO: 89;
(llll) the sense strand comprises a nucleobase sequence according to SEQ ID NO: 186 and the antisense strand comprises a nucleobase sequence according to SEQ ID NO: 90;
(mmmm) the sense strand comprises a nucleobase sequence according to SEQ ID NO: 187 and the antisense strand comprises a nucleobase sequence according to SEQ ID NO: 91;
(nnnn) the sense strand comprises a nucleobase sequence according to SEQ ID NO: 188 and the antisense strand comprises a nucleobase sequence according to SEQ ID NO: 92;
(oooo) the sense strand comprises a nucleobase sequence according to SEQ ID NO: 189 and the antisense strand comprises a nucleobase sequence according to SEQ ID NO: 93;
(pppp) the sense strand comprises a nucleobase sequence according to SEQ ID NO: 190 and the antisense strand comprises a nucleobase sequence according to SEQ ID NO: 94;
(qqqq) the sense strand comprises a nucleobase sequence according to SEQ ID NO: 191 and the antisense strand comprises a nucleobase sequence according to SEQ ID NO: 95; or
(rrrr) the sense strand comprises a nucleobase sequence according to SEQ ID NO: 192 and the antisense strand comprises a nucleobase sequence according to SEQ ID NO: 96.

Embodiment 177. The method or use of embodiment 176, wherein:
(a) the sense strand comprises a nucleobase sequence according to SEQ ID NO: 104 and the antisense strand comprises a nucleobase sequence according to SEQ ID NO: 8;
(b) the sense strand comprises a nucleobase sequence according to SEQ ID NO: 107 and the antisense strand comprises a nucleobase sequence according to SEQ ID NO: 11;
(c) the sense strand comprises a nucleobase sequence according to SEQ ID NO: 110 and the antisense strand comprises a nucleobase sequence according to SEQ ID NO: 14;
(d) the sense strand comprises a nucleobase sequence according to SEQ ID NO: 112 and the antisense strand comprises a nucleobase sequence according to SEQ ID NO: 16;
(e) the sense strand comprises a nucleobase sequence according to SEQ ID NO: 131 and the antisense strand comprises a nucleobase sequence according to SEQ ID NO: 35;
(f) the sense strand comprises a nucleobase sequence according to SEQ ID NO: 132 and the antisense strand comprises a nucleobase sequence according to SEQ ID NO: 36;
(g) the sense strand comprises a nucleobase sequence according to SEQ ID NO: 134 and the antisense strand comprises a nucleobase sequence according to SEQ ID NO: 38;
(h) the sense strand comprises a nucleobase sequence according to SEQ ID NO: 135 and the antisense strand comprises a nucleobase sequence according to SEQ ID NO: 39;
(i) the sense strand comprises a nucleobase sequence according to SEQ ID NO: 137 and the antisense strand comprises a nucleobase sequence according to SEQ ID NO: 41;
(j) the sense strand comprises a nucleobase sequence according to SEQ ID NO: 138 and the antisense strand comprises a nucleobase sequence according to SEQ ID NO: 42;
(k) the sense strand comprises a nucleobase sequence according to SEQ ID NO: 148 and the antisense strand comprises a nucleobase sequence according to SEQ ID NO: 52;
(l) the sense strand comprises a nucleobase sequence according to SEQ ID NO: 173 and the antisense strand comprises a nucleobase sequence according to SEQ ID NO: 77.
(m) the sense strand comprises a nucleobase sequence according to SEQ ID NO: 180 and the antisense strand comprises a nucleobase sequence according to SEQ ID NO: 84;
(n) the sense strand comprises a nucleobase sequence according to SEQ ID NO: 182 and the antisense strand comprises a nucleobase sequence according to SEQ ID NO: 86;
(o) the sense strand comprises a nucleobase sequence according to SEQ ID NO: 183 and the antisense strand comprises a nucleobase sequence according to SEQ ID NO: 87;
(p) the sense strand comprises a nucleobase sequence according to SEQ ID NO: 184 and the antisense strand comprises a nucleobase sequence according to SEQ ID NO: 88;
(q) the sense strand comprises a nucleobase sequence according to SEQ ID NO: 185 and the antisense strand comprises a nucleobase sequence according to SEQ ID NO: 89;
(r) the sense strand comprises a nucleobase sequence according to SEQ ID NO: 186 and the antisense strand comprises a nucleobase sequence according to SEQ ID NO: 90; or(s) the sense strand comprises a nucleobase sequence according to SEQ ID NO: 189 and the antisense strand comprises a nucleobase sequence according to SEQ ID NO: 93.

Embodiment 178. The method or use of embodiment 176 or 177, wherein:
(a) the sense strand comprises a modified nucleotide sequence according to SEQ ID NO: 289 and the antisense strand comprises a modified nucleotide sequence according to SEQ ID NO: 193;
(b) the sense strand comprises a modified nucleotide sequence according to SEQ ID NO: 290 and the antisense strand comprises a modified nucleotide sequence according to SEQ ID NO: 194;
(c) the sense strand comprises a modified nucleotide sequence according to SEQ ID NO: 291 and the antisense strand comprises a modified nucleotide sequence according to SEQ ID NO: 195;
(d) the sense strand comprises a modified nucleotide sequence according to SEQ ID NO: 292 and the antisense strand comprises a modified nucleotide sequence according to SEQ ID NO: 196;
(e) the sense strand comprises a modified nucleotide sequence according to SEQ ID NO: 293 and the antisense strand comprises a modified nucleotide sequence according to SEQ ID NO: 197;
(f) the sense strand comprises a modified nucleotide sequence according to SEQ ID NO: 294 and the antisense strand comprises a modified nucleotide sequence according to SEQ ID NO: 198;
(g) the sense strand comprises a modified nucleotide sequence according to SEQ ID NO: 295 and the antisense strand comprises a modified nucleotide sequence according to SEQ ID NO: 199;
(h) the sense strand comprises a modified nucleotide sequence according to SEQ ID NO: 296 and the antisense strand comprises a modified nucleotide sequence according to SEQ ID NO: 200;
(i) the sense strand comprises a modified nucleotide sequence according to SEQ ID NO: 297 and the antisense strand comprises a modified nucleotide sequence according to SEQ ID NO: 201;
(j) the sense strand comprises a modified nucleotide sequence according to SEQ ID NO: 298 and the antisense strand comprises a modified nucleotide sequence according to SEQ ID NO: 202;
(k) the sense strand comprises a modified nucleotide sequence according to SEQ ID NO: 299 and the antisense strand comprises a modified nucleotide sequence according to SEQ ID NO: 203;
(l) the sense strand comprises a modified nucleotide sequence according to SEQ ID NO: 300 and the antisense strand comprises a modified nucleotide sequence according to SEQ ID NO: 204;
(m) the sense strand comprises a modified nucleotide sequence according to SEQ ID NO: 301 and the antisense strand comprises a modified nucleotide sequence according to SEQ ID NO: 205;
(n) the sense strand comprises a modified nucleotide sequence according to SEQ ID NO: 302 and the antisense strand comprises a modified nucleotide sequence according to SEQ ID NO: 206;
(o) the sense strand comprises a modified nucleotide sequence according to SEQ ID NO: 303 and the antisense strand comprises a modified nucleotide sequence according to SEQ ID NO: 207;
(p) the sense strand comprises a modified nucleotide sequence according to SEQ ID NO: 304 and the antisense strand comprises a modified nucleotide sequence according to SEQ ID NO: 208;
(q) the sense strand comprises a modified nucleotide sequence according to SEQ ID NO: 305 and the antisense strand comprises a modified nucleotide sequence according to SEQ ID NO: 209;
(r) the sense strand comprises a modified nucleotide sequence according to SEQ ID NO: 306 and the antisense strand comprises a modified nucleotide sequence according to SEQ ID NO: 210;
(s) the sense strand comprises a modified nucleotide sequence according to SEQ ID NO: 307 and the antisense strand comprises a modified nucleotide sequence according to SEQ ID NO: 211;
(t) the sense strand comprises a modified nucleotide sequence according to SEQ ID NO: 308 and the antisense strand comprises a modified nucleotide sequence according to SEQ ID NO: 212;
(u) the sense strand comprises a modified nucleotide sequence according to SEQ ID NO: 309 and the antisense strand comprises a modified nucleotide sequence according to SEQ ID NO: 213;
(v) the sense strand comprises a modified nucleotide sequence according to SEQ ID NO: 310 and the antisense strand comprises a modified nucleotide sequence according to SEQ ID NO: 214;
(w) the sense strand comprises a modified nucleotide sequence according to SEQ ID NO: 311 and the antisense strand comprises a modified nucleotide sequence according to SEQ ID NO: 215;
(x) the sense strand comprises a modified nucleotide sequence according to SEQ ID NO: 312 and the antisense strand comprises a modified nucleotide sequence according to SEQ ID NO: 216;
(y) the sense strand comprises a modified nucleotide sequence according to SEQ ID NO: 313 and the antisense strand comprises a modified nucleotide sequence according to SEQ ID NO: 217;
(z) the sense strand comprises a modified nucleotide sequence according to SEQ ID NO: 314 and the antisense strand comprises a modified nucleotide sequence according to SEQ ID NO: 218;
(aa) the sense strand comprises a modified nucleotide sequence according to SEQ ID NO: 315 and the antisense strand comprises a modified nucleotide sequence according to SEQ ID NO: 219;
(bb) the sense strand comprises a modified nucleotide sequence according to SEQ ID NO: 316 and the antisense strand comprises a modified nucleotide sequence according to SEQ ID NO: 220;
(cc) the sense strand comprises a modified nucleotide sequence according to SEQ ID NO: 317 and the antisense strand comprises a modified nucleotide sequence according to SEQ ID NO: 221;
(dd) the sense strand comprises a modified nucleotide sequence according to SEQ ID NO: 318 and the antisense strand comprises a modified nucleotide sequence according to SEQ ID NO: 222;
(ee) the sense strand comprises a modified nucleotide sequence according to SEQ ID NO: 319 and the antisense strand comprises a modified nucleotide sequence according to SEQ ID NO: 223;
(ff) the sense strand comprises a modified nucleotide sequence according to SEQ ID NO: 320 and the antisense strand comprises a modified nucleotide sequence according to SEQ ID NO: 224;
(gg) the sense strand comprises a modified nucleotide sequence according to SEQ ID NO: 321 and the antisense strand comprises a modified nucleotide sequence according to SEQ ID NO: 225;
(hh) the sense strand comprises a modified nucleotide sequence according to SEQ ID NO: 322 and the antisense strand comprises a modified nucleotide sequence according to SEQ ID NO: 226;
(ii) the sense strand comprises a modified nucleotide sequence according to SEQ ID NO: 323 and the antisense strand comprises a modified nucleotide sequence according to SEQ ID NO: 227;
(jj) the sense strand comprises a modified nucleotide sequence according to SEQ ID NO: 324 and the antisense strand comprises a modified nucleotide sequence according to SEQ ID NO: 228;
(kk) the sense strand comprises a modified nucleotide sequence according to SEQ ID NO: 325 and the antisense strand comprises a modified nucleotide sequence according to SEQ ID NO: 229;
(ll) the sense strand comprises a modified nucleotide sequence according to SEQ ID NO: 326 and the antisense strand comprises a modified nucleotide sequence according to SEQ ID NO: 230;
(mm) the sense strand comprises a modified nucleotide sequence according to SEQ ID NO: 327 and the antisense strand comprises a modified nucleotide sequence according to SEQ ID NO: 231;
(nn) the sense strand comprises a modified nucleotide sequence according to SEQ ID NO: 328 and the antisense strand comprises a modified nucleotide sequence according to SEQ ID NO: 232;
(oo) the sense strand comprises a modified nucleotide sequence according to SEQ ID NO: 329 and the antisense strand comprises a modified nucleotide sequence according to SEQ ID NO: 233;
(pp) the sense strand comprises a modified nucleotide sequence according to SEQ ID NO: 330 and the antisense strand comprises a modified nucleotide sequence according to SEQ ID NO: 234;
(qq) the sense strand comprises a modified nucleotide sequence according to SEQ ID NO: 331 and the antisense strand comprises a modified nucleotide sequence according to SEQ ID NO: 235;
(rr) the sense strand comprises a modified nucleotide sequence according to SEQ ID NO: 332 and the antisense strand comprises a modified nucleotide sequence according to SEQ ID NO: 236;
(ss) the sense strand comprises a modified nucleotide sequence according to SEQ ID NO: 333 and the antisense strand comprises a modified nucleotide sequence according to SEQ ID NO: 237;
(tt) the sense strand comprises a modified nucleotide sequence according to SEQ ID NO: 334 and the antisense strand comprises a modified nucleotide sequence according to SEQ ID NO: 238;
(uu) the sense strand comprises a modified nucleotide sequence according to SEQ ID NO: 335 and the antisense strand comprises a modified nucleotide sequence according to SEQ ID NO: 239;
(vv) the sense strand comprises a modified nucleotide sequence according to SEQ ID NO: 336 and the antisense strand comprises a modified nucleotide sequence according to SEQ ID NO: 240;
(ww) the sense strand comprises a modified nucleotide sequence according to SEQ ID NO: 337 and the antisense strand comprises a modified nucleotide sequence according to SEQ ID NO: 241;
(xx) the sense strand comprises a modified nucleotide sequence according to SEQ ID NO: 338 and the antisense strand comprises a modified nucleotide sequence according to SEQ ID NO: 242;
(yy) the sense strand comprises a modified nucleotide sequence according to SEQ ID NO: 339 and the antisense strand comprises a modified nucleotide sequence according to SEQ ID NO: 243;
(zz) the sense strand comprises a modified nucleotide sequence according to SEQ ID NO: 340 and the antisense strand comprises a modified nucleotide sequence according to SEQ ID NO: 244;
(aaa) the sense strand comprises a modified nucleotide sequence according to SEQ ID NO: 341 and the antisense strand comprises a modified nucleotide sequence according to SEQ ID NO: 245;
(bbb) the sense strand comprises a modified nucleotide sequence according to SEQ ID NO: 342 and the antisense strand comprises a modified nucleotide sequence according to SEQ ID NO: 246;
(ccc) the sense strand comprises a modified nucleotide sequence according to SEQ ID NO: 343 and the antisense strand comprises a modified nucleotide sequence according to SEQ ID NO: 247;
(ddd) the sense strand comprises a modified nucleotide sequence according to SEQ ID NO: 344 and the antisense strand comprises a modified nucleotide sequence according to SEQ ID NO: 248;
(eee) the sense strand comprises a modified nucleotide sequence according to SEQ ID NO: 345 and the antisense strand comprises a modified nucleotide sequence according to SEQ ID NO: 249;
(fff) the sense strand comprises a modified nucleotide sequence according to SEQ ID NO: 346 and the antisense strand comprises a modified nucleotide sequence according to SEQ ID NO: 250;
(ggg) the sense strand comprises a modified nucleotide sequence according to SEQ ID NO: 347 and the antisense strand comprises a modified nucleotide sequence according to SEQ ID NO: 251;
(hhh) the sense strand comprises a modified nucleotide sequence according to SEQ ID NO: 348 and the antisense strand comprises a modified nucleotide sequence according to SEQ ID NO: 252;
(iii) the sense strand comprises a modified nucleotide sequence according to SEQ ID NO: 349 and the antisense strand comprises a modified nucleotide sequence according to SEQ ID NO: 253;
(jjj) the sense strand comprises a modified nucleotide sequence according to SEQ ID NO: 350 and the antisense strand comprises a modified nucleotide sequence according to SEQ ID NO: 254;
(kkk) the sense strand comprises a modified nucleotide sequence according to SEQ ID NO: 351 and the antisense strand comprises a modified nucleotide sequence according to SEQ ID NO: 255;
(lll) the sense strand comprises a modified nucleotide sequence according to SEQ ID NO: 352 and the antisense strand comprises a modified nucleotide sequence according to SEQ ID NO: 256;
(mmm) the sense strand comprises a modified nucleotide sequence according to SEQ ID NO: 353 and the antisense strand comprises a modified nucleotide sequence according to SEQ ID NO: 257;
(nnn) the sense strand comprises a modified nucleotide sequence according to SEQ ID NO: 354 and the antisense strand comprises a modified nucleotide sequence according to SEQ ID NO: 258;
(ooo) the sense strand comprises a modified nucleotide sequence according to SEQ ID NO: 355 and the antisense strand comprises a modified nucleotide sequence according to SEQ ID NO: 259;
(ppp) the sense strand comprises a modified nucleotide sequence according to SEQ ID NO: 356 and the antisense strand comprises a modified nucleotide sequence according to SEQ ID NO: 260;
(qqq) the sense strand comprises a modified nucleotide sequence according to SEQ ID NO: 357 and the antisense strand comprises a modified nucleotide sequence according to SEQ ID NO: 261;
(rrr) the sense strand comprises a modified nucleotide sequence according to SEQ ID NO: 358 and the antisense strand comprises a modified nucleotide sequence according to SEQ ID NO: 262;
(sss) the sense strand comprises a modified nucleotide sequence according to SEQ ID NO: 359 and the antisense strand comprises a modified nucleotide sequence according to SEQ ID NO: 263;
(ttt) the sense strand comprises a modified nucleotide sequence according to SEQ ID NO: 360 and the antisense strand comprises a modified nucleotide sequence according to SEQ ID NO: 264;
(uuu) the sense strand comprises a modified nucleotide sequence according to SEQ ID NO: 361 and the antisense strand comprises a modified nucleotide sequence according to SEQ ID NO: 265;
(vvv) the sense strand comprises a modified nucleotide sequence according to SEQ ID NO: 362 and the antisense strand comprises a modified nucleotide sequence according to SEQ ID NO: 266;
(www) the sense strand comprises a modified nucleotide sequence according to SEQ ID NO: 363 and the antisense strand comprises a modified nucleotide sequence according to SEQ ID NO: 267;
(xxx) the sense strand comprises a modified nucleotide sequence according to SEQ ID NO: 364 and the antisense strand comprises a modified nucleotide sequence according to SEQ ID NO: 268;
(yyy) the sense strand comprises a modified nucleotide sequence according to SEQ ID NO: 365 and the antisense strand comprises a modified nucleotide sequence according to SEQ ID NO: 269;
(zzz) the sense strand comprises a modified nucleotide sequence according to SEQ ID NO: 366 and the antisense strand comprises a modified nucleotide sequence according to SEQ ID NO: 270;
(aaaa) the sense strand comprises a modified nucleotide sequence according to SEQ ID NO: 367 and the antisense strand comprises a modified nucleotide sequence according to SEQ ID NO: 271;
(bbbb) the sense strand comprises a modified nucleotide sequence according to SEQ ID NO: 368 and the antisense strand comprises a modified nucleotide sequence according to SEQ ID NO: 272;
(cccc) the sense strand comprises a modified nucleotide sequence according to SEQ ID NO: 369 and the antisense strand comprises a modified nucleotide sequence according to SEQ ID NO: 273;
(dddd) the sense strand comprises a modified nucleotide sequence according to SEQ ID NO: 370 and the antisense strand comprises a modified nucleotide sequence according to SEQ ID NO: 274;
(eeee) the sense strand comprises a modified nucleotide sequence according to SEQ ID NO: 371 and the antisense strand comprises a modified nucleotide sequence according to SEQ ID NO: 275;
(ffff) the sense strand comprises a modified nucleotide sequence according to SEQ ID NO: 372 and the antisense strand comprises a modified nucleotide sequence according to SEQ ID NO: 276;
(gggg) the sense strand comprises a modified nucleotide sequence according to SEQ ID NO: 373 and the antisense strand comprises a modified nucleotide sequence according to SEQ ID NO: 277;
(hhhh) the sense strand comprises a modified nucleotide sequence according to SEQ ID NO: 374 and the antisense strand comprises a modified nucleotide sequence according to SEQ ID NO: 278;
(iiii) the sense strand comprises a modified nucleotide sequence according to SEQ ID NO: 375 and the antisense strand comprises a modified nucleotide sequence according to SEQ ID NO: 279;
(jjjj) the sense strand comprises a modified nucleotide sequence according to SEQ ID NO: 376 and the antisense strand comprises a modified nucleotide sequence according to SEQ ID NO: 280;
(kkkk) the sense strand comprises a modified nucleotide sequence according to SEQ ID NO: 377 and the antisense strand comprises a modified nucleotide sequence according to SEQ ID NO: 281;
(llll) the sense strand comprises a modified nucleotide sequence according to SEQ ID NO: 378 and the antisense strand comprises a modified nucleotide sequence according to SEQ ID NO: 282;
(mmmm) the sense strand comprises a modified nucleotide sequence according to SEQ ID NO: 379 and the antisense strand comprises a modified nucleotide sequence according to SEQ ID NO: 283;
(nnnn) the sense strand comprises a modified nucleotide sequence according to SEQ ID NO: 380 and the antisense strand comprises a modified nucleotide sequence according to SEQ ID NO: 284;
(oooo) the sense strand comprises a modified nucleotide sequence according to SEQ ID NO: 381 and the antisense strand comprises a modified nucleotide sequence according to SEQ ID NO: 285;
(pppp) the sense strand comprises a modified nucleotide sequence according to SEQ ID NO: 382 and the antisense strand comprises a modified nucleotide sequence according to SEQ ID NO: 286;
(qqqq) the sense strand comprises a modified nucleotide sequence according to SEQ ID NO: 383 and the antisense strand comprises a modified nucleotide sequence according to SEQ ID NO: 287;
(rrrr) the sense strand comprises a modified nucleotide sequence according to SEQ ID NO: 384 and the antisense strand comprises a modified nucleotide sequence according to SEQ ID NO: 288;
(ssss) the sense strand comprises a modified nucleotide sequence according to SEQ ID NO: 385 and the antisense strand comprises a modified nucleotide sequence according to SEQ ID NO: 193;
(tttt) the sense strand comprises a modified nucleotide sequence according to SEQ ID NO: 386 and the antisense strand comprises a modified nucleotide sequence according to SEQ ID NO: 194;
(uuuu) the sense strand comprises a modified nucleotide sequence according to SEQ ID NO: 387 and the antisense strand comprises a modified nucleotide sequence according to SEQ ID NO: 195;
(vvvv) the sense strand comprises a modified nucleotide sequence according to SEQ ID NO: 388 and the antisense strand comprises a modified nucleotide sequence according to SEQ ID NO: 196;
(wwww) the sense strand comprises a modified nucleotide sequence according to SEQ ID NO: 389 and the antisense strand comprises a modified nucleotide sequence according to SEQ ID NO: 197;
(xxxx) the sense strand comprises a modified nucleotide sequence according to SEQ ID NO: 390 and the antisense strand comprises a modified nucleotide sequence according to SEQ ID NO: 198;
(yyyy) the sense strand comprises a modified nucleotide sequence according to SEQ ID NO: 391 and the antisense strand comprises a modified nucleotide sequence according to SEQ ID NO: 199;
(zzzz) the sense strand comprises a modified nucleotide sequence according to SEQ ID NO: 392 and the antisense strand comprises a modified nucleotide sequence according to SEQ ID NO: 200;
(aaaaa) the sense strand comprises a modified nucleotide sequence according to SEQ ID NO: 393 and the antisense strand comprises a modified nucleotide sequence according to SEQ ID NO: 201;
(bbbbb) the sense strand comprises a modified nucleotide sequence according to SEQ ID NO: 394 and the antisense strand comprises a modified nucleotide sequence according to SEQ ID NO: 202;
(ccccc) the sense strand comprises a modified nucleotide sequence according to SEQ ID NO: 395 and the antisense strand comprises a modified nucleotide sequence according to SEQ ID NO: 203;
(ddddd) the sense strand comprises a modified nucleotide sequence according to SE Q ID NO: 396 and the antisense strand comprises a modified nucleotide sequence according to SEQ ID NO: 204;
(eeeee) the sense strand comprises a modified nucleotide sequence according to SEQ ID NO: 397 and the antisense strand comprises a modified nucleotide sequence according to SEQ ID NO: 205;
(fffff) the sense strand comprises a modified nucleotide sequence according to SEQ ID NO: 398 and the antisense strand comprises a modified nucleotide sequence according to SEQ ID NO: 206;
(ggggg) the sense strand comprises a modified nucleotide sequence according to SEQ ID NO: 399 and the antisense strand comprises a modified nucleotide sequence according to SEQ ID NO: 207;
(hhhhh) the sense strand comprises a modified nucleotide sequence according to SEQ ID NO: 400 and the antisense strand comprises a modified nucleotide sequence according to SEQ ID NO: 208;
(iiiii) the sense strand comprises a modified nucleotide sequence according to SEQ ID NO: 401 and the antisense strand comprises a modified nucleotide sequence according to SEQ ID NO: 209;
(jjjjj) the sense strand comprises a modified nucleotide sequence according to SEQ ID NO: 402 and the antisense strand comprises a modified nucleotide sequence according to SEQ ID NO: 210;
(kkkkk) the sense strand comprises a modified nucleotide sequence according to SEQ ID NO: 403 and the antisense strand comprises a modified nucleotide sequence according to SEQ ID NO: 211;
(lllll) the sense strand comprises a modified nucleotide sequence according to SEQ ID NO: 404 and the antisense strand comprises a modified nucleotide sequence according to SEQ ID NO: 212;
(mmmmm) the sense strand comprises a modified nucleotide sequence according to SEQ ID NO: 405 and the antisense strand comprises a modified nucleotide sequence according to SEQ ID NO: 213;
(nnnnn) the sense strand comprises a modified nucleotide sequence according to SEQ ID NO: 406 and the antisense strand comprises a modified nucleotide sequence according to SEQ ID NO: 214;
(ooooo) the sense strand comprises a modified nucleotide sequence according to SEQ ID NO: 407 and the antisense strand comprises a modified nucleotide sequence according to SEQ ID NO: 215;
(ppppp) the sense strand comprises a modified nucleotide sequence according to SEQ ID NO: 408 and the antisense strand comprises a modified nucleotide sequence according to SEQ ID NO: 216;
(qqqqq) the sense strand comprises a modified nucleotide sequence according to SEQ ID NO: 409 and the antisense strand comprises a modified nucleotide sequence according to SEQ ID NO: 217;
(rrrrr) the sense strand comprises a modified nucleotide sequence according to SEQ ID NO: 410 and the antisense strand comprises a modified nucleotide sequence according to SEQ ID NO: 218;
(sssss) the sense strand comprises a modified nucleotide sequence according to SEQ ID NO: 411 and the antisense strand comprises a modified nucleotide sequence according to SEQ ID NO: 219;
(ttttt) the sense strand comprises a modified nucleotide sequence according to SEQ ID NO: 412 and the antisense strand comprises a modified nucleotide sequence according to SEQ ID NO: 220;
(uuuuu) the sense strand comprises a modified nucleotide sequence according to SEQ ID NO: 413 and the antisense strand comprises a modified nucleotide sequence according to SEQ ID NO: 221;
(vvvvv) the sense strand comprises a modified nucleotide sequence according to SEQ ID NO: 414 and the antisense strand comprises a modified nucleotide sequence according to SEQ ID NO: 222;
(wwwww) the sense strand comprises a modified nucleotide sequence according to SEQ ID NO: 415 and the antisense strand comprises a modified nucleotide sequence according to SEQ ID NO: 223;
(xxxxx) the sense strand comprises a modified nucleotide sequence according to SEQ ID NO: 416 and the antisense strand comprises a modified nucleotide sequence according to SEQ ID NO: 224;
(yyyyy) the sense strand comprises a modified nucleotide sequence according to SEQ ID NO: 417 and the antisense strand comprises a modified nucleotide sequence according to SEQ ID NO: 225;
(zzzzz) the sense strand comprises a modified nucleotide sequence according to SEQ ID NO: 418 and the antisense strand comprises a modified nucleotide sequence according to SEQ ID NO: 226;
(aaaaaa) the sense strand comprises a modified nucleotide sequence according to SEQ ID NO: 419 and the antisense strand comprises a modified nucleotide sequence according to SEQ ID NO: 227;
(bbbbbb) the sense strand comprises a modified nucleotide sequence according to SEQ ID NO: 420 and the antisense strand comprises a modified nucleotide sequence according to SEQ ID NO: 228;
(cccccc) the sense strand comprises a modified nucleotide sequence according to SEQ ID NO: 421 and the antisense strand comprises a modified nucleotide sequence according to SEQ ID NO: 229;
(dddddd) the sense strand comprises a modified nucleotide sequence according to SEQ ID NO: 422 and the antisense strand comprises a modified nucleotide sequence according to SEQ ID NO: 230;
(eeeeee) the sense strand comprises a modified nucleotide sequence according to SEQ ID NO: 423 and the antisense strand comprises a modified nucleotide sequence according to SEQ ID NO: 231;
(ffffff) the sense strand comprises a modified nucleotide sequence according to SEQ ID NO: 424 and the antisense strand comprises a modified nucleotide sequence according to SEQ ID NO: 232;
(gggggg) the sense strand comprises a modified nucleotide sequence according to SEQ ID NO: 425 and the antisense strand comprises a modified nucleotide sequence according to SEQ ID NO: 233;
(hhhhhh) the sense strand comprises a modified nucleotide sequence according to SEQ ID NO: 426 and the antisense strand comprises a modified nucleotide sequence according to SEQ ID NO: 234;
(iiiiii) the sense strand comprises a modified nucleotide sequence according to SEQ ID NO: 427 and the antisense strand comprises a modified nucleotide sequence according to SEQ ID NO: 235;
(jjjjjj) the sense strand comprises a modified nucleotide sequence according to SEQ ID NO: 428 and the antisense strand comprises a modified nucleotide sequence according to SEQ ID NO: 236;
(kkkkkk) the sense strand comprises a modified nucleotide sequence according to SEQ ID NO: 429 and the antisense strand comprises a modified nucleotide sequence according to SEQ ID NO: 237;
(llllll) the sense strand comprises a modified nucleotide sequence according to SEQ ID NO: 430 and the antisense strand comprises a modified nucleotide sequence according to SEQ ID NO: 238;
(mmmmmm) the sense strand comprises a modified nucleotide sequence according to SEQ ID NO: 431 and the antisense strand comprises a modified nucleotide sequence according to SEQ ID NO: 239;
(nnnnnn) the sense strand comprises a modified nucleotide sequence according to SEQ ID NO: 432 and the antisense strand comprises a modified nucleotide sequence according to SEQ ID NO: 240;
(oooooo) the sense strand comprises a modified nucleotide sequence according to SEQ ID NO: 433 and the antisense strand comprises a modified nucleotide sequence according to SEQ ID NO: 241;
(pppppp) the sense strand comprises a modified nucleotide sequence according to SEQ ID NO: 434 and the antisense strand comprises a modified nucleotide sequence according to SEQ ID NO: 242;
(qqqqqq) the sense strand comprises a modified nucleotide sequence according to SEQ ID NO: 435 and the antisense strand comprises a modified nucleotide sequence according to SEQ ID NO: 243;
(rrrrrr) the sense strand comprises a modified nucleotide sequence according to SEQ ID NO: 436 and the antisense strand comprises a modified nucleotide sequence according to SEQ ID NO: 244;
(ssssss) the sense strand comprises a modified nucleotide sequence according to SEQ ID NO: 437 and the antisense strand comprises a modified nucleotide sequence according to SEQ ID NO: 245;
(tttttt) the sense strand comprises a modified nucleotide sequence according to SEQ ID NO: 438 and the antisense strand comprises a modified nucleotide sequence according to SEQ ID NO: 246;
(uuuuuu) the sense strand comprises a modified nucleotide sequence according to SEQ ID NO: 439 and the antisense strand comprises a modified nucleotide sequence according to SEQ ID NO: 247;
(vvvvvv) the sense strand comprises a modified nucleotide sequence according to SEQ ID NO: 440 and the antisense strand comprises a modified nucleotide sequence according to SEQ ID NO: 248;
(wwwwww) the sense strand comprises a modified nucleotide sequence according to SEQ ID NO: 441 and the antisense strand comprises a modified nucleotide sequence according to SEQ ID NO: 249;
(xxxxxx) the sense strand comprises a modified nucleotide sequence according to SEQ ID NO: 442 and the antisense strand comprises a modified nucleotide sequence according to SEQ ID NO: 250;
(yyyyyy) the sense strand comprises a modified nucleotide sequence according to SEQ ID NO: 443 and the antisense strand comprises a modified nucleotide sequence according to SEQ ID NO: 251;
(zzzzzz) the sense strand comprises a modified nucleotide sequence according to SEQ ID NO: 444 and the antisense strand comprises a modified nucleotide sequence according to SEQ ID NO: 252;
(aaaaaaa) the sense strand comprises a modified nucleotide sequence according to SEQ ID NO: 445 and the antisense strand comprises a modified nucleotide sequence according to SEQ ID NO: 253;
(bbbbbbb) the sense strand comprises a modified nucleotide sequence according to SEQ ID NO: 446 and the antisense strand comprises a modified nucleotide sequence according to SEQ ID NO: 254;
(ccccccc) the sense strand comprises a modified nucleotide sequence according to SEQ ID NO: 447 and the antisense strand comprises a modified nucleotide sequence according to SEQ ID NO: 255;
(ddddddd) the sense strand comprises a modified nucleotide sequence according to SEQ ID NO: 448 and the antisense strand comprises a modified nucleotide sequence according to SEQ ID NO: 256;
(eeeeeee) the sense strand comprises a modified nucleotide sequence according to SEQ ID NO: 449 and the antisense strand comprises a modified nucleotide sequence according to SEQ ID NO: 257;
(fffffff) the sense strand comprises a modified nucleotide sequence according to SEQ ID NO: 450 and the antisense strand comprises a modified nucleotide sequence according to SEQ ID NO: 258;
(ggggggg) the sense strand comprises a modified nucleotide sequence according to SEQ ID NO: 451 and the antisense strand comprises a modified nucleotide sequence according to SEQ ID NO: 259;
(hhhhhhh) the sense strand comprises a modified nucleotide sequence according to SEQ ID NO: 452 and the antisense strand comprises a modified nucleotide sequence according to SEQ ID NO: 260;
(iiiiii) the sense strand comprises a modified nucleotide sequence according to SEQ ID NO: 453 and the antisense strand comprises a modified nucleotide sequence according to SEQ ID NO: 261;
(jjjjjjj) the sense strand comprises a modified nucleotide sequence according to SEQ ID NO: 454 and the antisense strand comprises a modified nucleotide sequence according to SEQ ID NO: 262;
(kkkkkkk) the sense strand comprises a modified nucleotide sequence according to SEQ ID NO: 455 and the antisense strand comprises a modified nucleotide sequence according to SEQ ID NO: 263;
(lllllll) the sense strand comprises a modified nucleotide sequence according to SEQ ID NO: 456 and the antisense strand comprises a modified nucleotide sequence according to SEQ ID NO: 264;
(mmmmmmm) the sense strand comprises a modified nucleotide sequence according to SEQ ID NO: 457 and the antisense strand comprises a modified nucleotide sequence according to SEQ ID NO: 265;
(nnnnnnn) the sense strand comprises a modified nucleotide sequence according to SEQ ID NO: 458 and the antisense strand comprises a modified nucleotide sequence according to SEQ ID NO: 266;
(ooooooo) the sense strand comprises a modified nucleotide sequence according to SEQ ID NO: 459 and the antisense strand comprises a modified nucleotide sequence according to SEQ ID NO: 267;
(ppppppp) the sense strand comprises a modified nucleotide sequence according to SEQ ID NO: 460 and the antisense strand comprises a modified nucleotide sequence according to SEQ ID NO: 268;
(qqqqqqq) the sense strand comprises a modified nucleotide sequence according to SEQ ID NO: 461 and the antisense strand comprises a modified nucleotide sequence according to SEQ ID NO: 269;
(rrrrrrrr) the sense strand comprises a modified nucleotide sequence according to SEQ ID NO: 462 and the antisense strand comprises a modified nucleotide sequence according to SEQ ID NO: 270;
(sssssss) the sense strand comprises a modified nucleotide sequence according to SEQ ID NO: 463 and the antisense strand comprises a modified nucleotide sequence according to SEQ ID NO: 271;
(ttttttt) the sense strand comprises a modified nucleotide sequence according to SEQ ID NO: 464 and the antisense strand comprises a modified nucleotide sequence according to SEQ ID NO: 272;
(uuuuuuu) the sense strand comprises a modified nucleotide sequence according to SEQ ID NO: 465 and the antisense strand comprises a modified nucleotide sequence according to SEQ ID NO: 273;
(vvvvvvv) the sense strand comprises a modified nucleotide sequence according to SEQ ID NO: 466 and the antisense strand comprises a modified nucleotide sequence according to SEQ ID NO: 274;
(wwwwwww) the sense strand comprises a modified nucleotide sequence according to SEQ ID NO: 467 and the antisense strand comprises a modified nucleotide sequence according to SEQ ID NO: 275;
(xxxxxxx) the sense strand comprises a modified nucleotide sequence according to SEQ ID NO: 468 and the antisense strand comprises a modified nucleotide sequence according to SEQ ID NO: 276;
(yyyyyyy) the sense strand comprises a modified nucleotide sequence according to SEQ ID NO: 469 and the antisense strand comprises a modified nucleotide sequence according to SEQ ID NO: 277;
(zzzzzzz) the sense strand comprises a modified nucleotide sequence according to SEQ ID NO: 470 and the antisense strand comprises a modified nucleotide sequence according to SEQ ID NO: 278;
(aaaaaaaa) the sense strand comprises a modified nucleotide sequence according to SEQ ID NO: 471 and the antisense strand comprises a modified nucleotide sequence according to SEQ ID NO: 279;
(bbbbbbbb) the sense strand comprises a modified nucleotide sequence according to SEQ ID NO: 472 and the antisense strand comprises a modified nucleotide sequence according to SEQ ID NO: 280;
(cccccccc) the sense strand comprises a modified nucleotide sequence according to SEQ ID NO: 473 and the antisense strand comprises a modified nucleotide sequence according to SEQ ID NO: 281;
(dddddddd) the sense strand comprises a modified nucleotide sequence according to SEQ ID NO: 474 and the antisense strand comprises a modified nucleotide sequence according to SEQ ID NO: 282;
(eeeeeeee) the sense strand comprises a modified nucleotide sequence according to SEQ ID NO: 475 and the antisense strand comprises a modified nucleotide sequence according to SEQ ID NO: 283;
(ffffffff) the sense strand comprises a modified nucleotide sequence according to SEQ ID NO: 476 and the antisense strand comprises a modified nucleotide sequence according to SEQ ID NO: 284;
(gggggggg) the sense strand comprises a modified nucleotide sequence according to SEQ ID NO: 477 and the antisense strand comprises a modified nucleotide sequence according to SEQ ID NO: 285;
(hhhhhhhh) the sense strand comprises a modified nucleotide sequence according to SEQ ID NO: 478 and the antisense strand comprises a modified nucleotide sequence according to SEQ ID NO: 286;
(iiiiiii) the sense strand comprises a modified nucleotide sequence according to SEQ ID NO: 479 and the antisense strand comprises a modified nucleotide sequence according to SEQ ID NO: 287; or
(jjjjjjjj) the sense strand comprises a modified nucleotide sequence according to SEQ ID NO: 480 and the antisense strand comprises a modified nucleotide sequence according to SEQ ID NO: 288.

Embodiment 179. The method or use of embodiment 178, wherein:
(a) the sense strand comprises a modified nucleobase sequence according to SEQ ID NO: 296 and the antisense strand comprises a modified nucleobase sequence according to SEQ ID NO: 200;
(b) the sense strand comprises a modified nucleobase sequence according to SEQ ID NO: 299 and the antisense strand comprises a modified nucleobase sequence according to SEQ ID NO: 203;
(c) the sense strand comprises a modified nucleobase sequence according to SEQ ID NO: 302 and the antisense strand comprises a modified nucleobase sequence according to SEQ ID NO: 206;
(d) the sense strand comprises a modified nucleobase sequence according to SEQ ID NO: 304 and the antisense strand comprises a modified nucleobase sequence according to SEQ ID NO: 208;
(e) the sense strand comprises a modified nucleobase sequence according to SEQ ID NO: 323 and the antisense strand comprises a modified nucleobase sequence according to SEQ ID NO: 227;
(f) the sense strand comprises a modified nucleobase sequence according to SEQ ID NO: 324 and the antisense strand comprises a modified nucleobase sequence according to SEQ ID NO: 228;
(g) the sense strand comprises a modified nucleobase sequence according to SEQ ID NO: 326 and the antisense strand comprises a modified nucleobase sequence according to SEQ ID NO: 230;
(h) the sense strand comprises a modified nucleobase sequence according to SEQ ID NO: 327 and the antisense strand comprises a modified nucleobase sequence according to SEQ ID NO: 231;
(i) the sense strand comprises a modified nucleobase sequence according to SEQ ID NO: 329 and the antisense strand comprises a modified nucleobase sequence according to SEQ ID NO: 233;
(j) the sense strand comprises a modified nucleobase sequence according to SEQ ID NO: 330 and the antisense strand comprises a modified nucleobase sequence according to SEQ ID NO: 234;
(k) the sense strand comprises a modified nucleobase sequence according to SEQ ID NO: 340 and the antisense strand comprises a modified nucleobase sequence according to SEQ ID NO: 244;
(l) the sense strand comprises a modified nucleobase sequence according to SEQ ID NO: 365 and the antisense strand comprises a modified nucleobase sequence according to SEQ ID NO: 269;
(m) the sense strand comprises a modified nucleobase sequence according to SEQ ID NO: 372 and the antisense strand comprises a modified nucleobase sequence according to SEQ ID NO: 276;
(n) the sense strand comprises a modified nucleobase sequence according to SEQ ID NO: 374 and the antisense strand comprises a modified nucleobase sequence according to SEQ ID NO: 278;
(o) the sense strand comprises a modified nucleobase sequence according to SEQ ID NO: 375 and the antisense strand comprises a modified nucleobase sequence according to SEQ ID NO: 279;
(p) the sense strand comprises a modified nucleobase sequence according to SEQ ID NO: 376 and the antisense strand comprises a modified nucleobase sequence according to SEQ ID NO: 280;
(q) the sense strand comprises a modified nucleobase sequence according to SEQ ID NO: 377 and the antisense strand comprises a modified nucleobase sequence according to SEQ ID NO: 281;
(r) the sense strand comprises a modified nucleobase sequence according to SEQ ID NO: 378 and the antisense strand comprises a modified nucleobase sequence according to SEQ ID NO: 282;
(s) the sense strand comprises a modified nucleobase sequence according to SEQ ID NO: 381 and the antisense strand comprises a modified nucleobase sequence according to SEQ ID NO: 285;
(t) the sense strand comprises a modified nucleobase sequence according to SEQ ID NO: 392 and the antisense strand comprises a modified nucleobase sequence according to SEQ ID NO: 200;
(u) the sense strand comprises a modified nucleobase sequence according to SEQ ID NO: 395 and the antisense strand comprises a modified nucleobase sequence according to SEQ ID NO: 203;
(v) the sense strand comprises a modified nucleobase sequence according to SEQ ID NO: 398 and the antisense strand comprises a modified nucleobase sequence according to SEQ ID NO: 206;
(w) the sense strand comprises a modified nucleobase sequence according to SEQ ID NO: 400 and the antisense strand comprises a modified nucleobase sequence according to SEQ ID NO: 208;
(x) the sense strand comprises a modified nucleobase sequence according to SEQ ID NO: 419 and the antisense strand comprises a modified nucleobase sequence according to SEQ ID NO: 227;
(y) the sense strand comprises a modified nucleobase sequence according to SEQ ID NO: 420 and the antisense strand comprises a modified nucleobase sequence according to SEQ ID NO: 228;
(z) the sense strand comprises a modified nucleobase sequence according to SEQ ID NO: 422 and the antisense strand comprises a modified nucleobase sequence according to SEQ ID NO: 230;
(aa) the sense strand comprises a modified nucleobase sequence according to SEQ ID NO: 423 and the antisense strand comprises a modified nucleobase sequence according to SEQ ID NO: 231;
(bb) the sense strand comprises a modified nucleobase sequence according to SEQ ID NO: 425 and the antisense strand comprises a modified nucleobase sequence according to SEQ ID NO: 233;
(cc) the sense strand comprises a modified nucleobase sequence according to SEQ ID NO: 426 and the antisense strand comprises a modified nucleobase sequence according to SEQ ID NO: 234;
(dd) the sense strand comprises a modified nucleobase sequence according to SEQ ID NO: 436 and the antisense strand comprises a modified nucleobase sequence according to SEQ ID NO: 244;
(ee) the sense strand comprises a modified nucleobase sequence according to SEQ ID NO: 461 and the antisense strand comprises a modified nucleobase sequence according to SEQ ID NO: 269;
(ff) the sense strand comprises a modified nucleobase sequence according to SEQ ID NO: 468 and the antisense strand comprises a modified nucleobase sequence according to SEQ ID NO: 276;
(gg) the sense strand comprises a modified nucleobase sequence according to SEQ ID NO: 470 and the antisense strand comprises a modified nucleobase sequence according to SEQ ID NO: 278;
(hh) the sense strand comprises a modified nucleobase sequence according to SEQ ID NO: 471 and the antisense strand comprises a modified nucleobase sequence according to SEQ ID NO: 279;
(ii) the sense strand comprises a modified nucleobase sequence according to SEQ ID NO: 472 and the antisense strand comprises a modified nucleobase sequence according to SEQ ID NO: 280;
(jj) the sense strand comprises a modified nucleobase sequence according to SEQ ID NO: 473 and the antisense strand comprises a modified nucleobase sequence according to SEQ ID NO: 281;
(kk) the sense strand comprises a modified nucleobase sequence according to SEQ ID NO: 474 and the antisense strand comprises a modified nucleobase sequence according to SEQ ID NO: 282; or
(ll) the sense strand comprises a modified nucleobase sequence according to SEQ ID NO: 477 and the antisense strand comprises a modified nucleobase sequence according to SEQ ID NO: 285.

Embodiment 180. The method or use of embodiment 179, wherein the RNAi agent comprises a targeting ligand comprising a structure according to Formula I:

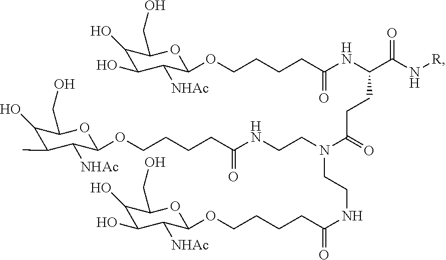

wherein R is a linker conjugated to the wherein the targeting ligand is conjugated to the 3′ end of the sense strand, wherein R comprises a structure according to Formula III:

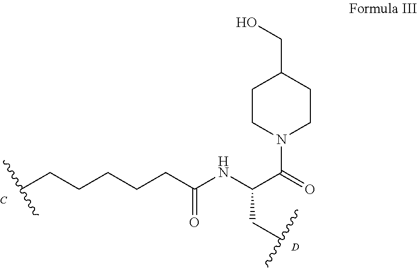

and wherein connection point C of Formula III is conjugated to Formula I, and connection point D of Formula III is conjugated to the 3′ end of the sense strand.

Embodiment 181. The method or use of any one of embodiments 126-178, wherein the sense strand comprises a 3′ overhang of 1-5 nucleotides in length.

Embodiment 182. The method or use of any one of embodiments 126-178 and 181, wherein the sense strand comprises a 5′ overhang of 1-5 nucleotides in length.

Embodiment 183. The method or use of any one of embodiments 126-178, wherein the antisense strand comprises a 3′ overhang of 1-5 nucleotides in length.

Embodiment 184. The method or use of any one of embodiments 126-178 and 183, wherein the antisense strand comprises a 5′ overhang of 1-5 nucleotides in length.

Embodiment 185. The method or use of any one of embodiments 126-184, wherein the sense strand or the antisense strand comprises at least one modified nucleotide or at least one modified internucleoside linkage.

Embodiment 186. The method or use of embodiment 185, wherein the sense strand and or the antisense strand comprises a modified internucleoside linkage at a first, second, penultimate, or final internucleoside linkage position.

Embodiment 187. The method or use of embodiment 185 or 186, wherein the at least one modified nucleotide or at least one modified internucleoside linkage comprises a 2′-O-methyl modified nucleotide, a nucleotide comprising a 5′-phosphorothioate group, a 2′-deoxy-2′-fluoro modified nucleotide, a 2′-deoxy-modified nucleotide, a locked nucleotide, an abasic nucleotide, deoxythymidine, an inverted deoxythymidine, a 2′-amino-modified nucleotide, a 2′-alkyl-modified nucleotide, a morpholino nucleotide, a phosphoramidate, or a non-natural base comprising nucleotide.

Embodiment 188. The method or use of any one of embodiments 139-178 and 181-187, wherein the targeting ligand is conjugated to the sense strand.

Embodiment 189. The method or use of embodiment 188, wherein the targeting ligand is conjugated to the 3′ end of the sense strand.

Embodiment 190. The method or use of embodiment 188, wherein the targeting ligand is conjugated to the 5′ end of the sense strand.

Embodiment 191. The method or use of any one of embodiments 139-178 and 181-186, wherein the targeting ligand is conjugated to the antisense strand.

Embodiment 192. The method or use of embodiment 191, wherein the targeting ligand is conjugated to the 3′ end of the antisense strand.

Embodiment 193. The method or use of embodiment 191, wherein the targeting ligand is conjugated to the 5′ end of the antisense strand.

Embodiment 194. The method or use of any one of embodiments 139-178 and 181-193, wherein the targeting ligand comprises a galactose trimer.

Embodiment 195. The method or use of any one of embodiments 139-178 and 181-194, wherein the targeting ligand comprises N-acetylgalactosamine (GalNAc).

Embodiment 196. The method or use of any one of embodiments 139-178 and 181-195, wherein the targeting ligand comprises a structure according to Formula I:

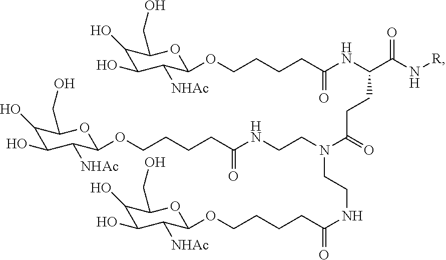

wherein R is:
(i) the sense strand or the antisense strand; or
(ii) a linker conjugated to the sense strand or the antisense strand.

Embodiment 197. The method or use of embodiment 196, wherein R is a linker conjugated to the sense strand or the antisense strand having a structure according to Formula III:

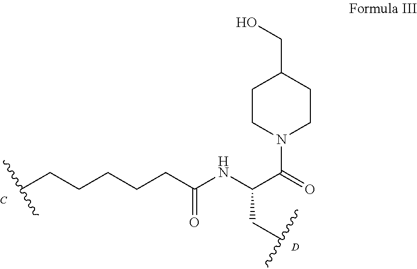

wherein:
(i) connection point C of Formula III is conjugated to Formula I, and connection point D of Formula III is conjugated to the sense strand or the antisense strand; or
(ii) connection point C of Formula III is conjugated to Formula I, and connection point D of Formula III is conjugated to a phosphate group which is conjugated to the sense strand or the antisense strand.

Embodiment 198. The method or use of embodiment 197, wherein Formula III or the phosphate group is conjugated to the 3′ end or the 5′ end of the sense strand or the antisense strand.

Embodiment 199. The method or use of embodiment 197, wherein Formula III or the phosphate group is conjugated to an internal nucleotide or nucleoside base of the sense strand or the antisense strand.

Embodiment 200. The method or use of any one of embodiments 139-178 and 181-195, wherein the RNAi agent comprises a targeting ligand and linker according to Formula V:

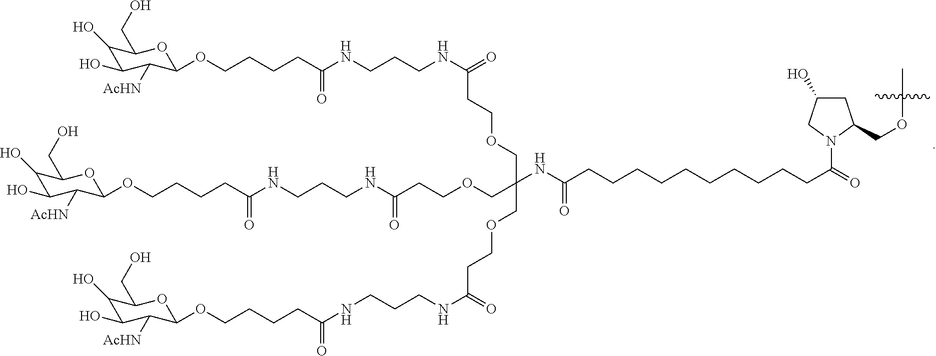 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

Embodiment 201. The method or use of any one of embodiments 126-200, wherein the RNAi agent is an siRNA molecule.

Embodiment 202. The method or use of any one of embodiments 126-200, wherein the RNAi agent is an shRNA molecule.

Embodiment 203. A method of producing the RNAi agent according to any one of embodiments 1-78, comprising annealing together the sense strand and the antisense strand.

Embodiment 204. The method or use of embodiment 203, comprising synthesizing the antisense strand and/or the sense strand.

### EXAMPLES

The following examples are included for illustrative purposes only and are not intended to limit the scope of the invention.

### Example 1: Bioinformatic Based in Silico Design of siRNA Screening Set

In silico modeling enabled identification and selection of siRNA constructs that target human GCK. The selected siRNA candidates were also screened in silico for potential off-target reactivity and cross-reactivity including in human, cynomolgus monkey, rhesus monkey, mouse, and rat. The bioinformatic off-target prediction assumed a canonical 19mer dsRNA structure. Positions 2-18 (5′ to 3′) of the sense and antisense strand were used for specificity calculations and positions 2-7 (5′ to 3′) were used for seed region analysis. Positions 1-19 (5′ to 3′) and positions 1-21 (5′ to 3′) of the antisense strand were used for cross-reactivity and human SN P analysis. All relevant sequences that target GCK mRNA were identified and assembled in silico. Further cross-reactivity for human, cynomolgus monkey, rhesus monkey mouse, and rat analysis was conducted using all 19 basis or positions 2-18 of the sense strand, and all 21 bases or positions 2-20 of the antisense strand. Sense strands and antisense strands were analyzed separately.

For each candidate siRNA, the off-target frequency, classified by mismatch number, was calculated. The identity between seed region siRNA candidates and the seed region of known miRNAs was investigated to exclude siRNA candidates having identical seed regions to known miRNAs.

The siRNA candidates were analyzed against the N CBI SN P database to identify siRNAs targeting regions with known SNPs. A total of 96 candidate siRNAs were selected based on the analysis. See duplexes including sense and antisense sequences in Table 3.

The 96 candidate siRNAs identified according to the method described in Example 1 are synthesized on approximately 0.2 μmol scale and purified using H PLC. Synthesized siRNA duplexes will include 2′-fluoro and 2′-O-methyl-residues as well as phosphorothioate linkages at selected positions. See duplexes to include modified sense and antisense sequences in Table 3.

### Example 2: In Vitro Screening of RNAi Agents

This Example describes in vitro siRNA screening of selected GCK RNAi agents for their ability to inhibit GCK expression GCK gene expression knockdown following GCK RNAi agent transfection was determined by qPCR.

Briefly, CaSki or NHPPH cells were transfected with either 20 nM or 2 nM of one of the GCK RNAi agents provided in Table E1, along with appropriate negative and positive controls. The RNAi agents were either unconjugated (CaSki cell assay) or conjugated to a GalNAc moiety by a linker as shown in Table 4 (NHPPH cell assay). For NHPPH cells, cells were glucose and serum starved for 24 h before induction of GCK expression by glucose, fructose, and insulin. Total RNA was extracted using a Qiagen RN easy 96 Kit (Qiagen, C at #74181) 48 hours post-transfection, which was subsequently converted to cDNA using SuperScript™ IV VILO™ reverse transcription mix. qRT-PCR was performed on the resulting cDNA using TaqMan™ probes for GCK and GAPDH. GCK fold change was calculated using the delta delta Ct method. The resulting knockdown of GSK expression is presented as a percentage in Table E1 (NHPPH cells) and Table E2 (CaSki cells).

TABLE E1
in vitro GCK mRNA expression in NHPPH cells

| Duplex | % knock down in | % knock down in |
|---|---|---|
| ID NO: | NHPPH (20 nM) | NHPPH (2 nM) |
| 572 | 47.03 ± 11.32 | 38.22 ± 14.51 |
| 574 | 71.06 ± 10.28 | 70.65 ± 8.86 |
| 634 | 6.71 ± 37.05 | 0 ± 47.46 |
| 636 | 0 ± 10.46 | 0 ± 22.13 |
| 655 | 42.72 ± 17.49 | 21.31 ± 7.49 |
| 749 | 0 ± 55 | 0 ± 17.36 |
| 780 | 11.86 ± 23.84 | 21.69 ± 10.88 |
| 784 | 61.33 ± 3.86 | 65.81 ± 7.41 |
| 786 | 49.64 ± 14.23 | 61.23 ± 10.8 |
| 787 | 64.91 ± 6.25 | 69.82 ± 7.75 |
| 789 | 80.83 ± 7.53 | 79.29 ± 1.59 |
| 828 | 0 ± 27.1 | 13.98 ± 9.17 |
| 829 | 46.7 ± 3.83 | 40.03 ± 14.03 |
| 835 | 82.37 ± 2.15 | 59.08 ± 9.24 |
| 836 | 69.33 ± 3.35 | 73.97 ± 15.07 |
| 841 | 74.92 ± 4.19 | 76.05 ± 9.72 |
| 882 | 50.24 ± 7.69 | 32.05 ± 8.13 |
| 953 | 61.66 ± 8.33 | 68.95 ± 6.11 |
| 955 | 23.21 ± 6.88 | 16.61 ± 26.99 |
| 962 | 63.37 ± 7.44 | 66.34 ± 9.75 |
| 966 | 45.94 ± 19.5 | 39.16 ± 13.89 |
| 969 | 54.84 ± 4.23 | 54.66 ± 6.64 |
| 1005 | 64.79 ± 6.66 | 75.44 ± 10.23 |
| 1007 | 35.97 ± 6.32 | 38.35 ± 7.84 |
| 1008 | 34.58 ± 16.28 | 36.65 ± 19.72 |
| 1009 | 52.42 ± 5.52 | 53.26 ± 8.21 |
| 1011 | 43.86 ± 4.44 | 7.93 ± 19.59 |
| 1012 | 26.17 ± 4.51 | 5.41 ± 7.74 |
| 1072 | 54.59 ± 15.75 | 14.26 ± 31.18 |
| 1076 | 65.95 ± 6.66 | 70.35 ± 9.54 |
| 1077 | 55.3 ± 1 | 59.76 ± 9.54 |
| 1078 | 70.97 ± 5.44 | 67.49 ± 3.04 |
| 1081 | 72.07 ± 7.84 | 79.25 ± 6.1 |
| 1084 | 60.7 ± 12.42 | 46.93 ± 4.86 |
| 1085 | 75.27 ± 21.21 | 68.23 ± 16.97 |
| 1088 | 74.84 ± 8.98 | 52.54 ± 21.76 |
| 1089 | 58.03 ± 11.78 | 53.14 ± 25.09 |
| 1107 | 82.89 ± 7.96 | 82.32 ± 1.22 |
| 1108 | 84.47 ± 5.54 | 78.94 ± 2.29 |
| 1109 | 72.65 ± 13.97 | 81.44 ± 3.8 |
| 1110 | 74.83 ± 7.44 | 74.85 ± 9 |
| 1111 | 76.39 ± 3.21 | 73.99 ± 4.13 |
| 1113 | 61.33 ± 7.45 | 52.27 ± 9.2 |
| 1114 | 61.85 ± 6.97 | 69.63 ± 16.13 |
| 1123 | 55.17 ± 27.48 | 38.87 ± 25.06 |
| 1124 | 73.33 ± 16.78 | 79.02 ± 7.61 |
| 1127 | 68.29 ± 7.88 | 60.61 ± 11.33 |
| 1130 | 34.96 ± 18.54 | 56.86 ± 13.32 |
| 1132 | 7.63 ± 55.32 | 0 ± 8.87 |
| 1134 | 70.4 ± 11.87 | 65.83 ± 3.31 |
| 1136 | 45.29 ± 3.1 | 33.97 ± 17.31 |
| 1138 | 68.08 ± 4.28 | 68.86 ± 5.25 |
| 1188 | 37.15 ± 17.02 | 19.45 ± 15.17 |
| 1291 | 47.16 ± 22.96 | 43.03 ± 17.7 |
| 1297 | 0 ± 20.05 | 0 ± 38.54 |
| 1298 | 44.38 ± 7.08 | 33.85 ± 7.7 |
| 1299 | 0 ± 25.23 | 0.69 ± 10.11 |
| 1300 | 39.99 ± 6.87 | 38.91 ± 17.49 |
| 1301 | 19.22 ± 12.8 | 0 ± 22.34 |
| 1340 | 71.56 ± 2.49 | 64.42 ± 6.08 |
| 1345 | 51.69 ± 4.27 | 40.09 ± 12.12 |
| 1347 | 39.67 ± 11.31 | 45.45 ± 26.54 |
| 1348 | 23.81 ± 13.02 | 21.53 ± 16.73 |
| 1349 | 59.66 ± 12.62 | 57.38 ± 15.66 |
| 1350 | 63.08 ± 18.09 | 48.16 ± 17.97 |
| 1355 | 5.22 ± 34.17 | 19.81 ± 6.07 |
| 1356 | 52.77 ± 9.63 | 60.29 ± 2.7 |
| 1359 | 0 ± 21.14 | 0 ± 21.92 |
| 1363 | 7.39 ± 15.29 | 0 ± 21.69 |
| 1365 | 17.94 ± 17.21 | 18.12 ± 14.96 |
| 1367 | 4.95 ± 24.99 | 5.29 ± 5.03 |
| 1399 | 51.43 ± 7.93 | 56.45 ± 7.45 |
| 1400 | 50.61 ± 10.95 | 43.85 ± 8.94 |
| 1513 | 41.89 ± 0.7 | 36.09 ± 18.58 |
| 1521 | 46.87 ± 10.42 | 34.85 ± 21.51 |
| 1526 | 55.64 ± 13.41 | 58.01 ± 3.61 |
| 1528 | 52.98 ± 5.07 | 56.2 ± 14.65 |
| 1688 | 55.37 ± 14.24 | 68.26 ± 0 |
| 1695 | 9.72 ± 14.72 | 30.27 ± 5.95 |
| 1696 | 10.56 ± 19.28 | 0 ± 7.53 |
| 1697 | 20.42 ± 14.16 | 13.96 ± 20.56 |
| 1701 | 2.18 ± 6.81 | 11.19 ± 23.65 |
| 1703 | 47.05 ± 11.42 | 64.9 ± 10.79 |
| 1704 | 45.08 ± 5.65 | 61.99 ± 11.75 |
| 1708 | 21.88 ± 13.7 | 7.36 ± 40.36 |
| 1774 | 62.49 ± 11.64 | 51.44 ± 7.62 |
| 1786 | 64.63 ± 4.24 | 67.13 ± 6.07 |
| 1789 | 59.44 ± 10.71 | 65.98 ± 9.89 |
| 1790 | 38.9 ± 6.4 | 40.47 ± 16.33 |
| 2180 | 38.86 ± 16.95 | 53.36 ± 12.18 |
| 2273 | 26.38 ± 17.7 | 0 ± 33.86 |
| 2297 | 29.91 ± 24.82 | 30.06 ± 6.27 |
| 2303 | 46 ± 11.96 | 57.49 ± 12.71 |
| 2492 | 50.5 ± 16.36 | 47.33 ± 8.88 |
| 2493 | 47.72 ± 8.8 | 58.84 ± 9.73 |
| 2609 | 6.79 ± 8.34 | 38.09 ± 30.67 |

TABLE E2
in vitro GCK mRNA expression in CaSki cells

| Duplex | % knock down in | % knock down in |
|---|---|---|
| ID NO: | CaSki (20 nM) | CaSki (2 nM) |
| 572 | 3.05 ± 5.89 | 0 ± 2.7 |
| 574 | 10.02 ± 2.76 | 0 ± 6.93 |
| 634 | 41.43 ± 4.28 | 5.79 ± 3.21 |
| 636 | 26.04 ± 2.82 | 23.91 ± 2.45 |
| 655 | 35.54 ± 6.46 | 23.33 ± 7.16 |
| 749 | 6.29 ± 3.84 | 8.43 ± 3.54 |
| 780 | 31.23 ± 2.3 | 14.77 ± 5.21 |
| 784 | 20.07 ± 2.13 | 14.3 ± 4.74 |
| 786 | 24.03 ± 2.64 | 10.69 ± 2.47 |
| 787 | 28.18 ± 4.74 | 7.6 ± 6.84 |
| 789 | 38.1 ± 10.19 | 17.1 ± 5.65 |
| 828 | 19.63 ± 6.66 | 5.65 ± 3.89 |
| 829 | 34.97 ± 3.11 | 13.98 ± 5.39 |
| 835 | 24.62 ± 9.87 | 17.82 ± 6.59 |
| 836 | 35.25 ± 2.34 | 25.19 ± 15.14 |
| 841 | 48.5 ± 10.12 | 22.8 ± 3.85 |
| 882 | 31.42 ± 2.3 | 18.06 ± 1.76 |
| 953 | 29.28 ± 8.54 | 14.53 ± 8.26 |
| 955 | 4.89 ± 4.56 | 7.45 ± 3.73 |
| 962 | 43.04 ± 3.24 | 30.77 ± 0.57 |
| 966 | 32.36 ± 2.34 | 14.74 ± 4.11 |
| 969 | 29.96 ± 5.19 | 12.12 ± 6.03 |
| 1005 | 26.38 ± 7.99 | 1.88 ± 4.82 |
| 1007 | 44.62 ± 4.33 | 31.39 ± 6.58 |
| 1008 | 36.56 ± 10.68 | 12.76 ± 2.79 |
| 1009 | 35.73 ± 5.23 | 21.33 ± 2.48 |
| 1011 | 50 ± 0.81 | 19.31 ± 1.71 |
| 1012 | 54.09 ± 5.24 | 17.12 ± 5.24 |
| 1072 | 57.12 ± 3.39 | 35.5 ± 7.46 |
| 1076 | 36.79 ± 2.63 | 19.74 ± 9 |
| 1077 | 28.09 ± 2.21 | 8.36 ± 9.81 |
| 1078 | 23.92 ± 6.16 | 2.52 ± 1.1 |
| 1081 | 43.74 ± 0.87 | 19.12 ± 2.59 |
| 1084 | 11.81 ± 7.08 | 0 ± 7.83 |
| 1085 | 17.73 ± 7.64 | 9.1 ± 9.17 |
| 1088 | 31.14 ± 8.87 | 14.36 ± 3.53 |
| 1089 | 25.1 ± 4.17 | 13.13 ± 3.65 |
| 1107 | 47.69 ± 10.87 | 18.85 ± 1.33 |
| 1108 | 32.55 ± 5.75 | 6.75 ± 10.64 |
| 1109 | 31.32 ± 6.96 | 6.06 ± 4.64 |
| 1110 | 18.08 ± 3.76 | 18.54 ± 15.33 |
| 1111 | 0 ± 17.31 | 6.42 ± 12.44 |
| 1113 | 32.15 ± 6.64 | 23.02 ± 2.88 |
| 1114 | 39.83 ± 2.3 | 38.67 ± N/A |
| 1123 | 37.88 ± 3.25 | 13.44 ± N/A |
| 1124 | 53.7 ± 1.24 | 36.34 ± N/A |
| 1127 | 59.5 ± 1.45 | 28.2 ± N/A |
| 1130 | 23.47 ± 8.81 | N/A |
| 1132 | 14.07 ± 8.4 | N/A |
| 1134 | 31.47 ± 9.45 | N/A |
| 1136 | 33.5 ± 5 | 9.54 ± 5.74 |
| 1138 | 19.72 ± 8.24 | 14.89 ± 5.42 |
| 1188 | 23.34 ± 6.8 | 24.89 ± 6.98 |
| 1291 | 21.51 ± 4.88 | 22.38 ± 8.34 |
| 1297 | 15.38 ± 9.42 | 30.44 ± 5.04 |
| 1298 | 32.11 ± 10.32 | 29.72 ± 6.12 |
| 1299 | 29.3 ± 8.34 | 29.32 ± 2.21 |
| 1300 | 34.56 ± 9.95 | 33.1 ± 1.92 |
| 1301 | 20.33 ± 6.06 | 11.2 ± 1.24 |
| 1340 | 13.06 ± 4.06 | 5.4 ± 8.42 |
| 1345 | 15.52 ± 12.86 | 6.44 ± 13.95 |
| 1347 | 30.83 ± 7.99 | 16.74 ± 10.3 |
| 1348 | 25.89 ± 3.05 | 3.09 ± 3.85 |
| 1349 | 38.87 ± 1.54 | 34.54 ± 4.78 |
| 1350 | 26.18 ± 5.01 | 15.96 ± 1.8 |
| 1355 | 10.14 ± 5.12 | 11.13 ± 25.27 |
| 1356 | 46.37 ± 5.48 | 29.78 ± 0.29 |
| 1359 | 40.31 ± 6.86 | N/A |
| 1363 | 36.6 ± 3.31 | N/A |
| 1365 | 26.31 ± 2.96 | N/A |
| 1367 | 0 ± 3.11 | 0 ± 2.95 |
| 1399 | 29.58 ± 6.19 | 0 ± 4.76 |
| 1400 | 8.53 ± 7.68 | 6.01 ± 5.84 |
| 1513 | 68.81 ± 7.53 | 53.31 ± 6.31 |
| 1521 | 69.22 ± 2.12 | 49.07 ± 3.65 |
| 1526 | 74.4 ± 11.08 | 44.34 ± 3.41 |
| 1528 | 76.13 ± 3.37 | 66.12 ± 7.05 |
| 1688 | 73.18 ± 5.13 | 60.29 ± 3.45 |
| 1695 | 51.92 ± 4.92 | 16.62 ± 11.49 |
| 1696 | 76.87 ± 7.36 | 67.71 ± 2.27 |
| 1697 | 69.87 ± 4.43 | 45.37 ± 9.8 |
| 1701 | 51.66 ± 1.33 | 47.84 ± 2.54 |
| 1703 | 87.34 ± 2.15 | 86.35 ± 1.66 |
| 1704 | 91.2 ± 1.4 | 88.69 ± 2.17 |
| 1708 | 86.3 ± 1.02 | 69.76 ± 3.37 |
| 1774 | 86.85 ± 1.97 | 89.02 ± 2.25 |
| 1786 | 89.59 ± 5.54 | 86.34 ± 1.62 |
| 1789 | 89.01 ± 2.86 | 91.95 ± 4.17 |
| 1790 | 91.95 ± 4.17 | 83.8 ± 3.98 |
| 2180 | 88.91 ± 2.05 | 84.44 ± 2.16 |
| 2273 | 67.66 ± 5.03 | 33.07 ± 10.73 |
| 2297 | 81.51 ± 2.86 | 76.84 ± 4.37 |
| 2303 | 87.45 ± 6.66 | 88.45 ± 3.29 |
| 2492 | 75.16 ± 9.35 | 60.18 ± 4.64 |
| 2493 | 83.91 ± 4.78 | 77.44 ± 9.74 |
| 2609 | 80.08 ± 7.07 | 75.28 ± 2.99 |

The 20 GalNAc-conjugated RNAi agents that result in the greatest knockdown percentage of GCK mRNA in CaSki or NHPPH were selected for analysis in an in vitro knockdown assessment in primary human hepatocytes (PHHs).

Primary human hepatocytes (PHHs) were transfected with 20 nM, 2 nM, and 0.2 nM of one of the GCK RNAi agents provided in Table E3 along with appropriate negative and positive controls. Cells were glucose and serum starved for 24 h before induction of GCK expression by glucose and insulin. The resulting knockdown of GSK mRNA expression is presented as a percentage in Table E3.

TABLE E3
in vitro GCK mRNA expression in human primary hepatocytes

| Duplex | % knock down in | % knock down in | % knock down in |
|---|---|---|---|
| ID NO: | HPP (20 nM) | HPP (2 nM) | HPP (0.2 nM) |
| 784 | 81.94 ± 0.06 | 79.73 ± 0.12 | 75.29 ± 0.23 |
| 789 | 81.46 ± 0.16 | 80.16 ± 0.11 | 69.63 ± 0.07 |
| 835 | 83.21 ± 0.14 | 77.14 ± 0.18 | 69.58 ± 0.23 |
| 841 | 87.13 ± 0.08 | 78.75 ± 0.15 | 68.02 ± 0.17 |
| 1085 | 75.69 ± 0.19 | 78.1 ± 0.12 | 58.08 ± 0.33 |
| 1088 | 72 ± 0.09 | 51.3 ± 0.36 | −4.8 ± 0.64 |
| 1107 | 78.36 ± 0.03 | 65.2 ± 0.13 | 69.8 ± 0.09 |
| 1108 | 80.33 ± 0.06 | 81.44 ± 0.06 | 70.01 ± 0.14 |
| 1110 | 65.7 ± 0.12 | 68.41 ± 0.06 | 66.35 ± 0.03 |
| 1111 | 76.9 ± 0.02 | 72.52 ± 0.03 | 64.26 ± 0.18 |
| 1138 | 83.24 ± 0.13 | 84.69 ± 0.02 | 46.29 ± 0.33 |
| 1528 | 57.13 ± 0.13 | 68.46 ± 0.05 | 69.46 ± 0.2 |
| 1703 | 51.12 ± 7.68 | NA | NA |
| 1704 | 64.82 ± 0.17 | 44.17 ± 0.21 | 52.17 ± 0.23 |
| 1774 | 54.63 ± 0.11 | 12.82 ± 0.17 | 38.16 ± 0.16 |
| 1786 | 79.31 ± 0.06 | 61.28 ± 0.04 | 51.72 ± 0.13 |
| 1789 | 62.91 ± 0.13 | 66.5 ± 0.04 | 56.16 ± 0.11 |
| 1790 | 66.15 ± 0.06 | 49.22 ± 0.26 | 44.7 ± 0.22 |
| 2180 | 61.4 ± 0.12 | 45.64 ± 0.24 | 12.97 ± 0.48 |
| 2303 | 52.45 ± 0.05 | 50.93 ± 0.16 | 52.05 ± 0.07 |

### Example 3: Determination of mRNA Inhibition by Selective RNAi Agents

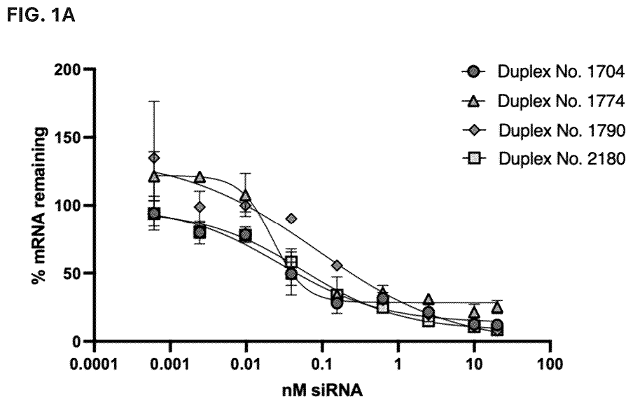

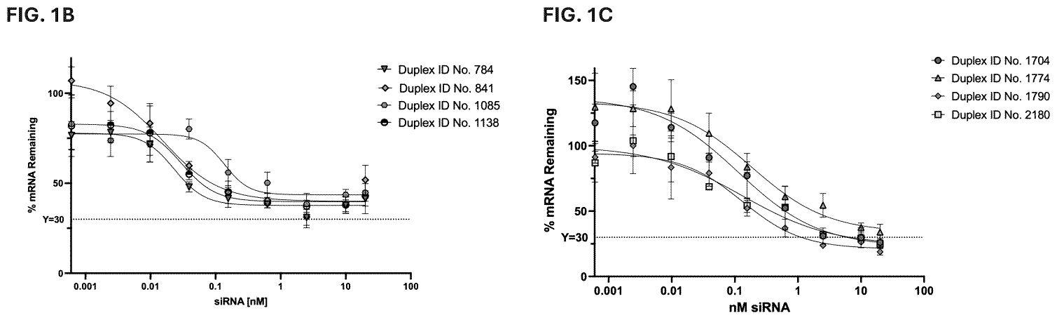

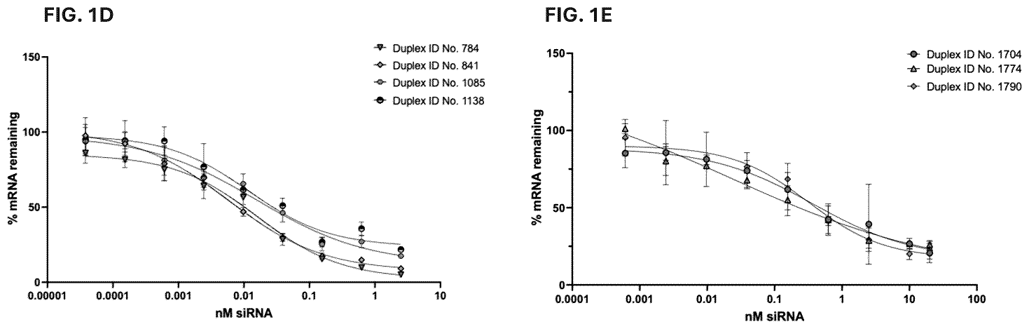

This Example describes the testing of selective RNAi agents in a CaSki cell line, which constitutively expresses GCK. Lysates were used in order to determine the extent of the knockdown of the GCK gene by measuring mRNA expression by RT-qPCR. The twenty RNAi agents that result in the greatest knockdown percentage of GCK mRNA are selected for re-synthesis as GalNAc-conjugates as described in Example 5 below. A dose curve showing four exemplary RNAi agents is shown in FIG. 1A. The bottom value and the IC50 of the selective RNAi agents is shown in Table E4.

TABLE E4
Bottom value and IC50 in CaSki cells

| Duplex ID NO: | Bottom value | IC50 |
|---|---|---|
| 784 | NT | NT |
| 841 | NT | NT |
| 1085 | NT | NT |
| 1138 | NT | NT |
| 1704 | 23.1 ± 15.58 | 0.02 ± 0.01 |
| 1774 | 19.3 ± 2.91 | 0.03 ± 0.01 |
| 1790 | 46.33 ± 1.24 | 0.04 ± 0.04 |
| 2180 | 30.64 ± 12.19 | 0.04 ± 0.03 |

### Example 4: Engineering of a HepG2 Cell Line that Constitutively Expresses GCK

HepG2 cells were transfected with GCK-ORF cDNA by PiggyBac, followed by puromycin selection. Clonal lines were generated and compared to pools; clones showed no difference in gene expression or stability to clonal pools; therefore, RNAi screening was performed with clonal pools (herein referred to as HepG2-GCK).

### Example 5: Production and Efficacy of GalNAc Conjugates

This Example describes the GalNAc conjugation of the twenty selected RNAi agents from Example 2. The sense strands of the RNAi agents are equipped with a triantennary GalNAc moiety on their 3′-ends, introduced by assembly on a commercially available GalNAc-loaded solid support (GalNAc-TEG cluster available through Primetech).

The GalNAc-conjugated RNAi agents are then tested for the ability to inhibit GCK mRNA in non-human primate primary hepatocytes (NHPPH) and HepG2-GCK. Dose curves for the eight select GalNAc-conjugated RNAi agents that result in the greatest knockdown percentage of GCK mRNA are shown in FIG. 1B and FIG. 1C (NHPPH cells) and FIG. 1D and FIG. 1E (HepG2-GCK cells). The bottom value and the IC50 of the selective RNAi agents is shown in Table E5 (NHPPH cells) and Table E6 (HepG2-GCK cells).

TABLE E5
Bottom value and IC50 in NHPPH cells

| Duplex ID NO: | Bottom value | IC50 |
|---|---|---|
| 784 | 30.11 ± 1.25 | 0.01 ± 0.01 |
| 841 | 36.13 ± 6.94 | 0.05 ± 0.06 |
| 1085 | 43.05 ± 6.53 | 0.13 ± 0.01 |
| 1138 | 51.22 ± 19.81 | 0.02 ± 0.02 |
| 1704 | 26.8 ± 1.09 | 0.14 ± 0.03 |
| 1774 | 29.6 ± 6.11 | 0.1 ± 0.13 |
| 1790 | 21.68 ± 4 | 0.15 ± 0.03 |
| 2180 | 28.22 ± 4.78 | 0.32 ± 0.26 |

TABLE E6
Bottom value and IC50 in HepG2-GCK cells

| Duplex ID NO: | Bottom value | IC50 |
|---|---|---|
| 784 | 10.2 ± 4.49 | 0.01 ± 0.01 |
| 841 | 16.09 ± 6.04 | 0.01 ± 0.01 |
| 1085 | 20.16 ± 2.33 | 0.02 ± 0.01 |
| 1138 | 23.79 ± 1.74 | 0.01 ± 0.01 |
| 1704 | 26.36 ± 8.12 | 0.41 ± 0.06 |
| 1774 | 27.33 ± 1.61 | 0.01 ± 0.02 |
| 1790 | 22.38 ± 3.33 | 0.32 ± 0.01 |
| 2180 | NT | NT |

These eight GalNAc-conjugated RNAi agents that result in the greatest knockdown percentage of GCK mRNA are selected for in vivo dose response analysis of therapeutic efficacy described in Example 6.

### Example 6: In vivo Screening of RNAi Agents

This Example describes in vivo screening of selected GalNAc conjugated-GCK RNAi agents for their ability to inhibit GCK expression and ability to reduce liver fat in model mice.

A. Design of exemplary GCK RNAi agents

The sense strands of several exemplary GCK RNAi agents included a triantennary GalNAc moiety on their 3′-ends. The (GalNAc moiety)+(linker) has a structure as shown in Table 4. The resulting GalNAc-conjugated GCK RNAi agents were used to conduct in vivo experiments.

B. In vivo screening of exemplary GCK RNAi agents

Animal experiments were conducted in accordance with the Institutional Animal Care and Use Committee (IACUC) guidelines at Charles River, Massachusetts. A diet induced obese (DIO) mouse model was used to evaluate GCK gene expression knockdown and liver fat reduction following GCK RNAi agent transfection. Briefly, C57BL/6Ntac male mice were fed a 60% High fat diet (Research Diet #D12492) for 16 weeks. After acclimatization for 2 weeks, mice were administered subcutaneous injections of either phosphate buffer saline (PBS, pH 7.4) as a control, or 10 mg/kg of selected GCK RNAi agents, and were fed the diet for an additional 4 weeks.

After 4 weeks, animals are euthanized and maximum blood is collected via abdominal aorta and processed to serum. Terminal serum from all groups is analyzed for alanine aminotransferase (ALT), aspartate aminotransferase (AST), liver triglycerides (TG), glucose, and liver triglycerides via plate based assay. Mouse livers are harvested and weighed. Left median lobe (LML) is collected in RNA later. Approximately 50 mg tissue from right lateral lobe is weighed and snap-frozen for liver triglyceride assay. All remaining liver is snap-frozen. Epididymal fat pads are harvested, weighed, and snap frozen. The harvested liver tissue was stored at −80° C. for future analysis.

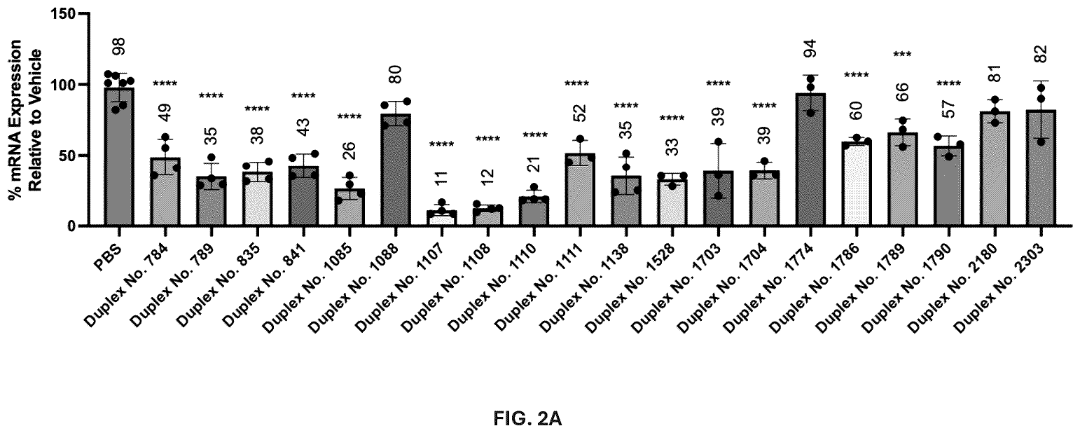

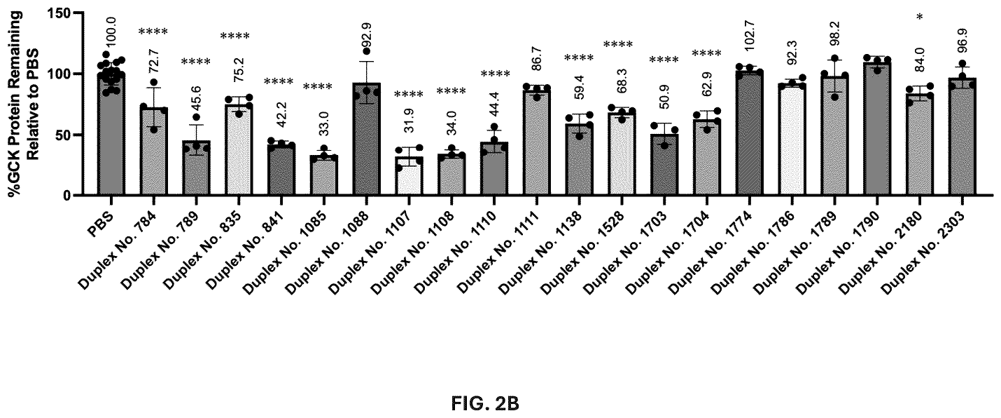

GCK gene expression knockdown at the mRNA level in the harvested liver tissue was determined by qPCR. Briefly, total RNA was extracted using QiaZol according to the manufacturer's instructions (Qiagen) and purified using a Qiagen R N easy 96 Kit (Qiagen, Cat #74181). RNA was subsequently converted to cDNA using SuperScript™ IV V ILO™ reverse transcription mix. RT-qPCR was performed on the resulting cDNA using TaqMan™ probes for GCK and GAPDH. GCK fold change was calculated using the delta delta Ct method. These values are shown in FIG. 2A and Table E7 as a percent of GCK mRNA expression in mice treated with selected GCK RNAi agents relative to mice treated with PBS.

GCK protein expression was determined by Jess Automated Western Blot System (Protein Simple) using an antibody to GCK (Protein Tech). Protein content was normalized using a BCA Protein Assay Kit (MilliporeSigma) before loading, and internal Vinculin was used as an internal control (Protein Simple). These values are shown in FIG. 2B and Table E7 as a percent of GCK protein remaining in mice treated with selected GCK RNAi agents relative to mice treated with PBS.

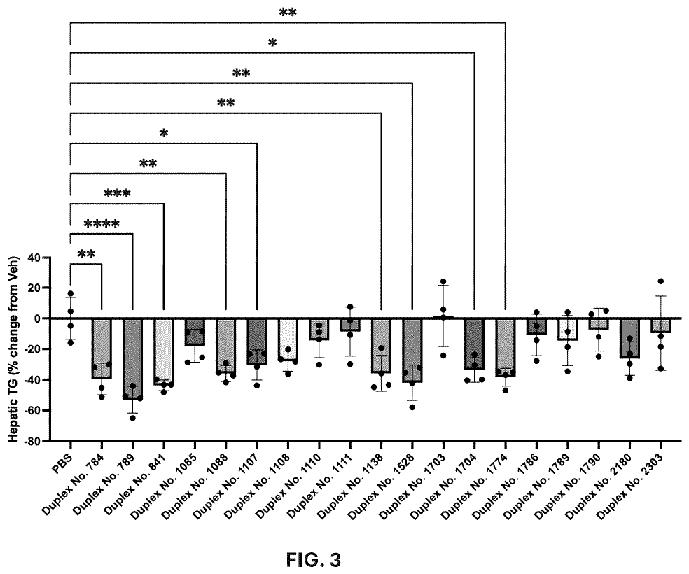

Liver fat in mice was measured using a colorimetric assay performed according to the manufacturer's protocol (Triglyceride Colorimetric Assay Kit, Cayman Chemicals). Reduction in liver fat, as determined as a measurement of hepatic triglycerides, in mice treated with the selected GalNAc conjugated-GCK RNAi agents was compared to that of mice treated with PBS. As shown in FIG. 3 and Table E7, a majority of GalNAc conjugated-GCK RNAi agents were able to reduce hepatic triglycerides in mice as compared to mice treated with PBS, with no significant change in fasted hepatic glycogen content.

TABLE E7
GCK mRNA expression knockdown, GCK protein
knockdown, and triglyceride reduction

| | | | Mean triglyceride |
|---|---|---|---|
| Duplex | Mean % mRNA | Mean % Protein | (% reduction) |
| ID NO: | knockdown | knockdown | relative to PBS |
| Vehicle (PBS) | — | — | — |
| 784 | 51.24 ± 12.64 | 27.3 ± 15.94 | 39.55 ± 9.4 |
| 789 | 65.01 ± 9.47 | 54.4 ± 12.71 | 52.98 ± 8.41 |
| 835 | 61.79 ± 7 | 24.8 ± 6.1 | −49.7 ± 28.72 |
| 841 | 57.4 ± 8.44 | 57.8 ± 2.78 | 43.66 ± 3.57 |
| 1085 | 73.62 ± 7.71 | 67 ± 4.28 | 17.86 ± 9.79 |
| 1088 | 18.41 ± 9.16 | 7.1 ± 17.23 | 35.92 ± 6.15 |
| 1107 | 88.95 ± 3.93 | 88.95 ± 3.93 | 30.37 ± 8.9 |
| 1108 | 86.64 ± 1.84 | 86.64 ± 1.84 | 27.89 ± 7.24 |
| 1110 | 79.25 ± 4.44 | 79.25 ± 4.44 | 14.38 ± 10.18 |
| 1111 | 21 ± 17.68 | 21 ± 17.68 | 24.56 ± 19.21 |
| 1138 | 64.55 ± 13.4 | 64.55 ± 13.4 | 35.85 ± 10.91 |
| 1528 | 44.2 ± 11.16 | 44.2 ± 11.16 | 41.97 ± 11.48 |
| 1703 | 82.4 ± 3.73 | 82.4 ± 3.73 | 2.05 ± 15.28 |
| 1704 | 72.1 ± 3.47 | 72.1 ± 3.47 | 33.61 ± 8.01 |
| 1774 | 6.9 ± 13.51 | 0 ± 3.49 | 38.4 ± 5.78 |
| 1786 | 49.2 ± 14.9 | 7.7 ± 3.31 | 19.22 ± 5.96 |
| 1789 | 36 ± 14.46 | 1.8 ± 13.12 | 18.7 ± 9.87 |
| 1790 | 55.2 ± 8.06 | 0 ± 4.74 | 13.27 ± 13.96 |
| 2180 | 71.5 ± 6.17 | 16 ± 6.11 | 29.24 ± 7.04 |
| 2303 | 49.5 ± 5.93 | 3.1 ± 8.65 | 14.61 ± 27.48 |

Serum alanine aminotransferase (ALT), aspartate aminotransferase (AST), plasma glucose, and hepatic glycogen were measured by colorimetric methods performed according to the manufacturer's protocol (Triglyceride Colorimetric Assay Kit, Cayman Chemicals; Glycogen-Glo Assay, Promega). These values are shown in Table E8.

TABLE E8
GCK mRNA expression knockdown, GCK protein
knockdown, and triglyceride reduction

| | Mean Glycogen | Mean Plasma Glucose |
|---|---|---|
| | (% of Vehicle) | (mg/dL) |
| Vehicle (PBS) | — | 195 ± 18.62 |
| Duplex No. 784 | 108.33 ± 10.01 | 188 ± 27.58 |
| Duplex No. 789 | 107.69 ± 8.05 | 195.5 ± 17.059 |
| Duplex No. 835 | 75 ± 4.71 | 206.5 ± 20.421 |
| Duplex No. 841 | 95.24 ± 5.21 | 199.25 ± 27.035 |
| Duplex No. 1085 | 140 ± 2.5 | 182 ± 2.708 |
| Duplex No. 1088 | 121.43 ± 7.73 | 225.25 ± 34.664 |
| Duplex No. 1107 | 88.24 ± 3.55 | 226.25 ± 36.087 |
| Duplex No. 1108 | 103.33 ± 5.82 | 237.5 ± 26.988 |
| Duplex No. 1110 | 103.23 ± 2.8 | 208.75 ± 24.622 |
| Duplex No. 1111 | 101.96 ± 9.44 | 160.75 ± 9.394 |
| Duplex No. 1138 | 81.25 ± 1.9 | 204.25 ± 22.096 |
| Duplex No. 1528 | 117.65 ± 16.43 | 162.25 ± 11.73 |
| Duplex No. 1703 | 88.24 ± 8.26 | 159 ± 18.111 |
| Duplex No. 1704 | 109.8 ± 6.75 | 185 ± 22.435 |
| Duplex No. 1774 | 103.92 ± 12.97 | 157.75 ± 21.899 |
| Duplex No. 1786 | 129.41 ± 12.53 | 183.75 ± 10.72 |
| Duplex No. 1789 | 70.59 ± 1.41 | 179 ± 9.695 |
| Duplex No. 1790 | 80.39 ± 8.84 | 172.75 ± 24.932 |
| Duplex No. 2180 | 86.27 ± 26.71 | 174.25 ± 7.274 |
| Duplex No. 2303 | 88.24 ± 12.54 | 189 ± 28.694 |

| | Mean AST (U/L) | Mean ALT (U/L) |
|---|---|---|
| Vehicle (PBS) | 208.5 ± 52.206 | 180.125 ± 50.7135 |
| Duplex No. 784 | 252 ± 97.43 | 136.5 ± 77.479 |
| Duplex No. 789 | 188.25 ± 52.322 | 91 ± 25.153 |
| Duplex No. 835 | 367.75 ± 69.744 | 300.5 ± 68.898 |
| Duplex No. 841 | 160.75 ± 24.377 | 73.5 ± 5 |
| Duplex No. 1085 | 192.75 ± 86.454 | 163.5 ± 61.674 |
| Duplex No. 1088 | 139 ± 42.965 | 107.5 ± 47.892 |
| Duplex No. 1107 | 163.75 ± 23.684 | 165.25 ± 58.317 |
| Duplex No. 1108 | 211.5 ± 27.815 | 188.5 ± 26.64 |
| Duplex No. 1110 | 243.75 ± 57.209 | 290.25 ± 93.756 |
| Duplex No. 1111 | 120.75 ± 30.445 | 110.5 ± 50.216 |
| Duplex No. 1138 | 119.5 ± 20.809 | 90.25 ± 33.629 |
| Duplex No. 1528 | 206.5 ± 31.204 | 135.5 ± 31.032 |
| Duplex No. 1703 | 669.75 ± 308.471 | 454.75 ± 148.95 |
| Duplex No. 1704 | 195.5 ± 47.135 | 111 ± 26.369 |
| Duplex No. 1774 | 188.75 ± 46.27 | 189 ± 48.874 |
| Duplex No. 1786 | 173.25 ± 34.131 | 168 ± 37.754 |
| Duplex No. 1789 | 191.5 ± 79.81 | 194 ± 130.494 |
| Duplex No. 1790 | 221.75 ± 42.406 | 230 ± 67.809 |
| Duplex No. 2180 | 556.75 ± 125.223 | 390.5 ± 93.746 |
| Duplex No. 2303 | 507 ± 157.647 | 457.5 ± 162.539 |

1-2. (canceled)

3. An RNAi agent that inhibits expression of glucokinase (GCK) comprising an antisense strand and a sense strand that at least partially complements the antisense strand, wherein:
(a) the antisense strand comprises a nucleobase sequence of any one of SEQ ID NOS: 200, 208, 227, 276, 278, 281, and 282; and/or
(b) the sense strand comprises a nucleobase sequence of any one of SEQ ID NOS: 296, 304, 323, 372, 374, 377, and 378.

4. The RNAi agent of claim 3, wherein one or more nucleotides of the antisense stand and/or the sense strand are modified.

5. The RNAi agent of claim 4, wherein the antisense strand has a modification pattern according to nsNfsnnnNfnnnnnnnNfnNfnnnnnsnsn, or the sense strand has a modification pattern according to nsnsnnnnNfnNfNfNfnnnnnnnnsnsn or nsnsnnnnNfnNfNfNfnnnnnnnnnn.

6-7. (canceled)

8. The RNAi agent of claim 3, wherein:
(a) the antisense strand comprises a nucleobase sequence of any one of SEQ ID NOS: 200, 208, 227, 276, 278, 281, and 282; and
(b) the sense strand comprises a nucleobase sequence of any one of SEQ ID NOS: 296, 304, 323, 372, 374, 377, and 378.

9. (canceled)

10. The RNAi agent of claim 3, wherein the antisense strand and the sense strand form a duplex of at least 14 bases in length.

11-13. (canceled)

14. The RNAi agent of claim 3, wherein the antisense strand comprises a nucleobase sequence of any one of SEQ ID NOS: 200, 208, 227, 276, 278, 281, and 282.

15-28. (canceled)

29. The RNAi agent of claim 3, wherein the antisense strand comprises a modified nucleotide sequence of any one of SEQ ID NOS: 200, 208, 227, 276, 278, 281, and 282.

30-41. (canceled)

42. The RNAi agent of claim 3, wherein the sense strand comprises a nucleobase sequence of any one of SEQ ID NOS: 296, 304, 323, 372, 374, 377, and 378.

43-48. (canceled)

49. The RNAi agent of claim 3, wherein the sense strand comprises a modified nucleotide sequence of any one of SEQ ID NOS: 296, 304, 323, 372, 374, 377, 378, 392, 400, 419, 468, 470, 473, and 474.

50-52. (canceled)

53. The RNAi agent of claim 3, wherein:
(a) the sense strand comprises a nucleobase sequence of SEQ ID NO: 296 and the antisense strand comprises a nucleobase sequence of SEQ ID NO: 200;
(b) the sense strand comprises a nucleobase sequence of SEQ ID NO: 304 and the antisense strand comprises a nucleobase sequence of SEQ ID NO: 208;
(c) the sense strand comprises a nucleobase sequence of SEQ ID NO: 323 and the antisense strand comprises a nucleobase sequence of SEQ ID NO: 227;
(d) the sense strand comprises a nucleobase sequence of SEQ ID NO: 378 and the antisense strand comprises a nucleobase sequence according to SEQ ID NO: 282;
(e) the sense strand comprises a nucleobase sequence of SEQ ID NO: 372 and the antisense strand comprises a nucleobase sequence of SEQ ID NO: 276;
(f) the sense strand comprises a nucleobase sequence of SEQ ID NO: 374 and the antisense strand comprises a nucleobase sequence of SEQ ID NO: 278;
(g) the sense strand comprises a nucleobase sequence of SEQ ID NO: 377 and the antisense strand comprises a nucleobase sequence of SEQ ID NO: 281.

54. The RNAi agent of claim 3, wherein:
(a) the sense strand comprises a modified nucleotide sequence according to of SEQ ID NO: 296 and the antisense strand comprises a modified nucleotide sequence according to of SEQ ID NO: 200;
(b) the sense strand comprises a modified nucleotide sequence of SEQ ID NO: 304 and the antisense strand comprises a modified nucleotide sequence of SEQ ID NO: 208;
(c) the sense strand comprises a modified nucleotide sequence of SEQ ID NO: 323 and the antisense strand comprises a modified nucleotide sequence of SEQ ID NO: 227;
(d) the sense strand comprises a modified nucleotide sequence of SEQ ID NO: 372 and the antisense strand comprises a modified nucleotide sequence of SEQ ID NO: 276;
(c) the sense strand comprises a modified nucleotide sequence of SEQ ID NO: 374 and the antisense strand comprises a modified nucleotide sequence of SEQ ID NO: 278;
(f) the sense strand comprises a modified nucleotide sequence of SEQ ID NO: 377 and the antisense strand comprises a modified nucleotide sequence of SEQ ID NO: 281; or
(g) the sense strand comprises a modified nucleotide sequence of SEQ ID NO: 378 and the antisense strand comprises a modified nucleotide sequence of SEQ ID NO: 282;

55. The RNAi agent of claim 3, wherein:
(a) the sense strand comprises a modified nucleotide sequence according to SEQ ID NO: 400 and the antisense strand comprises a modified nucleotide sequence according to SEQ ID NO: 208;
(b) the sense strand comprises a modified nucleotide sequence according to SEQ ID NO: 419 and the antisense strand comprises a modified nucleotide sequence according to SEQ ID NO: 227;
(c) the sense strand comprises a modified nucleotide sequence according to SEQ ID NO: 392 and the antisense strand comprises a modified nucleotide sequence according to SEQ ID NO: 200;
(d) the sense strand comprises a modified nucleotide sequence according to SEQ ID NO: 468 and the antisense strand comprises a modified nucleotide sequence according to SEQ ID NO: 276;
(e) the sense strand comprises a modified nucleotide sequence according to SEQ ID NO: 470 and the antisense strand comprises a modified nucleotide sequence according to SEQ ID NO: 278;
(f) the sense strand comprises a modified nucleotide sequence according to SEQ ID NO: 473 and the antisense strand comprises a modified nucleotide sequence according to SEQ ID NO: 281; or
(g) the sense strand comprises a modified nucleotide sequence according to SEQ ID NO: 474 and the antisense strand comprises a modified nucleotide sequence according to SEQ ID NO: 282.

56. The RNAi agent of claim 55, wherein the RNAi agent comprises a targeting ligand comprising a structure according to Formula I: 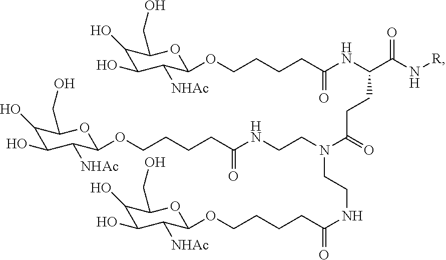
wherein R is a linker conjugated to the wherein the targeting ligand is conjugated to the 3′ end of the sense strand, wherein R comprises a structure according to Formula III: 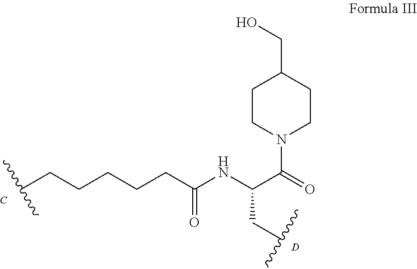
and wherein connection point C of Formula III is conjugated to Formula I, and connection point D of Formula III is conjugated to the 3′ end of the sense strand;
or wherein the RNAi agent comprises a targeting ligand comprising a structure according to Formula V:

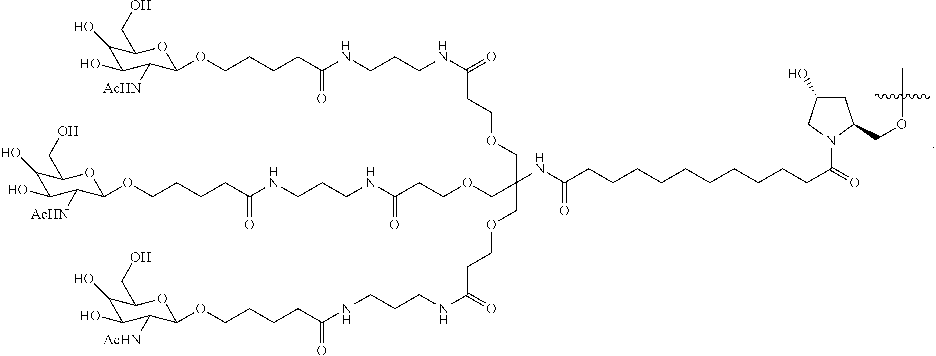 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

57-69. (canceled)

70. The RNAi agent of claim 3, wherein the RNAi agent comprises a targeting ligand comprising a galactose trimer.

71-73. (canceled)

74. The RNAi agent of claim 55, wherein the RNAi agent comprises a targeting ligand and linker according to Formula V:

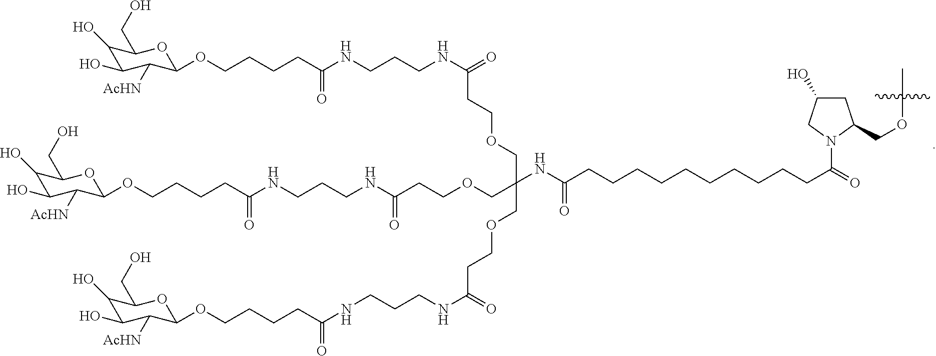 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

75-122. (canceled)

123. A pharmaceutical composition comprising the RNAi agent of claim 3 and a pharmaceutically acceptable carrier.

124. A lipid nanoparticle comprising the RNAi agent of claim 3.

125. (canceled)

126. A method of treating metabolic dysfunction-associated steatotic liver disease (MASLD) or metabolic dysfunction-associated steatotic liver (MASL) in a subject, comprising:
administering to the subject an effective amount of a liver-targeted modified oligonucleotide, a liver-targeted oligomeric duplex, or a liver-targeted RNAi agent that inhibits expression of glucokinase (GCK).

127. (canceled)

128. A method of inhibiting expression of glucokinase (GCK) in a subject, comprising:
administering to the subject an effective amount of the RNAi agent of claim 3.

129. (canceled)

130. A method of controlling or reducing liver fat accumulation in a subject, comprising:
administering to the subject an effective amount of a liver-targeted modified oligonucleotide, a liver-targeted oligomeric duplex, or a liver-targeted RNAi agent that inhibits expression of glucokinase (GCK).

131-202. (canceled)

203. A method of producing the RNAi agent according to claim 3, comprising annealing together the sense strand and the antisense strand.

204. (canceled)

205. The RNAi agent of claim 3, further comprising a targeting ligand that targets a hepatocyte.

206. The RNAi agent of claim 55, further comprising a targeting ligand that targets a hepatocyte.

207. The RNAi agent of claim 55, wherein the RNAi agent comprises a targeting ligand comprising a structure according to Formula I: 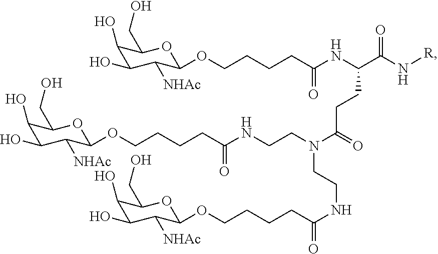
wherein R is a linker conjugated to the wherein the targeting ligand is conjugated to the 3′ end of the sense strand, wherein R comprises a structure according to Formula III: 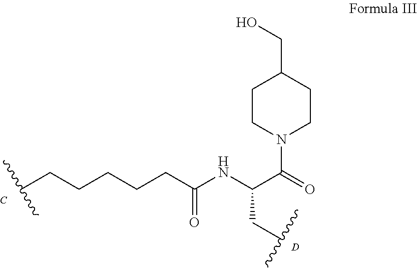
and wherein connection point C of Formula III is conjugated to Formula I, and connection point D of Formula III is conjugated to the 3′ end of the sense strand

208. A pharmaceutical composition comprising the RNAi agent of claim 56 and a pharmaceutically acceptable carrier.

209. The method of claim 126, wherein the RNAi agent comprises an antisense strand and a sense strand that at least partially complements the antisense strand, wherein:
(a) the antisense strand comprises a nucleobase sequence that differs by 0 or 1 nucleobases from any one of SEQ ID NOS: 200, 208, 227, 244, 276, 278, 281, and 282; and/or
(b) the sense strand comprises a nucleobase sequence that differs by 0 or 1 nucleobases from any one of SEQ ID NOS: 296, 304, 323, 340, 372, 374, 377, and 378.

210. The method of claim 130, wherein the RNAi agent comprises an antisense strand and a sense strand that at least partially complements the antisense strand, wherein:
(a) the antisense strand comprises a nucleobase sequence that differs by 0 or 1 nucleobases from any one of SEQ ID NOS: 200, 208, 227, 244, 276, 278, 281, and 282; and/or
(b) the sense strand comprises a nucleobase that differs by 0 or 1 nucleobases from any one of SEQ ID NOS: 296, 304, 323, 340, 372, 374, 377, and 378.
